(19) United States
(12) Patent Application Publication
(10) Pub. No.: US 2004/0170952 A1
(43) Pub. Date: Sep. 2, 2004

(54) Reagents and methods for the detection of genes associated with the major histocompatibility complex of domestic fowl, such as chicken

(75) Inventors: Rima Zoorob, Noisy Le Grand (FR); Charles Auffray, Sucy En Brie (FR); Anne-Marie Chausse, St Laurent En Gatines (FR)

Correspondence Address: NIXON & VANDERHYE P.C., 8th floor, 1100 North Glebe Road, Arlington, VA, 22201-4714, US

(73) Assignees: C.N.R.S.; I.N.R.A.

(21) Appl. No.: 10/043,160

(22) Filed: Jan. 14, 2002

Related U.S. Application Data
(63) Continuation of application No. 09/554,911, filed on Jul. 24, 2000, now abandoned.
Application No. 09/554,911 is a 371 of international application No. PCT/FR98/02501, filed on Nov. 23, 1998.

(30) Foreign Application Priority Data
Nov. 21, 1997 (FR) 97 14669

(51) Int. Cl. C12Q 1/70; C12Q 1/68; C07H 21/04

(52) U.S. Cl. 435/5; 435/6; 536/24.3

(57) ABSTRACT

The invention relates to nucleic acid molecules which make it possible to detect those genes of the MHC involved in the phenomena of resistance or susceptibility to the development of virus-induced tumours. The primers developed from said molecules can be used in a method of genotyping farmed birds, and particularly chickens, characterised in that it comprises:

amplification of a sample of nucleic acid originating from the animal to be studied using one or more pairs of primers capable of hybridising specifically with the nucleic acid of a polymorphic region of the Rfp-Y or B systems of the MHC of said birds, and

detection of the PCR products obtained.

The invention relates to the detection of genes associated with the major histocompatibility complex (MHC) of domestic fowl, such as chicken. By virtue of this, it relates to nucleic acid molecules which make it possible to detect those genes of the MHC involved in the phenomena of resistance or susceptibility to the development of virus-induced tumours. The invention also relates to the applications of said nucleic acid molecules, particularly for the development of genotyping tests in farmed birds, particularly chickens, and for the selection of animals of interest.

Infectious viral diseases are feared by breeders because of their contagious nature which leads to substantial animal losses.

Vaccination was an effective prophylaxis until the emergence of hypervirulent strains making it necessary to identify resistant haplotypes.

Various methods have, therefore, been proposed in an attempt to select those animals which are capable of resisting such diseases and those which are, in contrast, likely to be affected.

The most routinely used methods are based on serological polymorphisms or those of the RFLP type. However, these methods do not provide accurate knowledge about the phenomenon of resistance or susceptibility to disease, particularly due to lack of discrimination with respect to genes of the B or Rfp-Y systems of the MHC.

The work of the inventors on MHC gene sequencing showed the genetic complexity of this region, which led them to take account of another type of polymorphism, namely that based on the sequence of these genes and of related regions, such as those of their promoters and of the microsatellite regions. The inventors thus developed methods for obtaining highly specific oligonucleotide molecules of the polymorphisms observed, making it possible to identify the parts of genes, and even the sites involved in controlling resistance or susceptibility to the development of tumours.

The specific nature of these molecules with respect to a given gene of one of the systems of the MHC makes them particularly reliable discriminating tools for identifying accurately the capacity for resistance or susceptibility of the chicken studied, or of other birds, to a viral infection, and for studying at molecular level the sequences of the MHC involved.

The object of the invention is, therefore, to provide nucleic acid molecules which make it possible to detect specifically, in farmed birds and particularly in chickens, the genes associated with the MHC involved in the phenomena of resistance or susceptibility to the development of virus-induced tumours.

It also provides a method and a kit for the detection of genotypes which is easy to use on a routine basis.

The nucleic acid molecules of the invention are characterised in that they are molecules, isolated from their natural environment, of nucleic acids of genes coding for proteins involved in controlling resistance or susceptibility to the development of virus-induced tumours in farmed birds, such as those of Marek's disease in chickens, with, if necessary, the regions which are attached to them such as those of the promoter or microsatellite regions. The term gene as used in the description and claims includes these regions.

These nucleic acid molecules are characterised more particularly in that they have nucleic acid sequences of genes of the B system or of the Rfp-Y system of the MHC of farmed birds, with the exception of sequences of class II B-L genes, gene 17.5, gene 12.3 and of the class I B-FIV gene, or are capable of pairing with one of the strands of a gene capable of coding for a protein as defined above under conditions of low stringency.

The pairing under conditions of low stringency to which reference is made above is carried out at ambient temperature in a 0.1 SSC medium with washing at ambient temperature.

Class II B-L genes are described in Immunogenetics 31:179-187, 1990 and Eur. J. Immunol., 1993, 23: 1139-1145.

Gene 17.5 belongs to the gene superfamily coding for lectins and gene 12.3 to the gene family coding for proteins that bind guanine (guanine nucleotide-binding protein). This gene is described in Immunogenetics 39:221-229, 1994.

Gene 12.3 is described in P.N.A.S. USA, vol. 86, 4594-4598, June 1989, Genetics.

The class I B-FIV gene is described in Immnunogenetics 31:405-409, 1990.

The invention relates, in particular, to molecules of nucleic acids corresponding to those of the sequences of one of the following genes:

sequence of the Rfp-Y system

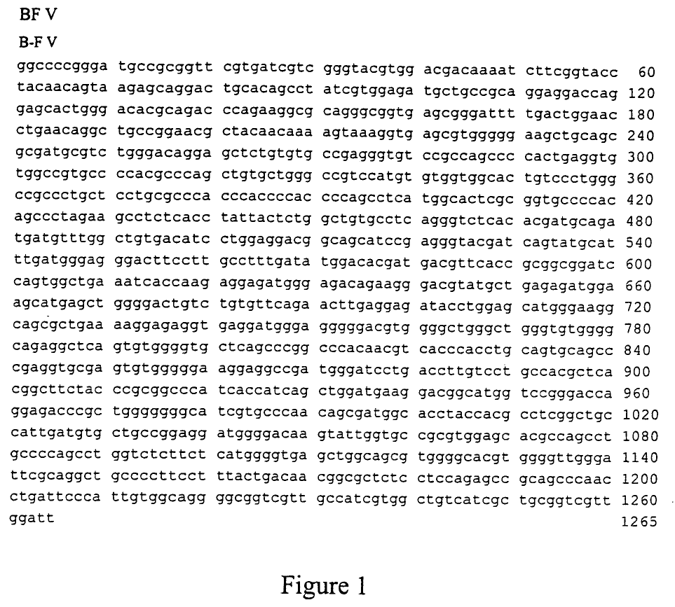

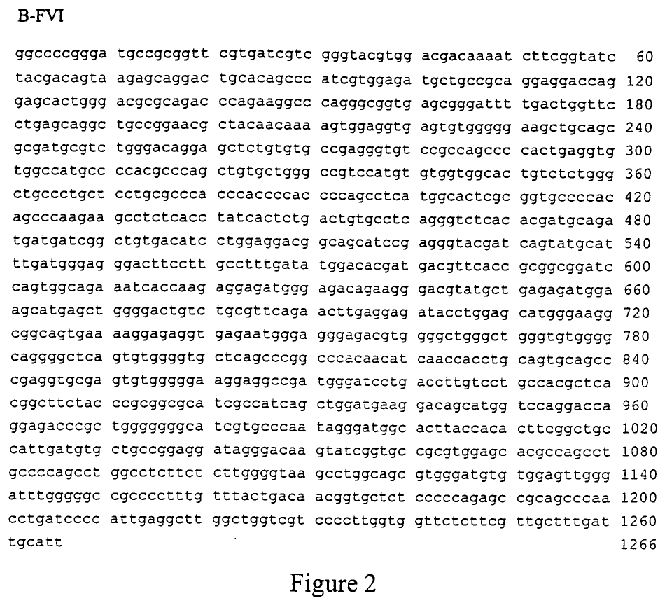

B-FV (FIG. 1); B-F VI (FIG. 2);

sequence of the B system,

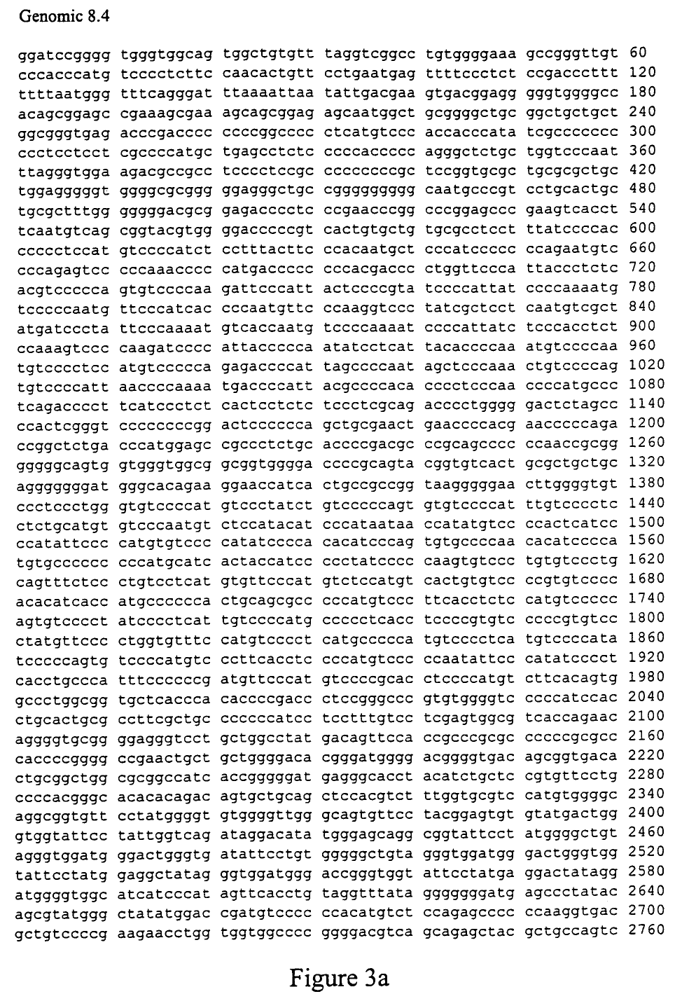

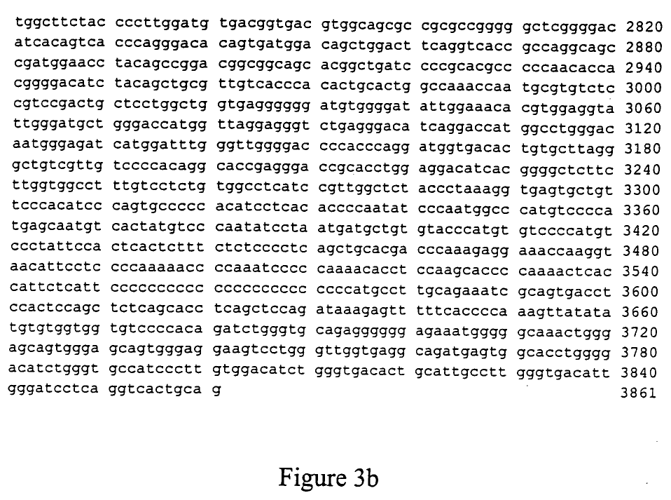

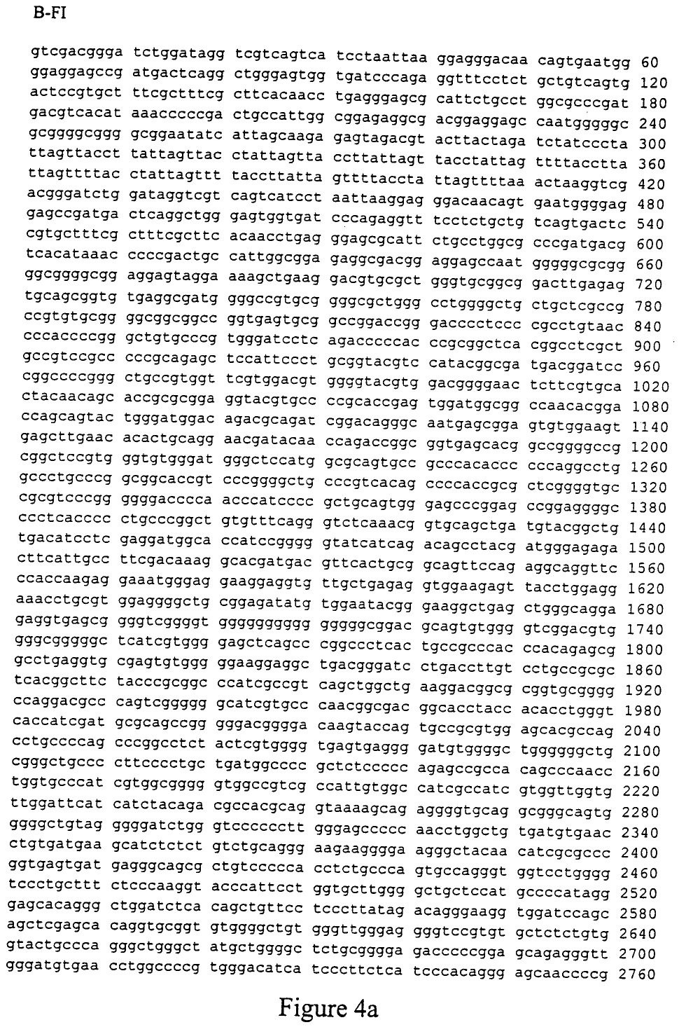

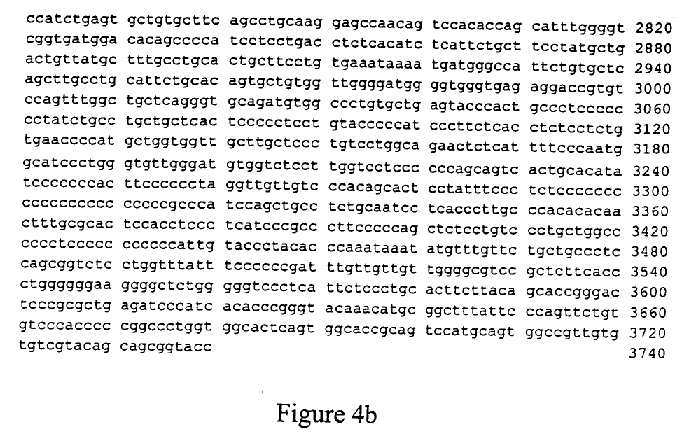

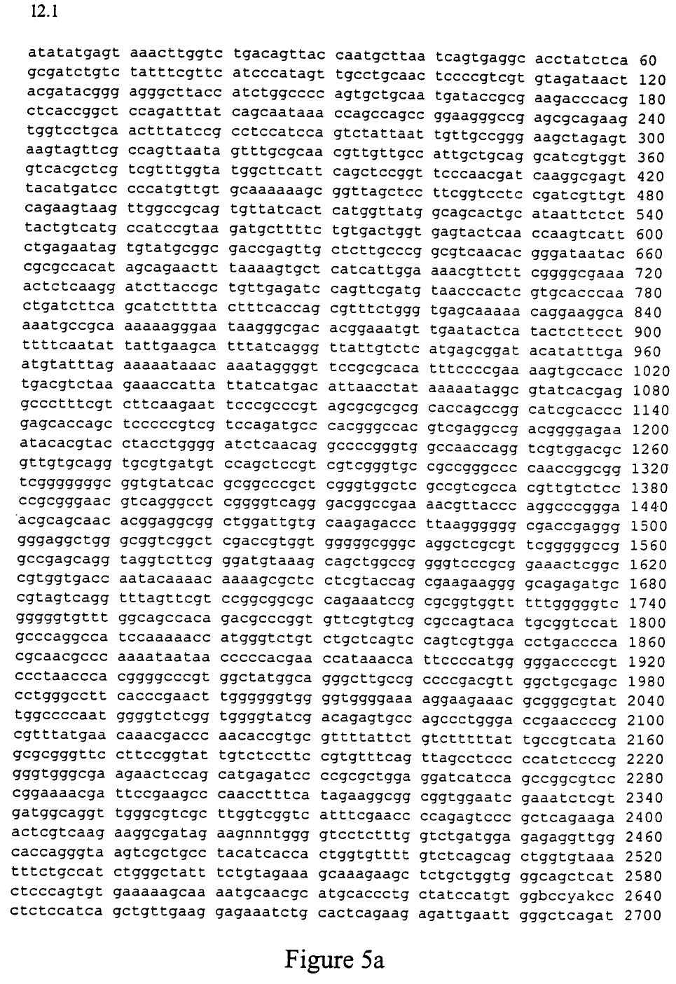

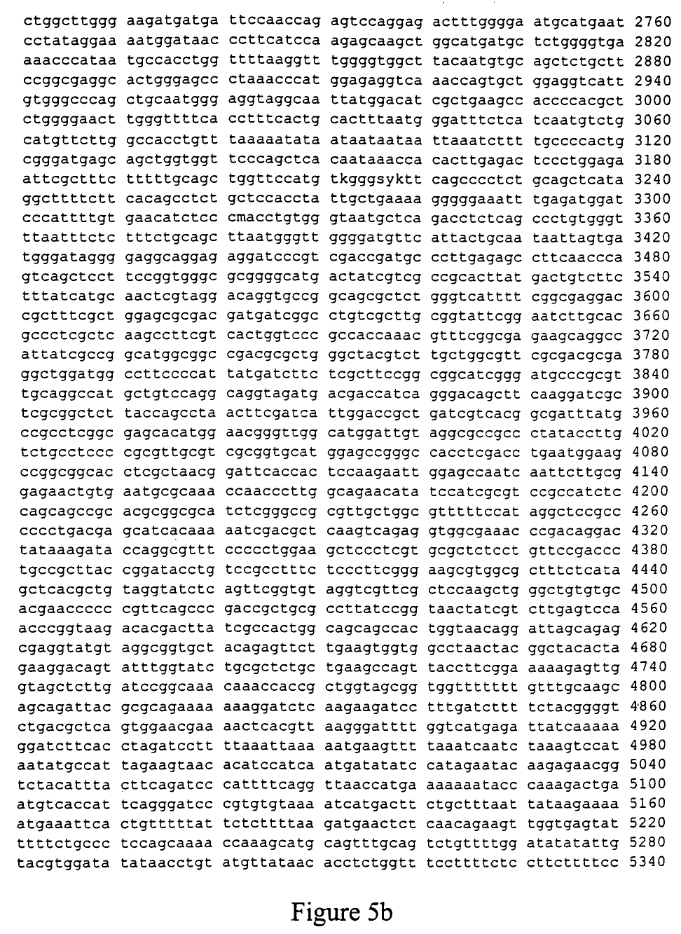

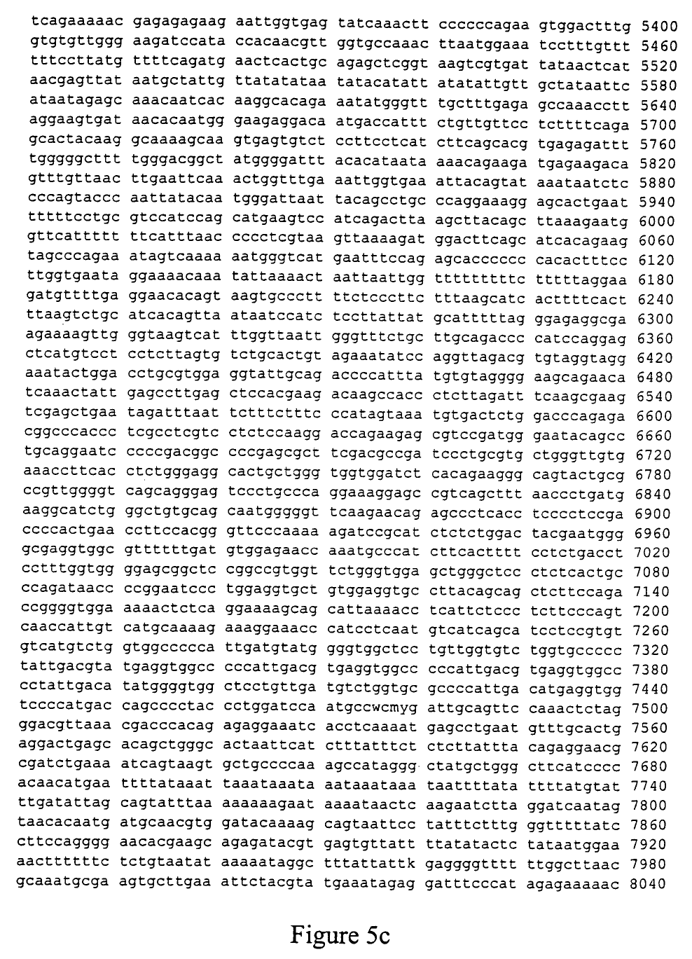

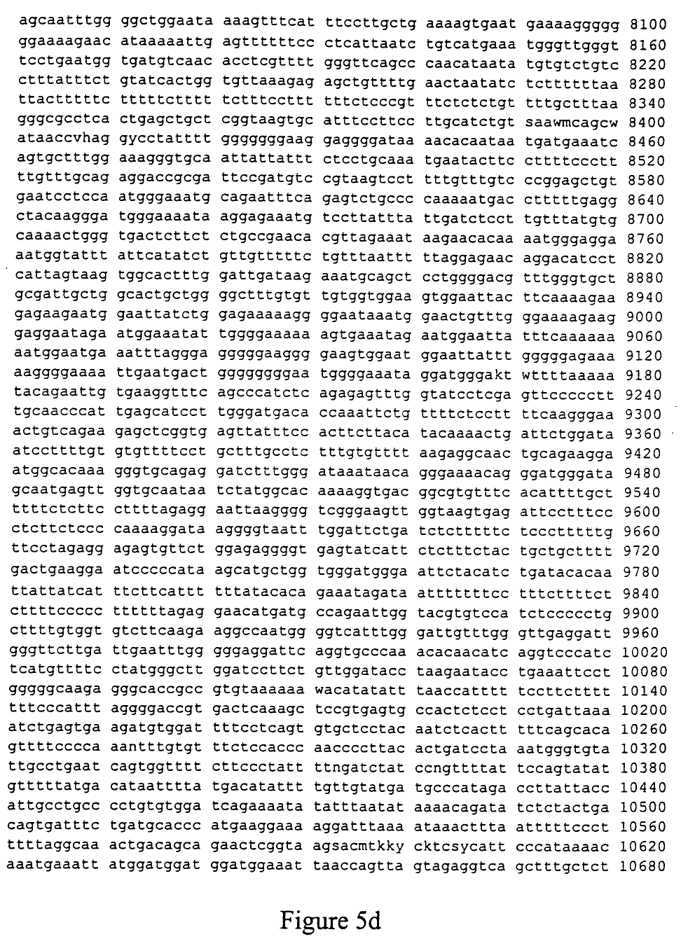

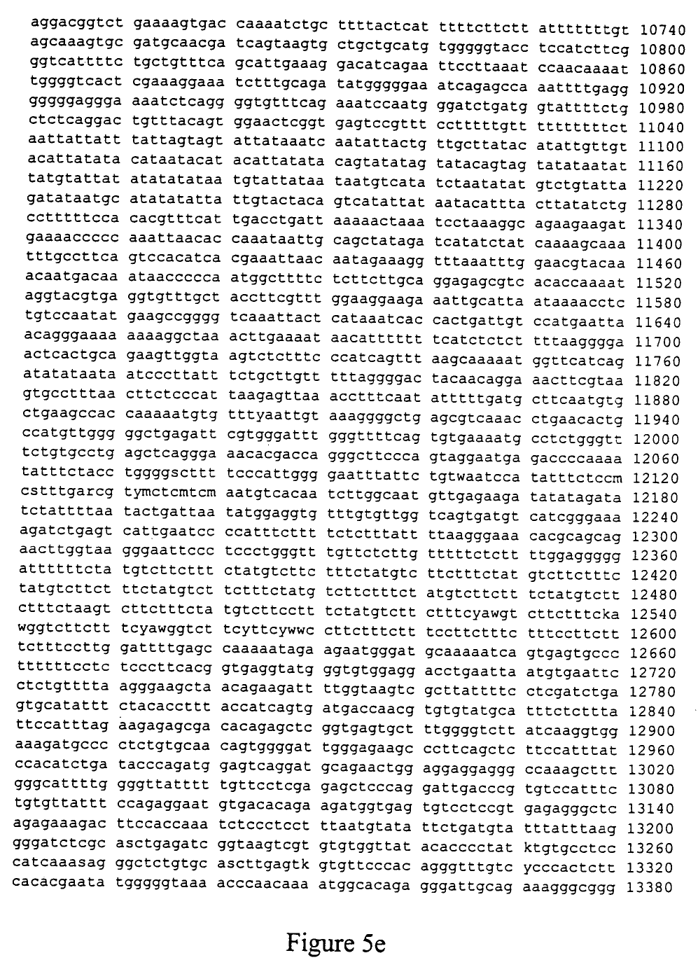

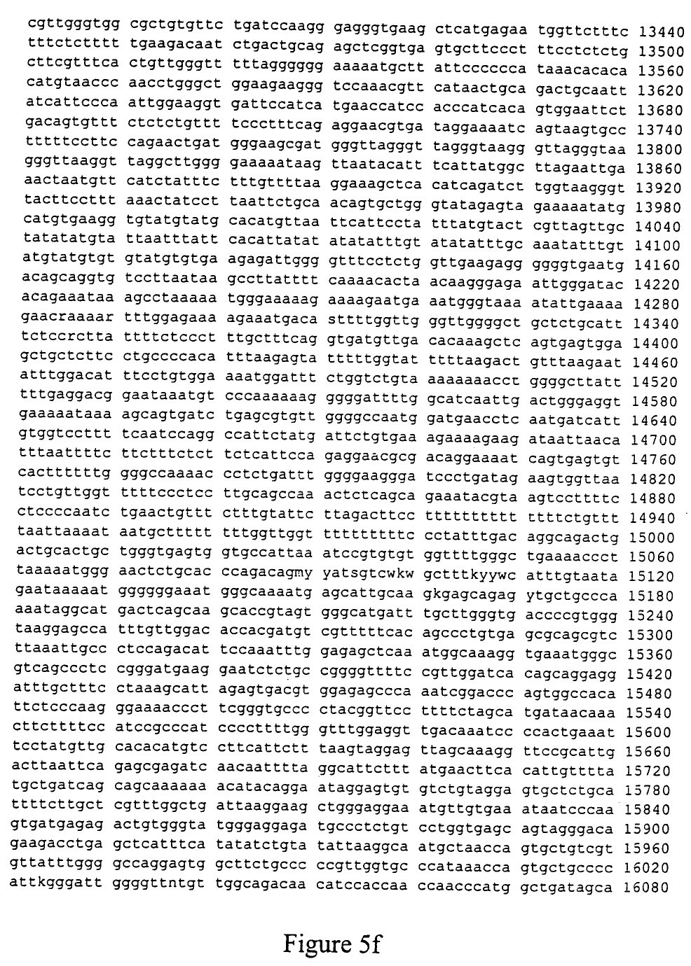

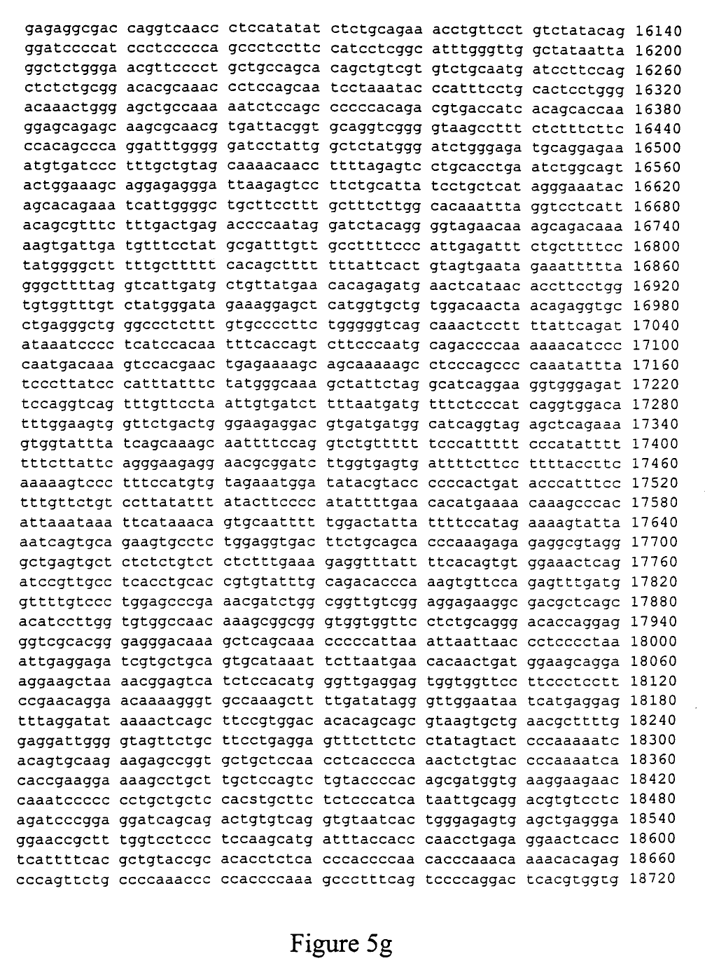

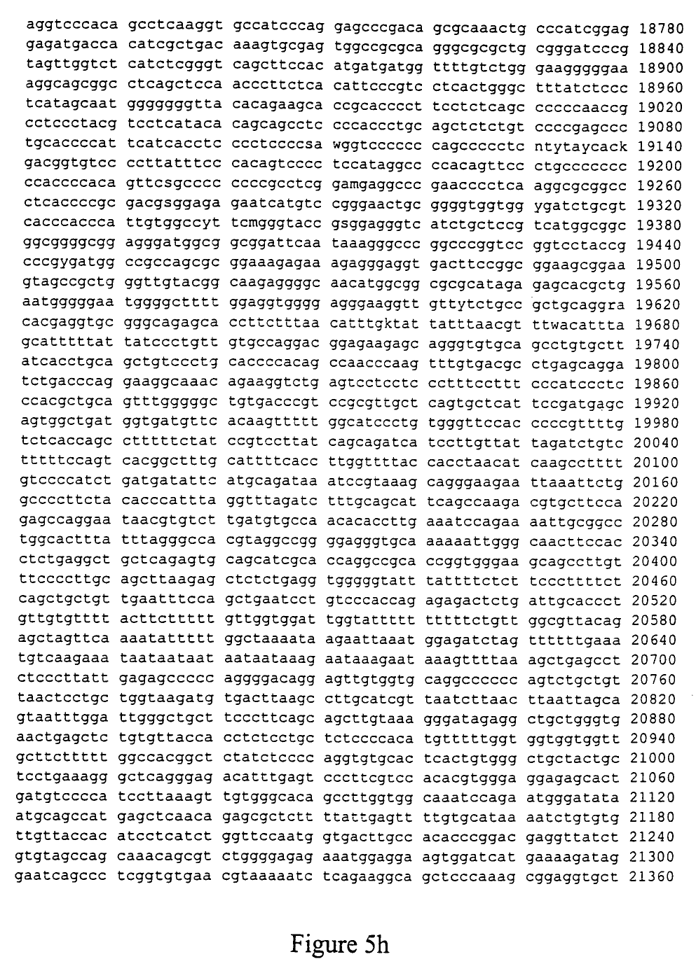

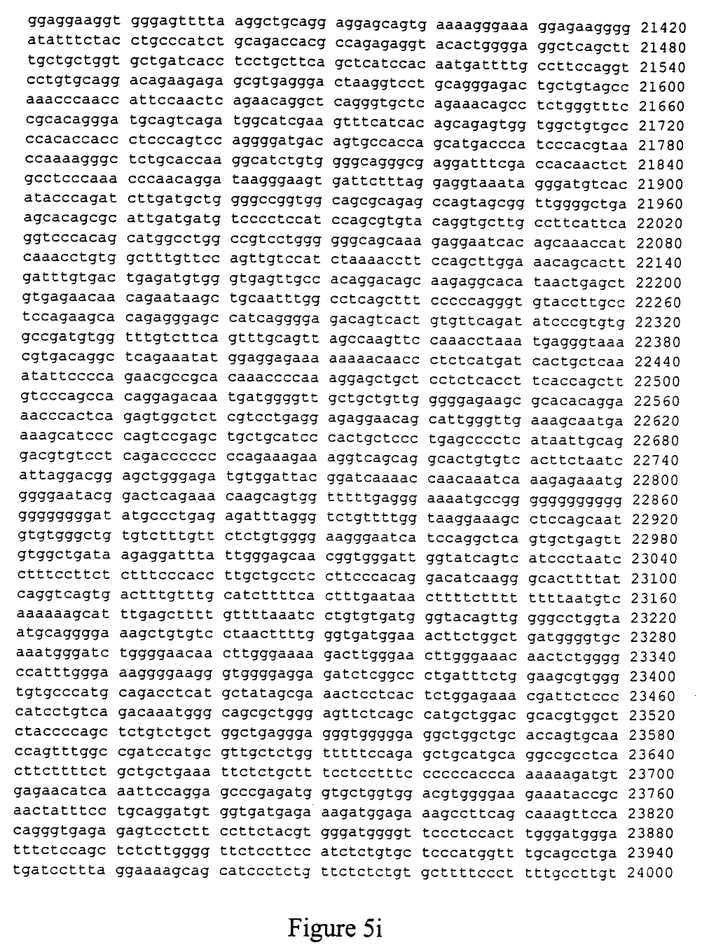

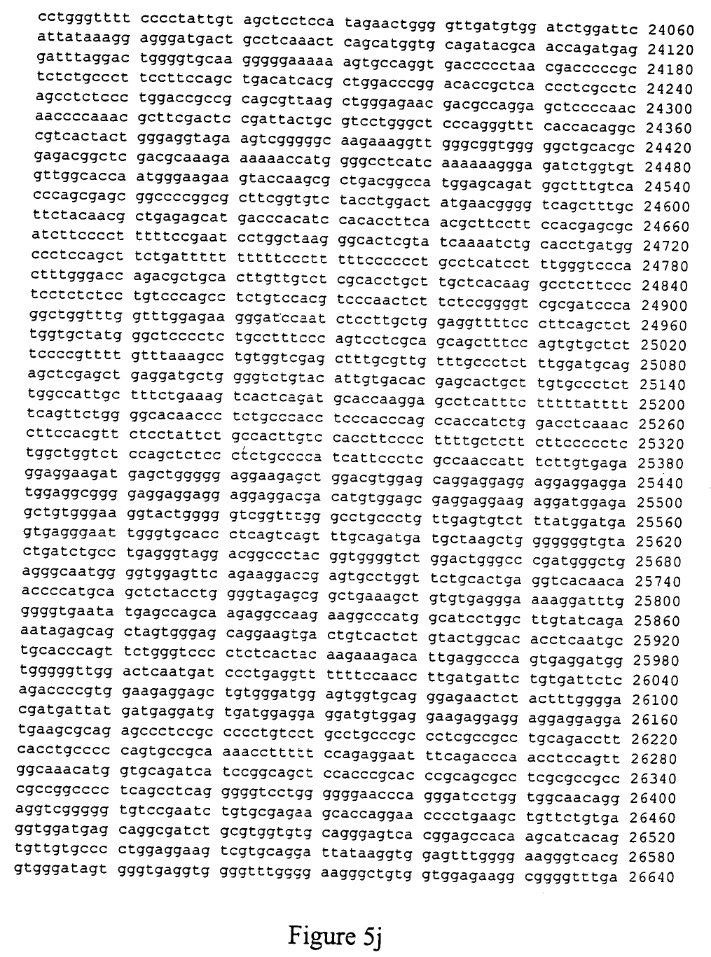

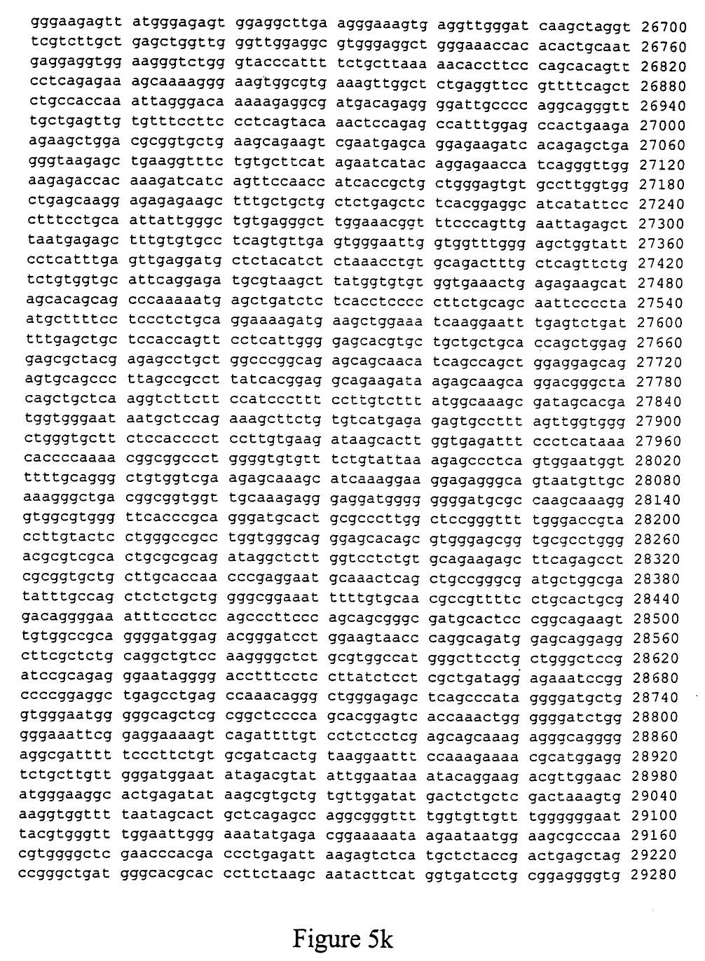

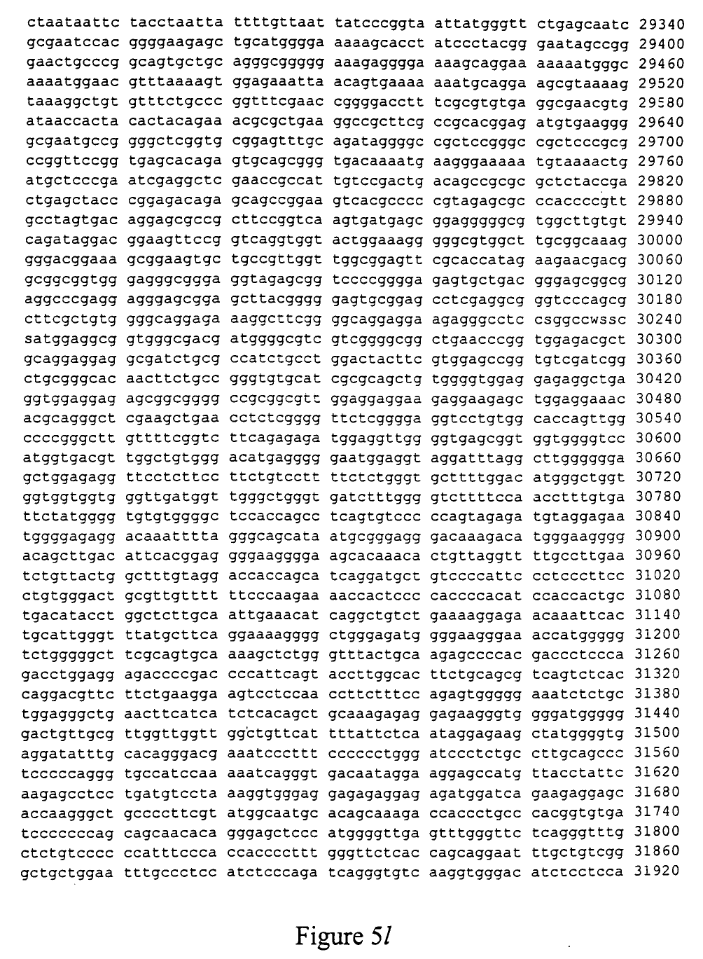

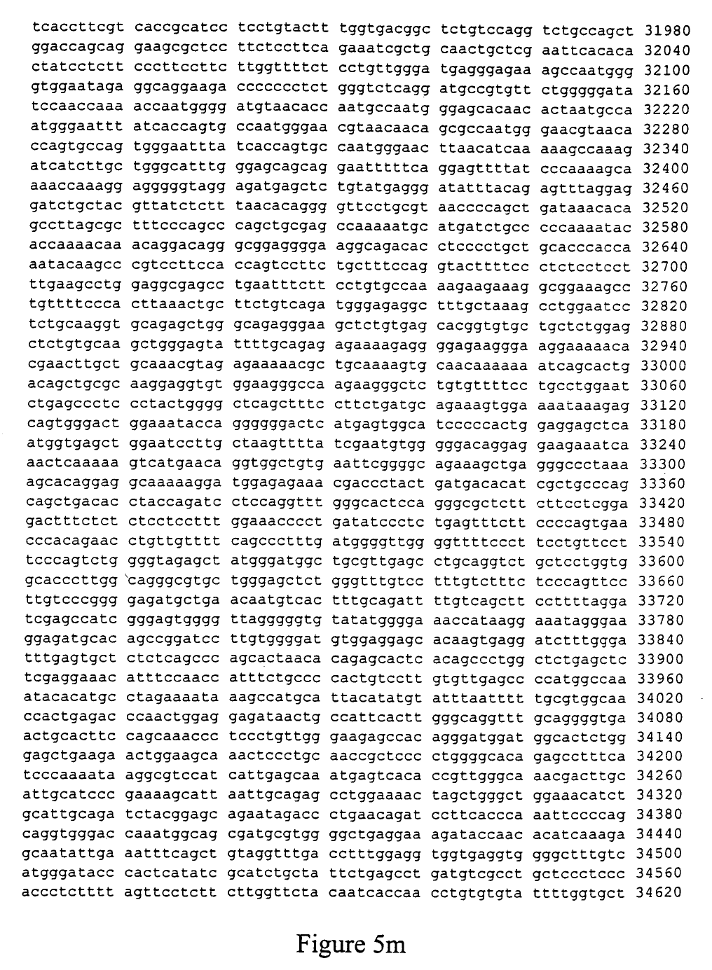

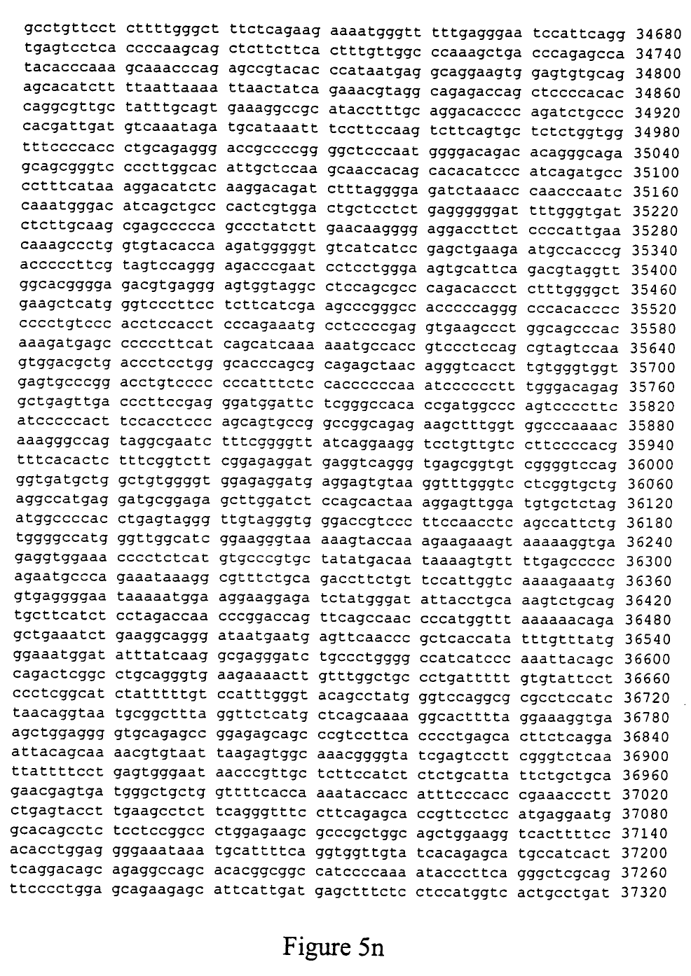

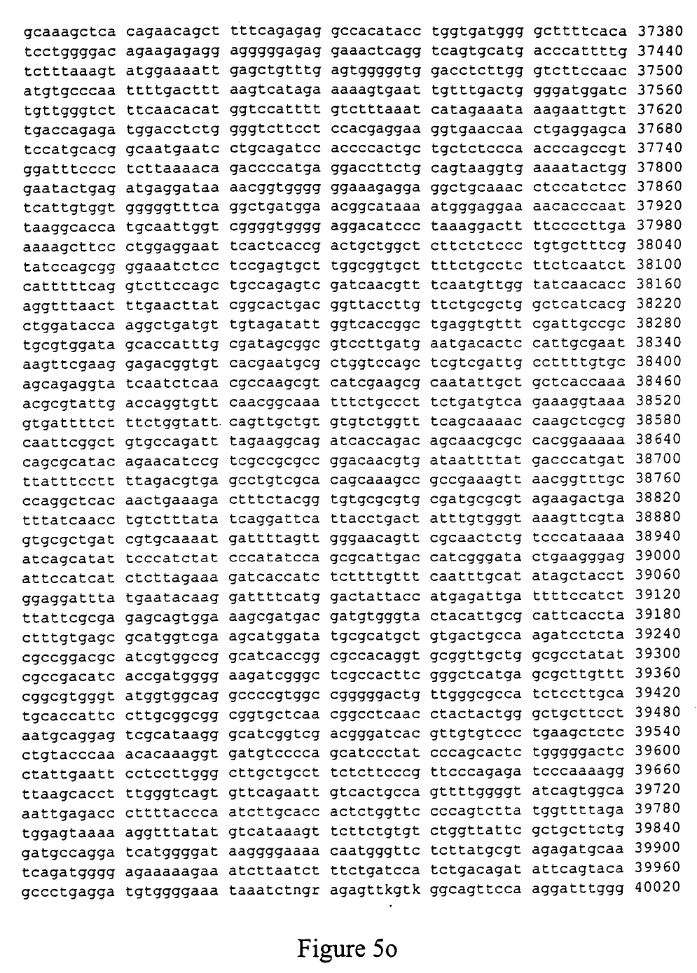

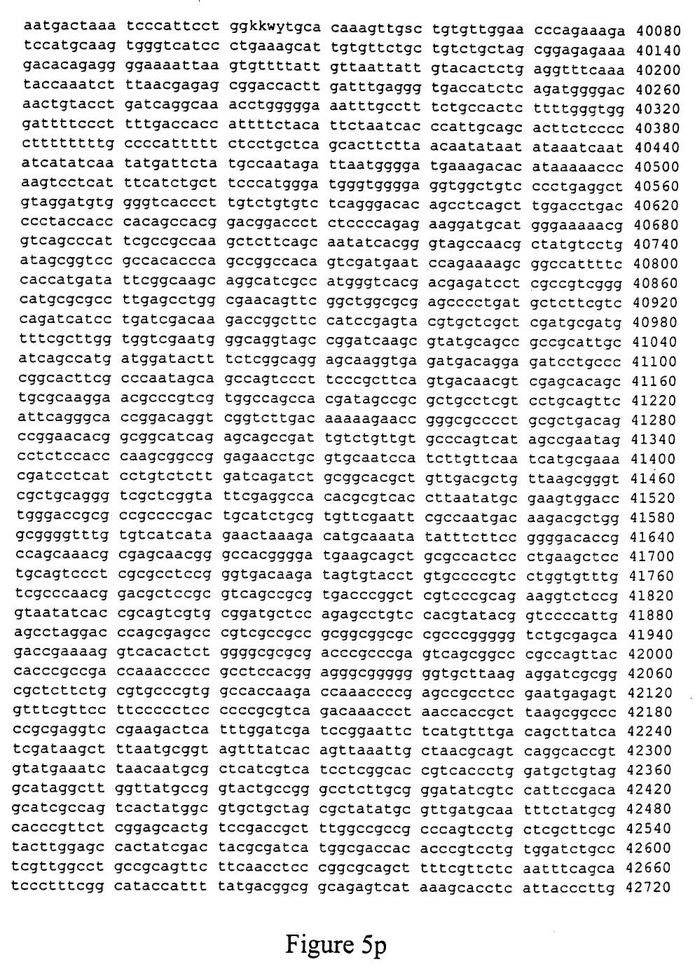

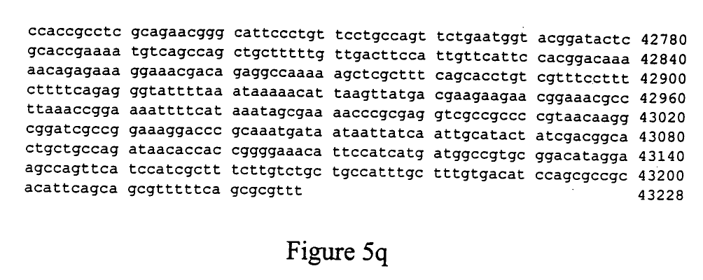

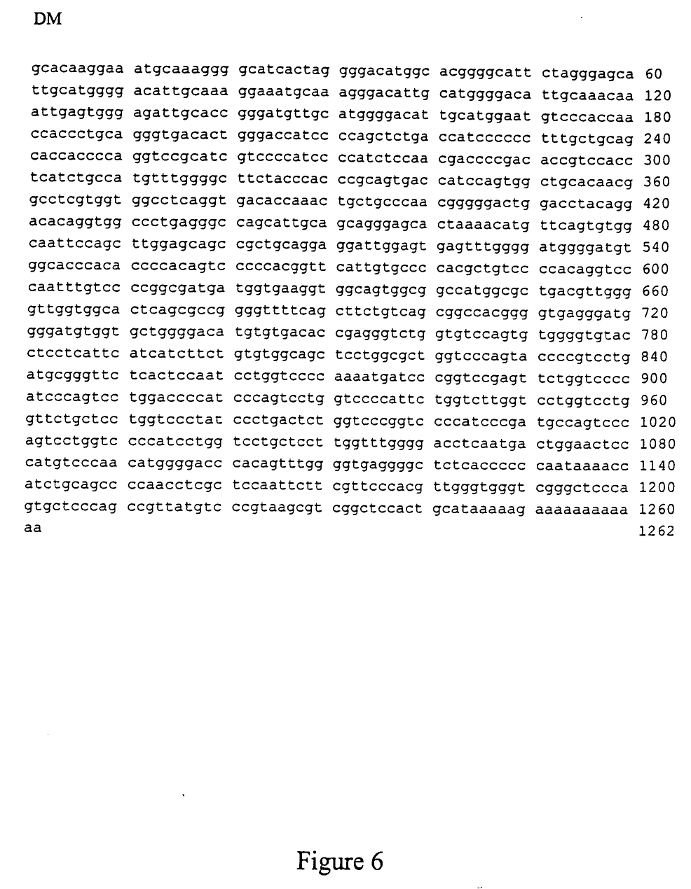

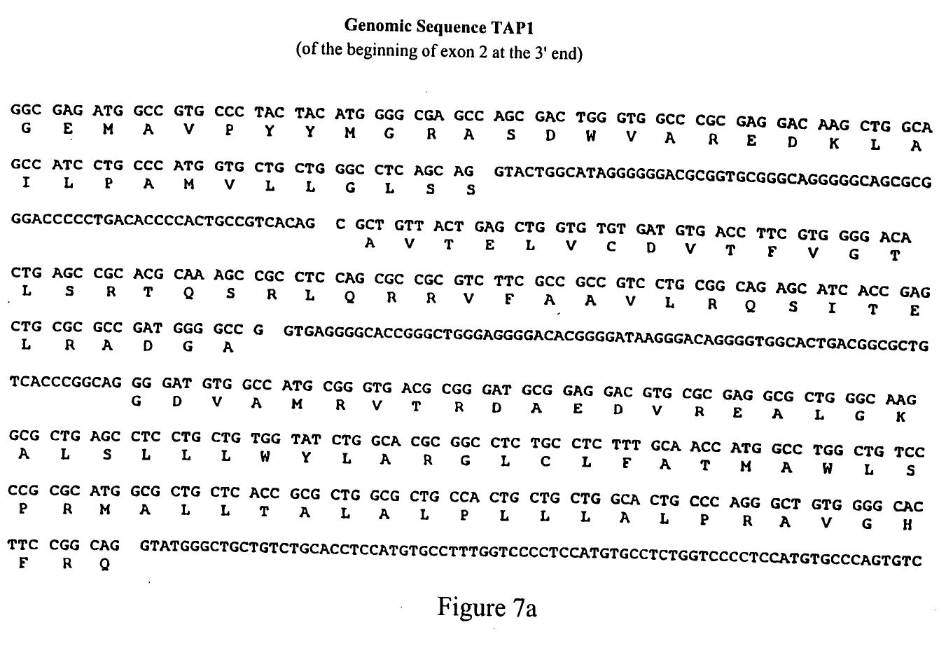

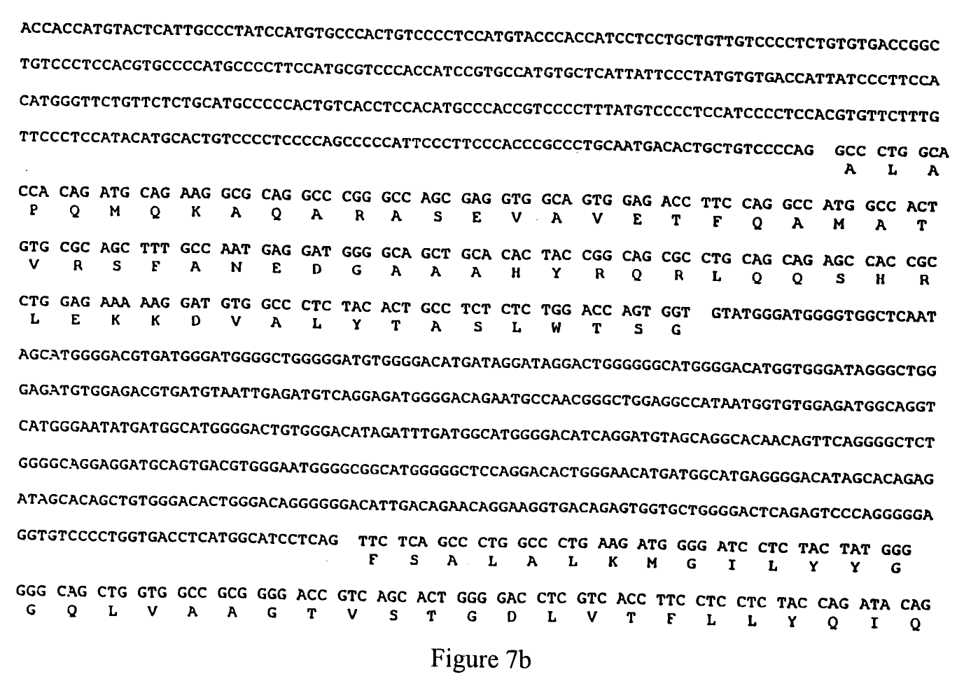

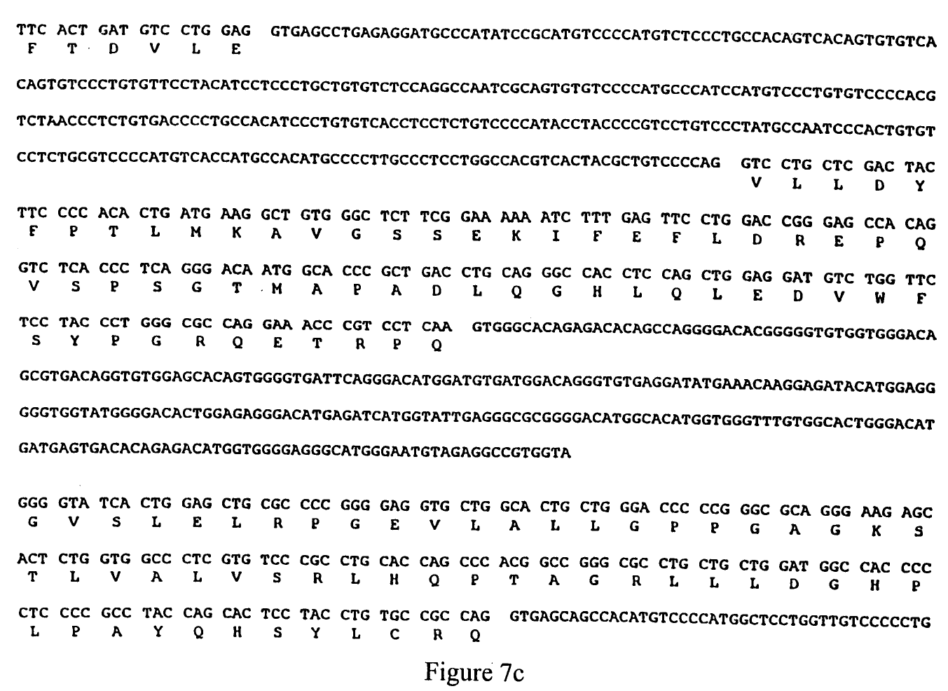

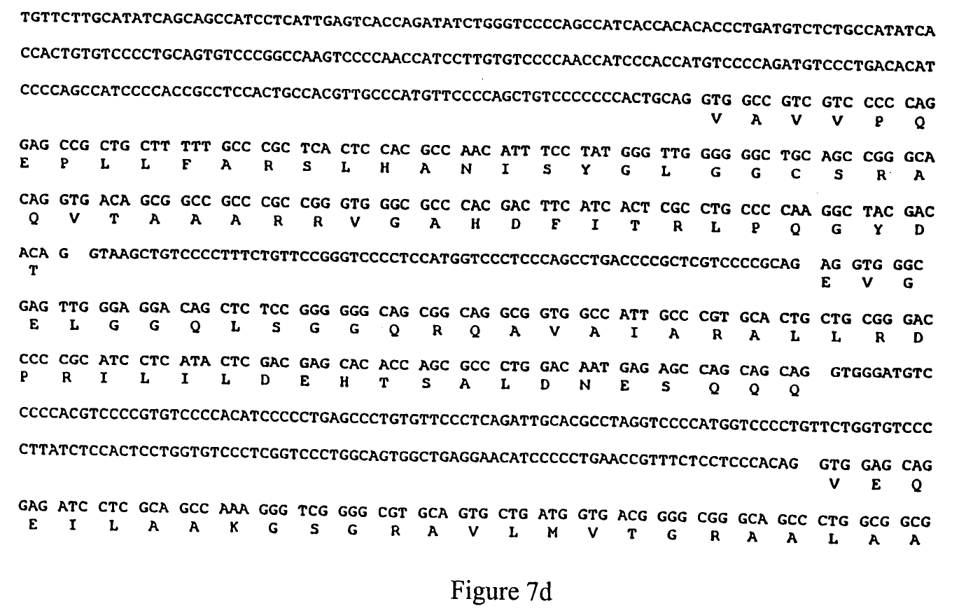

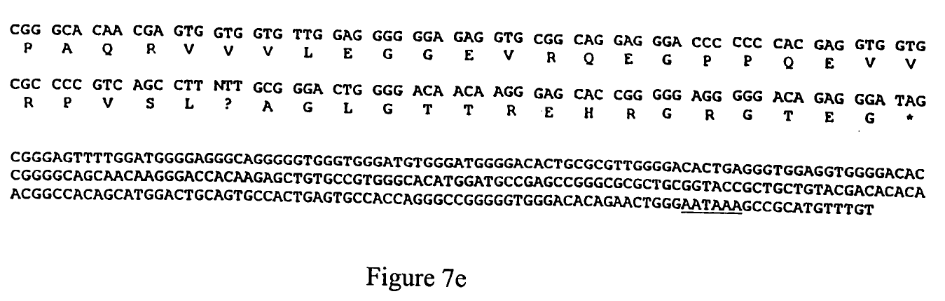

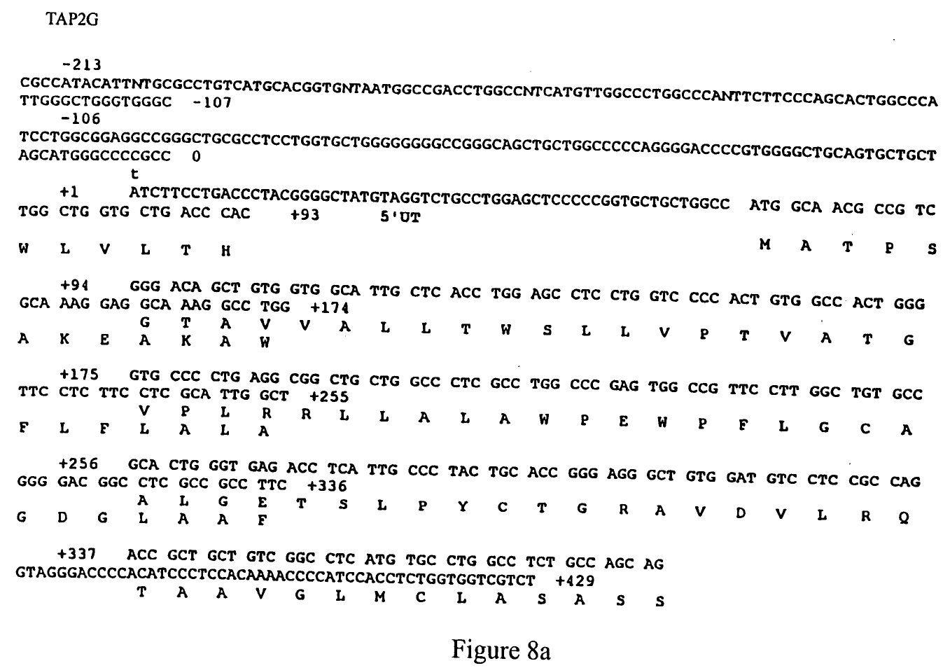

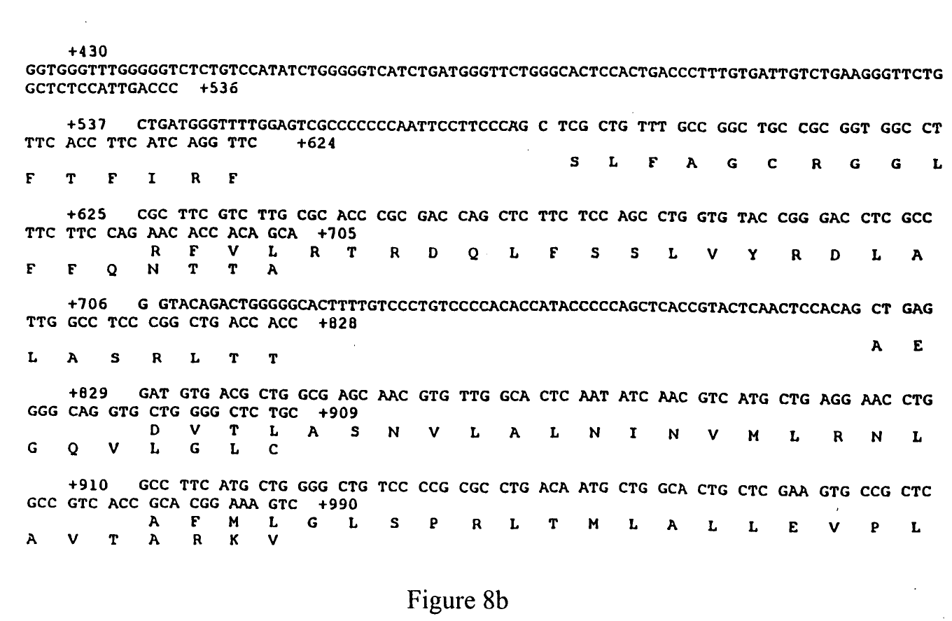

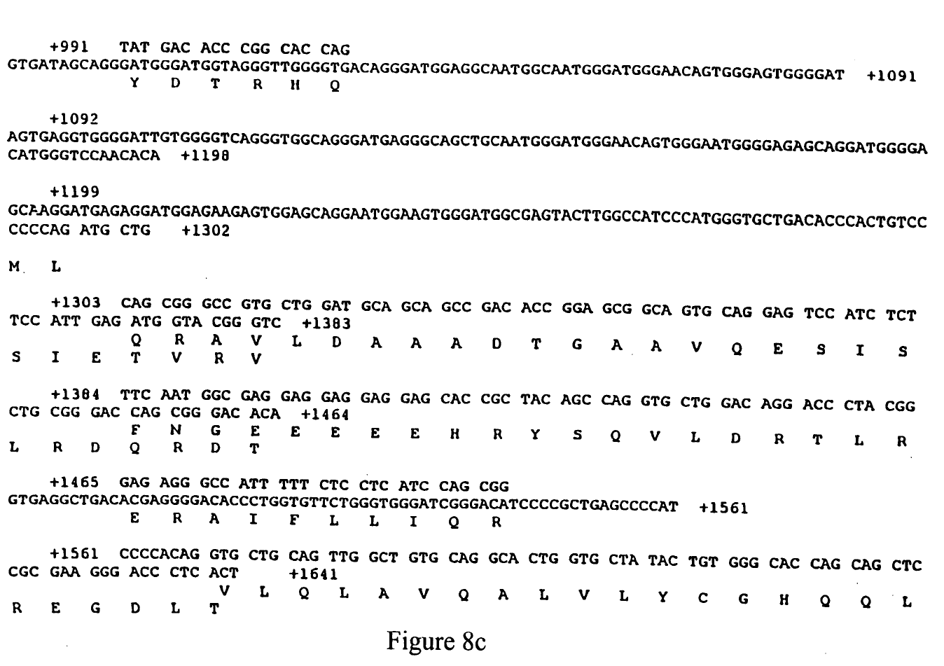

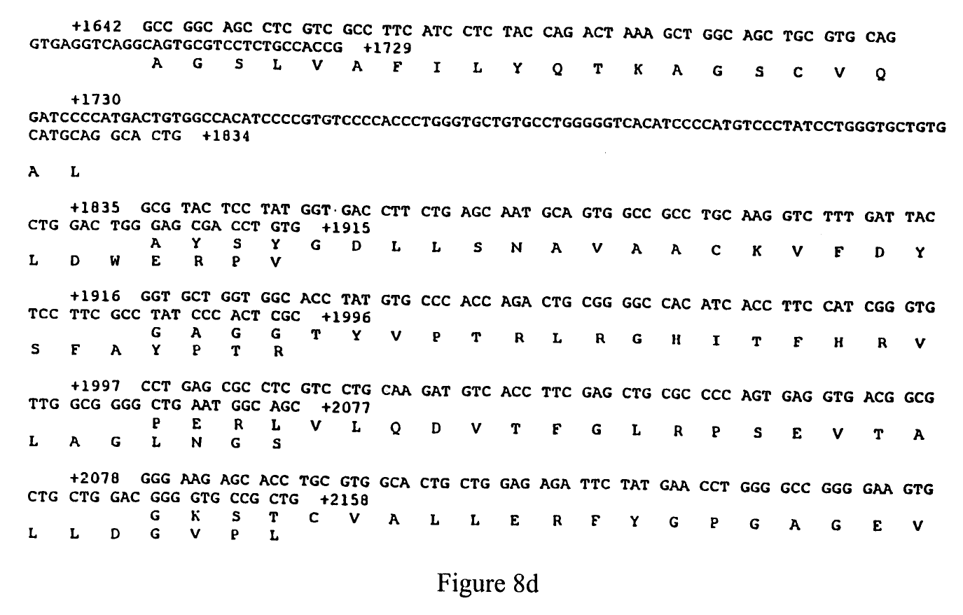

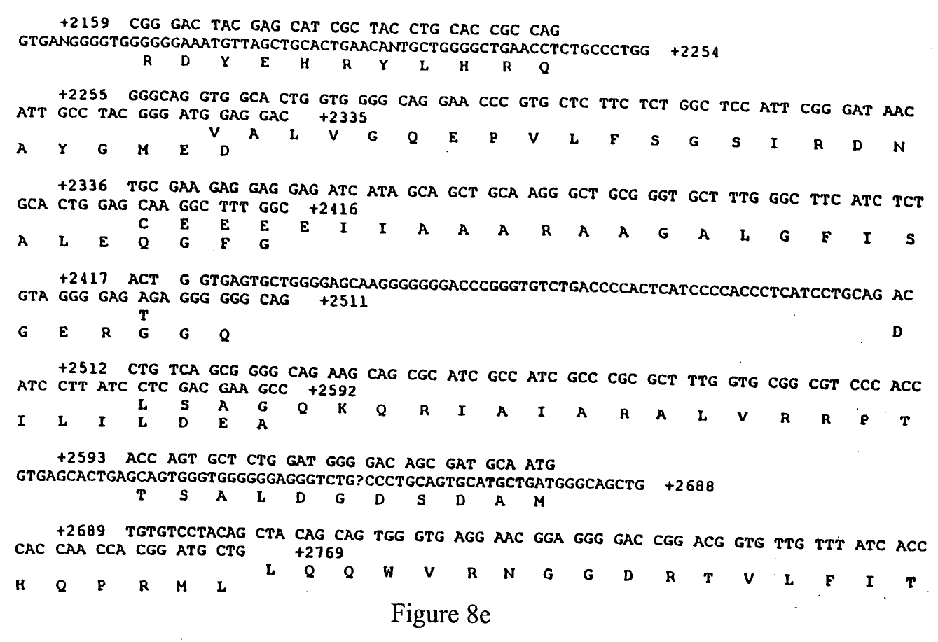

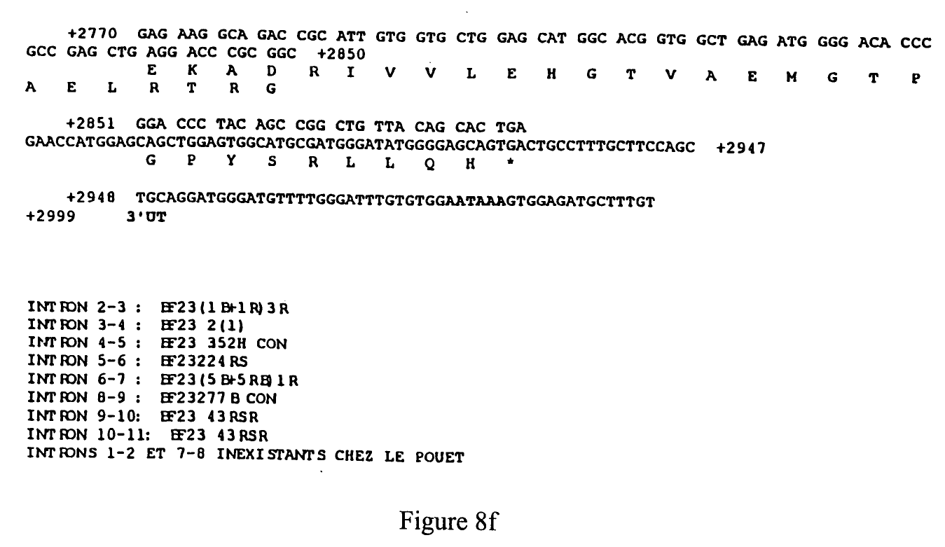

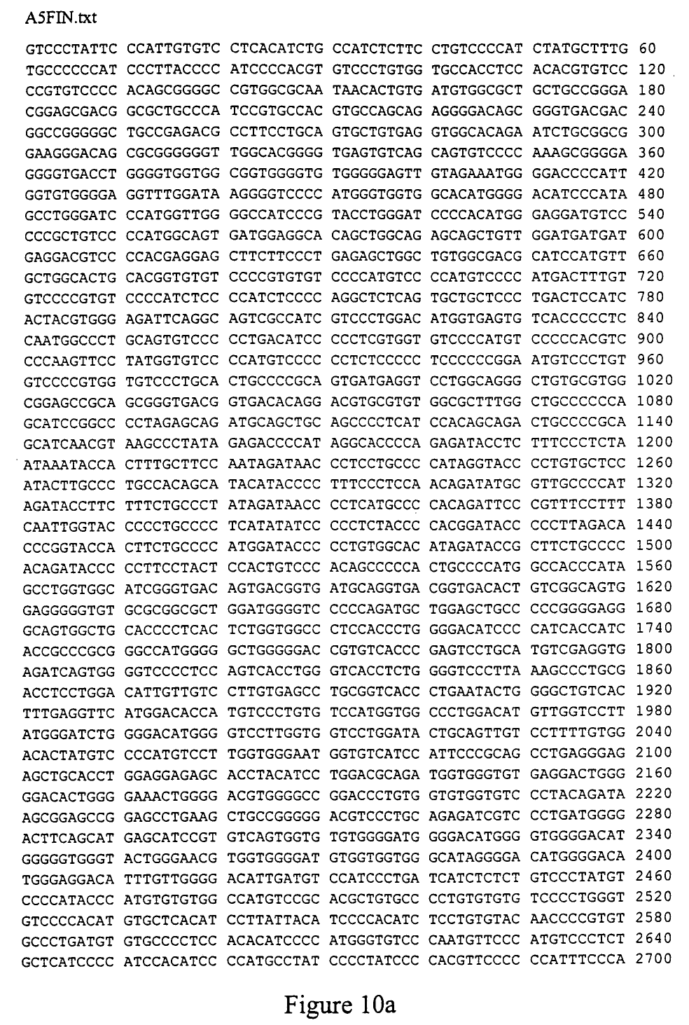

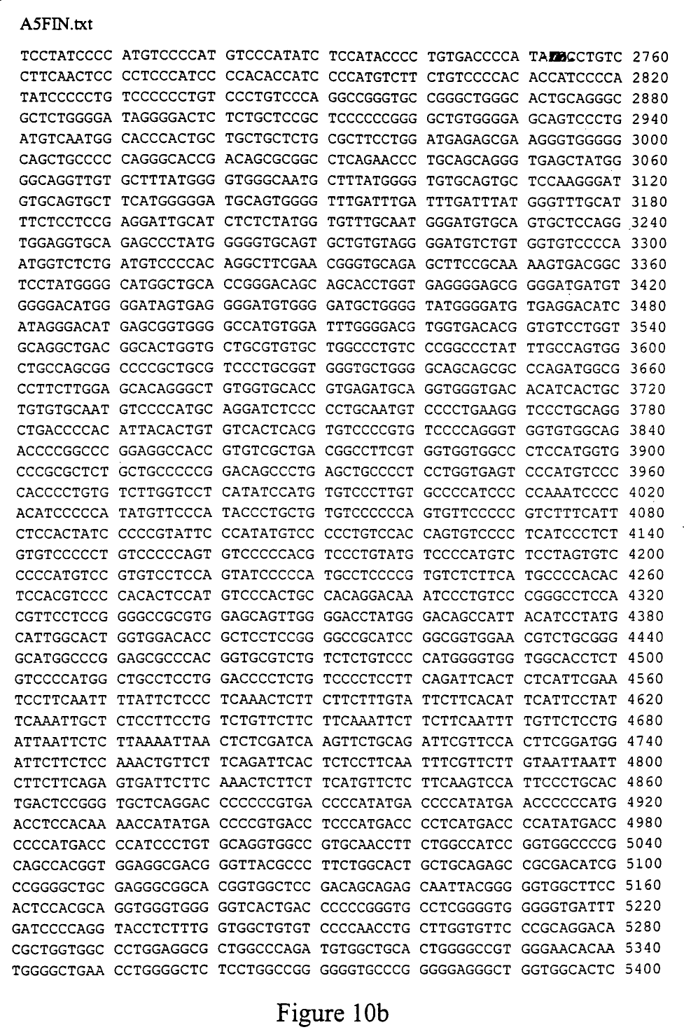

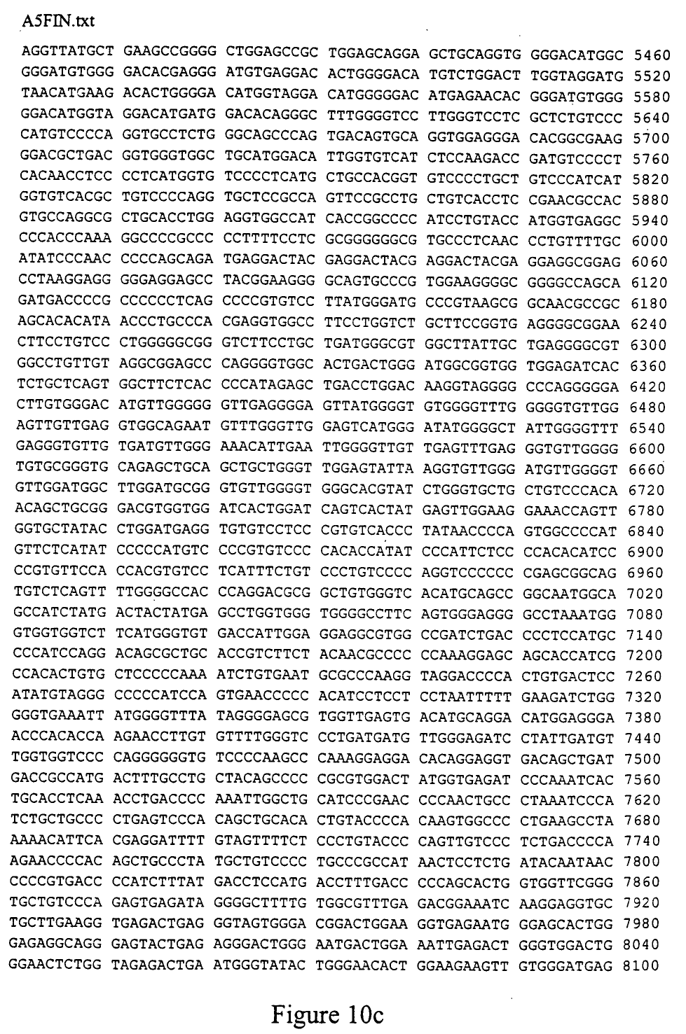

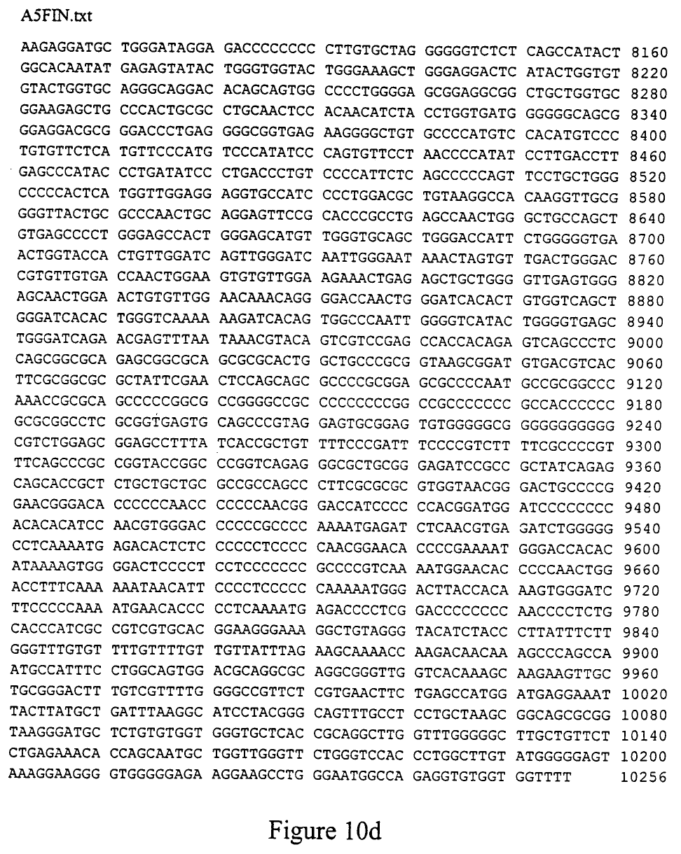

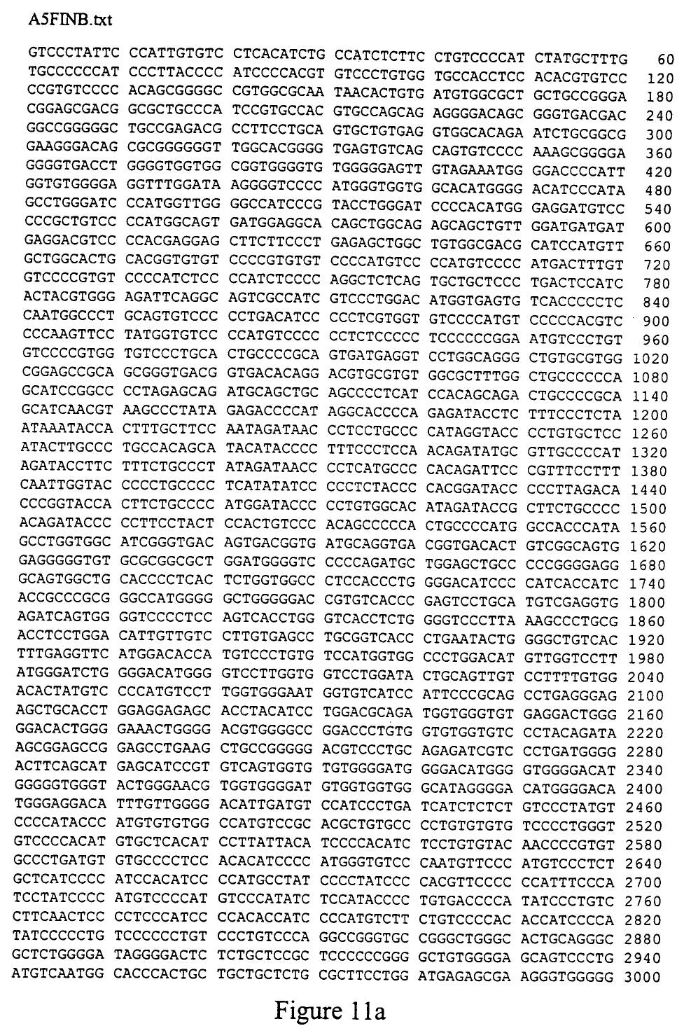

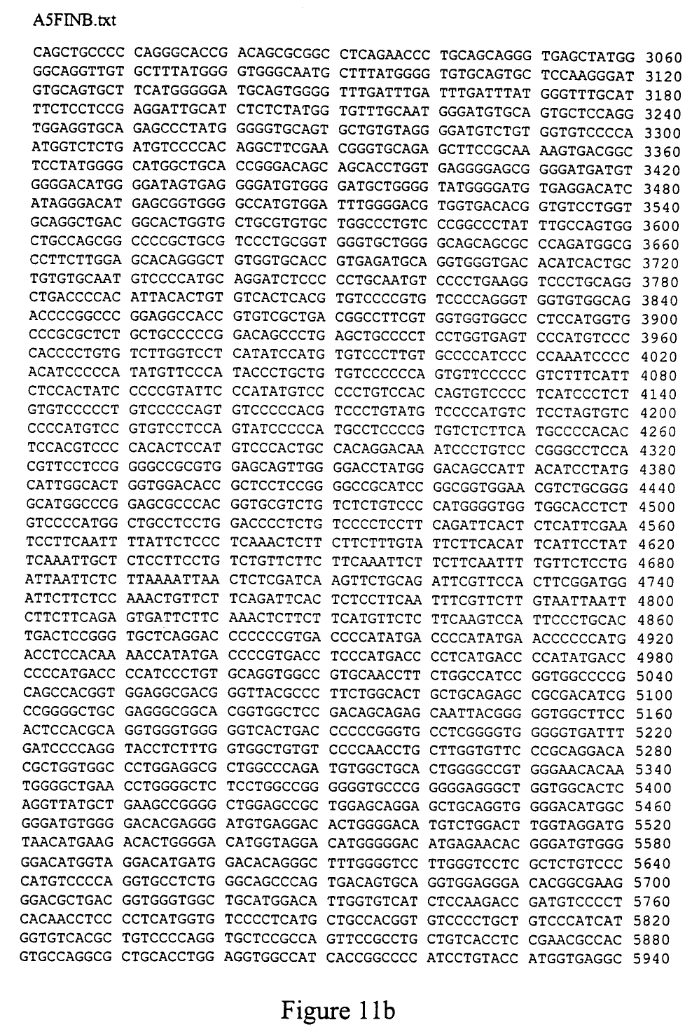

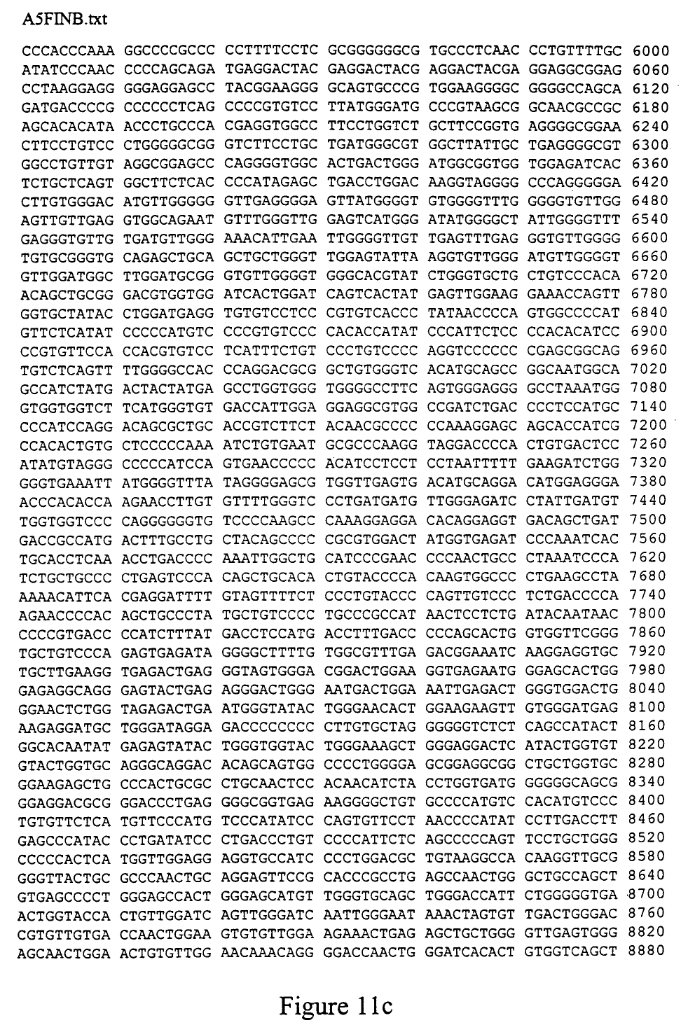

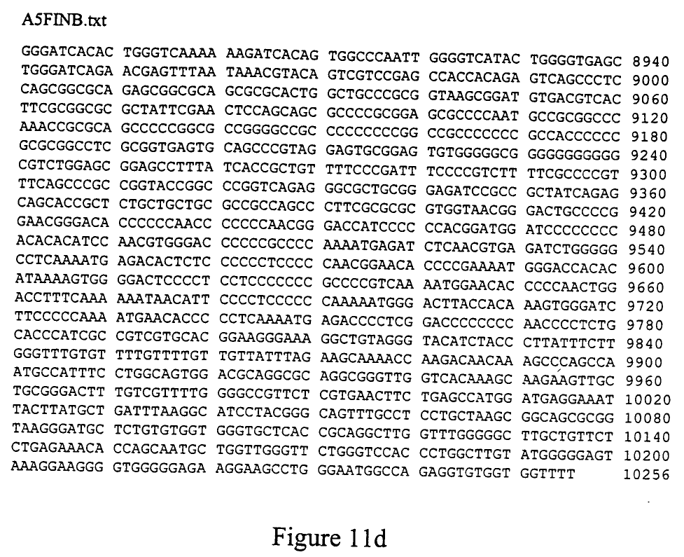

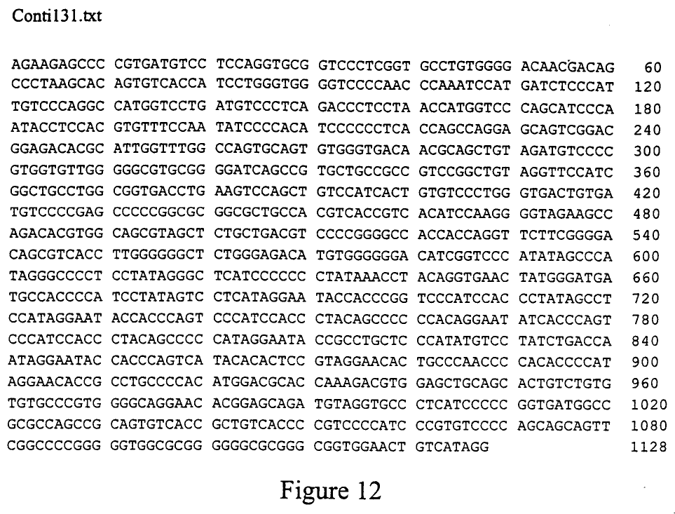

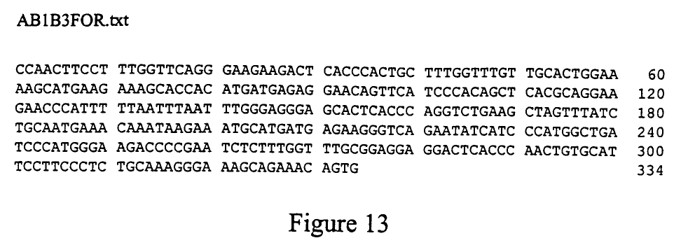

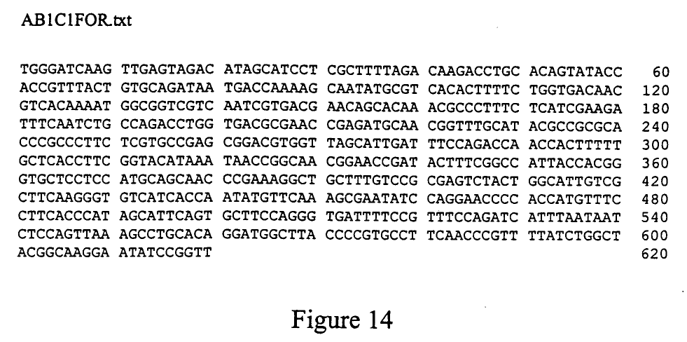

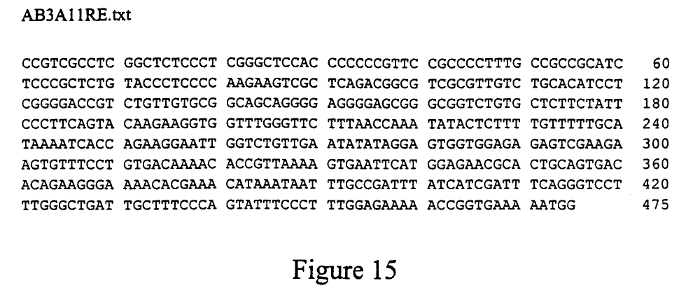

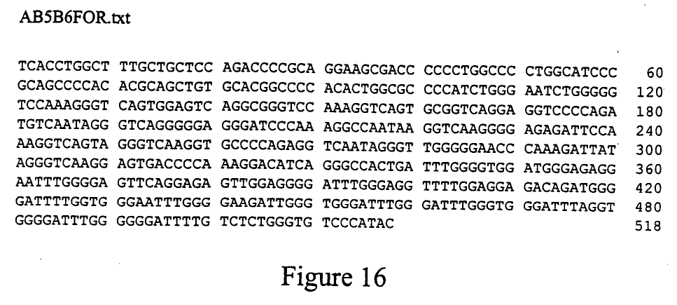

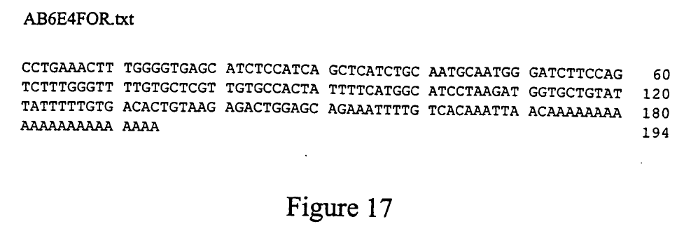

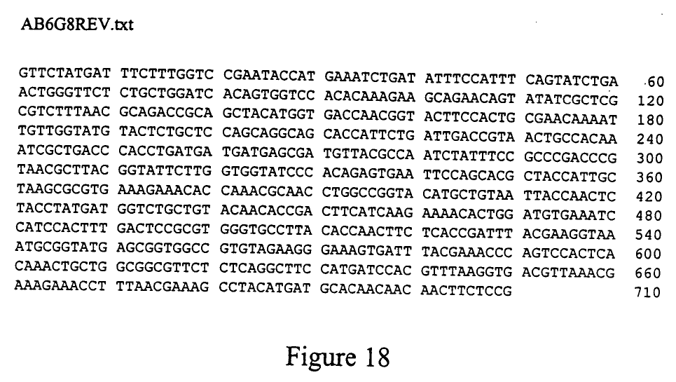

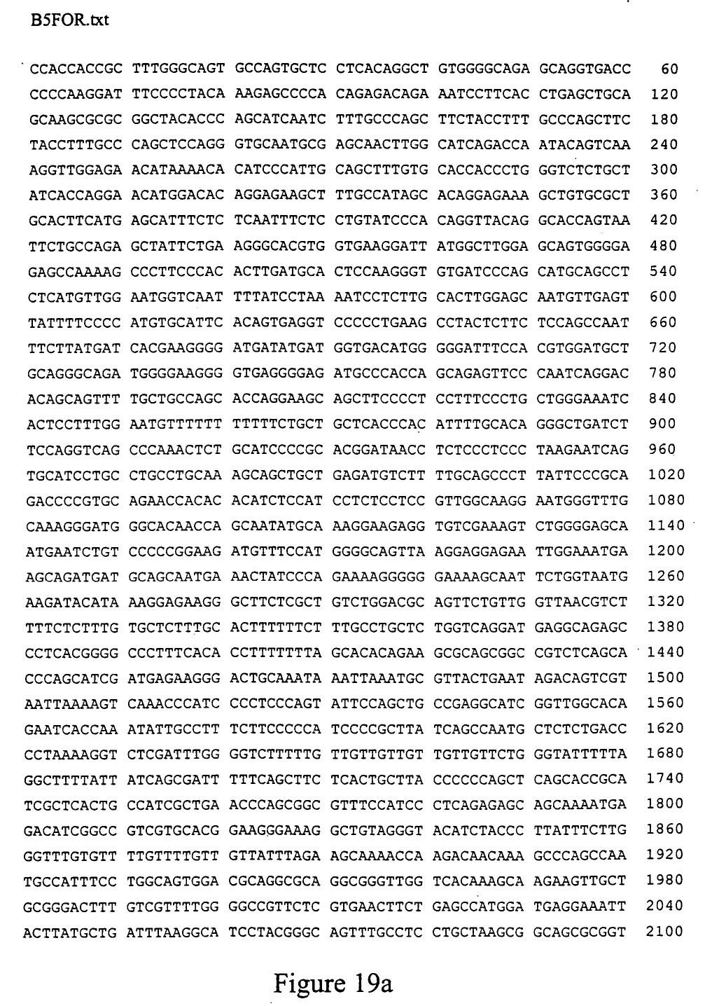

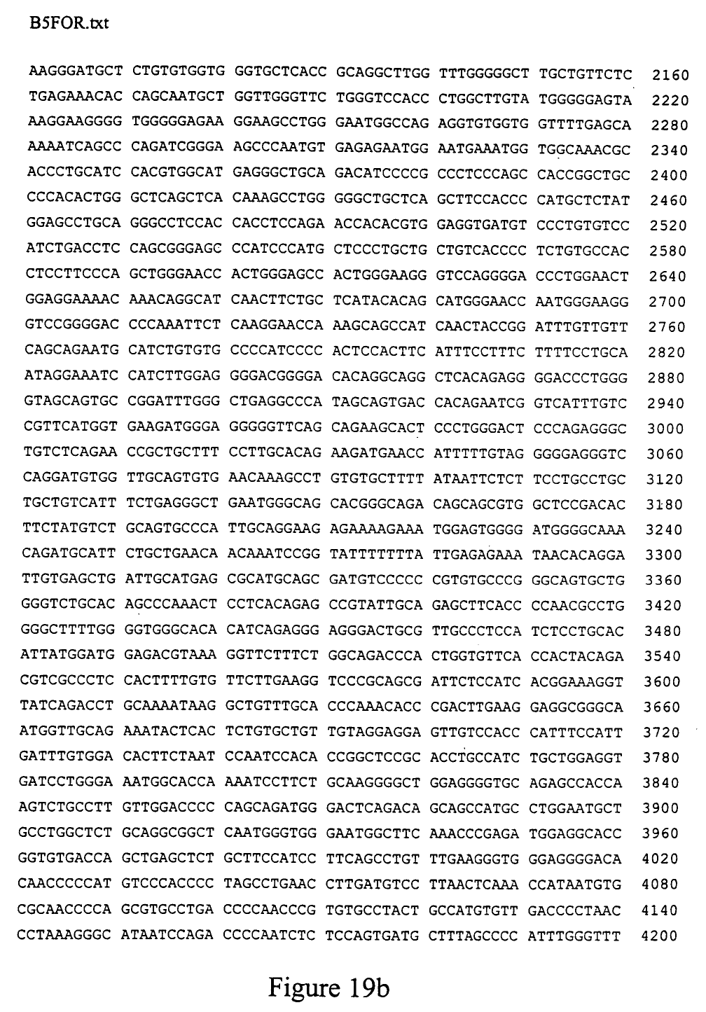

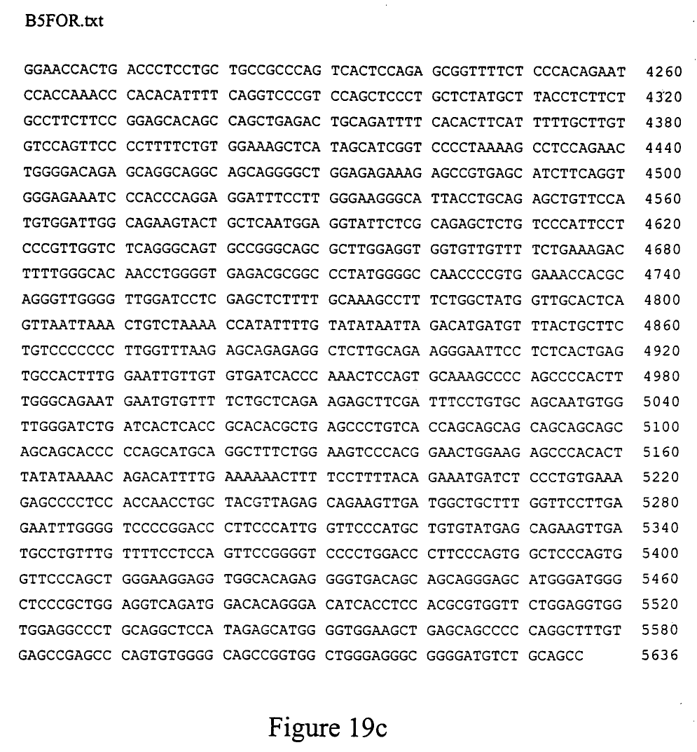

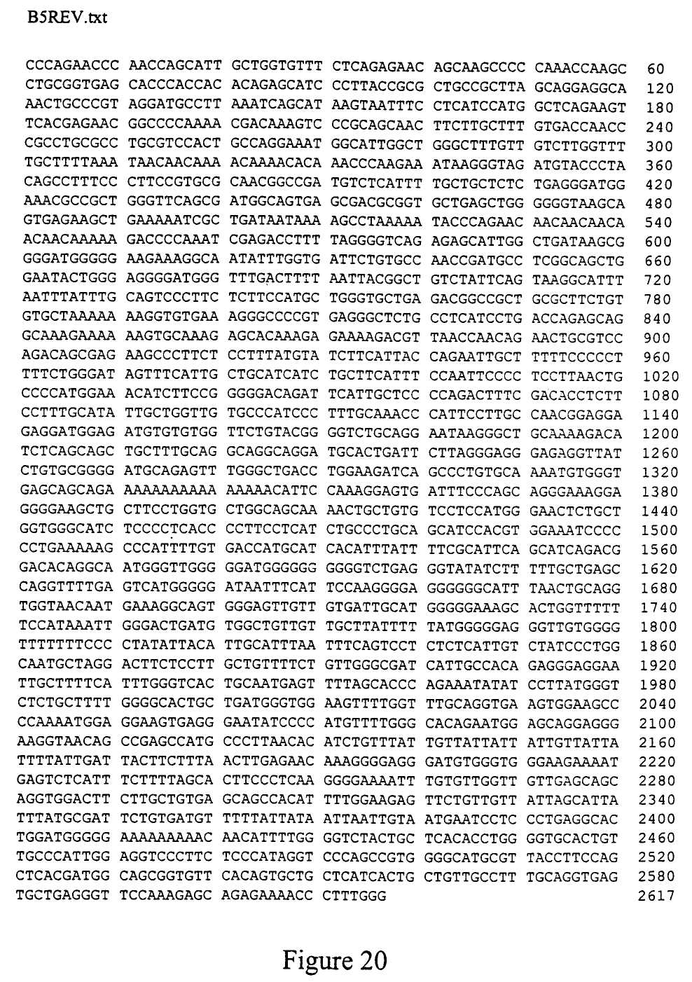

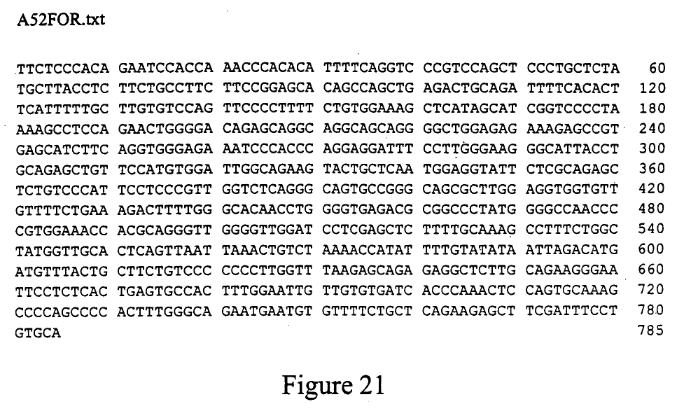

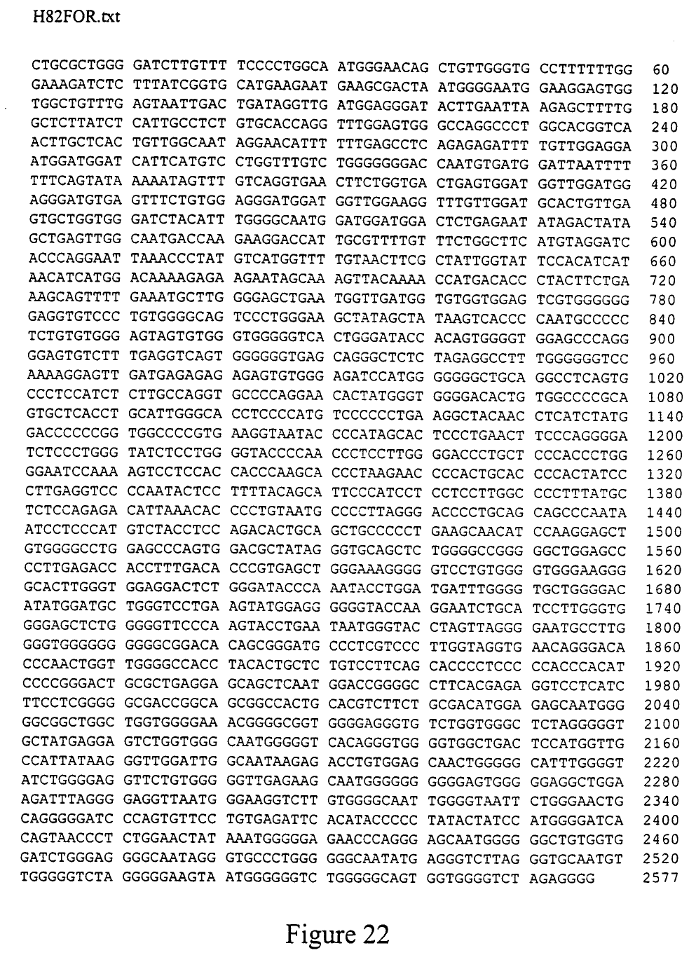

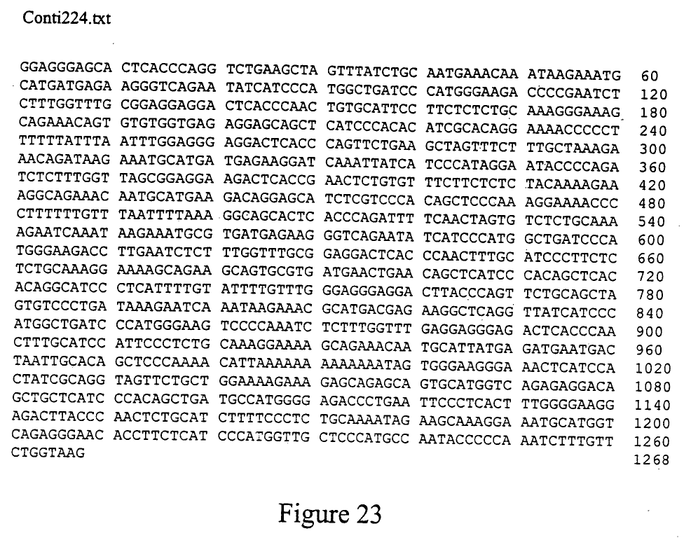

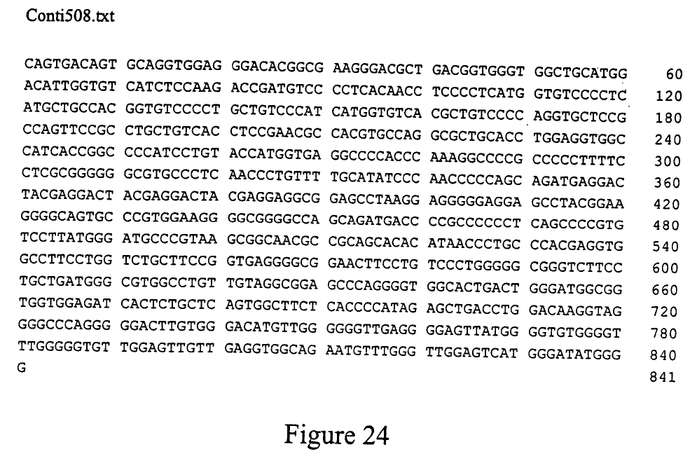

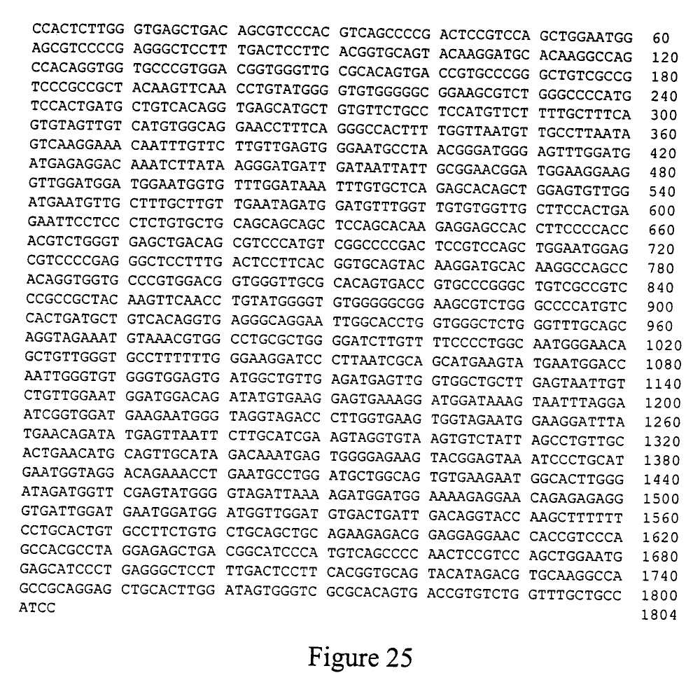

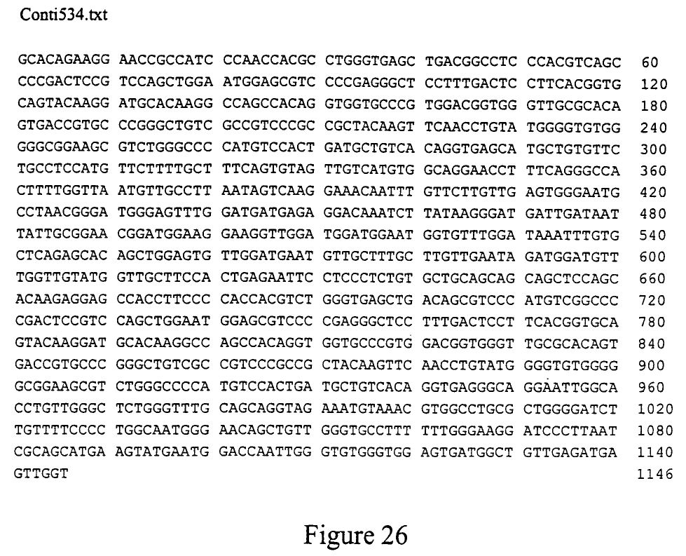

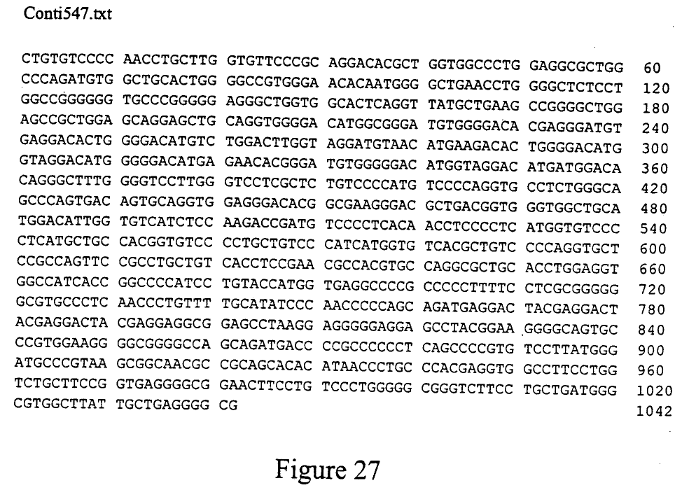

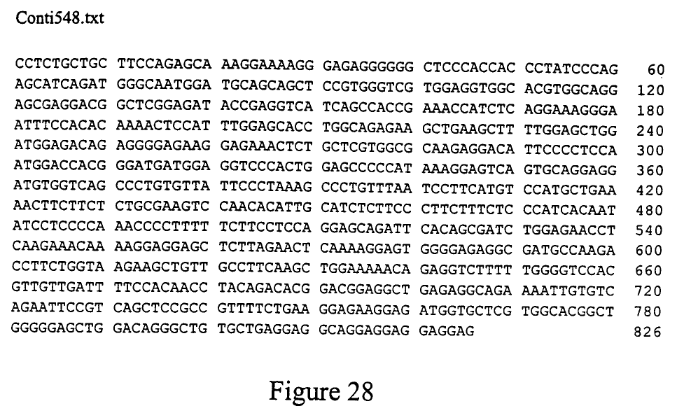

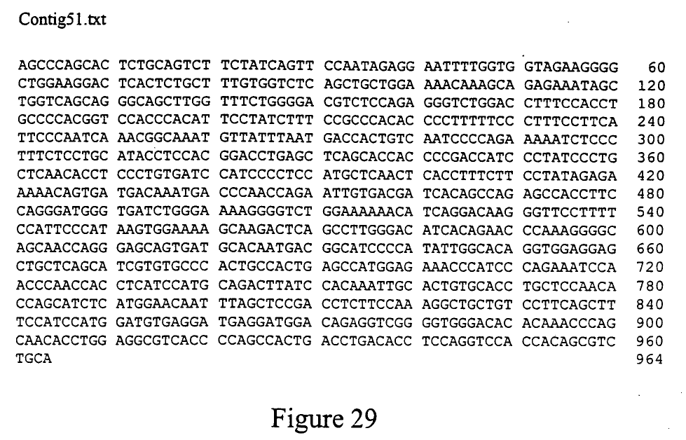

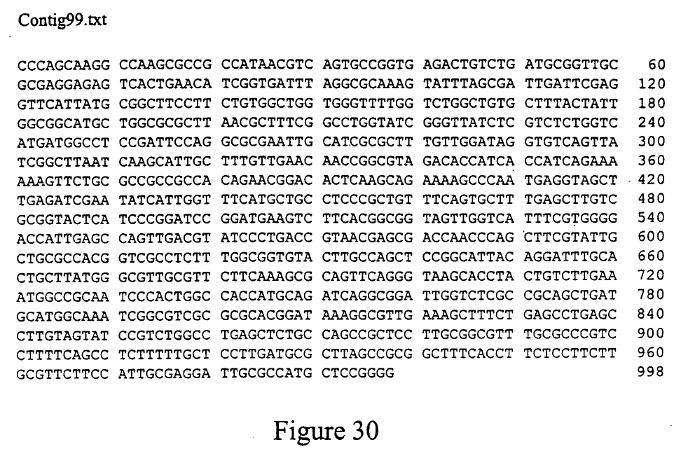

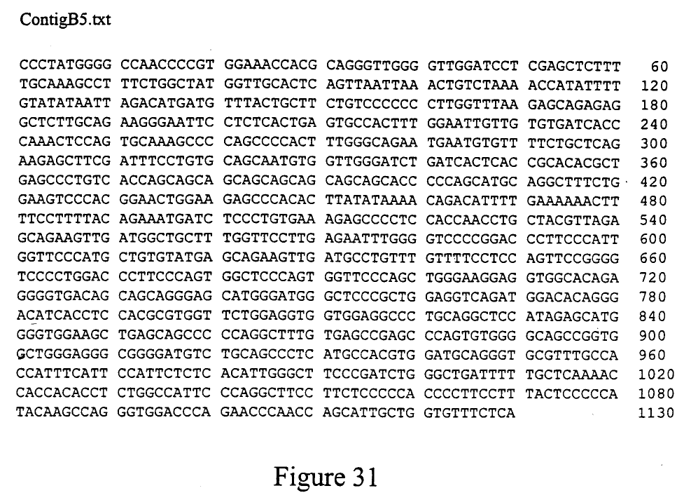

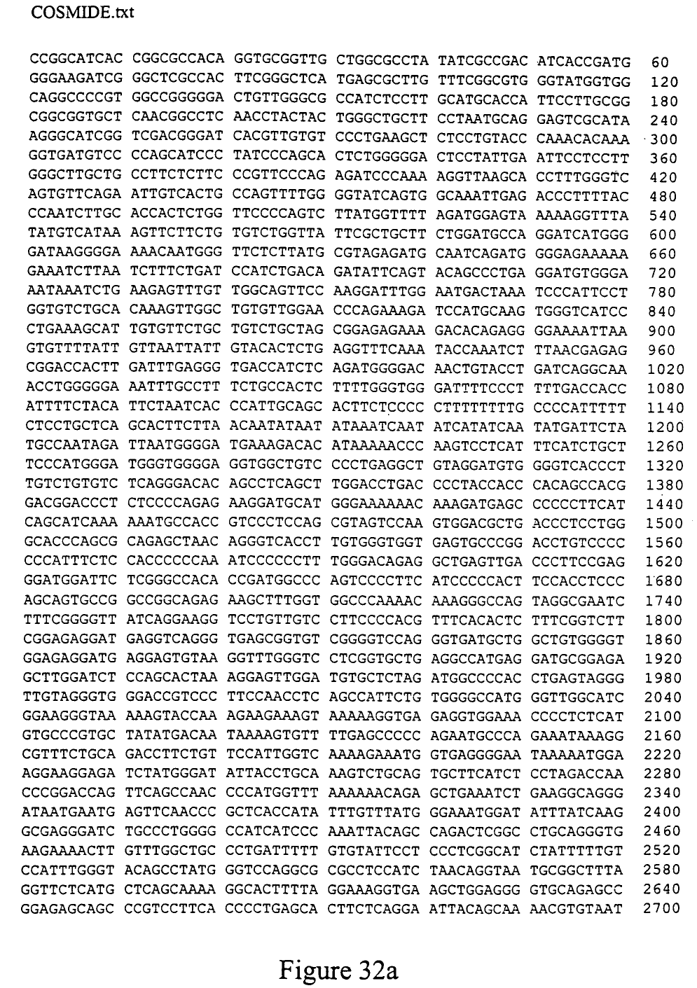

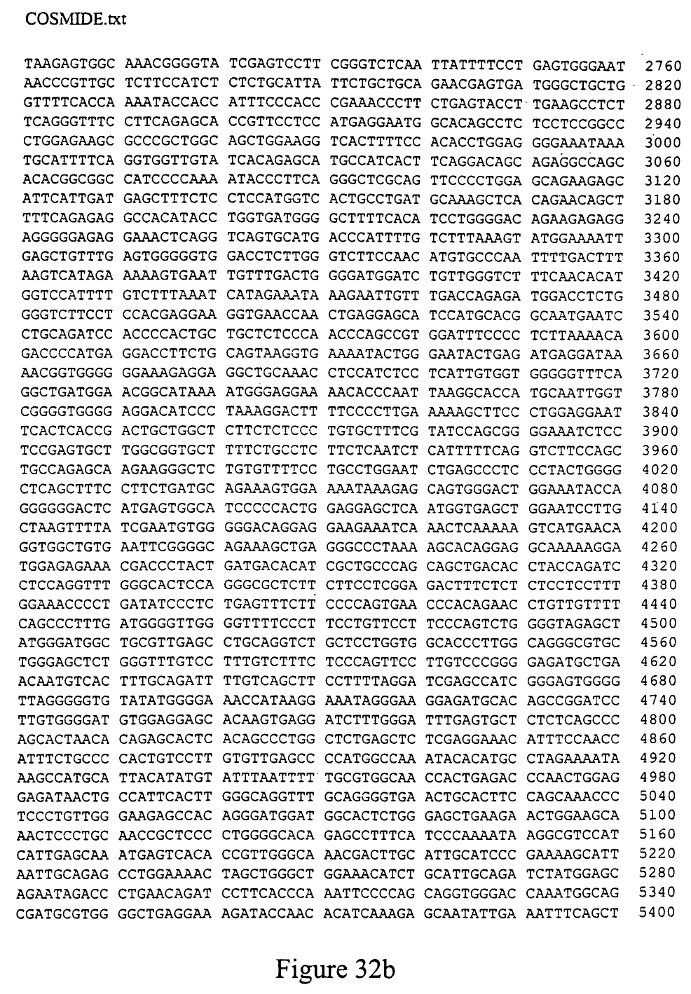

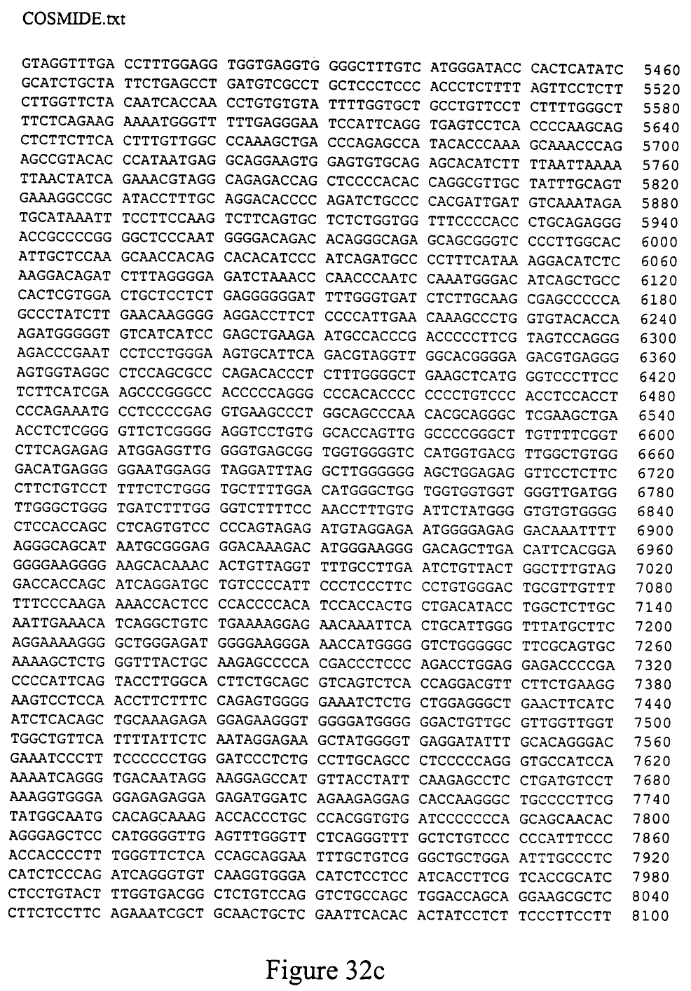

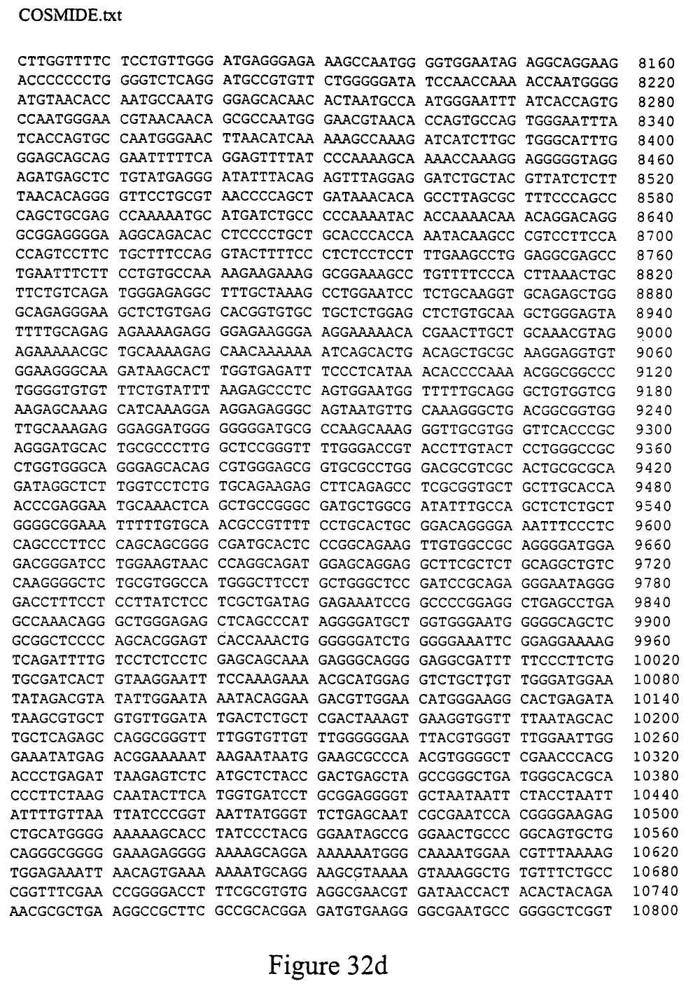

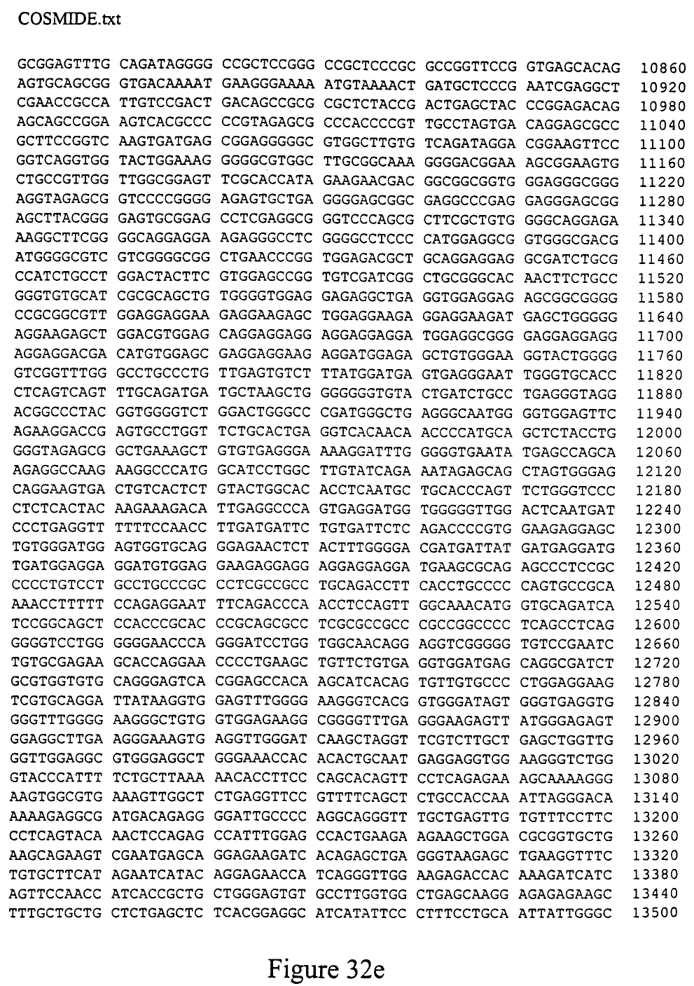

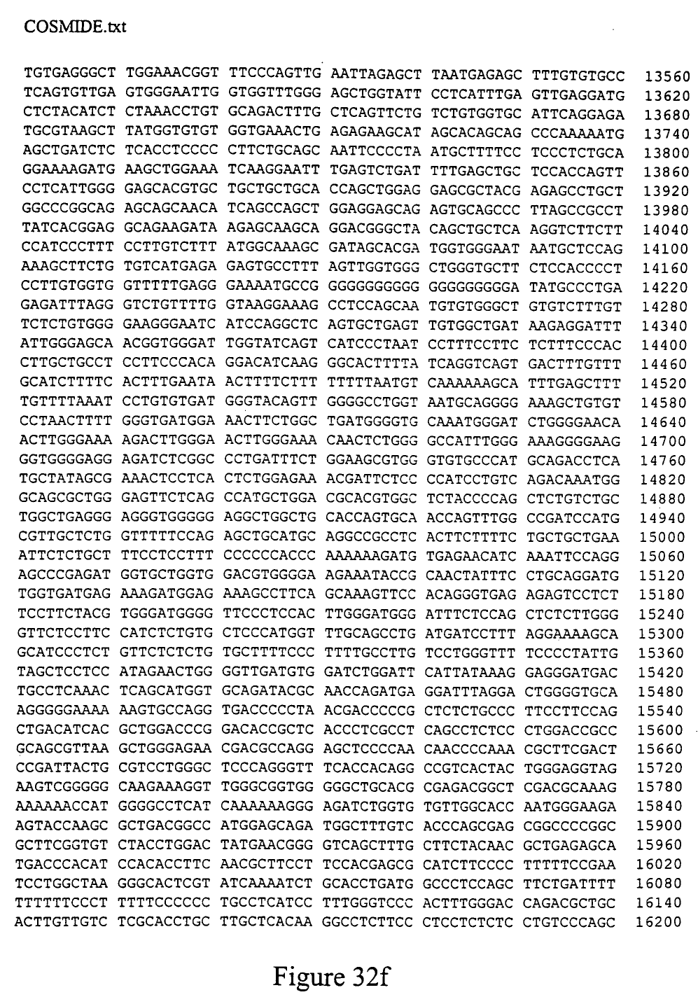

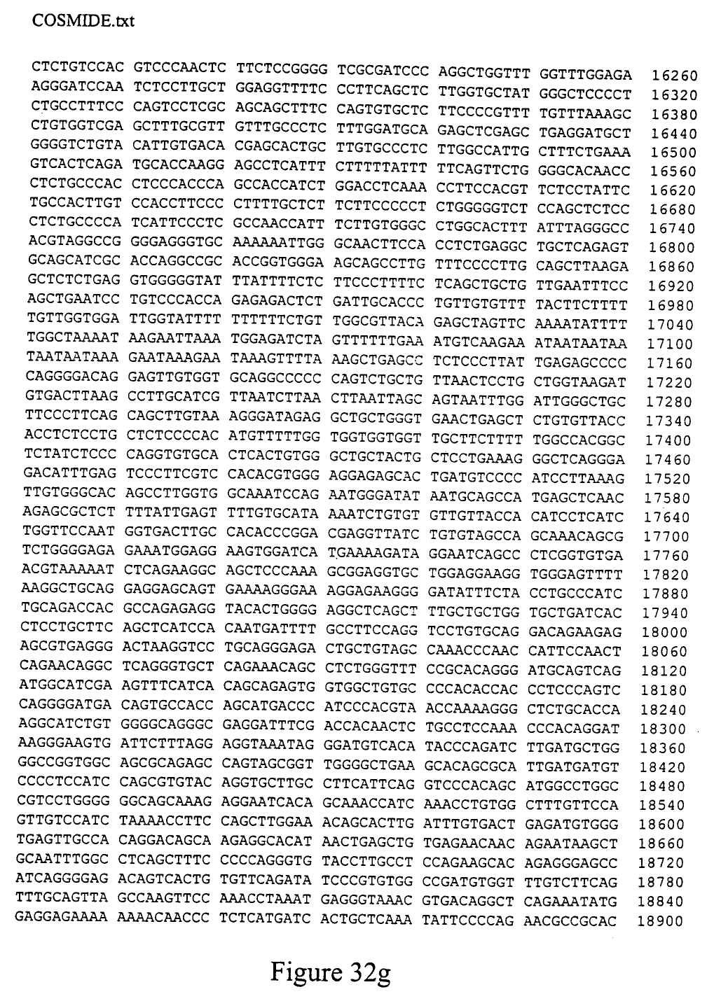

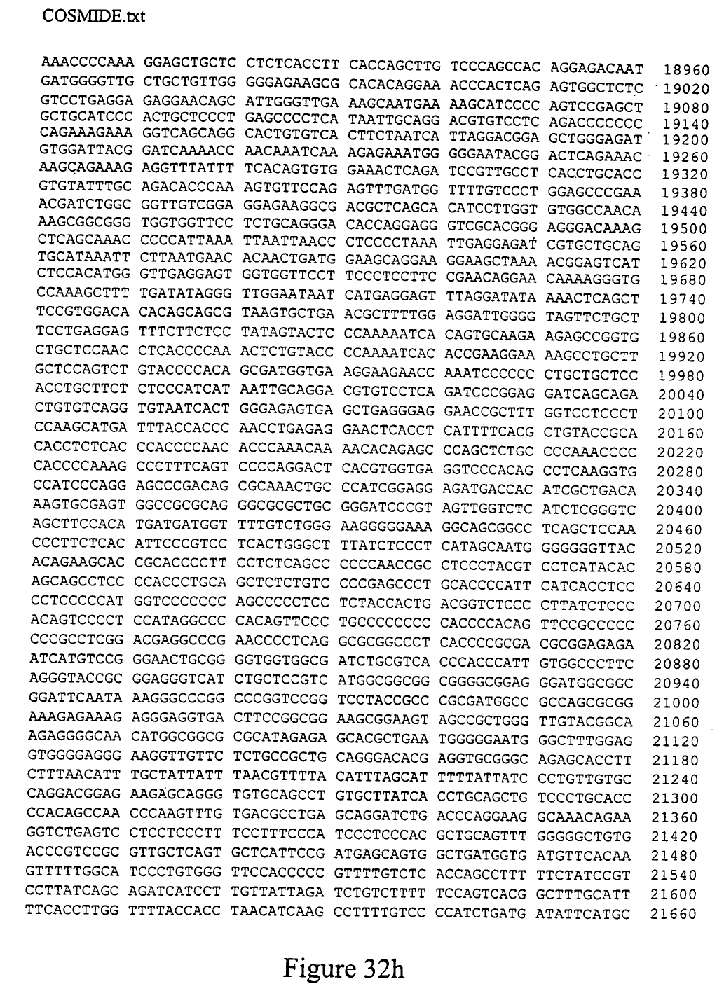

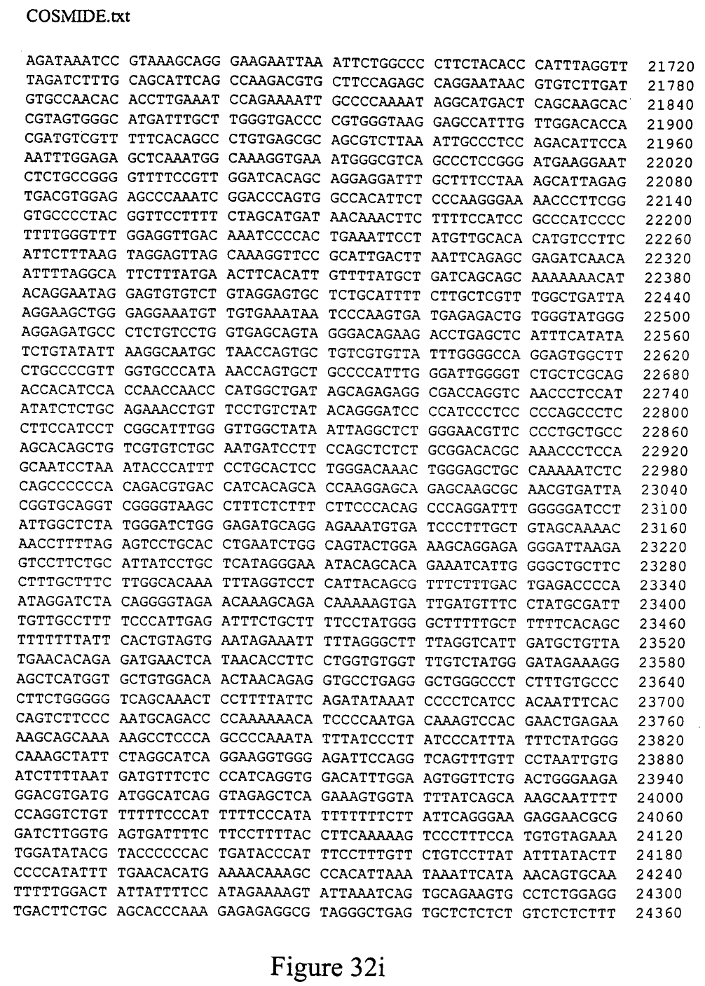

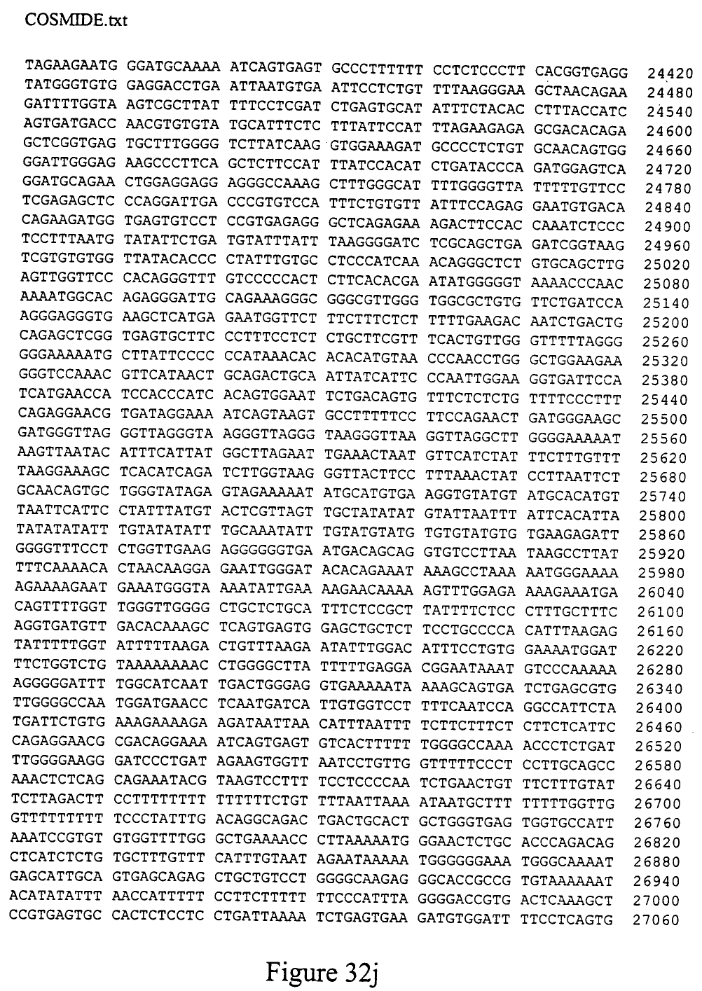

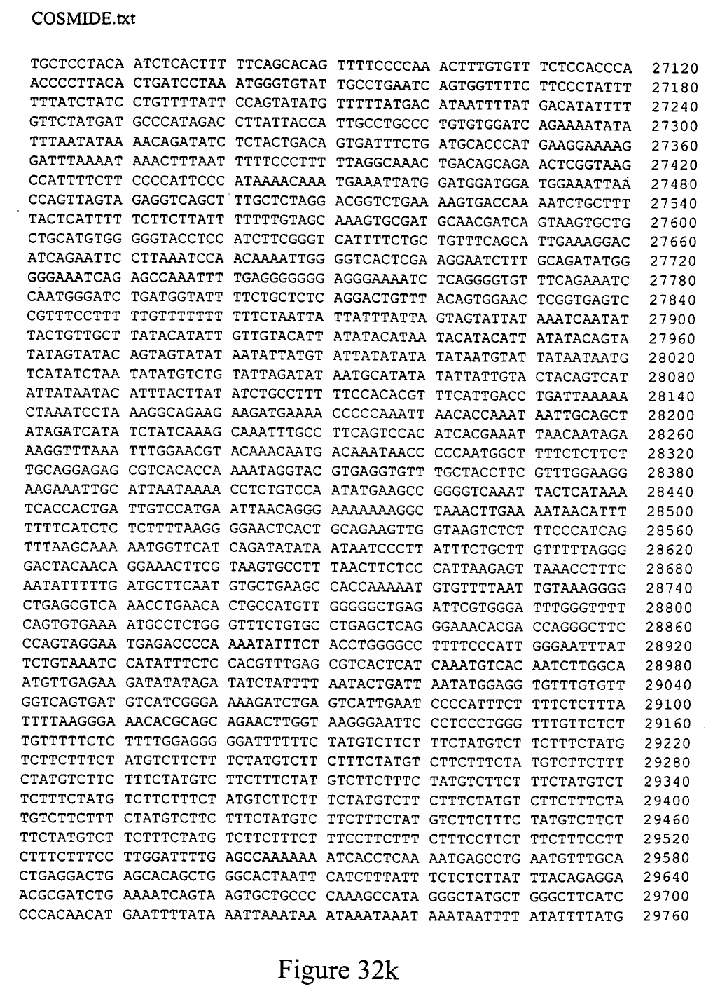

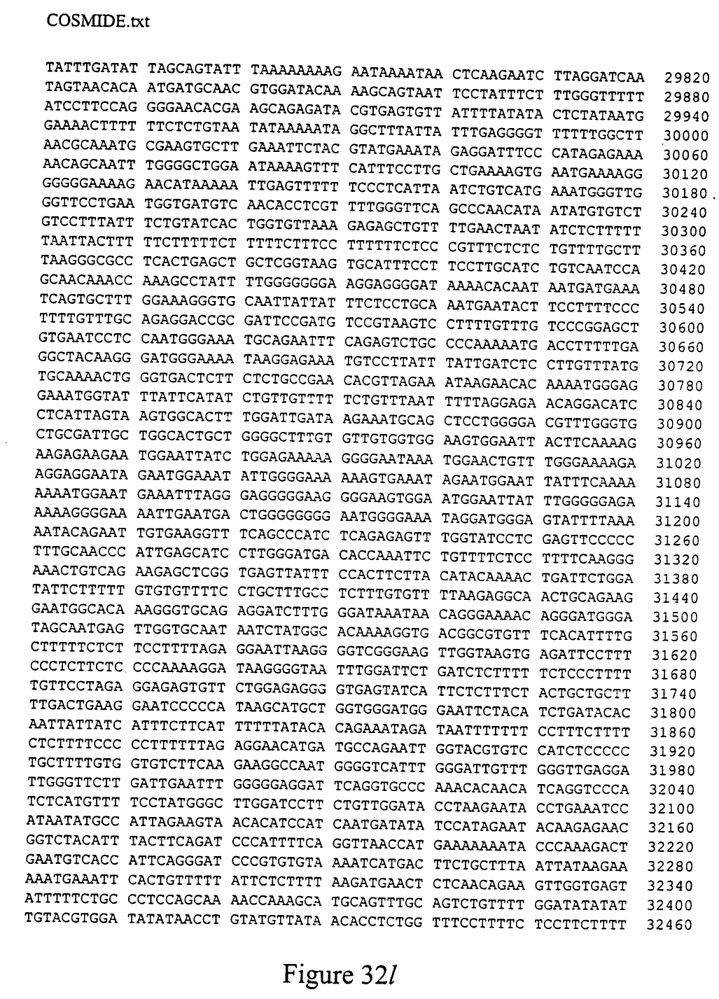

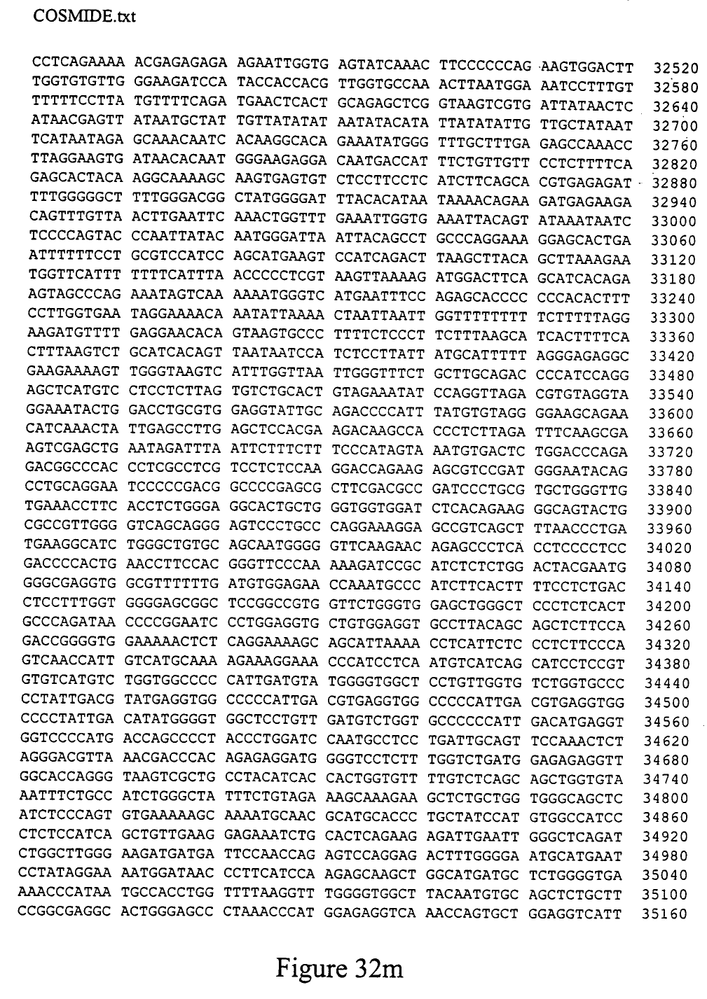

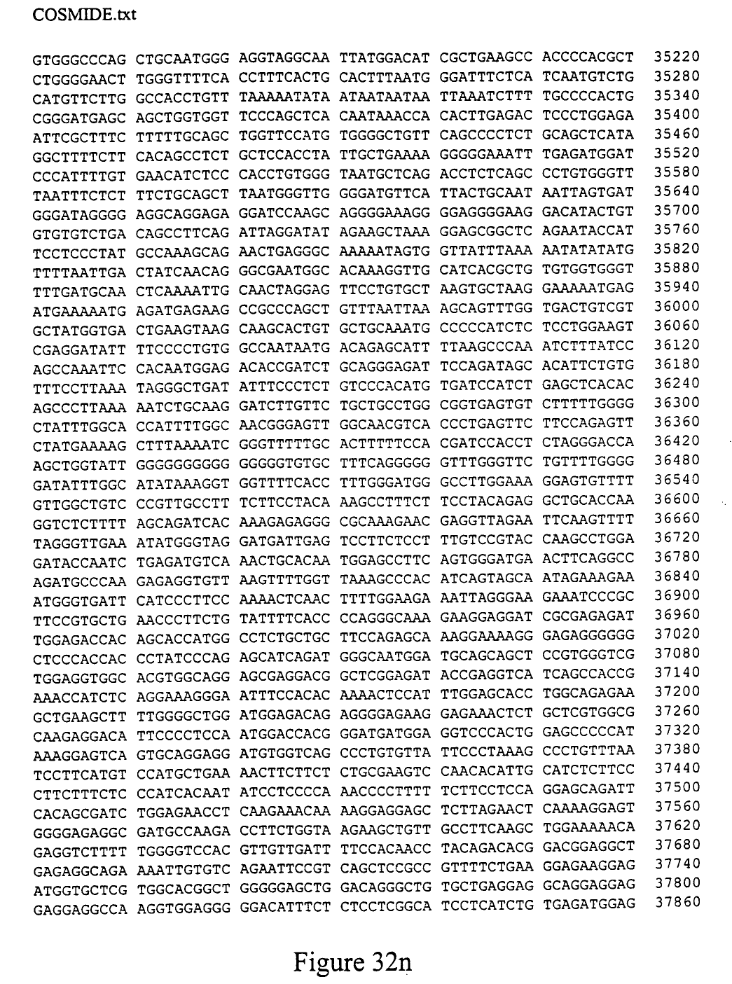

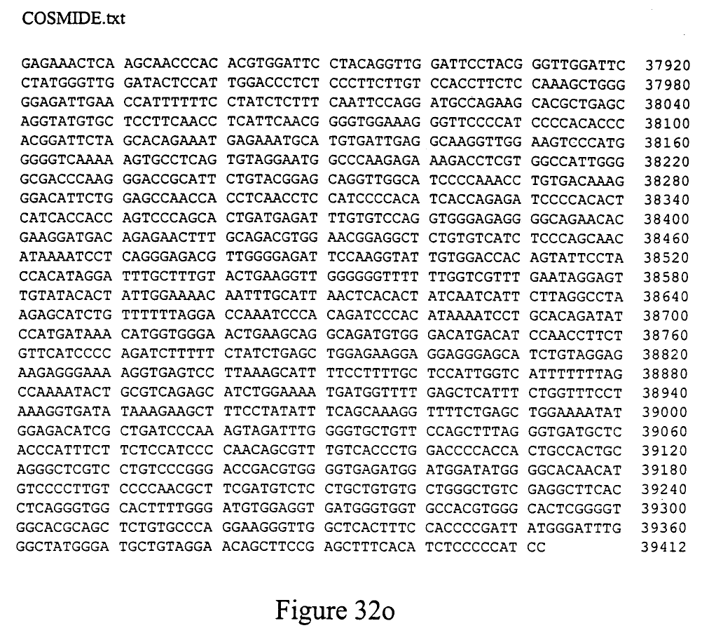

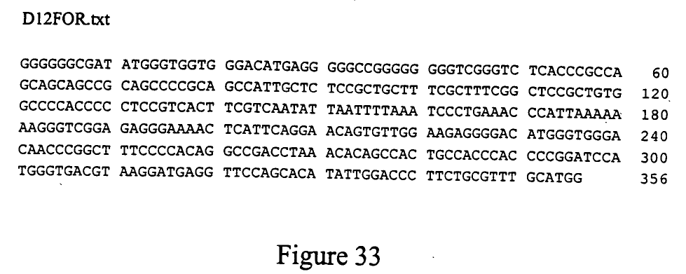

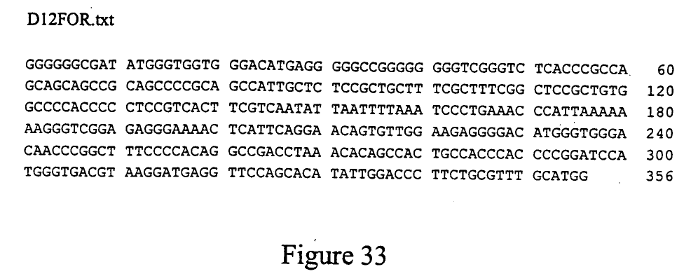

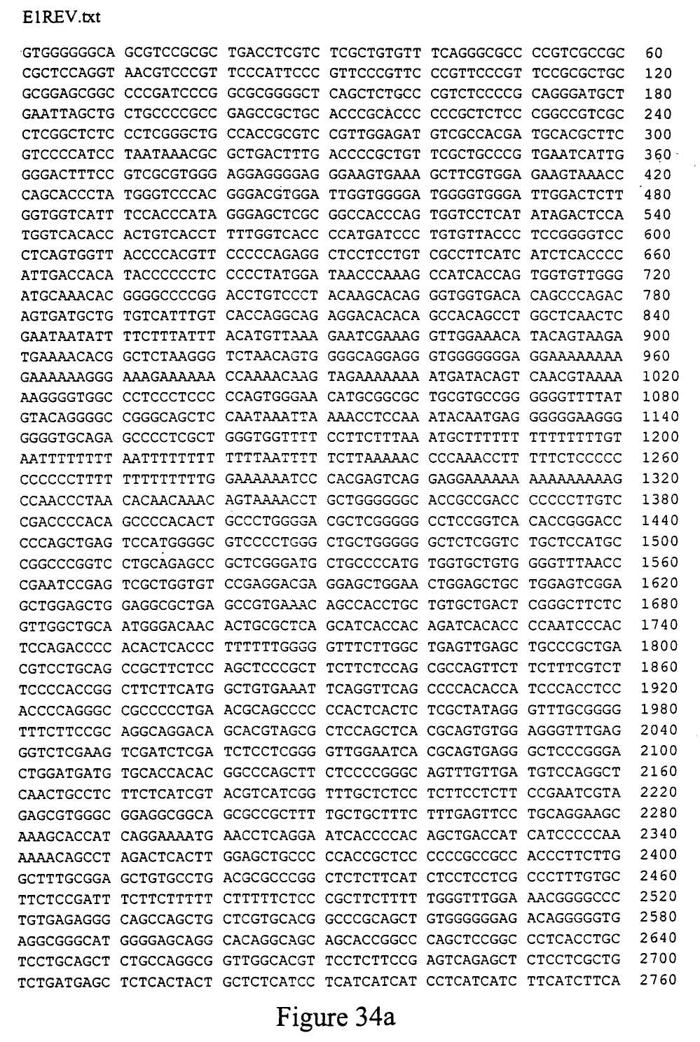

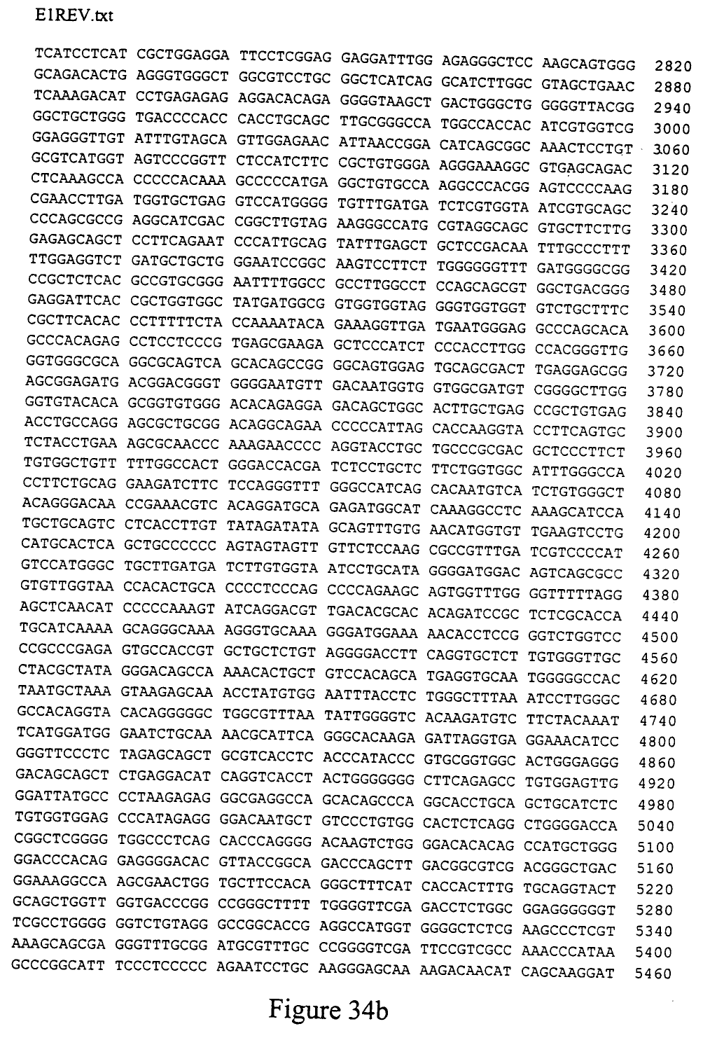

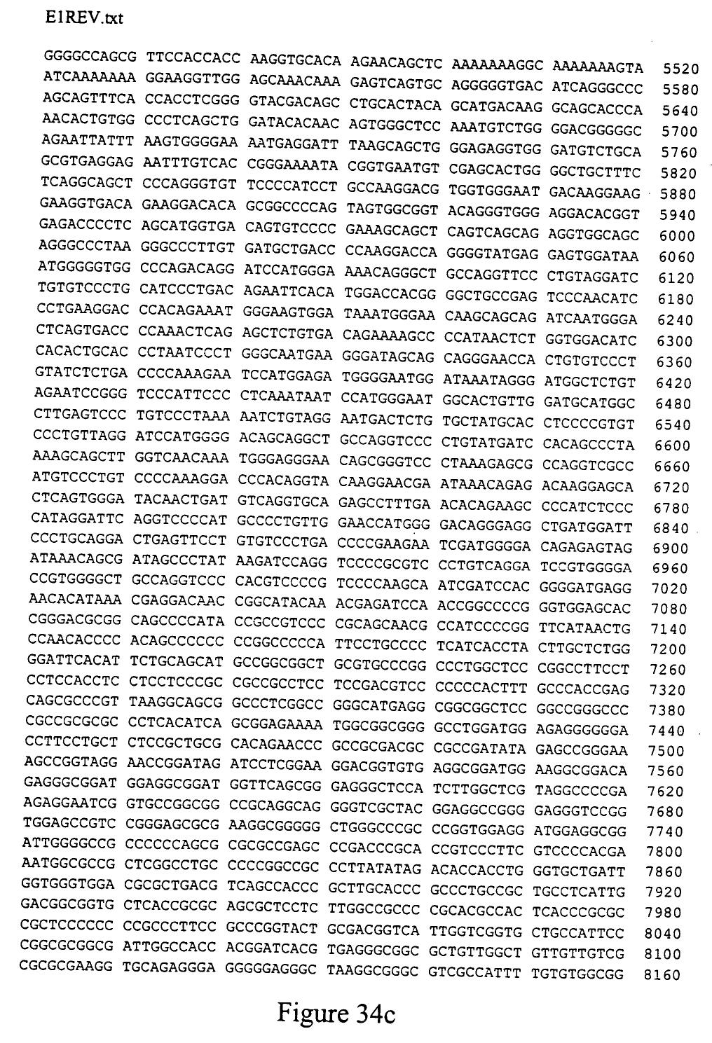

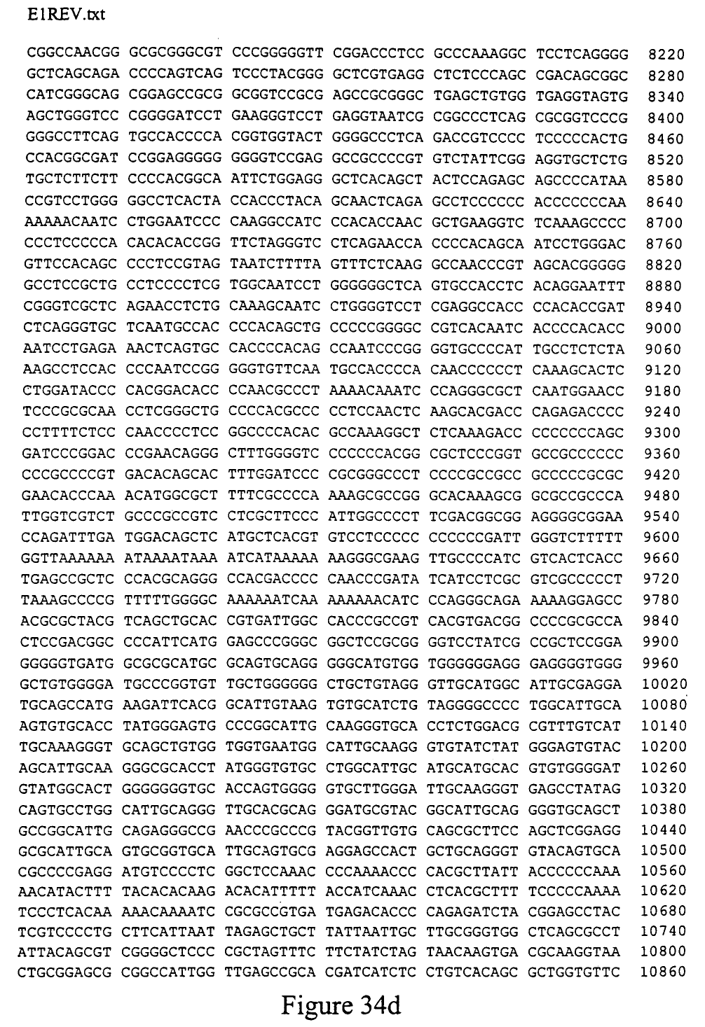

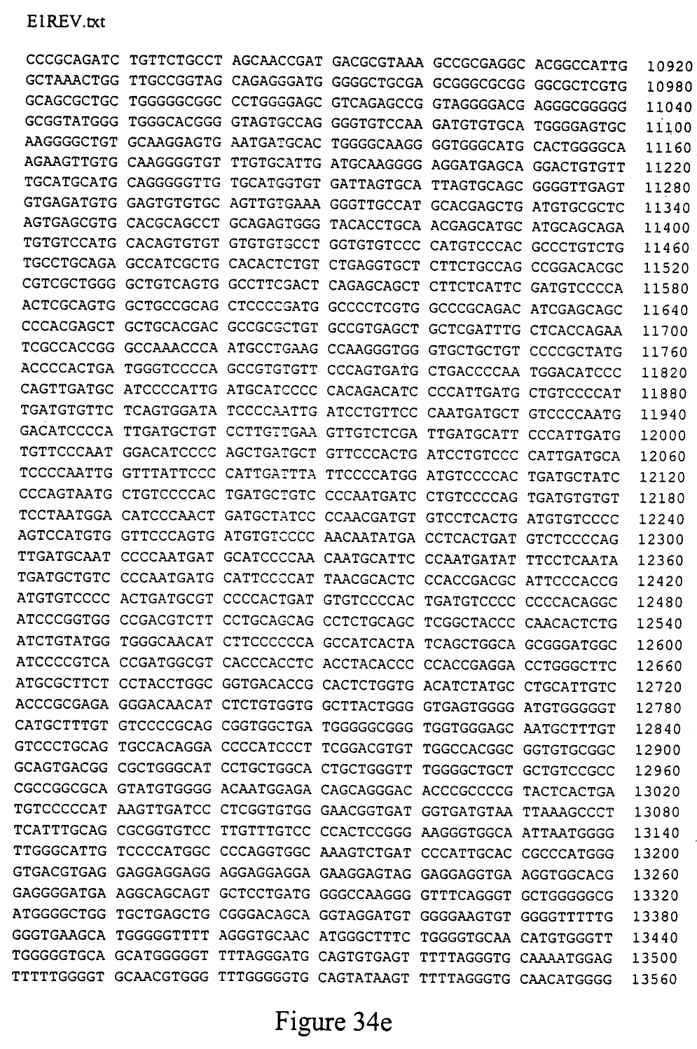

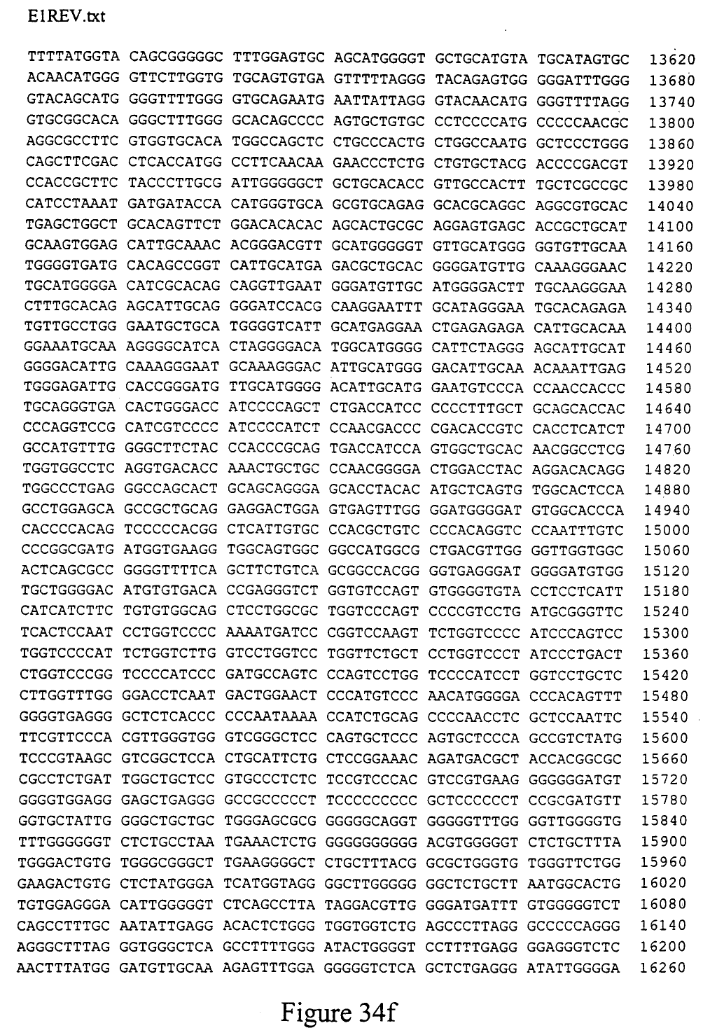

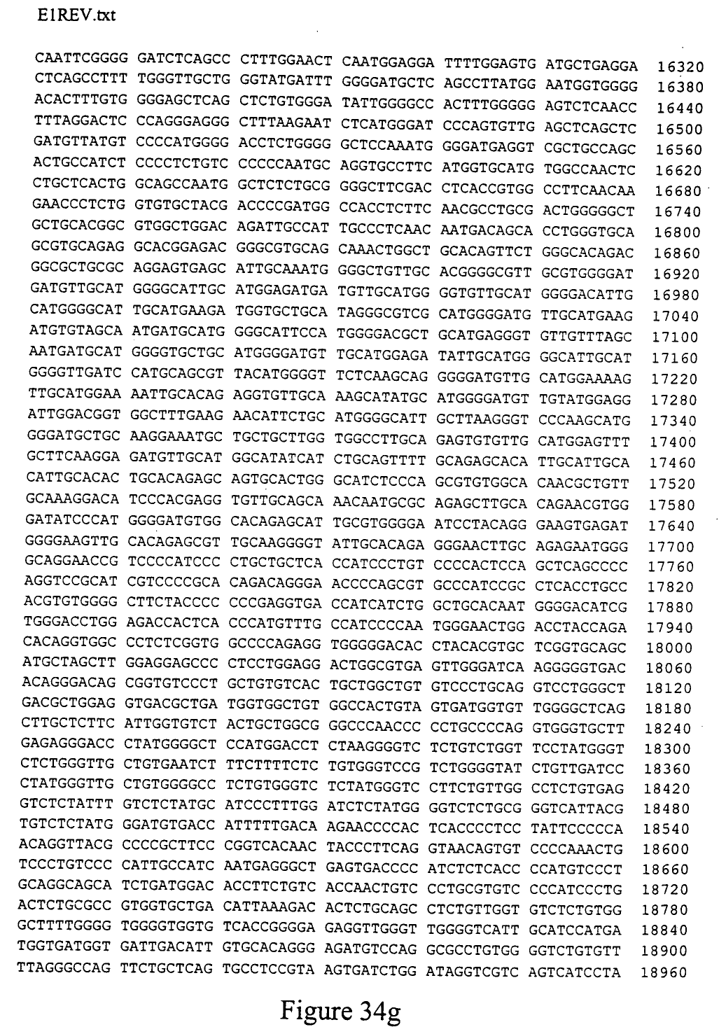

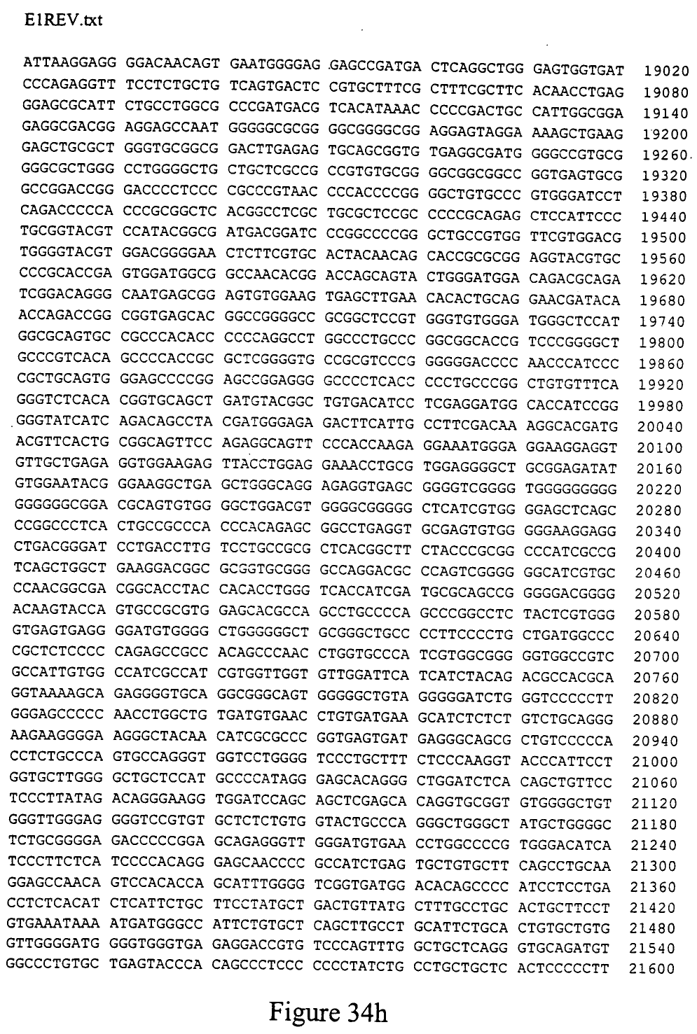

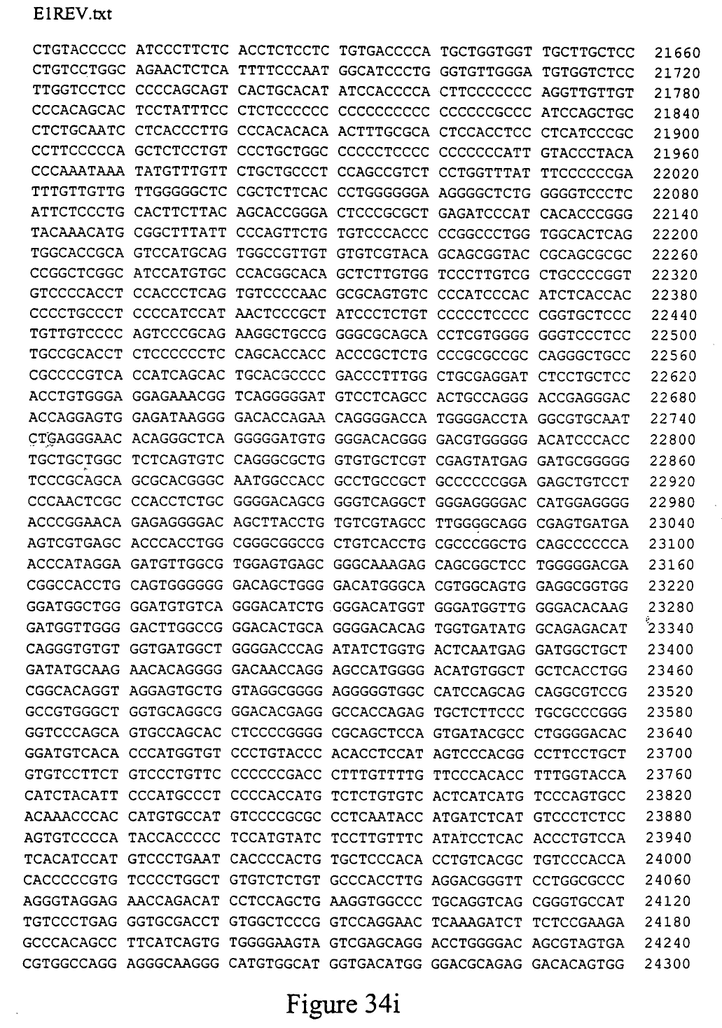

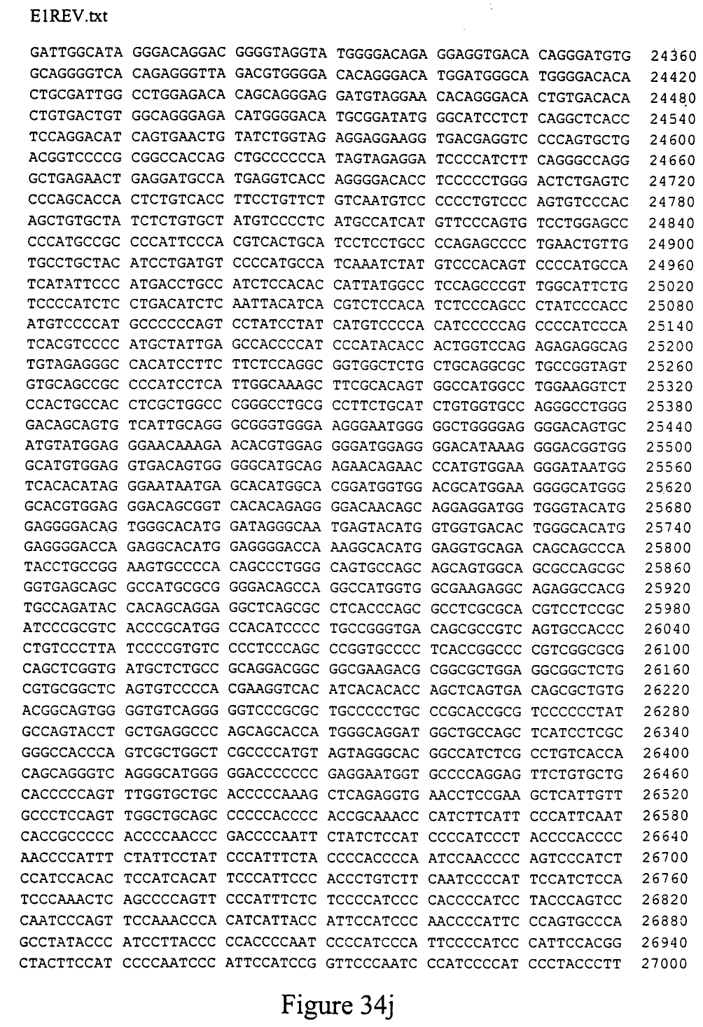

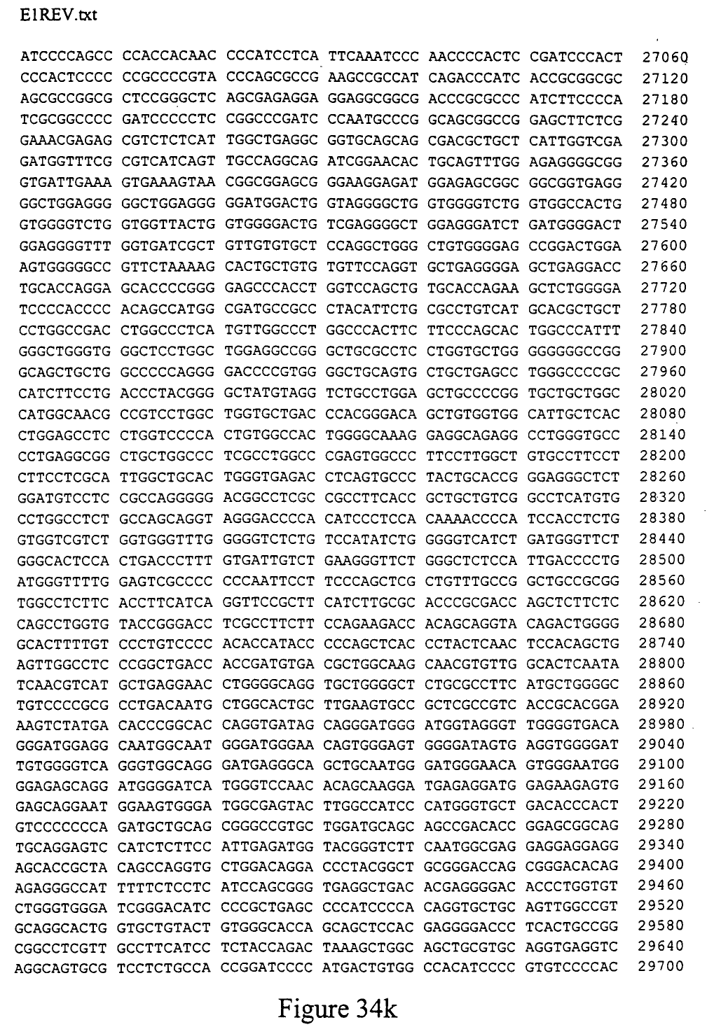

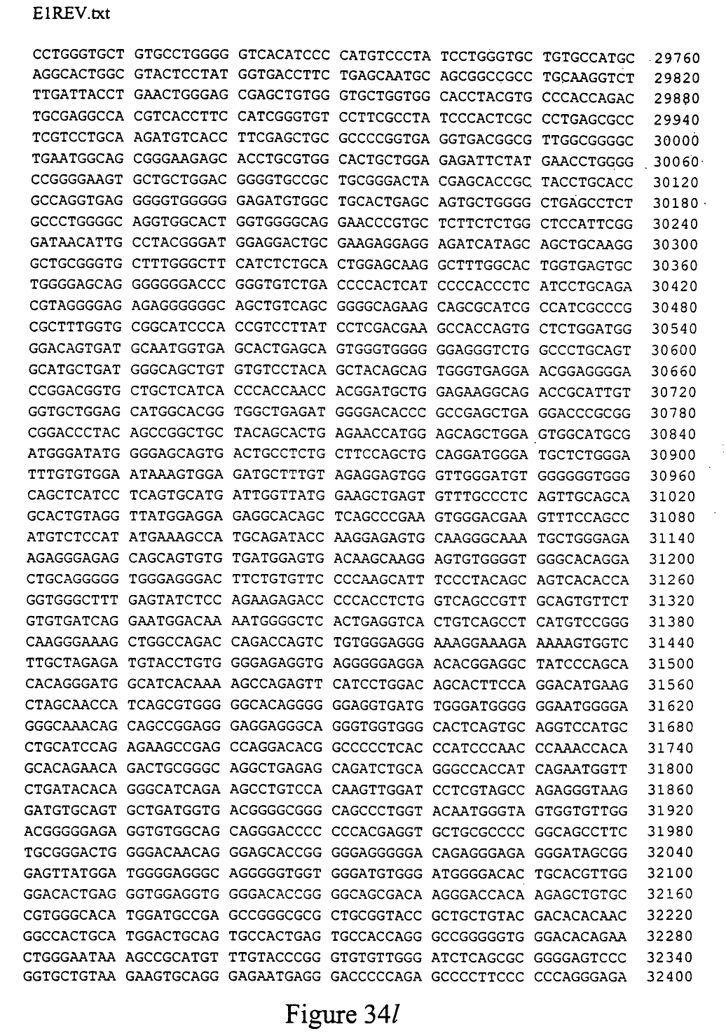

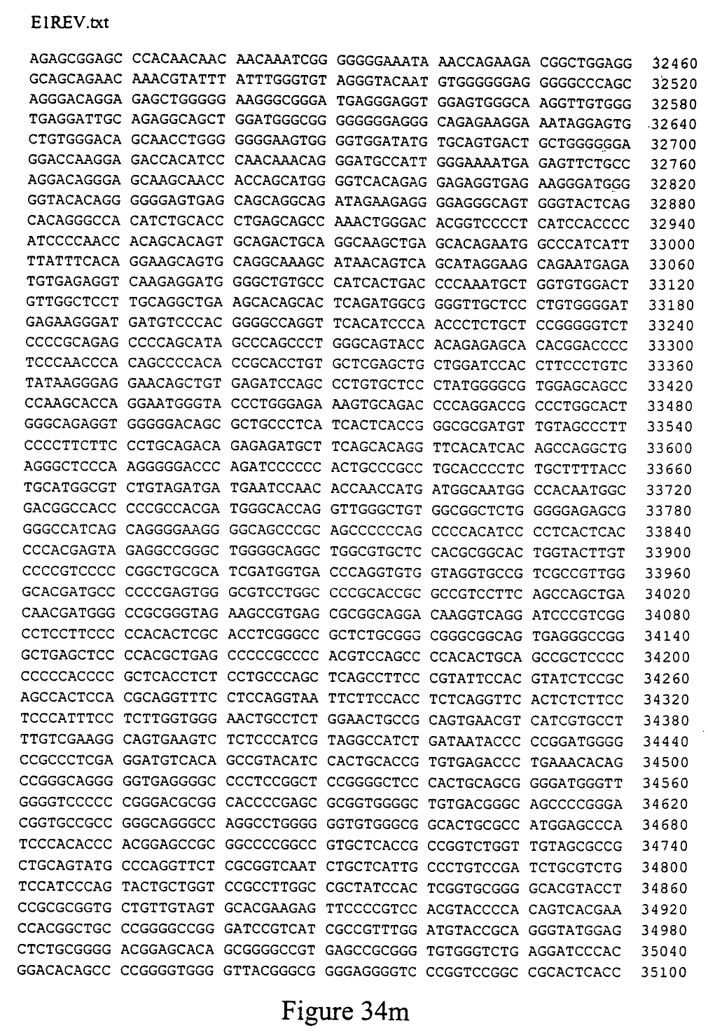

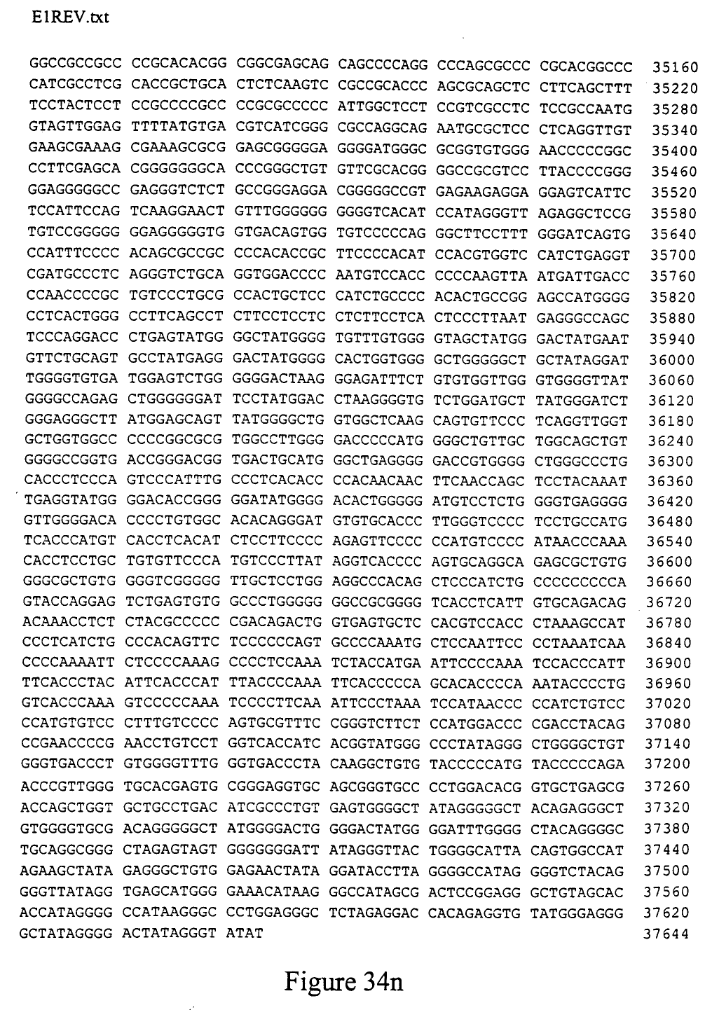

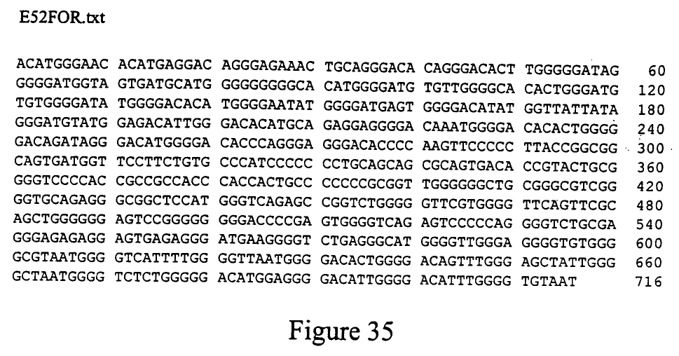

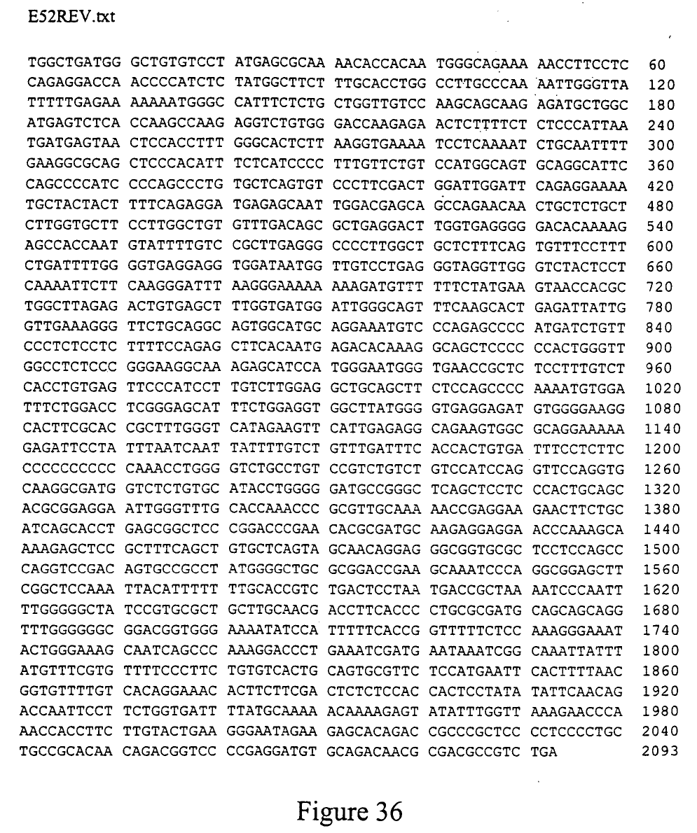

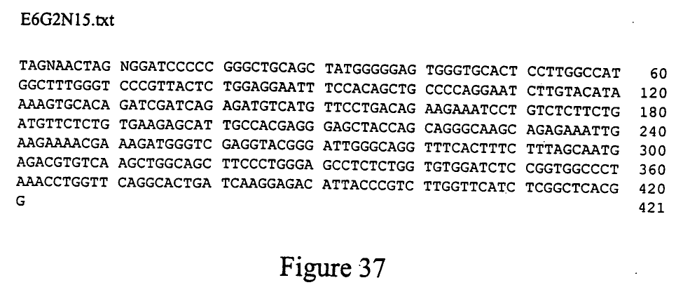

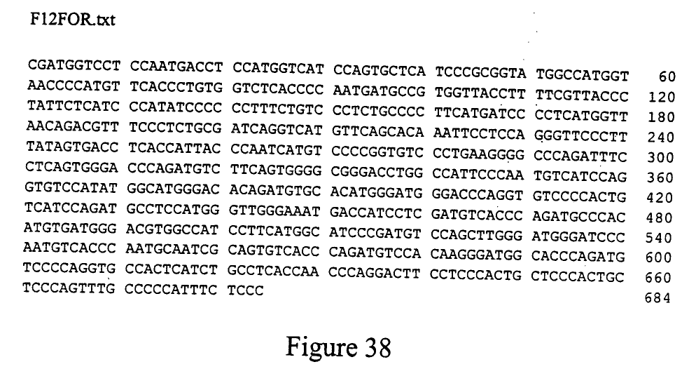

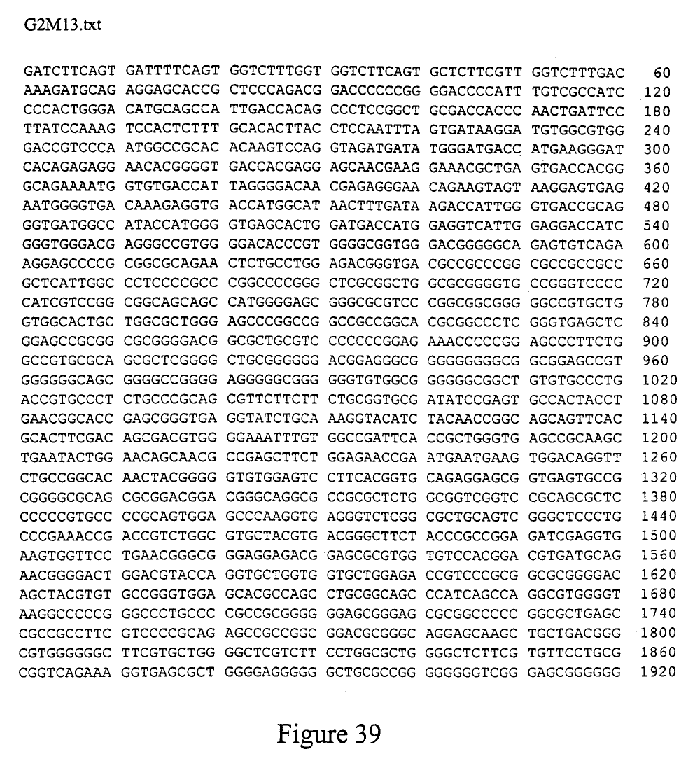

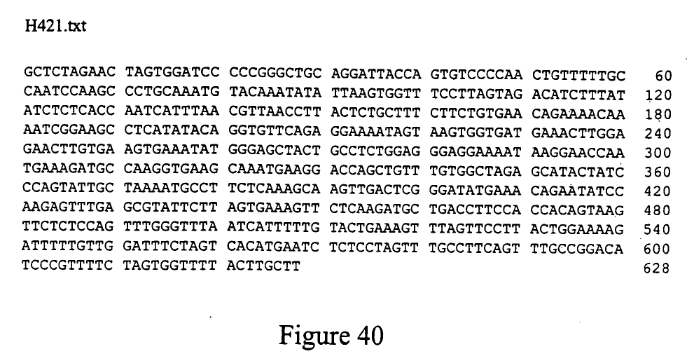

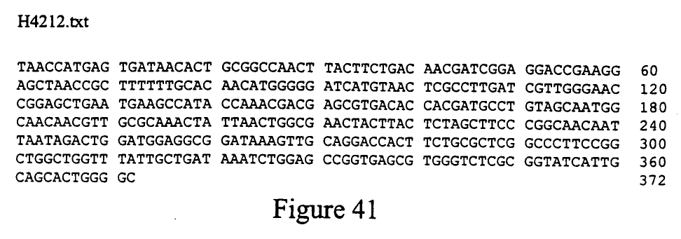

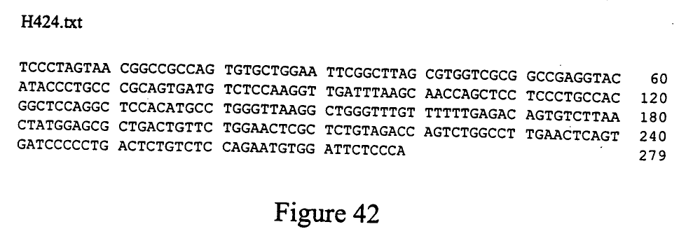

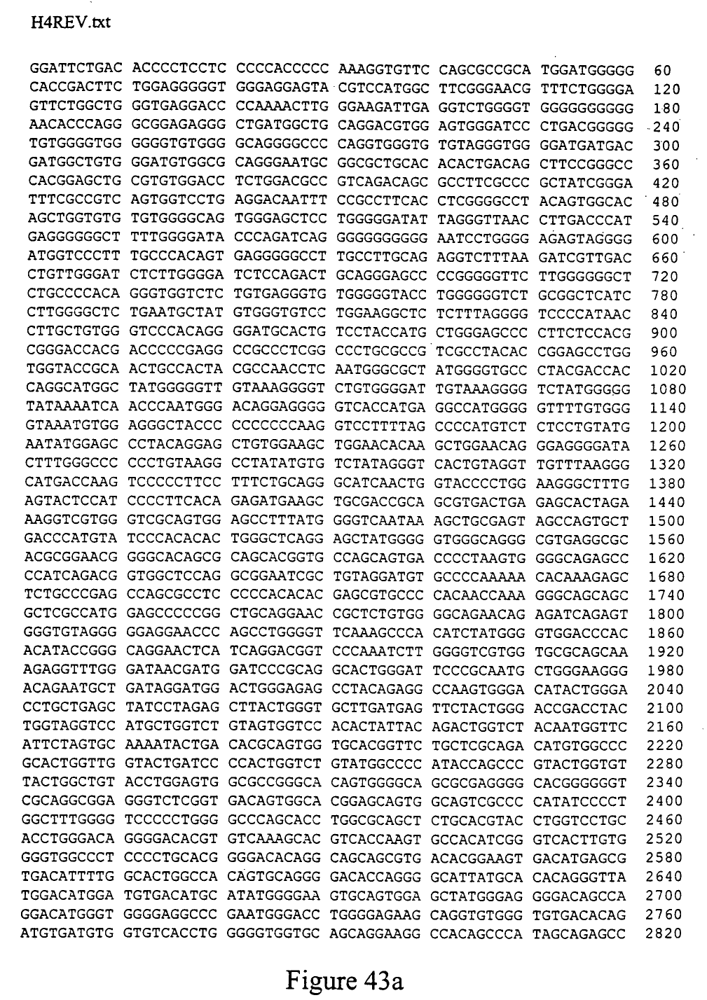

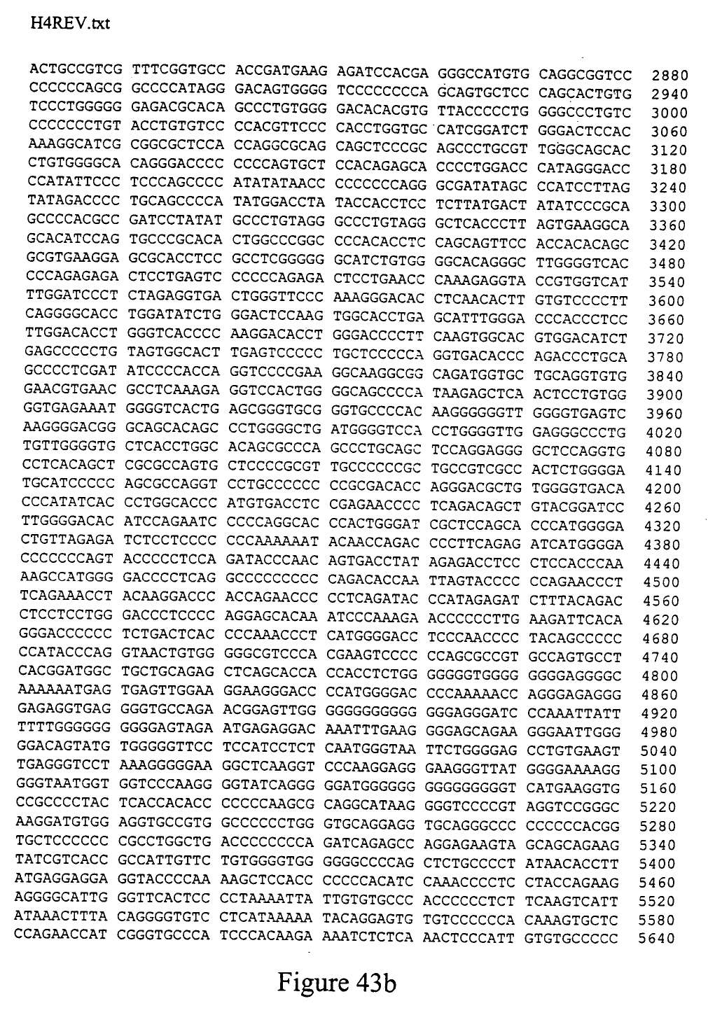

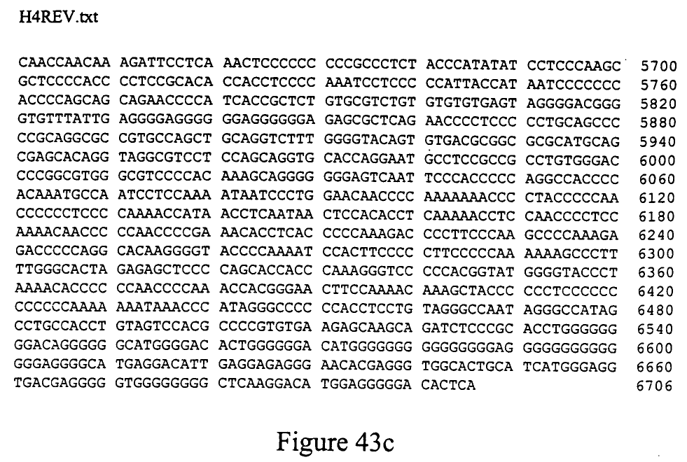

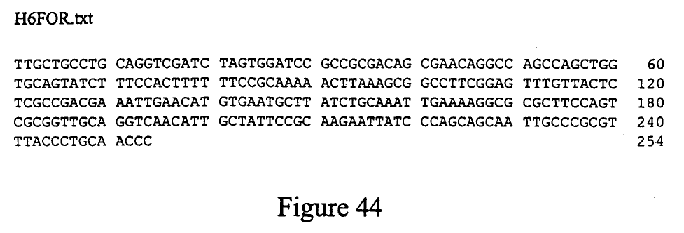

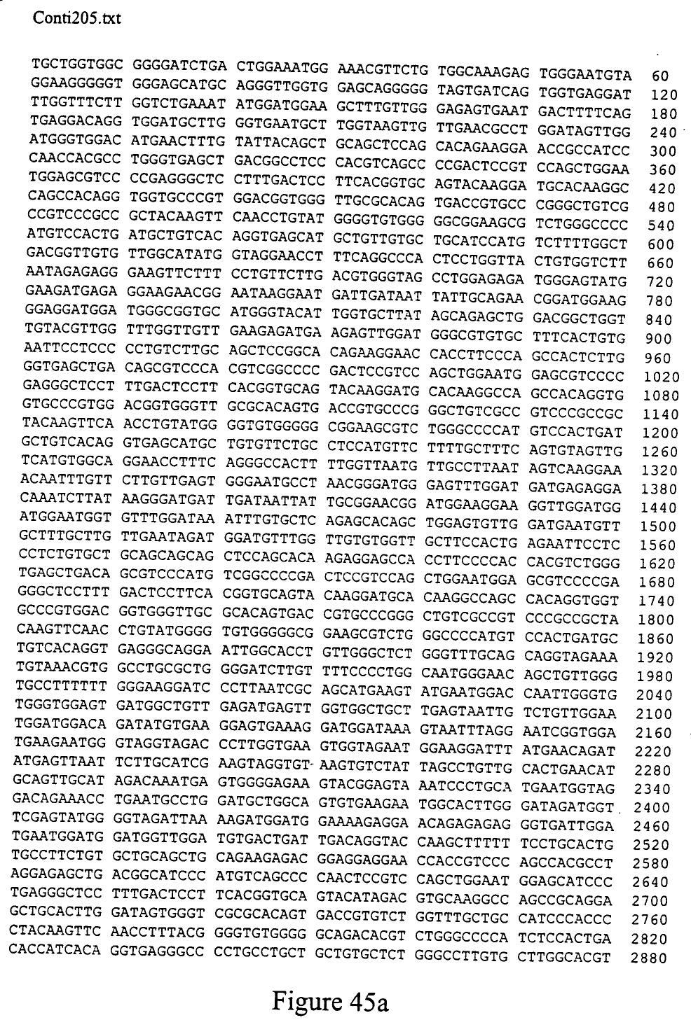

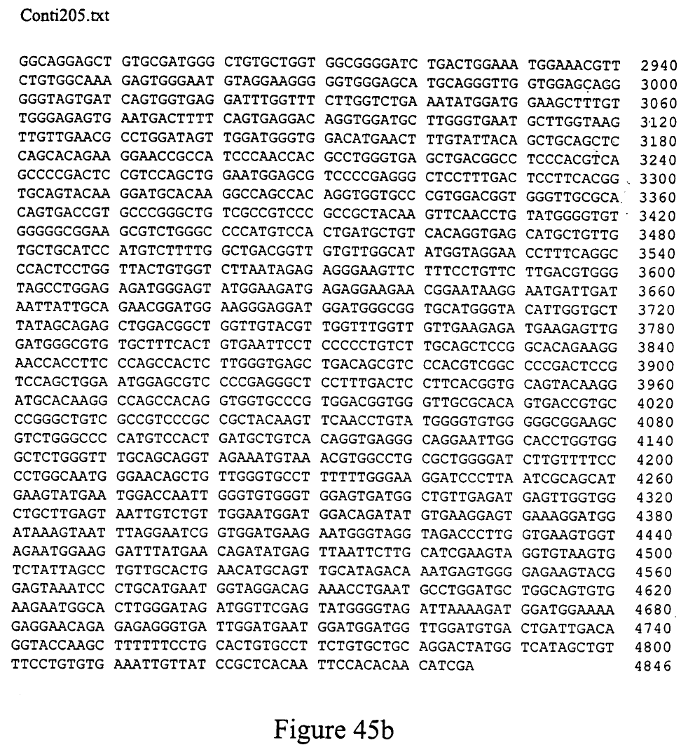

genomic 8.4 (FIG. 3); B-F I (FIG. 4); C121 (FIG. 5); DM (FIG. 6); TAP1 (of the beginning of exon 2 at the 3′ end) (FIG. 7); and TAP2G (FIG. 8), and other genes comprised in FIG. 10 and continuations 1 to 35.

By studying the nucleic acid sequences of the molecules defined above, it was possible to identify accurately the blocks of polymorphisms which must be detected in order to establish a reliable and accurate genotyping.

By comparing the sequences of these blocks originating from different genes of the same haplotype or from the same gene of different haplotypes, the inventors took into consideration divergent sequences, and developed for each gene complementary oligonucleotides of these divergent sequences.

Specific primers which are discriminating with respect to a given gene of the B or Rfp-Y system are obtained in this way.

The invention relates more particularly to oligonucleotide molecules corresponding to these sequences and comprising a part of the polymorphic region of the systems of the MHC of chickens or other farmed birds.

It will be recalled that the polymorphic region may be in the gene or in a related region such as the microsatellite regions or that of the promoter.

According to one embodiment of the invention, the polymorphisms are associated with the function of the systems of the MHC.

These are thus advantageously molecules corresponding to a part of an exon. Molecules corresponding to exon 2 (1 domain) of Y-F genes in chickens may be cited by way of example. A suitable pair of primers is composed of:

| | |
|---|---|
| Y-F VI | 1:GGCCCCGGGATGCCGCGGTTC |
| Y-F VI | 1, R:ATCCGCTCACCGCCCTGG |

According to another embodiment of the invention, the oligonucleotide molecules correspond to a part of a polymorphic region which is not associated with the function of the systems of the MHC. Preferred regions of this type are microsatellites.

Taking the gene B-FI, for example, oligonucleotide molecules that can be used for composing pairs of primers correspond to the following sequences:

| | |
|---|---|
| B-FI: | 5′ CCA GCA GTC ACT GCA CAT AT 3′ |
| B-FI, R: | 5′ AGG TGG AGT GCG CAA AGT T 3′; and |
| 12.1: | 5′ AGA CGC AGC AGA ACT TGG TAA 3′ |
| 12.1 R: | 5′ GGA AGG AAG ACC TTG GAA 3′ |

With the oligonucleotide molecules defined above and those developed from known genes but according to the step of the invention, highly specific sets of primers are obtained which make it possible to determine accurately the haplotype of the animal to be studied and to detect whether it is resistant to the development of virus-induced tumours or, in contrast, likely to be affected.

The invention also relates, therefore, to a method of genotyping farmed birds, particularly chickens.

This method is characterised in that it comprises:

amplification of a sample of nucleic acid originating from the animal to be studied using one or more pairs of primers capable of hybridising specifically with the nucleic acid of a polymorphic region of the Rfp-Y or B systems of the MHC of said birds and

detection of the PCR products obtained.

A simple comparison of the results obtained with a reference established beforehand makes it possible to determine rapidly the haplotype of the animal.

The sample of nucleic acid is composed particularly of genomic DNA extracted from biological material of the animal to be studied or composed of this material itself, particularly the blood of the animal. It may be, as a variant, cDNA, RNA or PNA (polypeptides nucleic acids).

The primers are developed from oligonucleotide molecules defined above and, generally speaking, from any gene (and related region) coding for a protein involved in controlling resistance or susceptibility to virus-induced tumours in farmed birds, and particularly chickens, particularly class II B-L genes, 17.5, 12.3 and class I B-FIV genes.

They are, for example, primers of microsatellite regions which make it possible to detect haplotypes of the B complex such as those developed from the gene B-FI, and mentioned above, or primers which make it possible to detect haplotypes of the Rfp-Y system, and developed from gene 17.5, such as the pair:

| | |
|---|---|
| 17.52: | CAG GAT CTG CAC TGG CCA ATA |
| 17.5, R1: | GAA TGG CGG TGC TTC CGT GCC TGG |

The PCR products are detected according to conventional methods. These methods include sequencing, electrophoresis, hybridisations with SSOP or SSCP analysis.

This method will be selected advantageously according to the nature of the polymorphism involved. Thus, in the case polymorphism of the microsatellite type, the PCR products will be detected advantageously according to their size using electrophoresis methods.

If the polymorphism concerns only a few nucleotides, or even a single nucleotide, the methods used more specifically for the purposes of differentiating the haplotypes of PCR products will be hybridisation (analysis on a membrane using specific probes of the haplotype sequences, SSOP or Sequence Specific Oligonucleotide Probe), differential migration of denatured samples (SSCP or Single Strand Conformational Polymorphism) or sequencing. Generally speaking, this latter method is preferred in view of the ease with which it is carried out.

The invention thus provides a simple and rapid method for establishing the genetic profile of a large number of animals to be studied, which makes it possible to determine the haplotypes and to select those of interest with a view to breeding.

Moreover, as it is possible to distinguish each type of gene using primers having the required specificity and to establish whether it belongs to the B or Rfp-Y system, it is possible to carry out more complete fundamental studies.

The invention also relates to a unit or kit for detecting the genotype of the chicken or other farmed bird according to the method defined above.

These units or kits are characterised in that they comprise the reagents required for carrying out at least one PCR and the detection test.

In particular, they comprise the primers for PCR, a positive indicator of the reaction, and directions for use.

The primers are in the freeze-dried form or in solution or, depending on the mode of detection, on a support. By convention, the support may be a multiwell assay plate or it make take the form of DNA chips.

The invention also relates to an experimental system which enables the resistance to the tumoral development to be studied in chicken.

It corresponds to animal lines which have genetically been screened on their CMH charateristics. Depending on said charateristics, the lines are either resistant, or susceptible with respect to virus-induced tumours; such as Marek's disease virus. This genetic selection, performed in a first step on serological criteria, has then be seeked for on the basis of the CMH genes polymorphism study.

This is a genetic material which is completely defined on a molecular point of view, and constitutes a precious tool for studying the polymorphism of microsatellite-type-sequences.

This material, as well as the cross-product between some lines between them, was used for determining CMH microsatellites sequences which are polymorphic and for studying whether such a polymorphism can be correlated to typing data already available for said lines.

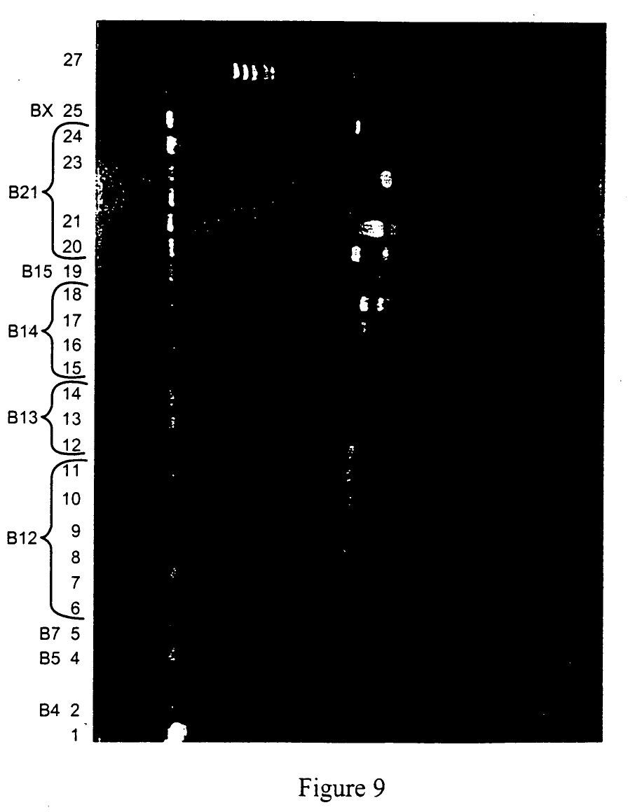

Other characteristics and advantages of the invention are explained in the examples that follow in which reference is made to FIG. 9 representing an electrophoresis photo of PCR products illustrating the genotyping test of the invention. It will be recalled that FIGS. 1 to 8, already mentioned above, illustrate the gene sequences according to the invention.

### EXAMPLE

Study of Rfp-Y haplotypes of chickens using microsatellite primers.

amplification with the Expand™ High Fidelity PCR System kit

with primers 17.5 R1/17.52

| genomic DNA: | 1 μg |
|---|---|
| take oligos: | 0.3 μM |
| dNTP: | 8 μl |
| qsp H₂O: | 50 μl |
| add 50 μl of Mix 2 whist mixing. | |
| Mix 2: | 0.75 μl of enzyme |
| | 10 μl of TP10X with MgCl₂ |
| | qsp H₂O 50 μl |

Amplification programme:

30 cycles

| 94 C | 94 C | 65 C | 72 C | 4 C |
|---|---|---|---|---|
| 2′ | 30″ | 1′ | 1′ | ∞ |

with BFI/BFIR, R:

| | |
|---|---|
| genomic DNA: | 1 μg |
| take oligos: | 0.3 μM |
| dNTP: | 8 μl |
| qsp H₂O: | 50 μl |

and add 50 μl of Mix 2 whilst mixing.

Amplification programme:

30 cycles

| 94 C | 94 C | 60 C | 72 C | 4 C |
|---|---|---|---|---|
| 2′ | 30″ | 1′ | 1′ | ∞ |

detection by electrophoresis on agarose gel or by sequencing.

The test was applied to 9 chicken haplotypes selected serologically for the B complex. These are haplotypes B4, B5, B7, B12, B13, B14, B15, B21 and an unknown haplotype BX.

Several individuals of the same type were studied for B12 (6 individuals), B13 (3 individuals), B14 (4 individuals), B21 (4 individuals) and a single individual for the other haplotypes.

FIG. 9 provides a photo of electrophoresis on 1% agarose gel of the PCR products obtained after the amplification stage.

Slopes 1 and 27 correspond to the size markers and slopes (2 to 25) to the PCR products of the following haplotypes: slope 2: B4; slope 4: B5; slope 5: B7; slopes 6 to 11: B12; slopes 12, 13, 14: B13; slopes 15, 16, 17, 18: B14; slope 19: B15; slopes 20, 21, 23, 24: B21; slope 25: BX (absence of detection for slopes 3 and 22).

An examination of this figure shows that the individuals which have the haplotype B12 give the same band and are thus very homogeneous. The same observation applies to the individuals B14. In contrast, with B21, it will be noted that the profiles are different, which shows the inefficiency of the serological approach. In view of the position of the band of BX, it can be established that this is a B4 haplotype.

The practical application of this method involves submitting the naturally resistant individuals to the protocol described above, taking into account the two Rfp-Y and B systems of the MHC, and selecting from the animals to be tested only those whose profile corresponds to that of the resistant animals.

The invention thus provides the means of verifying the homogeneity of the animals and of carrying out rigorous selections taking into account each system of the MHC and, within these systems, the genes sought.

1. Nucleic acid molecules, isolated from their natural environment, of genes coding for proteins involved in controlling resistance or susceptibility to the development of tumours in chickens, such as those of Marek's disease, and of regions related to the said genes, characterised in that they have nucleic acid sequences of genes of the B or Rfp-Y system corresponding to the major histocompatibility complex of farmed birds, with the exception of the sequences of class II B-L genes, gene 17.5, gene 12.3 and of the class I B-FIV gene, or are capable of pairing with one of the strands of a gene capable of coding for a protein as defined above under conditions of low stringency.

2. Nucleic acid molecules according to claim 1, characterised in that they correspond to one of the following sequences:
sequence of the Rfp-Y system
B-FV (FIG. 1); B-FVI (FIG. 2);
sequence of the B system,
genomic 8.4 (FIG. 3); B-FI (FIG. 4); C121 (FIG. 5), DM (FIG. 6), TAP1 (of the beginning of exon 2 at the 3′ end) (FIG. 7) and TAP2G (FIG. 8).

3. Nucleic acid molecules according to claim 1 or 2, characterised in that they correspond to a part of the sequences defined in claims 1 or 2, this part being specific and discriminating for a given gene of the B and Rfp-Y systems.

4. Nucleic acid molecules according to claim 3, characterised in that they are oligonucleotide molecules corresponding to a part of the polymorphic region of the systems of the major histocompatibility complex of chickens.

5. Nucleic acid molecules according to claim 4, characterised in that they are oligonucleotide molecules corresponding to a part of an exon.

6. Nucleic acid molecules according to claim 4, characterised in that they are oligonucleotide molecules corresponding to a part of the polymorphic region which is not associated with the function of the systems of the MHC, such as microsatellite regions.

7. Method of genotyping farmed birds and particularly chickens, characterised in that it comprises:
amplification of a sample of nucleic acid originating from the animal to be studied using one or more pairs of primers capable of hybridising specifically with the nucleic acid of a polymorphic region of the Rfp-Y or B systems of the MHC of the said birds, and
detection of the PCR products obtained.

8. Method according to claim 7, characterised in that the primers are developed from molecules according to any one of claims 3 to 6, and of any gene (and related region) coding for a protein involved in controlling resistance or susceptibility to virus-induced tumours in farmed birds and particularly chickens, particularly class II B-L genes, gene 17.5, gene 12.3 and gene B-FIV.

9. Method according to claim 7 or 8, characterised in that the detection of the PCR products is carried out by sequencing.

10. Unit or kit for genotyping farmed birds and particularly chickens, characterised in that they contain the necessary reagents for carrying out at least one PCR and the detection test, according to the method of claim 8 or 9, particularly the primers developed from the nucleic acid molecules according to any one of claims 3 to 6.


SEQUENCE LISTING (54 sequences)

SEQ ID NO: 1 - DNA, 1265 bp, Gallus sp.
ggccccgggatgccgcggttcgtgatcgtcgggtacgtggacgacaaaatcttcggtacctacaacagtaagagcaggactgcacagcctatcgtggagatgctgccgcaggaggaccaggagcactgggacacgcagacccagaaggcgcagggcggtgagcgggattttgactggaacctgaacaggctgccggaacgctacaacaaaagtaaaggtgagcgtgggggaagctgcagcgcgatgcgtctgggacaggagctctgtgtgccgagggtgtccgccagccccactgaggtgtggccgtgccccacgcccagctgtgctgggccgtccatgtgtggtggcactgtccctgggccgccctgctcctgcgcccacccaccccaccccagcctcatggcactcgcggtgccccacagccctagaagcctctcacctattactctggctgtgcctcagggtctcacacgatgcagatgatgtttggctgtgacatcctggaggacggcagcatccgagggtacgatcagtatgcatttgatgggagggacttccttgcctttgatatggacacgatgacgttcaccgcggcggatccagtggctgaaatcaccaagaggagatgggagacagaagggacgtatgctgagagatggaagcatgagctggggactgtctgtgttcagaacttgaggagatacctggagcatgggaaggcagcgctgaaaaggagaggtgaggatgggagggggacgtggggctgggctgggtgtggggcagaggctcagtgtggggtgctcagcccggcccacaacgtcacccacctgcagtgcagcccgaggtgcgagtgtgggggaaggaggccgatgggatcctgaccttgtcctgccacgctcacggcttctacccgcggcccatcaccatcagctggatgaaggacggcatggtccgggaccaggagacccgctgggggggcatcgtgcccaacagcgatggcacctaccacgcctcggctgccattgatgtgctgccggaggatggggacaagtattggtgccgcgtggagcacgccagcctgccccagcctggtctcttctcatggggtgagctggcagcgtggggcacgtggggttgggattcgcaggctgccccttcctttactgacaacggcgctctcctccagagccgcagcccaacctgattcccattgtggcaggggcggtcgttgccatcgtggctgtcatcgctgcggtcgttggatt

SEQ ID NO: 2 - DNA, 1266 bp, Gallus sp.
ggccccgggatgccgcggttcgtgatcgtcgggtacgtggacgacaaaatcttcggtatctacgacagtaagagcaggactgcacagcccatcgtggagatgctgccgcaggaggaccaggagcactgggacgcgcagacccagaaggcccagggcggtgagcgggattttgactggttcctgagcaggctgccggaacgctacaacaaaagtggaggtgagtgtgggggaagctgcagcgcgatgcgtctgggacaggagctctgtgtgccgagggtgtccgccagccccactgaggtgtggccatgccccacgcccagctgtgctgggccgtccatgtgtggtggcactgtctctgggctgccctgctcctgcgcccacccaccccaccccagcctcatggcactcgcggtgccccacagcccaagaagcctctcacctatcactctgactgtgcctcagggtctcacacgatgcagatgatgatcggctgtgacatcctggaggacggcagcatccgagggtacgatcagtatgcatttgatgggagggacttccttgcctttgatatggacacgatgacgttcaccgcggcggatccagtggcagaaatcaccaagaggagatgggagacagaagggacgtatgctgagagatggaagcatgagctggggactgtctgcgttcagaacttgaggagatacctggagcatgggaaggcggcagtgaaaaggagaggtgagaatgggagggagacgtggggctgggctgggtgtggggcaggggctcagtgtggggtgctcagcccggcccacaacatcaaccacctgcagtgcagcccgaggtgcgagtgtgggggaaggaggccgatgggatcctgaccttgtcctgccacgctcacggcttctacccgcggcgcatcgccatcagctggatgaaggacagcatggtccaggaccaggagacccgctgggggggcatcgtgcccaatagggatggcacttaccacacttcggctgccattgatgtgctgccggaggatagggacaagtatcggtgccgcgtggagcacgccagcctgccccagcctggcctcttctcttggggtaagcctggcagcgtgggatgtgtggagttgggatttgggggccgcccctttgtttactgacaacggtgctctcccccagagccgcagcccaacctgatccccattgaggcttggctggtcgtccccttggtggttctcttcgttgctttgattgcatt

SEQ ID NO: 3 - DNA, 3861 bp, Gallus sp.
ggatccggggtgggtggcagtggctgtgtttaggtcggcctgtggggaaagccgggttgtcccacccatgtcccctcttccaacactgttcctgaatgagttttccctctccgaccctttttttaatgggtttcagggatttaaaattaatattgacgaagtgacggagggggtggggccacagcggagccgaaagcgaaagcagcggagagcaatggctgcggggctgcggctgctgctggcgggtgagacccgaccccccccggccccctcatgtcccaccacccatatcgcccccccccctcctcctcgccccatgctgagcctctcccccacccccagggctctgctggtcccaatttagggtggaagacgccgcctcccctccgcccccccccgctccggtgcgctgcgcgctgctggagggggtggggcgcgggggagggctgccgggggggggcaatgcccgtcctgcactgctgcgctttgggggggacgcggagacccctcccgaacccggcccggagcccgaagtcaccttcaatgtcagcggtacgtggggacccccgtcactgtgctgtgcgcctcctttatccccacccccctccatgtccccatctcctttacttcccacaatgctcccatcccccccagaatgtccccagagtcccccaaacccccatgaccccccccacgacccctggttcccattaccctctcacgtcccccagtgtccccaagattcccattactccccgtatccccattatccccaaaatgtcccccaatgttcccatcaccccaatgttcccaaggtccctatcgctcctcaatgtcgctatgatccctattcccaaaatgtcaccaatgtccccaaaatccccattatctcccacctctccaaagtccccaagatccccattacccccaatatcctcattacaccccaaatgtccccaatgtcccctccatgtcccccagagaccccattagccccaatagctcccaaactgtccccagtgtccccattaaccccaaaatgaccccattacgccccacacccctcccaaccccatgccctcagaccccttcatccctctcactcctctctccctcgcagacccctgggggactctagccccactcgggtccccccccggactccccccagctgcgaactgaaccccacgaacccccagaccggctctgacccatggagccgccctctgcaccccgacgcccgcagccccccaaccgcgggggggcagtggtgggtggcggcggtggggaccccgcagtacggtgtcactgcgctgctgcagggggggatgggcacagaaggaaccatcactgccgccggtaagggggaacttggggtgtccctccctgggtgtccccatgtccctatctgtcccccagtgtgtccccatttgtcccctcctctgcatgtgtcccaatgtctccatacatcccataataaccatatgtccccactcatccccatattccccatgtgtccccatatccccacacatcccagtgtgccccaacacatccccatgtgcccccccccatgcatcactaccatccccctatcccccaagtgtccctgtgtccctgcagtttctccctgtcctcatgtgttcccatgtctccatgtcactgtgtccccgtgtccccacacatcaccatgccccccactgcagcgcccccatgtcccttcacctctccatgtcccccagtgtcccctatcccctcattgtccccatgccccctcacctccccgtgtcccccgtgtccctatgttcccctggtgtttccatgtcccctcatgcccccatgtcccctcatgtccccatatcccccagtgtccccatgtcccttcacctccccatgtcccccaatattcccatatcccctcacctgcccatttccccccgatgttcccatgtccccgcacctccccatgtcttcacagtggccctggcggtgctcacccacaccccgaccctccgggcccgtgtggggtcccccatccacctgcactgcgccttcgctgcccccccatcctcctttgtcctcgagtggcgtcaccagaacaggggtgcggggagggtcctgctggcctatgacagttccaccgcccgcgccccccgcgcccaccccggggccgaactgctgctggggacacgggatggggacggggtgacagcggtgacactgcggctggcgcggccatcaccgggggatgagggcacctacatctgctccgtgttcctgccccacgggcacacacagacagtgctgcagctccacgtctttggtgcgtccatgtggggcaggcggtgttcctatggggtgtggggttgggcagtgttcctacggagtgtgtatgactgggtggtattcctattggtcagataggacatatgggagcaggcggtattcctatggggctgtagggtggatgggactgggtgatattcctgtgggggctgtagggtggatgggactgggtggtattcctatggaggctatagggtggatgggaccgggtggtattcctatgaggactataggatggggtggcatcatcccatagttcacctgtaggtttatagggggggatgagccctatacagcgtatgggctatatggaccgatgtccccccacatgtctccagagccccccaaggtgacgctgtccccgaagaacctggtggtggccccggggacgtcagcagagctacgctgccagtctggcttctaccccttggatgtgacggtgacgtggcagcgccgcgccgggggctcggggacatcacagtcacccagggacacagtgatggacagctggacttcaggtcaccgccaggcagccgatggaacctacagccggacggcggcagcacggctgatccccgcacgcccccaacaccacggggacatctacagctgcgttgtcacccacactgcactggccaaaccaatgcgtgtctccgtccgactgctcctggctggtgaggggggatgtggggatattggaaacacgtggaggtattgggatgctgggaccatggttaggagggtctgagggacatcaggaccatggcctgggacaatgggagatcatggatttgggttggggaccccacccaggatggtgacactgtgcttagggctgtcgttgtccccacaggcaccgagggaccgcacctggaggacatcacggggctcttcttggtggcctttgtcctctgtggcctcatccgttggctctaccctaaaggtgagtgctgttcccacatcccagtgcccccacatcctcacaccccaatatcccaatggcccatgtccccatgagcaatgtcactatgtcccaatatcctaatgatgctgtgtacccatgtgtccccatgtccctattccactcactctttctctcccctcagctgcacgacccaaagaggaaaccaaggtaacattcctccccaaaaaccccaaatcccccaaaacacctccaagcaccccaaaactcaccattctcattccccccccccccccccccccccccatgccttgcagaaatcgcagtgacctccactccagctctcagcacctcagctccagataaagagtttttcaccccaaagttatatatgtgtggtggtgtccccacagatctgggtgcagaggggggagaaatgggggcaaactgggagcagtgggagcagtgggaggaagtcctgggttggtgaggcagatgagtggcacctggggacatctgggtgccatcccttgtggacatctgggtgacactgcattgccttgggtgacattgggatcctcaggtcactgcag

SEQ ID NO: 4 - DNA, 3740 bp, Gallus sp.
gtcgacgggatctggataggtcgtcagtcatcctaattaaggagggacaacagtgaatggggaggagccgatgactcaggctgggagtggtgatcccagaggtttcctctgctgtcagtgactccgtgctttcgctttcgcttcacaacctgagggagcgcattctgcctggcgcccgatgacgtcacataaacccccgactgccattggcggagaggcgacggaggagccaatgggggcgcggggcggggcggaatatcattagcaagagagtagacgtacttactagatctatccctattagttaccttattagttacctattagttaccttattagttacctattagttttaccttattagttttacctattagttttaccttattagttttacctattagttttaaactaaggtcgacgggatctggataggtcgtcagtcatcctaattaaggagggacaacagtgaatggggaggagccgatgactcaggctgggagtggtgatcccagaggtttcctctgctgtcagtgactccgtgctttcgctttcgcttcacaacctgagggagcgcattctgcctggcgcccgatgacgtcacataaacccccgactgccattggcggagaggcgacggaggagccaatgggggcgcggggcggggcggaggagtaggaaaagctgaaggacgtgcgctgggtgcggcggacttgagagtgcagcggtgtgaggcgatggggccgtgcggggcgctgggcctggggctgctgctcgccgccgtgtgcggggcggcggccggtgagtgcggccggaccgggacccctccccgcctgtaaccccaccccgggctgtgcccgtgggatcctcagacccccacccgcggctcacggcctcgctgccgtccgcccccgcagagctccattccctgcggtacgtccatacggcgatgacggatcccggccccgggctgccgtggttcgtggacgtggggtacgtggacggggaactcttcgtgcactacaacagcaccgcgcggaggtacgtgccccgcaccgagtggatggcggccaacacggaccagcagtactgggatggacagacgcagatcggacagggcaatgagcggagtgtggaagtgagcttgaacacactgcaggaacgatacaaccagaccggcggtgagcacggccggggccgcggctccgtgggtgtgggatgggctccatggcgcagtgccgcccacaccccccaggcctggccctgcccggcggcaccgtcccggggctgcccgtcacagccccaccgcgctcggggtgccgcgtcccggggggaccccaacccatccccgctgcagtgggagcccggagccggaggggcccctcaccccctgcccggctgtgtttcagggtctcaaacggtgcagctgatgtacggctgtgacatcctcgaggatggcaccatccgggggtatcatcagacagcctacgatgggagagacttcattgccttcgacaaaggcacgatgacgttcactgcggcagttccagaggcaggttcccaccaagaggaaatgggaggaaggaggtgttgctgagaggtggaagagttacctggaggaaacctgcgtggaggggctgcggagatatgtggaatacgggaaggctgagctgggcaggagaggtgagcggggtcggggtgggggggggggggggcggacgcagtgtggggtcggacgtggggcgggggctcatcgtggggagctcagcccggccctcactgccgcccacccacagagcggcctgaggtgcgagtgtgggggaaggaggctgacgggatcctgaccttgtcctgccgcgctcacggcttctacccgcggcccatcgccgtcagctggctgaaggacggcgcggtgcggggccaggacgcccagtcggggggcatcgtgcccaacggcgacggcacctaccacacctgggtcaccatcgatgcgcagccgggggacggggacaagtaccagtgccgcgtggagcacgccagcctgccccagcccggcctctactcgtggggtgagtgaggggatgtggggctggggggctgcgggctgccccttcccctgctgatggccccgctctcccccagagccgccacagcccaacctggtgcccatcgtggcgggggtggccgtcgccattgtggccatcgccatcgtggttggtgttggattcatcatctacagacgccacgcaggtaaaagcagaggggtgcaggcgggcagtgggggctgtagggggatctgggtccccccttgggagcccccaacctggctgtgatgtgaacctgtgatgaagcatctctctgtctgcagggaagaaggggaagggctacaacatcgcgcccggtgagtgatgagggcagcgctgtcccccacctctgcccagtgccagggtggtcctggggtccctgctttctcccaaggtacccattcctggtgcttggggctgctccatgccccatagggagcacagggctggatctcacagctgttcctcccttatagacagggaaggtggatccagcagctcgagcacaggtgcggtgtggggctgtgggttgggaggggtccgtgtgctctctgtggtactgcccagggctgggctatgctggggctctgcggggagacccccggagcagagggttgggatgtgaacctggccccgtgggacatcatcccttctcatcccacagggagcaaccccgccatctgagtgctgtgcttcagcctgcaaggagccaacagtccacaccagcatttggggtcggtgatggacacagccccatcctcctgacctctcacatctcattctgcttcctatgctgactgttatgctttgcctgcactgcttcctgtgaaataaaatgatgggccattctgtgctcagcttgcctgcattctgcacagtgctgtggttggggatggggtgggtgagaggaccgtgtccagtttggctgctcagggtgcagatgtggccctgtgctgagtacccactgccctccccccctatctgcctgctgctcactccccctcctgtacccccatcccttctcacctctcctctgtgaaccccatgctggtggttgcttgctccctgtcctggcagaactctcattttcccaatggcatccctgggtgttgggatgtggtctccttggtcctccccccagcagtcactgcacatatcccccccacttcccccctaggttgttgtcccacagcactcctatttccctctccccccccccccccccccccccgcccatccagctgcctctgcaatcctcacccttgcccacacacaactttgcgcactccacctccctcatcccgcccttcccccagctctcctgtccctgctggcccccctcccccccccccattgtaccctacacccaaataaatatgtttgttctgctgccctccagcggtctcctggtttatttccccccgatttgttgttgttggggcgtccgctcttcaccctggggggaaggggctctgggggtccctcattctccctgcacttcttacagcaccgggactcccgcgctgagatcccatcacacccgggtacaaacatgcggctttattcccagttctgtgtcccacccccggccctggtggcactcagtggcaccgcagtccatgcagtggccgttgtgtgtcgtacagcagcggtacc

SEQ ID NO: 5 - DNA, 43228 bp, Gallus sp.
  modified_base (1)..(43228): “n” represents a, t, c, g, other or unknown
atatatgagtaaacttggtctgacagttaccaatgcttaatcagtgaggcacctatctcagcgatctgtctatttcgttcatcccatagttgcctgcaactccccgtcgtgtagataactacgatacgggagggcttaccatctggccccagtgctgcaatgataccgcgaagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcccgcccgtagcgcgcgcgcaccagccggcatcgcacccgagcaccagctcccccgtcgtccagatgcccacgggccacgtcgaggccgacggggagaaatacacgtacctacctggggatctcaacaggccccgggtggccaaccaggtcgtggacgcgttgtgcaggtgcgtgatgtccagctccgtcgtcgggtgccgccgggccccaaccggcggtcgggggggcggtgtatcacgcggcccgctcgggtggctcgccgtcgccacgttgtctccccgcgggaacgtcagggcctcggggtcagggacggccgaaaacgttacccaggcccgggaacgcagcaacacggaggcggctggattgtgcaagagacccttaaggggggcgaccgaggggggaggctgggcggtcggctcgaccgtggtgggggcgggcaggctcgcgttcgggggccggccgagcaggtaggtcttcgggatgtaaagcagctggccggggtcccgcggaaactcggccgtggtgaccaatacaaaacaaaagcgctcctcgtaccagcgaagaaggggcagagatgccgtagtcaggtttagttcgtccggcggcgccagaaatccgcgcggtggtttttgggggtcgggggtgtttggcagccacagacgcccggtgttcgtgtcgcgccagtacatgcggtccatgcccaggccatccaaaaaccatgggtctgtctgctcagtccagtcgtggacctgaccccacgcaacgcccaaaataataacccccacgaaccataaaccattccccatgggggaccccgtccctaacccacggggcccgtggctatggcagggcttgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctcccccatctcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaagnnntggggtcctctttggtctgatggagagaggttggcaccagggtaagtcgctgcctacatcaccactggtgttttgtctcagcagctggtgtaaatttctgccatctgggctatttctgtagaaagcaaagaagctctgctggtgggcagctcatctcccagtgtgaaaaagcaaaatgcaacgcatgcaccctgctatccatgtggbccyakccctctccatcagctgttgaaggagaaatctgcactcagaagagattgaattgggctcagatctggcttgggaagatgatgattccaaccagagtccaggagactttggggaatgcatgaatcctataggaaaatggataacccttcatccaagagcaagctggcatgatgctctggggtgaaaacccataatgccacctggttttaaggtttggggtggcttacaatgtgcagctctgcttccggcgaggcactgggagccctaaacccatggagaggtcaaaccagtgctggaggtcattgtgggcccagctgcaatgggaggtaggcaattatggacatcgctgaagccaccccacgctctggggaacttgggttttcacctttcactgcactttaatgggatttctcatcaatgtctgcatgttcttggccacctgtttaaaaatataataataataattaaatcttttgccccactgcgggatgagcagctggtggttcccagctcacaataaaccacacttgagactccctggagaattcgctttctttttgcagctggttccatgtkgggsykttcagcccctctgcagctcataggcttttcttcacagcctctgctccacctattgctgaaaagggggaaatttgagatggatcccattttgtgaacatctcccmacctgtgggtaatgctcagacctctcagccctgtgggtttaatttctctttctgcagcttaatgggttggggatgttcattactgcaataattagtgatgggataggggaggcaggagaggatcccgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgatcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtccataatatgccattagaagtaacacatccatcaatgatatatccatagaatacaagagaacggtctacatttacttcagatcccattttcaggttaaccatgaaaaaaatacccaaagactgaatgtcaccattcagggatcccgtgtgtaaaatcatgacttctgctttaattataagaaaaatgaaattcactgtttttattctcttttaagatgaactctcaacagaagttggtgagtatttttctgccctccagcaaaaccaaagcatgcagtttgcagtctgttttggatatatattgtacgtggatatataacctgtatgttataacacctctggtttccttttctccttcttttcctcagaaaaacgagagagaagaattggtgagtatcaaacttccccccagaagtggactttggtgtgttgggaagatccataccacaacgttggtgccaaacttaatggaaatcctttgttttttccttatgttttcagatgaactcactgcagagctcggtaagtcgtgattataactcataacgagttataatgctattgttatatataatatacatattatatattgttgctataattcataatagagcaaacaatcacaaggcacagaaatatgggtttgctttgagagccaaaccttaggaagtgataacacaatgggaagaggacaatgaccatttctgttgttcctcttttcagagcactacaaggcaaaagcaagtgagtgtctccttcctcatcttcagcacgtgagagattttgggggcttttgggacggctatggggatttacacataataaaacagaagatgagaagacagtttgttaacttgaattcaaactggtttgaaattggtgaaattacagtataaataatctccccagtacccaattatacaatgggattaattacagcctgcccaggaaaggagcactgaattttttcctgcgtccatccagcatgaagtccatcagacttaagcttacagcttaaagaatggttcatttttttcatttaaccccctcgtaagttaaaagatggacttcagcatcacagaagtagcccagaaatagtcaaaaaatgggtcatgaatttccagagcacccccccacactttccttggtgaataggaaaacaaatattaaaactaattaattggtttttttttctttttaggaagatgttttgaggaacacagtaagtgcccttttctcccttctttaagcatcacttttcactttaagtctgcatcacagttaataatccatctccttattatgcatttttagggagaggcgaagaaaagttgggtaagtcatttggttaattgggtttctgcttgcagaccccatccaggagctcatgtcctcctcttagtgtctgcactgtagaaatatccaggttagacgtgtaggtaggaaatactggacctgcgtggaggtattgcagaccccatttatgtgtaggggaagcagaacatcaaactattgagccttgagctccacgaagacaagccaccctcttagatttcaagcgaagtcgagctgaatagatttaattctttctttcccatagtaaatgtgactctggacccagagacggcccaccctcgcctcgtcctctccaaggaccagaagagcgtccgatgggaatacagcctgcaggaatcccccgacggccccgagcgcttcgacgccgatccctgcgtgctgggttgtgaaaccttcacctctgggaggcactgctgggtggtggatctcacagaagggcagtactgcgccgttggggtcagcagggagtccctgcccaggaaaggagccgtcagctttaaccctgatgaaggcatctgggctgtgcagcaatgggggttcaagaacagagccctcacctcccctccgaccccactgaaccttccacgggttcccaaaaagatccgcatctctctggactacgaatggggcgaggtggcgttttttgatgtggagaaccaaatgcccatcttcacttttcctctgacctcctttggtggggagcggctccggccgtggttctgggtggagctgggctccctctcactgcccagataaccccggaatccctggaggtgctgtggaggtgccttacagcagctcttccagaccggggtggaaaaactctcaggaaaagcagcattaaaacctcattctccctcttcccagtcaaccattgtcatgcaaaagaaaggaaacccatcctcaatgtcatcagcatcctccgtgtgtcatgtctggtggcccccattgatgtatggggtggctcctgttggtgtctggtgccccctattgacgtatgaggtggcccccattgacgtgaggtggcccccattgacgtgaggtggcccctattgacatatggggtggctcctgttgatgtctggtgcgccccattgacatgaggtggtccccatgaccagcccctaccctggatccaatgccwcmygattgcagttccaaactctagggacgttaaacgacccacagagaggaaatcacctcaaaatgagcctgaatgtttgcactgaggactgagcacagctgggcactaattcatctttatttctctcttatttacagaggaacgcgatctgaaaatcagtaagtgctgccccaaagccatagggctatgctgggcttcatccccacaacatgaattttataaattaaataaataaataaataaataattttatattttatgtatttgatattagcagtatttaaaaaaaagaataaaataactcaagaatcttaggatcaatagtaacacaatgatgcaacgtggatacaaaagcagtaattcctatttctttgggtttttatccttccaggggaacacgaagcagagatacgtgagtgttattttatatactctataatggaaaacttttttctctgtaatataaaaataggctttattattkgaggggttttttggcttaacgcaaatgcgaagtgcttgaaattctacgtatgaaatagaggatttcccatagagaaaaacagcaatttggggctggaataaaagtttcatttccttgctgaaaagtgaatgaaaagggggggaaaagaacataaaaattgagttttttccctcattaatctgtcatgaaatgggttgggttcctgaatggtgatgtcaacacctcgttttgggttcagcccaacataatatgtgtctgtcctttatttctgtatcactggtgttaaagagagctgttttgaactaatatctcttttttaattactttttctttttctttttctttccttttttctcccgtttctctctgttttgctttaagggcgcctcactgagctgctcggtaagtgcatttccttccttgcatctgtsaawmcagcwataaccvhaggycctattttgggggggaaggaggggataaaacacaataatgatgaaatcagtgctttggaaagggtgcaattattatttctcctgcaaatgaatacttccttttcccttttgtttgcagaggaccgcgattccgatgtccgtaagtccttttgtttgtcccggagctgtgaatcctccaatgggaaatgcagaatttcagagtctgccccaaaaatgacctttttgaggctacaagggatgggaaaataaggagaaatgtccttatttattgatctccttgtttatgtgcaaaactgggtgactcttctctgccgaacacgttagaaataagaacacaaaatgggaggaaatggtatttattcatatctgttgtttttctgtttaatttttaggagaacaggacatcctcattagtaagtggcactttggattgataagaaatgcagctcctggggacgtttgggtgctgcgattgctggcactgctggggctttgtgttgtggtggaagtggaattacttcaaaagaagagaagaatggaattatctggagaaaaaggggaataaatggaactgtttgggaaaagaaggaggaatagaatggaaatattggggaaaaaagtgaaatagaatggaattatttcaaaaaaaatggaatgaaatttagggagggggaaggggaagtggaatggaattatttgggggagaaaaaggggaaaattgaatgactggggggggaatggggaaataggatgggaktwttttaaaaatacagaattgtgaaggtttcagcccatctcagagagtttggtatcctcgagttccccctttgcaacccattgagcatccttgggatgacaccaaattctgttttctccttttcaagggaaactgtcagaagagctcggtgagttatttccacttcttacatacaaaactgattctggataatccttttgtgtgttttcctgctttgcctctttgtgttttaagaggcaactgcagaaggaatggcacaaagggtgcagaggatctttgggataaataacagggaaaacagggatgggatagcaatgagttggtgcaataatctatggcacaaaaggtgacggcgtgtttcacattttgctttttctcttccttttagaggaattaaggggtcgggaagttggtaagtgagattcctttccctcttctccccaaaaggataaggggtaatttggattctgatctctttttctccctttttgttcctagaggagagtgttctggagaggggtgagtatcattctctttctactgctgcttttgactgaaggaatcccccataagcatgctggtgggatgggaattctacatctgatacacaattattatcatttcttcattttttatacacagaaatagataatttttttcctttcttttctcttttcccccttttttagaggaacatgatgccagaattggtacgtgtccatctccccctgcttttgtggtgtcttcaagaaggccaatggggtcatttgggattgtttgggttgaggattgggttcttgattgaatttgggggaggattcaggtgcccaaacacaacatcaggtcccatctcatgttttcctatgggcttggatccttctgttggatacctaagaatacctgaaattcctgggggcaagagggcaccgccgtgtaaaaaawacatatatttaaccatttttccttctttttttcccatttaggggaccgtgactcaaagctccgtgagtgccactctcctcctgattaaaatctgagtgaagatgtggattttcctcagtgtgctcctacaatctcactttttcagcacagttttccccaaantttgtgtttctccacccaaccccttacactgatcctaaatgggtgtattgcctgaatcagtggttttcttccctattttngatctatccngttttattccagtatatgtttttatgacataattttatgacatattttgttgtatgatgcccatagaccttattaccattgcctgcccctgtgtggatcagaaaatatatttaatataaaacagatatctctactgacagtgatttctgatgcacccatgaaggaaaaggatttaaaataaactttaatttttccctttttaggcaaactgacagcagaactcggtaagsacmtkkycktcsycattcccataaaacaaatgaaattatggatggatggatggaaattaaccagttagtagaggtcagctttgctctaggacggtctgaaaagtgaccaaaatctgcttttactcatttttcttcttatttttttgtagcaaagtgcgatgcaacgatcagtaagtgctgctgcatgtgggggtacctccatcttcgggtcattttctgctgtttcagcattgaaaggacatcagaattccttaaatccaacaaaattggggtcactcgaaaggaaatctttgcagatatgggggaaatcagagccaaattttgagggggggagggaaaatctcaggggtgtttcagaaatccaatgggatctgatggtattttctgctctcaggactgtttacagtggaactcggtgagtccgtttcctttttgttttttttttctaattattatttattagtagtattataaatcaatattactgttgcttatacatattgttgtacattatatacataatacatacattatatacagtatatagtatacagtagtatataatattatgtattatatatatataatgtattataataatgtcatatctaatatatgtctgtattagatataatgcatatatattattgtactacagtcatattataatacatttacttatatctgcctttttccacacgtttcattgacctgattaaaaactaaatcctaaaggcagaagaagatgaaaacccccaaattaacaccaaataattgcagctatagatcatatctatcaaaagcaaatttgccttcagtccacatcacgaaattaacaatagaaaggtttaaatttggaacgtacaaacaatgacaaataacccccaatggcttttctcttcttgcaggagagcgtcacaccaaaataggtacgtgaggtgtttgctaccttcgtttggaaggaagaaattgcattaataaaacctctgtccaatatgaagccggggtcaaattactcataaatcaccactgattgtccatgaattaacagggaaaaaaaaggctaaacttgaaaataacatttttttcatctctcttttaaggggaactcactgcagaagttggtaagtctctttcccatcagtttaagcaaaaatggttcatcagatatataataatcccttatttctgcttgtttttaggggactacaacaggaaacttcgtaagtgcctttaacttctcccattaagagttaaacctttcaatatttttgatgcttcaatgtgctgaagccaccaaaaatgtgtttyaattgtaaaggggctgagcgtcaaacctgaacactgccatgttgggggctgagattcgtgggatttgggttttcagtgtgaaaatgcctctgggtttctgtgcctgagctcagggaaacacgaccagggcttcccagtaggaatgagaccccaaaatatttctacctggggscttttcccattggggaatttattctgtwaatccatatttctccmcstttgarcgtymctcmtcmaatgtcacaatcttggcaatgttgagaagatatatagatatctattttaatactgattaatatggaggtgtttgtgttggtcagtgatgtcatcgggaaaagatctgagtcattgaatccccatttcttttctctttattttaagggaaacacgcagcagaacttggtaagggaattccctccctgggtttgttctcttgtttttctcttttggagggggattttttctatgtcttctttctatgtcttctttctatgtcttctttctatgtcttctttctatgtcttctttctatgtcttctttctatgtcttctttctatgtcttctttctatgtcttctttctaagtcttctttctatgtcttcctttctatgtcttctttcyawgtcttctttckawggtcttctttcyawggtcttcyttcywwccttctttctttccttctttctttccttctttctttccttggattttgagccaaaaatagaagaatgggatgcaaaaatcagtgagtgcccttttttcctctcccttcacggtgaggtatgggtgtggaggacctgaattaatgtgaattcctctgttttaagggaagctaacagaagattttggtaagtcgcttattttcctcgatctgagtgcatatttctacacctttaccatcagtgatgaccaacgtgtgtatgcatttctctttattccatttagaagagagcgacacagagctcggtgagtgctttggggtcttatcaaggtggaaagatgcccctctgtgcaacagtggggattgggagaagcccttcagctcttccatttatccacatctgatacccagatggagtcaggatgcagaactggaggaggagggccaaagctttgggcattttggggttatttttgttcctcgagagctcccaggattgacccgtgtccatttctgtgttatttccagaggaatgtgacacagaagatggtgagtgtcctccgtgagagggctcagagaaagacttccaccaaatctccctcctttaatgtatattctgatgtatttatttaaggggatctcgcasctgagatcggtaagtcgtgtgtggttatacacccctatktgtgcctcccatcaaasagggctctgtgcascttgagtkgtgttcccacagggtttgtcycccactcttcacacgaatatgggggtaaaacccaacaaaatggcacagagggattgcagaaagggcgggcgttgggtggcgctgtgttctgatccaagggagggtgaagctcatgagaatggttctttctttctctttttgaagacaatctgactgcagagctcggtgagtgcttccctttcctctctgcttcgtttcactgttgggtttttaggggggaaaaatgcttattccccccataaacacacacatgtaacccaacctgggctggaagaagggtccaaacgttcataactgcagactgcaattatcattcccaattggaaggtgattccatcatgaaccatccacccatcacagtggaattctgacagtgtttctctctgttttccctttcagaggaacgtgataggaaaatcagtaagtgcctttttccttccagaactgatgggaagcgatgggttagggttagggtaagggttagggtaagggttaaggttaggcttggggaaaaataagttaatacatttcattatggcttagaattgaaactaatgttcatctatttctttgttttaaggaaagctcacatcagatcttggtaagggttacttcctttaaactatccttaattctgcaacagtgctgggtatagagtagaaaaatatgcatgtgaaggtgtatgtatgcacatgttaattcattcctatttatgtactcgttagttgctatatatgtattaatttattcacattatatatatatttgtatatatttgcaaatatttgtatgtatgtgtgtatgtgtgaagagattggggtttcctctggttgaagaggggggtgaatgacagcaggtgtccttaataagccttattttcaaaacactaacaagggagaattgggatacacagaaataaagcctaaaaatgggaaaaagaaaagaatgaaatgggtaaaatattgaaaagaacraaaartttggagaaaagaaatgacasttttggttgggttggggctgctctgcatttctccrcttattttctccctttgctttcaggtgatgttgacacaaagctcagtgagtggagctgctcttcctgccccacatttaagagtatttttggtatttttaagactgtttaagaatatttggacatttcctgtggaaaatggatttctggtctgtaaaaaaaacctggggcttatttttgaggacggaataaatgtcccaaaaaagggggattttggcatcaattgactgggaggtgaaaaataaaagcagtgatctgagcgtgttggggccaatggatgaacctcaatgatcattgtggtccttttcaatccaggccattctatgattctgtgaaagaaaagaagataattaacatttaattttcttctttctcttctcattccagaggaacgcgacaggaaaatcagtgagtgtcacttttttggggccaaaaccctctgatttggggaagggatccctgatagaagtggttaatcctgttggtttttccctccttgcagccaaactctcagcagaaatacgtaagtccttttcctccccaatctgaactgtttctttgtattcttagacttccttttttttttttttctgttttaattaaaataatgcttttttttggttggttttttttttccctatttgacaggcagactgactgcactgctgggtgagtggtgccattaaatccgtgtgtggttttgggctgaaaacccttaaaaatgggaactctgcacccagacagmyyatsgtcwkwgctttkyywcatttgtaatagaataaaaatggggggaaatgggcaaaatgagcattgcaagkgagcagagytgctgcccaaaataggcatgactcagcaagcaccgtagtgggcatgatttgcttgggtgaccccgtgggtaaggagccatttgttggacaccacgatgtcgtttttcacagccctgtgagcgcagcgtcttaaattgccctccagacattccaaatttggagagctcaaatggcaaaggtgaaatgggcgtcagccctccgggatgaaggaatctctgccggggttttccgttggatcacagcaggaggatttgctttcctaaagcattagagtgacgtggagagcccaaatcggacccagtggccacattctcccaagggaaaacccttcgggtgcccctacggttccttttctagcatgataacaaacttcttttccatccgcccatccccttttgggtttggaggttgacaaatccccactgaaattcctatgttgcacacatgtccttcattctttaagtaggagttagcaaaggttccgcattgacttaattcagagcgagatcaacaattttaggcattctttatgaacttcacattgttttatgctgatcagcagcaaaaaaacatacaggaataggagtgtgtctgtaggagtgctctgcattttcttgctcgtttggctgattaaggaagctgggaggaaatgttgtgaaataatcccaagtgatgagagactgtgggtatgggaggagatgccctctgtcctggtgagcagtagggacagaagacctgagctcatttcatatatctgtatattaaggcaatgctaaccagtgctgtcgtgttatttggggccaggagtggcttctgcccccgttggtgcccataaaccagtgctgccccattkgggattggggttntgttggcagacaacatccaccaaccaacccatggctgatagcagagaggcgaccaggtcaaccctccatatatctctgcagaaacctgttcctgtctatacagggatccccatccctcccccagccctccttccatcctcggcatttgggttggctataattaggctctgggaacgttcccctgctgccagcacagctgtcgtgtctgcaatgatccttccagctctctgcggacacgcaaaccctccagcaatcctaaatacccatttcctgcactcctgggacaaactgggagctgccaaaaatctccagccccccacagacgtgaccatcacagcaccaaggagcagagcaagcgcaacgtgattacggtgcaggtcggggtaagcctttctctttcttcccacagcccaggatttgggggatcctattggctctatgggatctgggagatgcaggagaaatgtgatccctttgctgtagcaaaacaaccttttagagtcctgcacctgaatctggcagtactggaaagcaggagagggattaagagtccttctgcattatcctgctcatagggaaatacagcacagaaatcattggggctgcttcctttgctttcttggcacaaatttaggtcctcattacagcgtttctttgactgagaccccaataggatctacaggggtagaacaaagcagacaaaaagtgattgatgtttcctatgcgatttgttgccttttcccattgagatttctgcttttcctatggggctttttgctttttcacagctttttttattcactgtagtgaatagaaatttttagggcttttaggtcattgatgctgttatgaacacagagatgaactcataacaccttcctggtgtggtttgtctatgggatagaaaggagctcatggtgctgtggacaactaacagaggtgcctgagggctgggccctctttgtgccccttctgggggtcagcaaactccttttattcagatataaatcccctcatccacaatttcaccagtcttcccaatgcagaccccaaaaaacatccccaatgacaaagtccacgaactgagaaaagcagcaaaaagcctcccagccccaaatatttatcccttatcccatttatttctatgggcaaagctattctaggcatcaggaaggtgggagattccaggtcagtttgttcctaattgtgatcttttaatgatgtttctcccatcaggtggacatttggaagtggttctgactgggaagaggacgtgatgatggcatcaggtagagctcagaaagtggtatttatcagcaaagcaattttccaggtctgttttttcccatttttcccatatttttttcttattcagggaagaggaacgcggatcttggtgagtgattttcttccttttaccttcaaaaagtccctttccatgtgtagaaatggatatacgtaccccccactgatacccatttcctttgttctgtccttatatttatacttccccatattttgaacacatgaaaacaaagcccacattaaataaattcataaacagtgcaatttttggactattattttccatagaaaagtattaaatcagtgcagaagtgcctctggaggtgacttctgcagcacccaaagagagaggcgtagggctgagtgctctctctgtctctctttgaaagaggtttattttcacagtgtggaaactcagatccgttgcctcacctgcaccgtgtatttgcagacacccaaagtgttccagagtttgatggttttgtccctggagcccgaaacgatctggcggttgtcggaggagaaggcgacgctcagcacatccttggtgtggccaacaaagcggcgggtggtggttcctctgcagggacaccaggagggtcgcacgggagggacaaagctcagcaaacccccattaaattaattaaccctcccctaaattgaggagatcgtgctgcagtgcataaattcttaatgaacacaactgatggaagcaggaaggaagctaaaacggagtcatctccacatgggttgaggagtggtggttccttccctccttccgaacaggaacaaaagggtgccaaagcttttgatatagggttggaataatcatgaggagtttaggatataaaactcagcttccgtggacacacagcagcgtaagtgctgaacgcttttggaggattggggtagttctgcttcctgaggagtttcttctcctatagtactcccaaaaatcacagtgcaagaagagccggtgctgctccaacctcaccccaaactctgtaccccaaaatcacaccgaaggaaaagcctgcttgctccagtctgtaccccacagcgatggtgaaggaagaaccaaatccccccctgctgctccacstgcttctctcccatcataattgcaggacgtgtcctcagatcccggaggatcagcagactgtgtcaggtgtaatcactgggagagtgagctgagggaggaaccgctttggtcctccctccaagcatgatttaccacccaacctgagaggaactcacctcattttcacgctgtaccgcacacctctcacccaccccaacacccaaacaaaacacagagcccagttctgccccaaacccccaccccaaagccctttcagtccccaggactcacgtggtgaggtcccacagcctcaaggtgccatcccaggagcccgacagcgcaaactgcccatcggaggagatgaccacatcgctgacaaagtgcgagtggccgcgcagggcgcgctgcgggatcccgtagttggtctcatctcgggtcagcttccacatgatgatggttttgtctgggaagggggaaaggcagcggcctcagctccaacccttctcacattcccgtcctcactgggctttatctccctcatagcaatgggggggttacacagaagcaccgcaccccttcctctcagccccccaaccgcctccctacgtcctcatacacagcagcctccccaccctgcagctctctgtccccgagccctgcaccccattcatcacctcccctccccsawggtcccccccagccccctcntytaycackgacggtgtccccttatttcccacagtcccctccataggccccacagttccctgcccccccccaccccacagttcsgccccccccgcctcggamgaggcccgaacccctcaaggcgcggccctcaccccgcgacgsggagagaatcatgtccgggaactgcggggtggtggygatctgcgtcacccacccattgtggccyttcmgggtaccgsggagggtcatctgctccgtcatggcggcggcggggcggagggatggcggcggattcaataaagggcccggcccggtccggtcctaccgcccgygatggccgccagcgcggaaagagaaagagggaggtgacttccggcggaagcggaagtagccgctgggttgtacggcaagaggggcaacatggcggcgcgcatagagagcacgctgaatgggggaatggggcttttggaggtggggagggaaggttgttytctgccgctgcaggracacgaggtgcgggcagagcaccttctttaacatttgktattatttaacgtttwacatttagcatttttattatccctgttgtgccaggacggagaagagcagggtgtgcagcctgtgcttatcacctgcagctgtccctgcaccccacagccaacccaagtttgtgacgcctgagcaggatctgacccaggaaggcaaacagaaggtctgagtcctcctccctttcctttcccatccctcccacgctgcagtttgggggctgtgacccgtccgcgttgctcagtgctcattccgatgagcagtggctgatggtgatgttcacaagtttttggcatccctgtgggttccacccccgttttgtctcaccagcctttttctatccgtccttatcagcagatcatccttgttattagatctgtctttttccagtcacggctttgcattttcaccttggttttaccacctaacatcaagccttttgtccccatctgatgatattcatgcagataaatccgtaaagcagggaagaattaaattctggccccttctacacccatttaggtttagatctttgcagcattcagccaagacgtgcttccagagccaggaataacgtgtcttgatgtgccaacacaccttgaaatccagaaaattgcggcctggcactttatttagggccacgtaggccggggagggtgcaaaaaattgggcaacttccacctctgaggctgctcagagtgcagcatcgcaccaggccgcaccggtgggaagcagccttgtttccccttgcagcttaagagctctctgaggtgggggtatttattttctcttcccttttctcagctgctgttgaatttccagctgaatcctgtcccaccagagagactctgattgcaccctgttgtgttttacttctttttgttggtggattggtattttttttttctgttggcgttacagagctagttcaaaatatttttggctaaaataagaattaaatggagatctagttttttgaaatgtcaagaaataataataataataataaagaataaagaataaagttttaaagctgagcctctcccttattgagagcccccaggggacaggagttgtggtgcaggccccccagtctgctgttaactcctgctggtaagatgtgacttaagccttgcatcgttaatcttaacttaattagcagtaatttggattgggctgcttcccttcagcagcttgtaaagggatagaggctgctgggtgaactgagctctgtgttaccacctctcctgctctccccacatgtttttggtggtggtggttgcttctttttggccacggctctatctccccaggtgtgcactcactgtgggctgctactgctcctgaaagggctcagggagacatttgagtcccttcgtccacacgtgggaggagagcactgatgtccccatccttaaagttgtgggcacagccttggtggcaaatccagaatgggatataatgcagccatgagctcaacagagcgctcttttattgagttttgtgcataaaatctgtgtgttgttaccacatcctcatctggttccaatggtgacttgccacacccggacgaggttatctgtgtagccagcaaacagcgtctggggagagaaatggaggaagtggatcatgaaaagataggaatcagccctcggtgtgaacgtaaaaatctcagaaggcagctcccaaagcggaggtgctggaggaaggtgggagttttaaggctgcaggaggagcagtgaaaagggaaaggagaaggggatatttctacctgcccatctgcagaccacgccagagaggtacactggggaggctcagctttgctgctggtgctgatcacctcctgcttcagctcatccacaatgattttgccttccaggtcctgtgcaggacagaagagagcgtgagggactaaggtcctgcagggagactgctgtagccaaacccaaccattccaactcagaacaggctcagggtgctcagaaacagcctctgggtttccgcacagggatgcagtcagatggcatcgaagtttcatcacagcagagtggtggctgtgccccacaccaccctcccagtccaggggatgacagtgccaccagcatgacccatcccacgtaaccaaaagggctctgcaccaaggcatctgtggggcagggcgaggatttcgaccacaactctgcctcccaaacccaacaggataagggaagtgattctttaggaggtaaatagggatgtcacatacccagatcttgatgctggggccggtggcagcgcagagccagtagcggttggggctgaagcacagcgcattgatgatgtcccctccatccagcgtgtacaggtgcttgccttcattcaggtcccacagcatggcctggccgtcctggggggcagcaaagaggaatcacagcaaaccatcaaacctgtggctttgttccagttgtccatctaaaaccttccagcttggaaacagcacttgatttgtgactgagatgtgggtgagttgccacaggacagcaagaggcacataactgagctgtgagaacaacagaataagctgcaatttggcctcagctttcccccagggtgtaccttgcctccagaagcacagagggagccatcaggggagacagtcactgtgttcagatatcccgtgtggccgatgtggtttgtcttcagtttgcagttagccaagttccaaacctaaatgagggtaaacgtgacaggctcagaaatatggaggagaaaaaaaacaaccctctcatgatcactgctcaaatattccccagaacgccgcacaaaccccaaaggagctgctcctctcaccttcaccagcttgtcccagccacaggagacaatgatggggttgctgctgttgggggagaagcgcacacaggaaacccactcagagtggctctcgtcctgaggagaggaacagcattgggttgaaagcaatgaaaagcatccccagtccgagctgctgcatcccactgctccctgagcccctcataattgcaggacgtgtcctcagaccccccccagaaagaaaggtcagcaggcactgtgtcacttctaatcattaggacggagctgggagatgtggattacggatcaaaaccaacaaatcaaagagaaatgggggaatacggactcagaaacaagcagtggtttttgagggaaaatgccggggggggggggggggggggatatgccctgagagatttagggtctgttttggtaaggaaagcctccagcaatgtgtgggctgtgtctttgttctctgtggggaagggaatcatccaggctcagtgctgagttgtggctgataagaggatttattgggagcaacggtgggattggtatcagtcatccctaatcctttccttctctttcccaccttgctgcctccttcccacaggacatcaagggcacttttatcaggtcagtgactttgtttgcatcttttcactttgaataacttttcttttttttaatgtcaaaaaagcatttgagcttttgttttaaatcctgtgtgatgggtacagttggggcctggtaatgcaggggaaagctgtgtcctaacttttgggtgatggaaacttctggctgatggggtgcaaatgggatctggggaacaacttgggaaaagacttgggaacttgggaaacaactctggggccatttgggaaaggggaagggtggggaggagatctcggccctgatttctggaagcgtgggtgtgcccatgcagacctcatgctatagcgaaactcctcactctggagaaacgattctccccatcctgtcagacaaatgggcagcgctgggagttctcagccatgctggacgcacgtggctctaccccagctctgtctgctggctgagggagggtgggggaggctggctgcaccagtgcaaccagtttggccgatccatgcgttgctctggtttttccagagctgcatgcaggccgcctcacttcttttctgctgctgaaattctctgctttcctcctttccccccacccaaaaaagatgtgagaacatcaaattccaggagcccgagatggtgctggtggacgtggggaagaaataccgcaactatttcctgcaggatgtggtgatgagaaagatggagaaagccttcagcaaagttccacagggtgagagagtcctcttccttctacgtgggatggggttccctccacttgggatgggatttctccagctctcttggggttctccttccatctctgtgctcccatggtttgcagcctgatgatcctttaggaaaagcagcatccctctgttctctctgtgcttttcccttttgccttgtcctgggttttcccctattgtagctcctccatagaactggggttgatgtggatctggattcattataaaggagggatgactgcctcaaactcagcatggtgcagatacgcaaccagatgaggatttaggactggggtgcaagggggaaaaaagtgccaggtgaccccctaacgacccccgctctctgcccttccttccagctgacatcacgctggacccggacaccgctcaccctcgcctcagcctctccctggaccgccgcagcgttaagctgggagaacgacgccaggagctccccaacaaccccaaacgcttcgactccgattactgcgtcctgggctcccagggtttcaccacaggccgtcactactgggaggtagaagtcgggggcaagaaaggttgggcggtgggggctgcacgcgagacggctcgacgcaaagaaaaaaccatggggcctcatcaaaaaagggagatctggtgtgttggcaccaatgggaagaagtaccaagcgctgacggccatggagcagatggctttgtcacccagcgagcggccccggcgcttcggtgtctacctggactatgaacggggtcagctttgcttctacaacgctgagagcatgacccacatccacaccttcaacgcttccttccacgagcgcatcttcccctttttccgaatcctggctaagggcactcgtatcaaaatctgcacctgatggccctccagcttctgattttttttttccctttttcccccctgcctcatcctttgggtcccactttgggaccagacgctgcacttgttgtctcgcacctgcttgctcacaaggcctcttccctcctctctcctgtcccagcctctgtccacgtcccaactcttctccggggtcgcgatcccaggctggtttggtttggagaagggatccaatctccttgctggaggttttcccttcagctcttggtgctatgggctcccctctgcctttcccagtcctcgcagcagctttccagtgtgctcttccccgttttgtttaaagcctgtggtcgagctttgcgttgtttgccctctttggatgcagagctcgagctgaggatgctggggtctgtacattgtgacacgagcactgcttgtgccctcttggccattgctttctgaaagtcactcagatgcaccaaggagcctcatttctttttatttttcagttctggggcacaaccctctgcccacctcccacccagccaccatctggacctcaaaccttccacgttctcctattctgccacttgtccaccttccccttttgctcttcttccccctctggctggtctccagctctccctctgccccatcattccctcgccaaccatttcttgtgagaggaggaagatgagctgggggaggaagagctggacgtggagcaggaggaggaggaggaggatggaggcggggaggaggaggaggaggacgacatgtggagcgaggaggaagaggatggagagctgtgggaaggtactgggggtcggtttgggcctgccctgttgagtgtctttatggatgagtgagggaattgggtgcaccctcagtcagtttgcagatgatgctaagctgggggggtgtactgatctgcctgagggtaggacggccctacggtggggtctggactgggcccgatgggctgagggcaatggggtggagttcagaaggaccgagtgcctggttctgcactgaggtcacaacaaccccatgcagctctacctggggtagagcggctgaaagctgtgtgagggaaaaggatttgggggtgaatatgagccagcaagaggccaagaaggcccatggcatcctggcttgtatcagaaatagagcagctagtgggagcaggaagtgactgtcactctgtactggcacacctcaatgctgcacccagttctgggtcccctctcactacaagaaagacattgaggcccagtgaggatggtgggggttggactcaatgatccctgaggttttttccaaccttgatgattctgtgattctcagaccccgtggaagaggagctgtgggatggagtggtgcagggagaactctactttggggacgatgattatgatgaggatgtgatggaggaggatgtggaggaagaggaggaggaggaggatgaagcgcagagccctccgccccctgtcctgcctgcccgccctcgccgcctgcagaccttcacctgcccccagtgccgcaaaacctttttccagaggaatttcagacccaacctccagttggcaaacatggtgcagatcatccggcagctccacccgcacccgcagcgcctcgcgccgcccgccggcccctcagcctcagggggtcctggggggaacccagggatcctggtggcaacaggaggtcgggggtgtccgaatctgtgcgagaagcaccaggaacccctgaagctgttctgtgaggtggatgagcaggcgatctgcgtggtgtgcagggagtcacggagccacaagcatcacagtgttgtgcccctggaggaagtcgtgcaggattataaggtggagtttggggaagggtcacggtgggatagtgggtgaggtggggtttggggaagggctgtggtggagaaggcggggtttgagggaagagttatgggagagtggaggcttgaagggaaagtgaggttgggatcaagctaggttcgtcttgctgagctggttgggttggaggcgtgggaggctgggaaaccacacactgcaatgaggaggtggaagggtctgggtacccattttctgcttaaaaacaccttcccagcacagttcctcagagaaagcaaaagggaagtggcgtgaaagttggctctgaggttccgttttcagctctgccaccaaattagggacaaaaagaggcgatgacagaggggattgccccaggcagggtttgctgagttgtgtttccttccctcagtacaaactccagagccatttggagccactgaagaagaagctggacgcggtgctgaagcagaagtcgaatgagcaggagaagatcacagagctgagggtaagagctgaaggtttctgtgcttcatagaatcatacaggagaaccatcagggttggaagagaccacaaagatcatcagttccaaccatcaccgctgctgggagtgtgccttggtggctgagcaaggagagagaagctttgctgctgctctgagctctcacggaggcatcatattccctttcctgcaattattgggctgtgagggcttggaaacggtttcccagttgaattagagcttaatgagagctttgtgtgcctcagtgttgagtgggaattggtggtttgggagctggtattcctcatttgagttgaggatgctctacatctctaaacctgtgcagactttgctcagttctgtctgtggtgcattcaggagatgcgtaagcttatggtgtgtggtgaaactgagagaagcatagcacagcagcccaaaaatgagctgatctctcacctcccccttctgcagcaattcccctaatgcttttcctccctctgcaggaaaagatgaagctggaaatcaaggaatttgagtctgattttgagctgctccaccagttcctcattggggagcacgtgctgctgctgcaccagctggaggagcgctacgagagcctgctggcccggcagagcagcaacatcagccagctggaggagcagagtgcagcccttagccgccttatcacggaggcagaagataagagcaagcaggacgggctacagctgctcaaggtcttcttccatccctttccttgtctttatggcaaagcgatagcacgatggtgggaataatgctccagaaagcttctgtgtcatgagagagtgcctttagttggtgggctgggtgcttctccacccctccttgtgaagataagcacttggtgagatttccctcataaacaccccaaaacggcggccctggggtgtgtttctgtattaaagagccctcagtggaatggtttttgcagggctgtggtcgaagagcaaagcatcaaaggaaggagagggcagtaatgttgcaaagggctgacggcggtggttgcaaagagggaggatggggggggatgcgccaagcaaagggtggcgtgggttcacccgcagggatgcactgcgcccttggctccgggttttgggaccgtaccttgtactcctgggccgcctggtgggcagggagcacagcgtgggagcggtgcgcctgggacgcgtcgcactgcgcgcagataggctcttggtcctctgtgcagaagagcttcagagcctcgcggtgctgcttgcaccaacccgaggaatgcaaactcagctgccgggcgatgctggcgatatttgccagctctctgctggggcggaaatttttgtgcaacgccgttttcctgcactgcggacaggggaaatttccctccagcccttcccagcagcgggcgatgcactcccggcagaagttgtggccgcaggggatggagacgggatcctggaagtaacccaggcagatggagcaggaggcttcgctctgcaggctgtccaaggggctctgcgtggccatgggcttcctgctgggctccgatccgcagagggaataggggacctttcctccttatctcctcgctgataggagaaatccggccccggaggctgagcctgagccaaacagggctgggagagctcagcccataggggatgctggtgggaatgggggcagctcgcggctccccagcacggagtcaccaaactggggggatctgggggaaattcggaggaaaagtcagattttgtcctctcctcgagcagcaaagagggcaggggaggcgatttttcccttctgtgcgatcactgtaaggaatttccaaagaaaacgcatggaggtctgcttgttgggatggaatatagacgtatattggaataaatacaggaagacgttggaacatgggaaggcactgagatataagcgtgctgtgttggatatgactctgctcgactaaagtgaaggtggttttaatagcactgctcagagccaggcgggttttggtgttgtttggggggaattacgtgggtttggaattgggaaatatgagacggaaaaataagaataatggaagcgcccaacgtggggctcgaacccacgaccctgagattaagagtctcatgctctaccgactgagctagccgggctgatgggcacgcacccttctaagcaatacttcatggtgatcctgcggaggggtgctaataattctacctaattattttgttaattatcccggtaattatgggttctgagcaatcgcgaatccacggggaagagctgcatggggaaaaagcacctatccctacgggaatagccgggaactgcccggcagtgctgcagggcgggggaaagaggggaaaagcaggaaaaaaatgggcaaaatggaacgtttaaaagtggagaaattaacagtgaaaaaaatgcaggaagcgtaaaagtaaaggctgtgtttctgcccggtttcgaaccggggacctttcgcgtgtgaggcgaacgtgataaccactacactacagaaacgcgctgaaggccgcttcgccgcacggagatgtgaaggggcgaatgccggggctcggtgcggagtttgcagataggggccgctccgggccgctcccgcgccggttccggtgagcacagagtgcagcgggtgacaaaatgaagggaaaaatgtaaaactgatgctcccgaatcgaggctcgaaccgccattgtccgactgacagccgcgcgctctaccgactgagctacccggagacagagcagccggaagtcacgcccccgtagagcgcccaccccgttgcctagtgacaggagcgccgcttccggtcaagtgatgagcggagggggcgtggcttgtgtcagataggacggaagttccggtcaggtggtactggaaagggggcgtggcttgcggcaaaggggacggaaagcggaagtgctgccgttggttggcggagttcgcaccatagaagaacgacggcggcggtgggagggcgggaggtagagcggtccccggggagagtgctgacgggagcggcgaggcccgaggagggagcggagcttacgggggagtgcggagcctcgaggcgggtcccagcgcttcgctgtggggcaggagaaaggcttcggggcaggaggaagagggcctccsggccwsscsatggaggcggtgggcgacgatggggcgtcgtcggggcggctgaacccggtggagacgctgcaggaggaggcgatctgcgccatctgcctggactacttcgtggagccggtgtcgatcggctgcgggcacaacttctgccgggtgtgcatcgcgcagctgtggggtggaggagaggctgaggtggaggagagcggcggggccgcggcgttggaggaggaagaggaagagctggaggaaacacgcagggctcgaagctgaacctctcggggttctcggggaggtcctgtggcaccagttggccccgggcttgttttcggtcttcagagagatggaggttggggtgagcggtggtggggtccatggtgacgttggctgtgggacatgagggggaatggaggtaggatttaggcttggggggagctggagaggttcctcttccttctgtccttttctctgggtgcttttggacatgggctggtggtggtggtgggttgatggttgggctgggtgatctttggggtcttttccaacctttgtgattctatggggtgtgtggggctccaccagcctcagtgtcccccagtagagatgtaggagaatggggagaggacaaattttagggcagcataatgcgggagggacaaagacatgggaaggggacagcttgacattcacggaggggaaggggaagcacaaacactgttaggttttgccttgaatctgttactggctttgtaggaccaccagcatcaggatgctgtccccattccctcccttccctgtgggactgcgttgttttttcccaagaaaaccactccccaccccacatccaccactgctgacatacctggctcttgcaattgaaacatcaggctgtctgaaaaggagaacaaattcactgcattgggtttatgcttcaggaaaaggggctgggagatggggaagggaaaccatgggggtctgggggcttcgcagtgcaaaagctctgggtttactgcaagagccccacgaccctcccagacctggaggagaccccgaccccattcagtaccttggcacttctgcagcgtcagtctcaccaggacgttcttctgaaggaagtcctccaaccttctttccagagtgggggaaatctctgctggagggctgaacttcatcatctcacagctgcaaagagaggagaagggtggggatggggggactgttgcgttggttggttggctgttcattttattctcaataggagaagctatggggtgaggatatttgcacagggacgaaatccctttcccccctgggatccctctgccttgcagccctcccccagggtgccatccaaaaatcagggtgacaataggaaggagccatgttacctattcaagagcctcctgatgtcctaaaggtgggaggagagaggagagatggatcagaagaggagcaccaagggctgccccttcgtatggcaatgcacagcaaagaccaccctgcccacggtgtgatcccccccagcagcaacacagggagctcccatggggttgagtttgggttctcagggtttgctctgtccccccatttcccaccacccctttgggttctcaccagcaggaatttgctgtcgggctgctggaatttgccctccatctcccagatcagggtgtcaaggtgggacatctcctccatcaccttcgtcaccgcatcctcctgtactttggtgacggctctgtccaggtctgccagctggaccagcaggaagcgctccttctccttcagaaatcgctgcaactgctcgaattcacacactatcctcttcccttccttcttggttttctcctgttgggatgagggagaaagccaatggggtggaatagaggcaggaagaccccccctctgggtctcaggatgccgtgttctgggggatatccaaccaaaaccaatggggatgtaacaccaatgccaatgggagcacaacactaatgccaatgggaatttatcaccagtgccaatgggaacgtaacaacagcgccaatgggaacgtaacaccagtgccagtgggaatttatcaccagtgccaatgggaacttaacatcaaaaagccaaagatcatcttgctgggcatttgggagcagcaggaatttttcaggagttttatcccaaaagcaaaaccaaaggagggggtaggagatgagctctgtatgagggatatttacagagtttaggaggatctgctacgttatctctttaacacaggggttcctgcgtaaccccagctgataaacacagccttagcgctttcccagcccagctgcgagccaaaaatgcatgatctgcccccaaaatacaccaaaacaaacaggacagggcggaggggaaggcagacacctcccctgctgcacccaccaaatacaagcccgtccttccaccagtccttctgctttccaggtacttttccctctcctcctttgaagcctggaggcgagcctgaatttcttcctgtgccaaaagaagaaaggcggaaagcctgttttcccacttaaactgcttctgtcagatgggagaggctttgctaaagcctggaatcctctgcaaggtgcagagctgggcagagggaagctctgtgagcacggtgtgctgctctggagctctgtgcaagctgggagtattttgcagagagaaaagaggggagaagggaaggaaaaacacgaacttgctgcaaacgtagagaaaaacgctgcaaaagtgcaacaaaaaaatcagcactgacagctgcgcaaggaggtgtggaagggccaagaagggctctgtgttttcctgcctggaatctgagccctccctactggggctcagctttccttctgatgcagaaagtggaaaataaagagcagtgggactggaaataccaggggggactcatgagtggcatcccccactggaggagctcaatggtgagctggaatccttgctaagttttatcgaatgtgggggacaggaggaagaaatcaaactcaaaaagtcatgaacaggtggctgtgaattcggggcagaaagctgagggccctaaaagcacaggaggcaaaaaggatggagagaaacgaccctactgatgacacatcgctgcccagcagctgacacctaccagatcctccaggtttgggcactccagggcgctcttcttcctcggagactttctctctcctcctttggaaacccctgatatccctctgagtttcttccccagtgaacccacagaacctgttgttttcagccctttgatggggttggggttttcccttcctgttccttcccagtctggggtagagctatgggatggctgcgttgagcctgcaggtctgctcctggtggcacccttggcagggcgtgctgggagctctgggtttgtcctttgtctttctcccagttccttgtcccggggagatgctgaacaatgtcactttgcagattttgtcagcttccttttaggatcgagccatcgggagtggggttagggggtgtatatggggaaaccataaggaaatagggaaggagatgcacagccggatccttgtggggatgtggaggagcacaagtgaggatctttgggatttgagtgctctctcagcccagcactaacacagagcactcacagccctggctctgagctctcgaggaaacatttccaaccatttctgccccactgtccttgtgttgagccccatggccaaatacacatgcctagaaaataaagccatgcattacatatgtatttaatttttgcgtggcaaccactgagacccaactggaggagataactgccattcacttgggcaggtttgcaggggtgaactgcacttccagcaaaccctccctgttgggaagagccacagggatggatggcactctgggagctgaagaactggaagcaaactccctgcaaccgctcccctggggcacagagcctttcatcccaaaataaggcgtccatcattgagcaaatgagtcacaccgttgggcaaacgacttgcattgcatcccgaaaagcattaattgcagagcctggaaaactagctgggctggaaacatctgcattgcagatctacggagcagaatagaccctgaacagatccttcacccaaattccccagcaggtgggaccaaatggcagcgatgcgtggggctgaggaaagataccaacacatcaaagagcaatattgaaatttcagctgtaggtttgacctttggaggtggtgaggtggggctttgtcatgggatacccactcatatcgcatctgctattctgagcctgatgtcgcctgctccctcccaccctcttttagttcctcttcttggttctacaatcaccaacctgtgtgtattttggtgctgcctgttcctcttttgggctttctcagaagaaaatgggtttttgagggaatccattcaggtgagtcctcaccccaagcagctcttcttcactttgttggcccaaagctgacccagagccatacacccaaagcaaacccagagccgtacacccataatgaggcaggaagtggagtgtgcagagcacatcttttaattaaaattaactatcagaaacgtaggcagagaccagctccccacaccaggcgttgctatttgcagtgaaaggccgcatacctttgcaggacaccccagatctgccccacgattgatgtcaaatagatgcataaatttccttccaagtcttcagtgctctctggtggtttccccaccctgcagagggaccgccccggggctcccaatggggacagacacagggcagagcagcgggtccccttggcacattgctccaagcaaccacagcacacatcccatcagatgcccctttcataaaggacatctcaaggacagatctttaggggagatctaaacccaacccaatccaaatgggacatcagctgcccactcgtggactgctcctctgaggggggattttgggtgatctcttgcaagcgagcccccagccctatcttgaacaaggggaggaccttctccccattgaacaaagccctggtgtacaccaagatgggggtgtcatcatccgagctgaagaatgccacccgacccccttcgtagtccagggagacccgaatcctcctgggaagtgcattcagacgtaggttggcacggggagacgtgagggagtggtaggcctccagcgcccagacaccctctttggggctgaagctcatgggtcccttcctcttcatcgaagcccgggccacccccagggcccacacccccccctgtcccacctccacctcccagaaatgcctccccgaggtgaagccctggcagcccacaaagatgagccccccttcatcagcatcaaaaaatgccaccgtccctccagcgtagtccaagtggacgctgaccctcctgggcacccagcgcagagctaacagggtcaccttgtgggtggtgagtgcccggacctgtcccccccatttctccaccccccaaatcccccctttgggacagaggctgagttgacccttccgagggatggattctcgggccacaccgatggcccagtccccttcatcccccacttccacctcccagcagtgccggccggcagagaagctttggtggcccaaaacaaagggccagtaggcgaatctttcggggttatcaggaaggtcctgttgtccttccccacgtttcacactctttcggtcttcggagaggatgaggtcagggtgagcggtgtcggggtccagggtgatgctggctgtggggtggagaggatgaggagtgtaaggtttgggtcctcggtgctgaggccatgaggatgcggagagcttggatctccagcactaaaggagttggatgtgctctagatggccccacctgagtagggttgtagggtgggaccgtcccttccaacctcagccattctgtggggccatgggttggcatcggaagggtaaaaagtaccaaagaagaaagtaaaaaggtgagaggtggaaacccctctcatgtgcccgtgctatatgacaataaaagtgttttgagcccccagaatgcccagaaataaaggcgtttctgcagaccttctgttccattggtcaaaagaaatggtgaggggaataaaaatggaaggaaggagatctatgggatattacctgcaaagtctgcagtgcttcatctcctagaccaacccggaccagttcagccaaccccatggtttaaaaaacagagctgaaatctgaaggcagggataatgaatgagttcaacccgctcaccatatttgtttatgggaaatggatatttatcaaggcgagggatctgccctggggccatcatcccaaattacagccagactcggcctgcagggtgaagaaaacttgtttggctgccctgatttttgtgtattcctccctcggcatctatttttgtccatttgggtacagcctatgggtccaggcgcgcctccatctaacaggtaatgcggctttaggttctcatgctcagcaaaaggcacttttaggaaaggtgaagctggaggggtgcagagccggagagcagcccgtccttcacccctgagcacttctcaggaattacagcaaaacgtgtaattaagagtggcaaacggggtatcgagtccttcgggtctcaattattttcctgagtgggaataacccgttgctcttccatctctctgcattattctgctgcagaacgagtgatgggctgctggttttcaccaaaataccaccatttcccacccgaaacccttctgagtaccttgaagcctcttcagggtttccttcagagcaccgttcctccatgaggaatggcacagcctctcctccggccctggagaagcgcccgctggcagctggaaggtcacttttccacacctggaggggaaataaatgcattttcaggtggttgtatcacagagcatgccatcacttcaggacagcagaggccagcacacggcggccatccccaaaatacccttcagggctcgcagttcccctggagcagaagagcattcattgatgagctttctcctccatggtcactgcctgatgcaaagctcacagaacagcttttcagagaggccacatacctggtgatggggcttttcacatcctggggacagaagagaggagggggagaggaaactcaggtcagtgcatgacccattttgtctttaaagtatggaaaattgagctgtttgagtgggggtggacctcttgggtcttccaacatgtgcccaattttgactttaagtcatagaaaaagtgaattgtttgactggggatggatctgttgggtctttcaacacatggtccattttgtctttaaatcatagaaataaagaattgtttgaccagagatggacctctggggtcttcctccacgaggaaggtgaaccaactgaggagcatccatgcacggcaatgaatcctgcagatccaccccactgctgctctcccaacccagccgtggatttcccctcttaaaacagaccccatgaggaccttctgcagtaaggtgaaaatactgggaatactgagatgaggataaaacggtggggggaaagaggaggctgcaaacctccatctcctcattgtggtgggggtttcaggctgatggaacggcataaaatgggaggaaaacacccaattaaggcaccatgcaattggtcggggtggggaggacatccctaaaggacttttccccttgaaaaagcttccctggaggaattcactcaccgactgctggctcttctctccctgtgctttcgtatccagcggggaaatctcctccgagtgcttggcggtgcttttctgcctcttctcaatctcatttttcaggtcttccagctgccagagtcgatcaacgtttcaatgttggtatcaacaccaggtttaactttgaacttatcggcactgacggttaccttgttctgcgctggctcatcacgctggataccaaggctgatgttgtagatattggtcaccggctgaggtgtttcgattgccgctgcgtggatagcaccatttgcgatagcggcgtccttgatgaatgacactccattgcgaataagttcgaaggagacggtgtcacgaatgcgctggtccagctcgtcgattgccttttgtgcagcagaggtatcaatctcaacgccaagcgtcatcgaagcgcaatattgctgctcaccaaaacgcgtattgaccaggtgttcaacggcaaatttctgcccttctgatgtcagaaaggtaaagtgattttctttctggtattcagttgctgtgtgtctggtttcagcaaaaccaagctcgcgcaattcggctgtgccagatttagaaggcagatcaccagacagcaacgcgccacggaaaaacagcgcatacagaacatccgtcgccgcgccggacaacgtgataattttatgacccatgatttatttccttttagacgtgagcctgtcgcacagcaaagccgccgaaagttaacggtttgcccaggctcacaactgaaagactttctacggtgtgcgcgtgcgatgcgcgtagaagactgatttatcaacctgtctttatatcaggattcattacctgactatttgtgggtaaagttcgtagtgcgctgatcgtgcaaaatgattttagttgggaacagttcgcaactctgtcccataaaaatcagcatattcccatctatcccatatccagcgcattgaccatcgggatactgaagggagattccatcatctcttagaaagatcaccatctcttttgtttcaatttgcatatagctacctggaggatttatgaatacaaggattttcatggactattaccatgagattgattttccatctttattcgcgagagcagtggaaagcgatgacgatgtgggtactacattgcgcattcacctactttgtgagcgcatggtcgaagcatggatatgcgcatgctgtgactgccaagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggcatcggtcgacgggatcacgttgtgtccctgaagctctcctgtacccaaacacaaaggtgatgtccccagcatccctatcccagcactctgggggactcctattgaattcctccttgggcttgctgccttctcttcccgttcccagagatcccaaaaggttaagcacctttgggtcagtgttcagaattgtcactgccagttttggggtatcagtggcaaattgagacccttttacccaatcttgcaccactctggttccccagtcttatggttttagatggagtaaaaaggtttatatgtcataaagttcttctgtgtctggttattcgctgcttctggatgccaggatcatggggataaggggaaaacaatgggttctcttatgcgtagagatgcaatcagatggggagaaaaagaaatcttaatctttctgatccatctgacagatattcagtacagccctgaggatgtggggaaataaatctngragagttkgtkggcagttccaaggatttgggaatgactaaatcccattcctggkkwytgcacaaagttgsctgtgttggaacccagaaagatccatgcaagtgggtcatccctgaaagcattgtgttctgctgtctgctagcggagagaaagacacagaggggaaaattaagtgttttattgttaattattgtacactctgaggtttcaaataccaaatctttaacgagagcggaccacttgatttgagggtgaccatctcagatggggacaactgtacctgatcaggcaaacctgggggaaatttgcctttctgccactcttttgggtgggattttcccttttgaccaccattttctacattctaatcacccattgcagcacttctcccccttttttttgccccatttttctcctgctcagcacttcttaacaatataatataaatcaatatcatatcaatatgattctatgccaatagattaatggggatgaaagacacataaaaacccaagtcctcatttcatctgcttcccatgggatgggtggggaggtggctgtcccctgaggctgtaggatgtggggtcacccttgtctgtgtctcagggacacagcctcagcttggacctgacccctaccacccacagccacggacggaccctctccccagagaaggatgcatgggaaaaacggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatctgcggcacgctgttgacgctgttaagcgggtcgctgcagggtcgctcggtattcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagctgcgccactccctgaagctcctgcagtccctcgcgcctccgggtgacaagatagtgtacctgtgccccgtcctggtgtttgtcgcccaacggacgctccgcgtcagccgcgtgacccggctcgtcccgcagaaggtctccggtaatatcaccgcagtcgtgcggatgctccagagcctgtccacgtatacggtccccattgagcctaggacccagcgagcccgtcgccgccgcggcggcgccgcccgggggtctgcgagcagaccgaaaaggtcacactctggggcgcgcgacccgcccgagtcagcggcccgccagttaccacccgccgaccaaacccccgcctccacggagggcgggggggtgcttaagaggatcgcggcgctcttctgcgtgcccgtggccaccaagaccaaaccccgagccgcctccgaatgagagtgtttcgttccttccccctccccccgcgtcagacaaaccctaaccaccgcttaagcggcccccgcgaggtccgaagactcatttggatcgatccggaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctgcctcgttggcctgccgcagttcttcaacctcccggcgcagcttttcgttctcaatttcagcatccctttcggcataccattttatgacggcggcagagtcataaagcacctcattacccttgccaccgcctcgcagaacgggcattccctgttcctgccagttctgaatggtacggatactcgcaccgaaaatgtcagccagctgctttttgttgacttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacaaggcggatcgccggaaaggacccgcaaatgataataattatcaattgcatactatcgacggcactgctgccagataacaccaccggggaaacattccatcatgatggccgtgcggacataggaagccagttcatccatcgctttcttgtctgctgccatttgctttgtgacatccagcgccgcacattcagcagcgtttttcagcgcgttt

SEQ ID NO: 6 - DNA, 1262 bp, Gallus sp.
gcacaaggaaatgcaaaggggcatcactaggggacatggcacggggcattctagggagcattgcatggggacattgcaaaggaaatgcaaagggacattgcatggggacattgcaaacaaattgagtgggagattgcaccgggatgttgcatggggacattgcatggaatgtcccaccaaccaccctgcagggtgacactgggaccatccccagctctgaccatcccccctttgctgcagcaccaccccaggtccgcatcgtccccatccccatctccaacgaccccgacaccgtccacctcatctgccatgtttggggcttctacccacccgcagtgaccatccagtggctgcacaacggcctcgtggtggcctcaggtgacaccaaactgctgcccaacgggggactggacctacaggacacaggtggccctgagggccagcattgcagcagggagcactaaaacatgttcagtgtggcaattccagcttggagcagccgctgcaggaggattggagtgagtttggggatggggatgtggcacccacaccccacagtcccccacggttcattgtgccccacgctgtccccacaggtcccaatttgtccccggcgatgatggtgaaggtggcagtggcggccatggcgctgacgttggggttggtggcactcagcgccggggttttcagcttctgtcagcggccacggggtgagggatggggatgtggtgctggggacatgtgtgacaccgagggtctggtgtccagtgtggggtgtacctcctcattcatcatcttctgtgtggcagctcctggcgctggtcccagtaccccgtcctgatgcgggttctcactccaatcctggtccccaaaatgatcccggtccgagttctggtccccatcccagtcctggaccccatcccagtcctggtccccattctggtcttggtcctggtcctggttctgctcctggtccctatccctgactctggtcccggtccccatcccgatgccagtcccagtcctggtccccatcctggtcctgctccttggtttggggacctcaatgactggaactcccatgtcccaacatggggacccacagtttggggtgaggggctctcaccccccaataaaaccatctgcagccccaacctcgctccaattcttcgttcccacgttgggtgggtcgggctcccagtgctcccagccgttatgtcccgtaagcgtcggctccactgcataaaaagaaaaaaaaaaaa

SEQ ID NO: 7 - DNA, 4138 bp, Gallus sp.
  CDS (1..104, 178..307, 383..589, 1020..1217, 1727..1855,2174..2359, 2643..2817, 3109..3266, 3338..3476, 3656..3868)
  modified_base (1)..(4138): “n” represents a, t, c, g, other or unknown
ggcgagatggccgtgccctactacatggggcgagccagcgactgggtggcccgcgaggacaagctggcagccatcctgcccatggtgctgctgggcctcagcaggtactggcataggggggacgcggtgcgggcagggggcagcgcgggaccccctgacaccccactgccgtcacagcgctgttactgagctggtgtgtgatgtgaccttcgtggggacactgagccgcacgcaaagccgcctccagcgccgcgtcttcgccgccgtcctgcggcagagcatcaccgagctgcgcgccgatggggccggtgaggggcaccgggctgggaggggacacggggataagggacaggggtggcactgacggcgctgtcacccggcaggggatgtggccatgcgggtgacgcgggatgcggaggacgtgcgcgaggcgctgggcaaggcgctgagcctcctgctgtggtatctggcacgcggcctctgcctctttgcaaccatggcctggctgtccccgcgcatggcgctgctcaccgcgctggcgctgccactgctgctggcactgcccagggctgtggggcacttccggcaggtatgggctgctgtctgcacctccatgtgcctttggtcccctccatgtgcctctggtcccctccatgtgcccagtgtcaccaccatgtactcattgccctatccatgtgcccactgtcccctccatgtacccaccatcctcctgctgttgtcccctctgtgtgaccggctgtccctccacgtgccccatgccccttccatgcgtcccaccatccgtgccatgtgctcattattccctatgtgtgaccattatcccttccacatgggttctgttctctgcatgcccccactgtcacctccacatgcccaccgtcccctttatgtcccctccatcccctccacgtgttctttgttccctccatacatgcactgtcccctccccagcccccattcccttcccacccgccctgcaatgacactgctgtccccaggccctggcaccacagatgcagaaggcgcaggcccgggccagcgaggtggcagtggagaccttccaggccatggccactgtgcgcagctttgccaatgaggatggggcagctgcacactaccggcagcgcctgcagcagagccaccgcctggagaaaaaggatgtggccctctacactgcctctctctggaccagtggtgtatgggatggggtggctcaatagcatggggacgtgatgggatggggctgggggatgtggggacatgataggataggactggggggcatggggacatggtgggatagggctgggagatgtggagacgtgatgtaattgagatgtcaggagatggggacagaatgccaacgggctggaggccataatggtgtggagatggcaggtcatgggaatatgatggcatggggactgtgggacatagatttgatggcatggggacatcaggatgtagcaggcacaacagttcaggggctctggggcaggaggatgcagtgacgtgggaatggggcggcatgggggctccaggacactgggaacatgatggcatgaggggacatagcacagagatagcacagctgtgggacactgggacaggggggacattgacagaacaggaaggtgacagagtggtgctggggactcagagtcccagggggaggtgtcccctggtgacctcatggcatcctcagttctcagccctggccctgaagatggggatcctctactatggggggcagctggtggccgcggggaccgtcagcactggggacctcgtcaccttcctcctctaccagatacagttcactgatgtcctggaggtgagcctgagaggatgcccatatccgcatgtccccatgtctccctgccacagtcacagtgtgtcacagtgtccctgtgttcctacatcctccctgctgtgtctccaggccaatcgcagtgtgtccccatgcccatccatgtccctgtgtccccacgtctaaccctctgtgacccctgccacatccctgtgtcacctcctctgtccccatacctaccccgtcctgtccctatgccaatcccactgtgtcctctgcgtccccatgtcaccatgccacatgccccttgccctcctggccacgtcactacgctgtccccaggtcctgctcgactacttccccacactgatgaaggctgtgggctcttcggaaaaaatctttgagttcctggaccgggagccacaggtctcaccctcagggacaatggcacccgctgacctgcagggccacctccagctggaggatgtctggttctcctaccctgggcgccaggaaacccgtcctcaagtgggcacagagacacagccaggggacacgggggtgtggtgggacagcgtgacaggtgtggagcacagtggggtgattcagggacatggatgtgatggacagggtgtgaggatatgaaacaaggagatacatggagggggtggtatggggacactggagagggacatgagatcatggtattgagggcgcggggacatggcacatggtgggtttgtggcactgggacatgatgagtgacacagagacatggtggggagggcatgggaatgtagaggccgtggtaggggtatcactggagctgcgccccggggaggtgctggcactgctgggacccccgggcgcagggaagagcactctggtggccctcgtgtcccgcctgcaccagcccacggccgggcgcctgctgctggatggccaccccctccccgcctaccagcactcctacctgtgccgccaggtgagcagccacatgtccccatggctcctggttgtccccctgtgttcttgcatatcagcagccatcctcattgagtcaccagatatctgggtccccagccatcaccacacaccctgatgtctctgccatatcaccactgtgtcccctgcagtgtcccggccaagtccccaaccatccttgtgtccccaaccatcccaccatgtccccagatgtccctgacacatccccagccatccccaccgcctccactgccacgttgcccatgttccccagctgtcccccccactgcaggtggccgtcgtcccccaggagccgctgctttttgcccgctcactccacgccaacatttcctatgggttggggggctgcagccgggcacaggtgacagcggccgcccgccgggtgggcgcccacgacttcatcactcgcctgccccaaggctacgacacaggtaagctgtcccctttctgttccgggtcccctccatggtccctcccagcctgaccccgctcgtccccgcagaggtgggcgagttgggaggacagctctccggggggcagcggcaggcggtggccattgcccgtgcactgctgcgggacccccgcatcctcatactcgacgagcacaccagcgccctggacaatgagagccagcagcaggtgggatgtcccccacgtccccgtgtccccacatccccctgagccctgtgttccctcagattgcacgcctaggtccccatggtcccctgttctggtgtccccttatctccactcctggtgtccctcggtccctggcagtggctgaggaacatccccctgaaccgtttctcctcccacaggtggagcaggagatcctcgcagccaaagggtcggggcgtgcagtgctgatggtgacggggcgggcagccctggcggcgcgggcacaacgagtggtggtgttggaggggggagaggtgcggcaggagggacccccccacgaggtggtgcgccccgtcagccttnttgcgggactggggacaacaagggagcaccgggggagggggacagagggatagcgggagttttggatggggagggcagggggtgggtgggatgtgggatggggacactgcgcgttggggacactgagggtggaggtggggacaccggggcagcaacaagggaccacaagagctgtgccgtgggcacatggatgccgagccgggcgcgctgcggtaccgctgctgtacgacacacaacggccacagcatggactgcagtgccactgagtgccaccagggccgggggtgggacacagaactgggaataaagccgcatgtttgt

SEQ ID NO: 8 - DNA, 3133 bp, Gallus sp.
  CDS (273..589, 787..898, 969..1175, 1463..1660, 1736..1864,1963..2325, 2395..2553, 2622..2759, 2835..3011)
  modified_base (1)..(3133): “n” represents a, t, c, g, other or unknown
cgccatacattntgcgcctgtcatgcacggtgntaatggccgacctggccntcatgttggccctggcccanttcttcccagcactggcccattgggctgggtgggctcctggcggaggccgggctgcgcctcctggtgctggggggggccgggcagctgctggcccccaggggaccccgtggggctgcagtgctgctagcatgggccccgccatcttcctgaccctacggggctatgtaggtctgcctggagctcccccggtgctgctggccatggcaacgccgtcctggctggtgctgacccacgggacagctgtggtggcattgctcacctggagcctcctggtccccactgtggccactggggcaaaggaggcaaaggcctgggtgcccctgaggcggctgctggccctcgcctggcccgagtggccgttccttggctgtgccttcctcttcctcgcattggctgcactgggtgagacctcattgccctactgcaccgggagggctgtggatgtcctccgccagggggacggcctcgccgccttcaccgctgctgtcggcctcatgtgcctggcctctgccagcaggtagggaccccacatccctccacaaaaccccatccacctctggtggtcgtctggtgggtttgggggtctctgtccatatctgggggtcatctgatgggttctgggcactccactgaccctttgtgattgtctgaagggttctggctctccattgacccctgatgggttttggagtcgcccccccaattccttcccagctcgctgtttgccggctgccgcggtggcctccgcttcgtcttgcgcacccgcgaccagctcttctccagcctggtgtaccgggacctcgccttcttccagaacaccacagcaggtacagactgggggcacttttgtccctgtccccacaccatacccccagctcaccgtactcaactccacagctgagttggcctcccggctgaccaccgatgtgacgctggcgagcaacgtgttggcactcaatatcaacgtcatgctgaggaacctggggcaggtgctggggctctgcgccttcatgctggggctgtccccgcgcctgacaatgctggcactgctcgaagtgccgctcgccgtcaccgcacggaaagtctatgacacccggcaccaggtgatagcagggatgggatggtagggttggggtgacagggatggaggcaatggcaatgggatgggaacagtgggagtggggatagtgaggtggggattgtggggtcagggtggcagggatgagggcagctgcaatgggatgggaacagtgggaatggggagagcaggatggggacatgggtccaacacagcaaggatgagaggatggagaagagtggagcaggaatggaagtgggatggccgagtacttggccatcccatgggtgctgacacccactgtccccccagatgctgcagcgggccgtgctggatgcagcagccgacaccggagcggcagtgcaggagtccatctcttccattgagatggtacgggtcttcaatggcgaggaggaggaggagcaccgctacagccaggtgctggacaggaccctacggctgcgggaccagcgggacacagagagggccatttttctcctcatccagcgggtgaggctgacacgaggggacaccctggtgttctgggtgggatcgggacatccccgctgagccccatccccacaggtgctgcagttggctgtgcaggcactggtgctatactgtgggcaccagcagctccgcgaagggaccctcactgccggcagcctcgtcgccttcatcctctaccagactaaagctggcagctgcgtgcaggatccccatgactgtggccacatccccgtgtccccaccctgggtgctgtgcctgggggtcacatccccatgtccctatcctgggtgctgtgcatgcaggcactggcgtactcctatggtgaccttctgagcaatgcagtggccgcctgcaaggtctttgattacctggactgggagcgacctgtgggtgctggtggcacctatgtgcccaccagactgcggggccacatcaccttccatcgggtgtccttcgcctatcccactcgccctgagcgcctcgtcctgcaagatgtcaccttcgagctgcgccccagtgaggtgacggcgttggcggggctgaatggcagcgggaagagcacctgcgtggcactgctggagagattctatgaacctggggccggggaagtgctgctggacggggtgccgctgcgggactacgagcatcgctacctgcaccgccaggtganggggtggggggaaatgttagctgcactgaacantgctggggctgaacctctgccctgggggcaggtggcactggtggggcaggaacccgtgctcttctctggctccattcgggataacattgcctacgggatggaggactgcgaagaggaggagatcatagcagctgcaagggctgcgggtgctttgggcttcatctctgcactggagcaaggctttggcactggtgagtgctggggagcaagggggggacccgggtgtctgaccccactcatccccaccctcatcctgcagacgtaggggagagaggggggcagctgtcagcggggcagaagcagcgcatcgccatcgcccgcgctttggtgcggcgtcccaccatccttatcctcgacgaagccaccagtgctctggatggggacagcgatgcaatggtgagcactgagcagtgggtggggggagggtctgnccctgcagtgcatgctgatgggcagctgtgtgtcctacagctacagcagtgggtgaggaacggaggggaccggacggtgttgtttatcacccaccaaccacggatgctggagaaggcagaccgcattgtggtgctggagcatggcacggtggctgagatggggacacccgccgagctgaggacccgcggcggaccctacagccggctgttacagcactgagaaccatggagcagctggagtggcatgcgatgggatatggggagcagtgactgcctttgcttccagctgcaggatgggatgttttgggatttgtgtggaataaagtggagatgctttgt

SEQ ID NO: 9 - DNA, 10256 bp, Gallus sp.
gtccctattcccattgtgtcctcacatctgccatctcttcctgtccccatctatgctttgtgccccccatcccttaccccatccccacgtgtccctgtggtgccacctccacacgtgtccccgtgtccccacagcggggccgtggcgcaataacactgtgatgtggcgctgctgccgggacggagcgacggcgctgcccatccgtgccacgtgccagcagaggggacagcgggtgacgacggccgggggctgccgagacgccttcctgcagtgctgtgaggtggcacagaatctgcggcggaagggacagcgcggggggttggcacggggtgagtgtcagcagtgtccccaaagcggggaggggtgacctggggtggtggcggtggggtgtgggggagttgtagaaatggggaccccattggtgtggggaggtttggataaggggtccccatgggtggtggcacatggggacatcccatagcctgggatcccatggttggggccatcccgtacctgggatccccacatgggaggatgtcccccgctgtccccatggcagtgatggaggcacagctggcagagcagctgttggatgatgatgaggacgtccccacgaggagcttcttccctgagagctggctgtggcgacgcatccatgttgctggcactgcacggtgtgtccccgtgtgtccccatgtccccatgtccccatgactttgtgtccccgtgtccccatctccccatctccccaggctctcagtgctgctccctgactccatcactacgtgggagattcaggcagtcgccatcgtccctggacatggtgagtgtcaccccctccaatggccctgcagtgtccccctgacatccccctcgtggtgtccccatgtcccccacgtccccaagttcctatggtgtccccatgtcccccctctccccctccccccggaatgtccctgtgtccccgtggtgtccctgcactgccccgcagtgatgaggtcctggcagggctgtgcgtggcggagccgcagcgggtgacggtgacacaggacgtgcgtgtggcgctttggctgccccccagcatccggcccctagagcagatgcagctgcagcccctcatccacagcagactgccccgcagcatcaacgtaagccctatagagaccccataggcaccccagagatacctctttccctctaataaataccactttgcttccaatagataaccctcctgccccataggtacccctgtgctccatacttgccctgccacagcatacatacccctttccctccaacagatatgcgttgccccatagataccttctttctgccctatagataacccctcatgccccacagattcccgtttcctttcaattggtaccccctgcccctcatatatccccctctaccccacggatacccccttagacacccggtaccacttctgccccatggataccccctgtggcacatagataccgcttctgccccacagatacccccttcctactccactgtcccacagcccccactgccccatggccacccatagcctggtggcatcgggtgacagtgacggtgatgcaggtgacggtgacactgtcggcagtggagggggtgtgcgcggcgctggatggggtcccccagatgctggagctgcccccggggagggcagtggctgcacccctcactctggtggccctccaccctggggacatccccatcaccatcaccgcccgcgggccatgggggctgggggaccgtgtcacccgagtcctgcatgtcgaggtgagatcagtggggtcccctccagtcacctgggtcacctctggggtcccttaaagccctgcgacctcctggacattgttgtccttgtgagcctgcggtcaccctgaatactggggctgtcactttgaggttcatggacaccatgtccctgtgtccatggtggccctggacatgttggtccttatgggatctggggacatggggtccttggtggtcctggatactgcagttgtccttttgtggacactatgtccccatgtccttggtgggaatggtgtcatccattcccgcagcctgagggagagctgcacctggaggagagcacctacatcctggacgcagatggtgggtgtgaggactgggggacactggggaaactggggacgtggggccggaccctgtggtgtggtgtccctacagataagcggagccggagcctgaagctgccgggggacgtccctgcagagatcgtccctgatggggacttcagcatgagcatccgtgtcagtggtgtgtggggatggggacatggggtggggacatgggggtgggtactgggaacgtggtggggatgtggtggtgggcataggggacatggggacatgggaggacatttgttggggacattgatgtccatccctgatcatctctctgtccctatgtccccatacccatgtgtgtggccatgtccgcacgctgtgcccctgtgtgtgtcccctgggtgtccccacatgtgctcacatccttattacatccccacatctcctgtgtacaaccccgtgtgccctgatgtgtgcccctccacacatccccatgggtgtcccaatgttcccatgtccctctgctcatccccatccacatccccatgcctatcccctatccccacgttccccccatttcccatcctatccccatgtccccatgtcccatatctccatacccctgtgaccccatatccctgtccttcaactcccctcccatccccacaccatccccatgtcttctgtccccacaccatccccatatccccctgtcccccctgtccctgtcccaggccgggtgccgggctgggcactgcagggcgctctggggataggggactctctgctccgctccccccggggctgtggggagcagtccctgatgtcaatggcacccactgctgctgctctgcgcttcctggatgagagcgaagggtgggggcagctgcccccagggcaccgacagcgcggcctcagaaccctgcagcagggtgagctatggggcaggttgtgctttatggggtgggcaatgctttatggggtgtgcagtgctccaagggatgtgcagtgcttcatgggggatgcagtggggtttgatttgatttgatttatgggtttgcatttctcctccgaggattgcatctctctatggtgtttgcaatgggatgtgcagtgctccaggtggaggtgcagagccctatgggggtgcagtgctgtgtaggggatgtctgtggtgtccccaatggtctctgatgtccccacaggcttcgaacgggtgcagagcttccgcaaaagtgacggctcctatggggcatggctgcaccgggacagcagcacctggtgaggggagcggggatgatgtggggacatggggatagtgaggggatgtggggatgctggggtatggggatgtgaggacatcatagggacatgagcggtggggccatgtggatttggggacgtggtgacacggtgtcctggtgcaggctgacggcactggtgctgcgtgtgctggccctgtcccggccctatttgccagtggctgccagcggccccgctgcgtccctgcggtgggtgctggggcagcagcgcccagatggcgccttcttggagcacagggctgtggtgcaccgtgagatgcaggtgggtgacacatcactgctgtgtgcaatgtccccatgcaggatctccccctgcaatgtcccctgaaggtccctgcaggctgaccccacattacactgtgtcactcacgtgtccccgtgtccccagggtggtgtggcagaccccggcccggaggccaccgtgtcgctgacggccttcgtggtggtggccctccatggtgcccgcgctctgctgcccccggacagccctgagctgcccctcctggtgagtcccatgtccccacccctgtgtcttggtcctcatatccatgtgtcccttgtgccccatcccccaaatccccacatcccccatatgttcccataccctgctgtgtccccccagtgttcccccgtctttcattctccactatcccccgtattcccatatgtccccctgtccaccagtgtcccctcatccctctgtgtccccctgtcccccagtgtcccccacgtccctgtatgtccccatgtctcctagtgtcccccatgtccgtgtcctccagtatcccccatgcctccccgtgtctcttcatgccccacactccacgtccccacactccatgtcccactgccacaggacaaatccctgtcccgggcctccacgttcctccggggccgcgtggagcagttggggacctatgggacagccattacatcctatgcattggcactggtggacaccgctcctccggggccgcatccggcggtggaacgtctgcggggcatggcccggagcgcccacggtgcgtctgtctctgtccccatggggtggtggcacctctgtccccatggctgcctcctggacccctctgtcccctccttcagattcactctcattcgaatccttcaattttattctccctcaaactcttcttctttgtattcttcacattcattcctattcaaattgctctccttcctgtctgttcttcttcaaattcttcttcaattttgttctcctgattaattctcttaaaattaactctcgatcaagttctgcagattcgttccacttcggatggattcttctccaaactgttcttcagattcactctccttcaatttcgttcttgtaattaattcttcttcagagtgattcttcaaactcttcttcatgttctcttcaagtccattccctgcactgactccgggtgctcaggacccccccgtgaccccatatgaccccatatgaaccccccatgacctccacaaaaccatatgaccccgtgacctcccatgacccctcatgaccccatatgaccccccatgaccccatccctgtgcaggtggccgtgcaaccttctggccatccggtggccccgcagccacggtggaggcgacgggttacgcccttctggcactgctgcagagccgcgacatcgccggggctgcgagggcggcacggtggctccgacagcagagcaattacgggggtggcttccactccacgcaggtgggtgggggtcactgaccccccgggtgcctcggggtgggggtgatttgatccccaggtacctctttggtggctgtgtccccaacctgcttggtgttcccgcaggacacgctggtggccctggaggcgctggcccagatgtggctgcactggggccgtgggaacacaatggggctgaacctggggctctcctggccggggggtgcccgggggagggctggtggcactcaggttatgctgaagccggggctggagccgctggagcaggagctgcaggtggggacatggcgggatgtggggacacgagggatgtgaggacactggggacatgtctggacttggtaggatgtaacatgaagacactggggacatggtaggacatgggggacatgagaacacgggatgtgggggacatggtaggacatgatggacacagggctttggggtccttgggtcctcgctctgtccccatgtccccaggtgcctctgggcagcccagtgacagtgcaggtggagggacacggcgaagggacgctgacggtgggtggctgcatggacattggtgtcatctccaagaccgatgtcccctcacaacctcccctcatggtgtcccctcatgctgccacggtgtcccctgctgtcccatcatggtgtcacgctgtccccaggtgctccgccagttccgcctgctgtcacctccgaacgccacgtgccaggcgctgcacctggaggtggccatcaccggccccatcctgtaccatggtgaggccccacccaaaggccccgcccccttttcctcgcggggggcgtgccctcaaccctgttttgcatatcccaacccccagcagatgaggactacgaggactacgaggactacgaggaggcggagcctaaggagggggaggagcctacggaaggggcagtgcccgtggaaggggcggggccagcagatgaccccgcccccctcagccccgtgtccttatgggatgcccgtaagcggcaacgccgcagcacacataaccctgcccacgaggtggccttcctggtctgcttccggtgaggggcggaacttcctgtccctgggggcgggtcttcctgctgatgggcgtggcttattgctgaggggcgtggcctgttgtaggcggagcccaggggtggcactgactgggatggcggtggtggagatcactctgctcagtggcttctcaccccatagagctgacctggacaaggtaggggcccagggggacttgtgggacatgttggggggttgaggggagttatggggtgtggggtttgggggtgttggagttgttgaggtggcagaatgtttgggttggagtcatgggatatggggctattggggtttgagggtgttgtgatgttgggaaacattgaattggggttgttgagtttgagggtgttggggtgtgcgggtgcagagctgcagctgctgggttggagtattaaggtgttgggatgttggggtgttggatggcttggatgcgggtgttggggtgggcacgtatctgggtgctgctgtcccacaacagctgcgggacgtggtggatcactggatcagtcactatgagttggaaggaaaccagttggtgctatacctggatgaggtgtgtcctcccgtgtcaccctataaccccagtggccccatgttctcatatcccccatgtccccgtgtccccacaccatatcccattctccccacacatccccgtgttccaccacgtgtcctcatttctgtccctgtccccaggtcccccccgagcggcagtgtctcagttttggggccacccaggacgcggctgtgggtcacatgcagccggcaatggcagccatctatgactactatgagcctggtgggtggggccttcagtgggaggggcctaaatgggtggtggtcttcatgggtgtgaccattggaggaggcgtggccgatctgacccctccatgccccatccaggacagcgctgcaccgtcttctacaacgccccccaaaggagcagcaccatcgccacactgtgctcccccaaaatctgtgaatgcgcccaaggtaggaccccactgtgactccatatgtagggcccccatccagtgaacccccacatcctcctcctaatttttgaagatctgggggtgaaattatggggtttataggggagcgtggttgagtgacatgcaggacatggagggaacccacaccaagaaccttgtgttttgggtccctgatgatgttgggagatcctattgatgttggtggtccccaggggggtgtccccaagcccaaaggaggacacaggaggtgacagctgatgaccgccatgactttgcctgctacagcccccgcgtggactatggtgagatcccaaatcactgcacctcaaacctgaccccaaattggctgcatcccgaaccccaactgccctaaatcccatctgctgcccctgagtcccacagctgcacactgtaccccacaagtggcccctgaagcctaaaaacattcacgaggattttgtagttttctccctgtaccccagttgtccctctgaccccaagaaccccacagctgccctatgctgtcccctgcccgccataactcctctgatacaataacccccgtgaccccatctttatgacctccatgacctttgacccccagcactggtggttcgggtgctgtcccagagtgagataggggcttttgtggcgtttgagacggaaatcaaggaggtgctgcttgaaggtgagactgagggtagtgggacggactggaaggtgagaatgggagcactgggagaggcagggagtactgagagggactgggaatgactggaaattgagactgggtggactgggaactctggtagagactgaatgggtatactgggaacactggaagaagttgtgggatgagaagaggatgctgggataggagacccccccccttgtgctaggggggtctctcagccatactggcacaatatgagagtatactgggtggtactgggaaagctgggaggactcatactggtgtgtactggtgcagggcaggacacagcagtggcccctggggagcggaggcggctgctggtgcggaagagctgcccactgcgcctgcaactccacaacatctacctggtgatggggggcagcgggaggacgcgggaccctgaggggcggtgagaaggggctgtgccccatgtccacatgtccctgtgttctcatgttcccatgtcccatatcccagtgttcctaaccccatatccttgaccttgagcccataccctgatatccctgaccctgtccccattctcagcccccagttcctgctgggcccccactcatggttggaggaggtgccatcccctggacgctgtaaggccacaaggttgcggggttactgcgcccaactgcaggagttccgcacccgcctgagccaactgggctgccagctgtgagcccctgggagccactgggagcatgttgggtgcagctgggaccattctgggggtgaactggtaccactgttggatcagttgggatcaattgggaataaactagtgttgactgggaccgtgttgtgaccaactggaagtgtgttggaagaaactgagagctgctggggttgagtgggagcaactggaactgtgttggaacaaacaggggaccaactgggatcacactgtggtcagctgggatcacactgggtcaaaaaagatcacagtggcccaattggggtcatactggggtgagctgggatcagaacgagtttaataaacgtacagtcgtccgagccaccacagagtcagccctccagcggcgcagagcggcgcagcgcgcactggctgcccgcggtaagcggatgtgacgtcacttcgcggcgcgctattcgaactccagcagcgccccgcggagcgccccaatgccgcggcccaaaccgcgcagcccccggcgccggggccgcccccccccggccgcccccccgccaccccccgcgcggcctcgcggtgagtgcagcccgtaggagtgcggagtgtgggggcgggggggggggcgtctggagcggagcctttatcaccgctgttttcccgatttccccgtcttttcgccccgtttcagcccgccggtaccggcccggtcagagggcgctgcgggagatccgccgctatcagagcagcaccgctctgctgctgcgccgccagcccttcgcgcgcgtggtaacgggactgccccggaacgggacaccccccaacccccccaacgggaccatccccccacggatggatccccccccacacacatccaacgtgggaccccccgccccaaaatgagatctcaacgtgagatctgggggcctcaaaatgagacactctcccccctcccccaacggaacaccccgaaaatgggaccacacataaaagtggggactcccctcctcccccccgccccgtcaaaatggaacacccccaactggacctttcaaaaaataacattcccctccccccaaaaatgggacttaccacaaagtgggatcttcccccaaaatgaacaccccctcaaaatgagacccctcggaccccccccaacccctctgcacccatcgccgtcgtgcacggaagggaaaggctgtagggtacatctacccttatttcttgggtttgtgttttgttttgttgttatttagaagcaaaaccaagacaacaaagcccagccaatgccatttcctggcagtggacgcaggcgcaggcgggttggtcacaaagcaagaagttgctgcgggactttgtcgttttggggccgttctcgtgaacttctgagccatggatgaggaaattacttatgctgatttaaggcatcctacgggcagtttgcctcctgctaagcggcagcgcggtaagggatgctctgtgtggtgggtgctcaccgcaggcttggtttgggggcttgctgttctctgagaaacaccagcaatgctggttgggttctgggtccaccctggcttgtatgggggagtaaaggaaggggtgggggagaaggaagcctgggaatggccagaggtgtggtggtttt

SEQ ID NO: 10 - DNA, 10256 bp, Gallus sp.; identical to SEQ ID NO: 9

SEQ ID NO: 11 - DNA, 1128 bp, Gallus sp.
agaagagccccgtgatgtcctccaggtgcggtccctcggtgcctgtggggacaacgacagccctaagcacagtgtcaccatcctgggtggggtccccaacccaaatccatgatctcccattgtcccaggccatggtcctgatgtccctcagaccctcctaaccatggtcccagcatcccaatacctccacgtgtttccaatatccccacatcccccctcaccagccaggagcagtcggacggagacacgcattggtttggccagtgcagtgtgggtgacaacgcagctgtagatgtccccgtggtgttgggggcgtgcggggatcagccgtgctgccgccgtccggctgtaggttccatcggctgcctggcggtgacctgaagtccagctgtccatcactgtgtccctgggtgactgtgatgtccccgagcccccggcgcggcgctgccacgtcaccgtcacatccaaggggtagaagccagacacgtggcagcgtagctctgctgacgtccccggggccaccaccaggttcttcggggacagcgtcaccttggggggctctgggagacatgtggggggacatcggtcccatatagcccatagggcccctcctatagggctcatccccccctataaacctacaggtgaactatgggatgatgccaccccatcctatagtcctcataggaataccacccggtcccatccaccctatagcctccataggaataccacccagtcccatccaccctacagccccccacaggaatatcacccagtcccatccaccctacagcccccataggaataccgcctgctcccatatgtcctatctgaccaataggaataccacccagtcatacacactccgtaggaacactgcccaaccccacaccccataggaacaccgcctgccccacatggacgcaccaaagacgtggagctgcagcactgtctgtgtgtgcccgtggggcaggaacacggagcagatgtaggtgccctcatcccccggtgatggccgcgccagccgcagtgtcaccgctgtcaccccgtccccatcccgtgtccccagcagcagttcggccccgggggtggcgcggggggcgcgggcggtggaactgtcatagg

SEQ ID NO: 12 - DNA, 334 bp, Gallus sp.
ccaacttcctttggttcagggaagaagactcacccactgctttggtttgttgcactggaaaagcatgaagaaagcaccacatgatgagaggaacagttcatcccacagctcacgcaggaagaacccatttttaatttaatttgggagggagcactcacccaggtctgaagctagtttatctgcaatgaaacaaataagaaatgcatgatgagaagggtcagaatatcatcccatggctgatcccatgggaagaccccgaatctctttggtttgcggaggaggactcacccaactgtgcattccttccctctgcaaagggaaagcagaaacagtg

SEQ ID NO: 13 - DNA, 620 bp, Gallus sp.
tgggatcaagttgagtagacatagcatcctcgcttttagacaagacctgcacagtataccaccgtttactgtgcagataatgaccaaaagcaatatgcgtcacacttttctggtgacaacgtcacaaaatggcggtcgtcaatcgtgacgaacagcacaaacgccctttctcatcgaagatttcaatctgccagacctggtgacgcgaaccgagatgcaacggtttgcatacgccgcgcacccgcccttctcgtgccgagcggacgtggttagcattgatttccagaccaaccactttttgctcaccttcggtacataaataaccggcaacggaaccgatactttcggccattaccacgggtgctcctccatgcagcaacccgaaaggctgctttgtccgcgagtctactggcattgtcgcttcaagggtgtcatcaccaatatgttcaaagcgaatatccaggaaccccaccatgtttccttcacccatagcattcagtgcttccagggtgattttccgtttccagatcatttaataatctccagttaaagcctgcacaggatggcttaccccgtgccttcaacccgttttatctggctacggcaaggaatatccggtt

SEQ ID NO: 14 - DNA, 475 bp, Gallus sp.
ccgtcgcctcggctctccctcgggctccacccccccgttccgcccctttgccgccgcatctcccgctctgtaccctccccaagaagtcgctcagacggcgtcgcgttgtctgcacatcctcggggaccgtctgttgtgcggcagcaggggaggggagcgggcggtctgtgctcttctattcccttcagtacaagaaggtggtttgggttctttaaccaaatatactcttttgtttttgcataaaatcaccagaaggaattggtctgttgaatatataggagtggtggagagagtcgaagaagtgtttcctgtgacaaaacaccgttaaaagtgaattcatggagaacgcactgcagtgacacagaagggaaaacacgaaacataaataatttgccgatttatcatcgatttcagggtcctttgggctgattgctttcccagtatttccctttggagaaaaaccggtgaaaaatgg

SEQ ID NO: 15 - DNA, 518 bp, Gallus sp.
tcacctggctttgctgctccagaccccgcaggaagcgacccccctggcccctggcatcccgcagccccacacgcagctgtgcacggccccacactggcgccccatctgggaatctgggggtccaaagggtcagtggagtcaggcgggtccaaaggtcagtgcggtcaggaggtccccagatgtcaatagggtcagggggagggatcccaaaggccaataaggtcaaggggagagattccaaaggtcagtagggtcaaggtgccccagaggtcaatagggttgggggaacccaaagattatagggtcaaggagtgaccccaaaggacatcagggccactgatttggggtggatgggagaggaatttggggagttcaggagagttggaggggatttgggaggttttggaggagacagatggggattttggtgggaatttggggaagattgggtgggatttgggatttgggtgggatttaggtggggatttggggggattttgtctctgggtgtcccatac

SEQ ID NO: 16 - DNA, 194 bp, Gallus sp.
cctgaaactttggggtgagcatctccatcagctcatctgcaatgcaatgggatcttccagtctttgggttttgtgctcgttgtgccactattttcatggcatcctaagatggtgctgtattatttttgtgacactgtaagagactggagcagaaattttgtcacaaattaacaaaaaaaaaaaaaaaaaaaaaa

SEQ ID NO: 17 - DNA, 710 bp, Gallus sp.
gttctatgatttctttggtccgaataccatgaaatctgatatttccatttcagtatctgaactgggttctctgctggatcacagtggtccacacaaagaagcagaacagtatatcgctcgcgtctttaacgcagaccgcagctacatggtgaccaacggtacttccactgcgaacaaaattgttggtatgtactctgctccagcaggcagcaccattctgattgaccgtaactgccacaaatcgctgacccacctgatgatgatgagcgatgttacgccaatctatttccgcccgacccgtaacgcttacggtattcttggtggtatcccacagagtgaattccagcacgctaccattgctaagcgcgtgaaagaaacaccaaacgcaacctggccggtacatgctgtaattaccaactctacctatgatggtctgctgtacaacaccgacttcatcaagaaaacactggatgtgaaatccatccactttgactccgcgtgggtgccttacaccaacttctcaccgatttacgaaggtaaatgcggtatgagcggtggccgtgtagaagggaaagtgatttacgaaacccagtccactcacaaactgctggcggcgttctctcaggcttccatgatccacgtttaaggtgacgttaaacgaaagaaacctttaacgaaagcctacatgatgcacaacaacaacttctccg

SEQ ID NO: 18 - DNA, 5636 bp, Gallus sp.
ccaccaccgctttgggcagtgccagtgctcctcacaggctgtggggcagagcaggtgaccccccaaggatttcccctacaaagagccccacagagacagaaatccttcacctgagctgcagcaagcgcgcggctacacccagcatcaatctttgcccagcttctacctttgcccagcttctacctttgcccagctccagggtgcaatgcgagcaacttggcatcagaccaatacagtcaaaggttggagaacataaaacacatcccattgcagctttgtgcaccaccctgggtctctgctatcaccaggaacatggacacaggagaagctttgccatagcacaggagaaagctgtgcgctgcacttcatgagcatttctctcaatttctcctgtatcccacaggttacaggcaccagtaattctgccagagctattctgaagggcacgtggtgaaggattatggcttggagcagtggggagagccaaaagcccttcccacacttgatgcactccaagggtgtgatcccagcatgcagcctctcatgttggaatggtcaattttatcctaaaatcctcttgcacttggagcaatgttgagttattttccccatgtgcattcacagtgaggtccccctgaagcctactcttctccagccaatttcttatgatcacgaaggggatgatatgatggtgacatgggggatttccacgtggatgctgcagggcagatggggaaggggtgaggggagatgcccaccagcagagttcccaatcaggacacagcagttttgctgccagcaccaggaagcagcttcccctcctttccctgctgggaaatcactcctttggaatgtttttttttttctgctgctcacccacattttgcacagggctgatcttccaggtcagcccaaactctgcatccccgcacggataacctctccctccctaagaatcagtgcatcctgcctgcctgcaaagcagctgctgagatgtcttttgcagcccttattcccgcagaccccgtgcagaaccacacacatctccatcctctcctccgttggcaaggaatgggtttgcaaagggatgggcacaaccagcaatatgcaaaggaagaggtgtcgaaagtctggggagcaatgaatctgtcccccggaagatgtttccatggggcagttaaggaggagaattggaaatgaagcagatgatgcagcaatgaaactatcccagaaaaggggggaaaagcaattctggtaatgaagatacataaaggagaagggcttctcgctgtctggacgcagttctgttggttaacgtcttttctctttgtgctctttgcacttttttctttgcctgctctggtcaggatgaggcagagccctcacggggccctttcacacctttttttagcacacagaagcgcagcggccgtctcagcacccagcatcgatgagaagggactgcaaataaattaaatgcgttactgaatagacagtcgtaattaaaagtcaaacccatcccctcccagtattccagctgccgaggcatcggttggcacagaatcaccaaatattgcctttcttcccccatccccgcttatcagccaatgctctctgacccctaaaaggtctcgatttggggtctttttgttgttgttgttgttgttctgggtatttttaggcttttattatcagcgatttttcagcttctcactgcttaccccccagctcagcaccgcatcgctcactgccatcgctgaacccagcggcgtttccatccctcagagagcagcaaaatgagacatcggccgtcgtgcacggaagggaaaggctgtagggtacatctacccttatttcttgggtttgtgttttgttttgttgttatttagaagcaaaaccaagacaacaaagcccagccaatgccatttcctggcagtggacgcaggcgcaggcgggttggtcacaaagcaagaagttgctgcgggactttgtcgttttggggccgttctcgtgaacttctgagccatggatgaggaaattacttatgctgatttaaggcatcctacgggcagtttgcctcctgctaagcggcagcgcggtaagggatgctctgtgtggtgggtgctcaccgcaggcttggtttgggggcttgctgttctctgagaaacaccagcaatgctggttgggttctgggtccaccctggcttgtatgggggagtaaaggaaggggtgggggagaaggaagcctgggaatggccagaggtgtggtggttttgagcaaaaatcagcccagatcgggaagcccaatgtgagagaatggaatgaaatggtggcaaacgcaccctgcatccacgtggcatgagggctgcagacatccccgccctcccagccaccggctgccccacactgggctcagctcacaaagcctgggggctgctcagcttccaccccatgctctatggagcctgcagggcctccaccacctccagaaccacacgtggaggtgatgtccctgtgtccatctgacctccagcgggagcccatcccatgctccctgctgctgtcacccctctgtgccacctccttcccagctgggaaccactgggagccactgggaagggtccaggggaccctggaactggaggaaaacaaacaggcatcaacttctgctcatacacagcatgggaaccaatgggaagggtccggggaccccaaattctcaaggaaccaaagcagccatcaactaccggatttgttgttcagcagaatgcatctgtgtgccccatccccactccacttcatttcctttcttttcctgcaataggaaatccatcttggaggggacggggacacaggcaggctcacagaggggaccctggggtagcagtgccggatttgggctgaggcccatagcagtgaccacagaatcggtcatttgtccgttcatggtgaagatgggagggggttcagcagaagcactccctgggactcccagagggctgtctcagaaccgctgctttccttgcacagaagatgaaccatttttgtagggggagggtccaggatgtggttgcagtgtgaacaaagcctgtgtgcttttataattctcttcctgcctgctgctgtcatttctgagggctgaatgggcagcacgggcagacagcagcgtggctccgacacttctatgtctgcagtgcccattgcaggaagagaaaagaaatggagtggggatggggcaaacagatgcattctgctgaacaacaaatccggtatttttttattgagagaaataacacaggattgtgagctgattgcatgagcgcatgcagcgatgtccccccgtgtgcccgggcagtgctggggtctgcacagcccaaactcctcacagagccgtattgcagagcttcaccccaacgcctggggcttttggggtgggcacacatcagagggagggactgcgttgccctccatctcctgcacattatggatggagacgtaaaggttctttctggcagacccactggtgttcaccactacagacgtcgccctccacttttgtgttcttgaaggtcccgcagcgattctccatcacggaaaggttatcagacctgcaaaataaggctgtttgcacccaaacacccgacttgaaggaggcgggcaatggttgcagaaatactcactctgtgctgttgtaggaggagttgtccacccatttccattgatttgtggacacttctaatccaatccacaccggctccgcacctgccatctgctggaggtgatcctgggaaatggcaccaaaatccttctgcaaggggctggaggggtgcagagccaccaagtctgccttgttggacccccagcagatgggactcagacagcagccatgcctggaatgctgcctggctctgcaggcggctcaatgggtgggaatggcttcaaacccgagatggaggcaccggtgtgaccagctgagctctgcttccatccttcagcctgtttgaagggtgggaggggacacaacccccatgtcccacccctagcctgaaccttgatgtccttaactcaaaccataatgtgcgcaaccccagcgtgcctgaccccaacccgtgtgcctactgccatgtgttgacccctaaccctaaagggcataatccagaccccaatctctccagtgatgctttagccccatttgggtttggaaccactgaccctcctgctgccgcccagtcactccagagcggttttctcccacagaatccaccaaacccacacattttcaggtcccgtccagctccctgctctatgcttacctcttctgccttcttccggagcacagccagctgagactgcagattttcacacttcatttttgcttgtgtccagttccccttttctgtggaaagctcatagcatcggtcccctaaaagcctccagaactggggacagagcaggcaggcagcaggggctggagagaaagagccgtgagcatcttcaggtgggagaaatcccacccaggaggatttccttgggaagggcattacctgcagagctgttccatgtggattggcagaagtactgctcaatggaggtattctcgcagagctctgtcccattcctcccgttggtctcagggcagtgccgggcagcgcttggaggtggtgttgttttctgaaagacttttgggcacaacctggggtgagacgcggccctatggggccaaccccgtggaaaccacgcagggttggggttggatcctcgagctcttttgcaaagcctttctggctatggttgcactcagttaattaaactgtctaaaaccatattttgtatataattagacatgatgtttactgcttctgtcccccccttggtttaagagcagagaggctcttgcagaagggaattcctctcactgagtgccactttggaattgttgtgtgatcacccaaactccagtgcaaagccccagccccactttgggcagaatgaatgtgttttctgctcagaagagcttcgatttcctgtgcagcaatgtggttgggatctgatcactcaccgcacacgctgagccctgtcaccagcagcagcagcagcagcagcagcacccccagcatgcaggctttctggaagtcccacggaactggaagagcccacacttatataaaacagacattttgaaaaaacttttccttttacagaaatgatctccctgtgaaagagcccctccaccaacctgctacgttagagcagaagttgatggctgctttggttccttgagaatttggggtccccggacccttcccattggttcccatgctgtgtatgagcagaagttgatgcctgtttgttttcctccagttccggggtcccctggacccttcccagtggctcccagtggttcccagctgggaaggaggtggcacagaggggtgacagcagcagggagcatgggatgggctcccgctggaggtcagatggacacagggacatcacctccacgcgtggttctggaggtggtggaggccctgcaggctccatagagcatggggtggaagctgagcagcccccaggctttgtgagccgagcccagtgtggggcagccggtggctgggagggcggggatgtctgcagcc

SEQ ID NO: 19 - DNA, 2617 bp, Gallus sp.
cccagaacccaaccagcattgctggtgtttctcagagaacagcaagcccccaaaccaagcctgcggtgagcacccaccacacagagcatcccttaccgcgctgccgcttagcaggaggcaaactgcccgtaggatgccttaaatcagcataagtaatttcctcatccatggctcagaagttcacgagaacggccccaaaacgacaaagtcccgcagcaacttcttgctttgtgaccaacccgcctgcgcctgcgtccactgccaggaaatggcattggctgggctttgttgtcttggttttgcttttaaataacaacaaaacaaaacacaaacccaagaaataagggtagatgtaccctacagcctttcccttccgtgcgcaacggccgatgtctcattttgctgctctctgagggatggaaacgccgctgggttcagcgatggcagtgagcgacgcggtgctgagctggggggtaagcagtgagaagctgaaaaatcgctgataataaaagcctaaaaatacccagaacaacaacaacaacaacaaaaagaccccaaatcgagaccttttaggggtcagagagcattggctgataagcggggatgggggaagaaaggcaatatttggtgattctgtgccaaccgatgcctcggcagctggaatactgggaggggatgggtttgacttttaattacggctgtctattcagtaaggcatttaatttatttgcagtcccttctcttccatgctgggtgctgagacggccgctgcgcttctgtgtgctaaaaaaaggtgtgaaagggccccgtgagggctctgcctcatcctgaccagagcaggcaaagaaaaaagtgcaaagagcacaaagagaaaagacgttaaccaacagaactgcgtccagacagcgagaagcccttctcctttatgtatcttcattaccagaattgctttttcccccttttctgggatagtttcattgctgcatcatctgcttcatttccaattcccctccttaactgccccatggaaacatcttccgggggacagattcattgctccccagactttcgacacctcttcctttgcatattgctggttgtgcccatccctttgcaaacccattccttgccaacggaggagaggatggagatgtgtgtggttctgtacggggtctgcaggaataagggctgcaaaagacatctcagcagctgctttgcaggcaggcaggatgcactgattcttagggagggagaggttatctgtgcggggatgcagagtttgggctgacctggaagatcagccctgtgcaaaatgtgggtgagcagcagaaaaaaaaaaaaaaaacattccaaaggagtgatttcccagcagggaaaggaggggaagctgcttcctggtgctggcagcaaaactgctgtgtcctccatgggaactctgctggtgggcatctcccctcaccccttcctcatctgccctgcagcatccacgtggaaatcccccctgaaaaagcccattttgtgaccatgcatcacatttattttcgcattcagcatcagacggacacaggcaatgggttgggggatggggggggggtctgagggtatatctttttgctgagccaggttttgagtcatgggggataatttcattccaaggggaggggggcatttaactgcaggtggtaacaatgaaaggcagtgggagttgttgtgattgcatgggggaaagcactggttttttccataaattgggactgatgtggctgttgttgcttatttttatgggggagggttgtggggtttttttcccctatattacattgcatttaatttcagtcctctctcattgtctatccctggcaatgctaggacttctccttgctgttttctgttgggcgatcattgccacagagggaggaattgcttttcatttgggtcactgcaatgagttttagcacccagaaatatatccttatgggtctctgcttttggggcactgctgatgggtggaagttttggtttgcaggtgaagtggaagccccaaaatggaggaagtgagggaatatccccatgttttgggcacagaatggagcaggagggaaggtaacagccgagccatgcccttaacacatctgtttattgttattattattgttattattttattgattacttctttaacttgagaacaaaggggagggatgtgggtgggaagaaaatgagtctcatttcttttagcacttccctcaaggggaaaatttgtgttggttgttgagcagcaggtggacttcttgctgtgagcagccacattttggaagagttctgttgttattagcattatttatgcgattctgtgatgtttttattataattaattgtaatgaatcctccctgaggcactggatgggggaaaaaaaaacaacattttggggtctactgctcacacctggggtgcactgttgcccattggaggtcccttctcccataggtcccagccgtggggcatgcgttaccttccagctcacgatggcagcggtgttcacagtgctgctcatcactgctgttgcctttgcaggtgagtgctgagggttccaaagagcagagaaaaccctttggg

SEQ ID NO: 20 - DNA, 785 bp, Gallus sp.
ttctcccacagaatccaccaaacccacacattttcaggtcccgtccagctccctgctctatgcttacctcttctgccttcttccggagcacagccagctgagactgcagattttcacacttcatttttgcttgtgtccagttccccttttctgtggaaagctcatagcatcggtcccctaaaagcctccagaactggggacagagcaggcaggcagcaggggctggagagaaagagccgtgagcatcttcaggtgggagaaatcccacccaggaggatttccttgggaagggcattacctgcagagctgttccatgtggattggcagaagtactgctcaatggaggtattctcgcagagctctgtcccattcctcccgttggtctcagggcagtgccgggcagcgcttggaggtggtgttgttttctgaaagacttttgggcacaacctggggtgagacgcggccctatggggccaaccccgtggaaaccacgcagggttggggttggatcctcgagctcttttgcaaagcctttctggctatggttgcactcagttaattaaactgtctaaaaccatattttgtatataattagacatgatgtttactgcttctgtcccccccttggtttaagagcagagaggctcttgcagaagggaattcctctcactgagtgccactttggaattgttgtgtgatcacccaaactccagtgcaaagccccagccccactttgggcagaatgaatgtgttttctgctcagaagagcttcgatttcctgtgca

SEQ ID NO: 21 - DNA, 2577 bp, Gallus sp.
ctgcgctggggatcttgttttcccctggcaatgggaacagctgttgggtgccttttttgggaaagatctctttatcggtgcatgaagaatgaagcgactaatggggaatggaaggagtggtggctgtttgagtaattgactgataggttgatggagggatacttgaattaagagcttttggctcttatctcattgcctctgtgcaccaggtttggagtgggccaggccctggcacggtcaacttgctcactgttggcaataggaacattttttgagcctcagagagattttgttggaggaatggatggatcattcatgtcctggtttgtctgggggggaccaatgtgatggattaatttttttcagtataaaaatagtttgtcaggtgaacttctggtgactgagtggatggttggatggagggatgtgagtttctgtggagggatggatggttggaaggtttgttggatgcactgttgagtgctggtgggatctacatttggggcaatggatggatggactctgagaatatagactatagctgagttggcaatgaccaagaaggaccattgcgttttgtttctggcttcatgtaggatcacccaggaattaaaccctatgtcatggttttgtaacttcgctattggtattccacatcataacatcatggacaaaagagaagaatagcaaagttacaaaaccatgacaccctacttctgaaagcagttttgaaatgcttggggagctgaatggttgatggtgtggtggagtcgtgggggggaggtgtccctgtggggcagtccctgggaagctatagctataagtcaccccaatgccccctctgtgtgggagtagtgtgggtgggggtcactgggataccacagtggggtggagcccaggggagtgtctttgaggtcagtggggggtgagcagggctctctagaggcctttggggggtccaaaaggagttgatgagagagagagtgtgggagatccatgggggggctgcaggcctcagtgccctccatctcttgccaggtgccccaggaacactatgggtggggacactgtggccccgcagtgctcacctgcattgggcacctccccatgtcccccctgaaggctacaacctcatctatggaccccccggtggccccgtgaaggtaataccccatagcactccctgaacttcccaggggatctccctgggtatctcctggggtaccccaaccctccttggggaccctgctcccaccctggggaatccaaaagtcctccaccacccaagcaccctaagaaccccactgcaccccactatcccttgaggtccccaatactccttttacagcattcccatcctcctccttggcccctttatgctctccagagacattaaacacccctgtaatgccccttagggacccctgcagcagcccaataatcctcccatgtctacctccagacactgcagctgccccctgaagcaacatccaaggagctgtggggcctggagcccagtggacgctatagggtgcagctctggggccgggggctggagccccttgagaccacctttgacacccgtgagctgggaaagggggtcctgtggggtgggaaggggcacttgggtggaggactctgggatacccaaatacctggatgatttggggtgctggggacatatggatgctgggtcctgaagtatggaggggggtaccaaggaatctgcatccttgggtggggagctctgggggttcccaagtacctgaataatgggtacctagttaggggaatgccttggggtggggggggggcggacacagcgggatgccctcgtcccttggtaggtgaacagggacacccaactggttggggccacctacactgctctgtccttcagcacccctcccccacccacatccccgggactgcgctgaggagcagctcaatggaccggggccttcacgagaggtcctcatcttcctcgggggcgaccggcagcggccactgcacgtcttctgcgacatggagagcaatgggggcggctggctggtggggaaacggggcggtggggagggtgtctggtgggctctagggggtgctatgaggagtctggtgggcaatgggggtcacagggtggggtggctgactccatggttgccattataagggttggattggcaataagagacctgtggagcaactgggggcatttggggtatctggggaggttctgtgggggttgagaagcaatggggggggggagtgggggaggctggaagatttaggggaggttaatgggaaggtcttgtggggcaattggggtaattctgggaactgcagggggatcccagtgttcctgtgagattcacataccccctatactatccatggggatcacagtaaccctctggaactataaatgggggagaacccagggagcaatggggggctgtggtggatctgggaggggcaatagggtgccctggggggcaatatgagggtcttagggtgcaatgttgggggtctagggggaagtaatggggggtctgggggcagtggtggggtctagagggg

SEQ ID NO: 22 - DNA, 1268 bp, Gallus sp.
ggagggagcactcacccaggtctgaagctagtttatctgcaatgaaacaaataagaaatgcatgatgagaagggtcagaatatcatcccatggctgatcccatgggaagaccccgaatctctttggtttgcggaggaggactcacccaactgtgcattccttctctctgcaaagggaaagcagaaacagtgtgtggtgagaggagcagctcatcccacacatcgcacaggaaaacccccttttttatttaatttggagggaggactcacccagttctgaagctagtttctttgctaaagaaacagataagaaatgcatgatgagaaggatcaaattatcatcccataggaataccccagatctctttggttagcggaggaagactcaccgaactctgtgtttcttctctctacaaaagaaaggcagaaacaatgcatgaagacaggagcatctcgtcccacagctcccaaaggaaaaccccttttttgtttaattttaaaggcagcactcacccagattttcaactagtgtctctgcaaaagaatcaaataagaaatgcgtgatgagaagggtcagaatatcatcccatggctgatcccatgggaagaccttgaatctctttggtttgcggaggactcacccaactttgcatcccttctctctgcaaaggaaaagcagaagcagtgcgtgatgaactgaacagctcatcccacagctcacacaggcatccctcattttgtattttgtttgggagggaggacttacccagttctgcagctagtgtccctgataaagaatcaaataagaaacgcatgacgagaaggctcaggttatcatcccatggctgatcccatgggaagtccccaaatctctttggtttgaggagggagactcacccaactttgcatccattccctctgcaaaggaaaagcagaaacaatgcattatgagatgaatgactaattgcacagctcccaaaacattaaaaaaaaaaaaatagtgggaagggaaactcatccactatcgcaggtagttctgctggaaaagaaagagcagagcagtgcatggtcagagaggacagctgctcatcccacagctgatgccatggggagaccctgaattccctcactttggggaaggagacttacccaactctgcatcttttccctctgcaaaatagaagcaaaggaaatgcatggtcagagggaacaccttctcatcccatggttgctcccatgccaatacccccaaatctttgttctggtaag

SEQ ID NO: 23 - DNA, 841 bp, Gallus sp.
cagtgacagtgcaggtggagggacacggcgaagggacgctgacggtgggtggctgcatggacattggtgtcatctccaagaccgatgtcccctcacaacctcccctcatggtgtcccctcatgctgccacggtgtcccctgctgtcccatcatggtgtcacgctgtccccaggtgctccgccagttccgcctgctgtcacctccgaacgccacgtgccaggcgctgcacctggaggtggccatcaccggccccatcctgtaccatggtgaggccccacccaaaggccccgcccccttttcctcgcggggggcgtgccctcaaccctgttttgcatatcccaacccccagcagatgaggactacgaggactacgaggactacgaggaggcggagcctaaggagggggaggagcctacggaaggggcagtgcccgtggaaggggcggggccagcagatgaccccgcccccctcagccccgtgtccttatgggatgcccgtaagcggcaacgccgcagcacacataaccctgcccacgaggtggccttcctggtctgcttccggtgaggggcggaacttcctgtccctgggggcgggtcttcctgctgatgggcgtggcctgttgtaggcggagcccaggggtggcactgactgggatggcggtggtggagatcactctgctcagtggcttctcaccccatagagctgacctggacaaggtaggggcccagggggacttgtgggacatgttggggggttgaggggagttatggggtgtggggtttgggggtgttggagttgttgaggtggcagaatgtttgggttggagtcatgggatatgggg

SEQ ID NO: 24 - DNA, 1804 bp, Gallus sp.
ccactcttgggtgagctgacagcgtcccacgtcagccccgactccgtccagctggaatggagcgtccccgagggctcctttgactccttcacggtgcagtacaaggatgcacaaggccagccacaggtggtgcccgtggacggtgggttgcgcacagtgaccgtgcccgggctgtcgccgtcccgccgctacaagttcaacctgtatggggtgtgggggcggaagcgtctgggccccatgtccactgatgctgtcacaggtgagcatgctgtgttctgcctccatgttcttttgctttcagtgtagttgtcatgtggcaggaacctttcagggccacttttggttaatgttgccttaatagtcaaggaaacaatttgttcttgttgagtgggaatgcctaacgggatgggagtttggatgatgagaggacaaatcttataagggatgattgataattattgcggaacggatggaaggaaggttggatggatggaatggtgtttggataaatttgtgctcagagcacagctggagtgttggatgaatgttgctttgcttgttgaatagatggatgtttggttgtgtggttgcttccactgagaattcctccctctgtgctgcagcagcagctccagcacaagaggagccaccttccccaccacgtctgggtgagctgacagcgtcccatgtcggccccgactccgtccagctggaatggagcgtccccgagggctcctttgactccttcacggtgcagtacaaggatgcacaaggccagccacaggtggtgcccgtggacggtgggttgcgcacagtgaccgtgcccgggctgtcgccgtcccgccgctacaagttcaacctgtatggggtgtgggggcggaagcgtctgggccccatgtccactgatgctgtcacaggtgagggcaggaattggcacctggtgggctctgggtttgcagcaggtagaaatgtaaacgtggcctgcgctggggatcttgttttcccctggcaatgggaacagctgttgggtgccttttttgggaaggatcccttaatcgcagcatgaagtatgaatggaccaattgggtgtgggtggagtgatggctgttgagatgagttggtggctgcttgagtaattgtctgttggaatggatggacagatatgtgaaggagtgaaaggatggataaagtaatttaggaatcggtggatgaagaatgggtaggtagacccttggtgaagtggtagaatggaaggatttatgaacagatatgagttaattcttgcatcgaagtaggtgtaagtgtctattagcctgttgcactgaacatgcagttgcatagacaaatgagtggggagaagtacggagtaaatccctgcatgaatggtaggacagaaacctgaatgcctggatgctggcagtgtgaagaatggcacttgggatagatggttcgagtatggggtagattaaaagatggatggaaaagaggaacagagagagggtgattggatgaatggatggatggttggatgtgactgattgacaggtaccaagcttttttcctgcactgtgccttctgtgctgcagctgcagaagagacggaggaggaaccaccgtcccagccacgcctaggagagctgacggcatcccatgtcagccccaactccgtccagctggaatggagcatccctgagggctcctttgactccttcacggtgcagtacatagacgtgcaaggccagccgcaggagctgcacttggatagtgggtcgcgcacagtgaccgtgtctggtttgctgccatcc

SEQ ID NO: 25 - DNA, 1146 bp, Gallus sp.
gcacagaaggaaccgccatcccaaccacgcctgggtgagctgacggcctcccacgtcagccccgactccgtccagctggaatggagcgtccccgagggctcctttgactccttcacggtgcagtacaaggatgcacaaggccagccacaggtggtgcccgtggacggtgggttgcgcacagtgaccgtgcccgggctgtcgccgtcccgccgctacaagttcaacctgtatggggtgtgggggcggaagcgtctgggccccatgtccactgatgctgtcacaggtgagcatgctgtgttctgcctccatgttcttttgctttcagtgtagttgtcatgtggcaggaacctttcagggccacttttggttaatgttgccttaatagtcaaggaaacaatttgttcttgttgagtgggaatgcctaacgggatgggagtttggatgatgagaggacaaatcttataagggatgattgataattattgcggaacggatggaaggaaggttggatggatggaatggtgtttggataaatttgtgctcagagcacagctggagtgttggatgaatgttgctttgcttgttgaatagatggatgtttggttgtatggttgcttccactgagaattcctccctctgtgctgcagcagcagctccagcacaagaggagccaccttccccaccacgtctgggtgagctgacagcgtcccatgtcggccccgactccgtccagctggaatggagcgtccccgagggctcctttgactccttcacggtgcagtacaaggatgcacaaggccagccacaggtggtgcccgtggacggtgggttgcgcacagtgaccgtgcccgggctgtcgccgtcccgccgctacaagttcaacctgtatggggtgtgggggcggaagcgtctgggccccatgtccactgatgctgtcacaggtgagggcaggaattggcacctgttgggctctgggtttgcagcaggtagaaatgtaaacgtggcctgcgctggggatcttgttttcccctggcaatgggaacagctgttgggtgccttttttgggaaggatcccttaatcgcagcatgaagtatgaatggaccaattgggtgtgggtggagtgatggctgttgagatgagttggt

SEQ ID NO: 26 - DNA, 1042 bp, Gallus sp.
ctgtgtccccaacctgcttggtgttcccgcaggacacgctggtggccctggaggcgctggcccagatgtggctgcactggggccgtgggaacacaatggggctgaacctggggctctcctggccggggggtgcccgggggagggctggtggcactcaggttatgctgaagccggggctggagccgctggagcaggagctgcaggtggggacatggcgggatgtggggacacgagggatgtgaggacactggggacatgtctggacttggtaggatgtaacatgaagacactggggacatggtaggacatgggggacatgagaacacgggatgtgggggacatggtaggacatgatggacacagggctttggggtccttgggtcctcgctctgtccccatgtccccaggtgcctctgggcagcccagtgacagtgcaggtggagggacacggcgaagggacgctgacggtgggtggctgcatggacattggtgtcatctccaagaccgatgtcccctcacaacctcccctcatggtgtcccctcatgctgccacggtgtcccctgctgtcccatcatggtgtcacgctgtccccaggtgctccgccagttccgcctgctgtcacctccgaacgccacgtgccaggcgctgcacctggaggtggccatcaccggccccatcctgtaccatggtgaggccccgcccccttttcctcgcggggggcgtgccctcaaccctgttttgcatatcccaacccccagcagatgaggactacgaggactacgaggactacgaggaggcggagcctaaggagggggaggagcctacggaaggggcagtgcccgtggaaggggcggggccagcagatgaccccgcccccctcagccccgtgtccttatgggatgcccgtaagcggcaacgccgcagcacacataaccctgcccacgaggtggccttcctggtctgcttccggtgaggggcggaacttcctgtccctgggggcgggtcttcctgctgatgggcgtggcttattgctgaggggcg

SEQ ID NO: 27 - DNA, 826 bp, Gallus sp.
cctctgctgcttccagagcaaaggaaaagggagaggggggctcccaccaccctatcccagagcatcagatgggcaatggatgcagcagctccgtgggtcgtggaggtggcacgtggcaggagcgaggacggctcggagataccgaggtcatcagccaccgaaaccatctcaggaaagggaatttccacacaaaactccatttggagcacctggcagagaagctgaagcttttggagctggatggagacagaggggagaaggagaaactctgctcgtggcgcaagaggacattcccctccaatggaccacgggatgatggaggtcccactggagcccccataaaggagtcagtgcaggaggatgtggtcagccctgtgttattccctaaagccctgtttaatccttcatgtccatgctgaaaacttcttctctgcgaagtccaacacattgcatctcttcccttctttctcccatcacaatatcctccccaaacccctttttcttcctccaggagcagattcacagcgatctggagaacctcaagaaacaaaaggaggagctcttagaactcaaaaggagtggggagaggcgatgccaagaccttctggtaagaagctgttgccttcaagctggaaaaacagaggtctttttggggtccacgttgttgattttccacaacctacagacacggacggaggctgagaggcagaaaattgtgtcagaattccgtcagctccgccgttttctgaaggagaaggagatggtgctcgtggcacggctgggggagctggacagggctgtgctgaggaggcaggaggaggaggag

SEQ ID NO: 28 - DNA, 964 bp, Gallus sp.
agcccagcactctgcagtcttctatcagttccaatagaggaattttggtggtagaaggggctggaaggactcactctgctttgtggtctcagctgctggaaaacaaagcagagaaatagctggtcagcagggcagcttggtttctggggacgtctccagagggtctggacctttccacctgccccacggtccacccacattcctatctttccgcccacacccctttttccctttccttcattcccaatcaaacggcaaatgttatttaatgaccactgtcaatccccagaaaaatctccctttctcctgcatacctccacggacctgagctcagcaccaccccgaccatccctatccctgctcaacacctccctgtgatccatcccctccatgctcaactcacctttcttcctatagagaaaaacagtgatgacaaatgacccaaccagaattgtgacgatcacagccagagccaccttccagggatgggtgatctgggaaaaggggtctggaaaaaacatcaggacaagggttccttttccattcccataagtggaaaagcaagactcagccttgggacatcacagaacccaaaggggcagcaaccagggagcagtgatgcacaatgacggcatccccatattggcacaggtggaggagctgctcagcatcgtgtgcccactgccactgagccatggagaaacccatcccagaaatccaacccaaccacctcatccatgcagacttatccacaaattgcactgtgcacctgctccaacaccagcatctcatggaacaatttagctccgacctcttccaaaggctgctgtccttcagctttccatccatggatgtgaggatgaggatggacagaggtcggggtgggacacacaaacccagcaacacctggaggcgtcaccccagccactgacctgacacctccaggtccaccacagcgtctgca

SEQ ID NO: 29 - DNA, 998 bp, Gallus sp.
cccagcaaggccaagcgccgccataacgtcagtgccggtgagactgtctgatgcggttgcgcgaggagagtcactgaacatcggtgatttaggcgcaaagtatttagcgattgattcgaggttcattatgcggcttccttctgtggctggtgggttttggtctggctgtgctttactattggcggcatgctggcgcgcttaacgctttcggcctggtatcgggttatctcgtctctggtcatgatggcctccgattccaggcgcgaattgcatcgcgctttgttggataggtgtcagttatcggcttaatcaagcattgctttgttgaacaaccggcgtagacaccatcaccatcagaaaaaagttctgcgccgccgccacagaacggacactcaagcagaaaagcccaatgaggtagcttgagatcgaatatcattggtttcatgctgcctcccgctgtttcagtgctttgagcttgtcgcggtactcatcccggatccggatgaagtcttcacggcggtagttggtcatttcgtggggaccattgagccagttgacgtatccctgaccgtaacgagcgaccaacccagcttcgtattgctgcgccacggtcgcctctttggcggtgtacttgccagctccggcattacaggatttgcactgcttatgggcgttgcgttcttcaaagcgcagttcagggtaagcacctactgtcttgaaatggccgcaatcccactggccaccatgcagatcaggcggattggtctcgccgcagctgatgcatggcaaatcggcgtcgcgcgcacggataaaggcgttgaaagctttctgagcctgagccttgtagtatccgtctggcctgagctctgccagccgctccttgcggcgtttgcgcccgtccttttcagcctctttttgctccttgatgcgcttagccgcggctttcaccttctccttcttgcgttcttccattgcgaggattgcgccatgctccgggg

SEQ ID NO: 30 - DNA, 1130 bp, Gallus sp.
ccctatggggccaaccccgtggaaaccacgcagggttggggttggatcctcgagctcttttgcaaagcctttctggctatggttgcactcagttaattaaactgtctaaaaccatattttgtatataattagacatgatgtttactgcttctgtcccccccttggtttaagagcagagaggctcttgcagaagggaattcctctcactgagtgccactttggaattgttgtgtgatcacccaaactccagtgcaaagccccagccccactttgggcagaatgaatgtgttttctgctcagaagagcttcgatttcctgtgcagcaatgtggttgggatctgatcactcaccgcacacgctgagccctgtcaccagcagcagcagcagcagcagcagcacccccagcatgcaggctttctggaagtcccacggaactggaagagcccacacttatataaaacagacattttgaaaaaacttttccttttacagaaatgatctccctgtgaaagagcccctccaccaacctgctacgttagagcagaagttgatggctgctttggttccttgagaatttggggtccccggacccttcccattggttcccatgctgtgtatgagcagaagttgatgcctgtttgttttcctccagttccggggtcccctggacccttcccagtggctcccagtggttcccagctgggaaggaggtggcacagaggggtgacagcagcagggagcatgggatgggctcccgctggaggtcagatggacacagggacatcacctccacgcgtggttctggaggtggtggaggccctgcaggctccatagagcatggggtggaagctgagcagcccccaggctttgtgagccgagcccagtgtggggcagccggtggctgggagggcggggatgtctgcagccctcatgccacgtggatgcagggtgcgtttgccaccatttcattccattctctcacattgggcttcccgatctgggctgatttttgctcaaaaccaccacacctctggccattcccaggcttccttctcccccaccccttcctttactcccccatacaagccagggtggacccagaacccaaccagcattgctggtgtttctca

SEQ ID NO: 31 - DNA, 39412 bp, Gallus sp.
ccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggcatcggtcgacgggatcacgttgtgtccctgaagctctcctgtacccaaacacaaaggtgatgtccccagcatccctatcccagcactctgggggactcctattgaattcctccttgggcttgctgccttctcttcccgttcccagagatcccaaaaggttaagcacctttgggtcagtgttcagaattgtcactgccagttttggggtatcagtggcaaattgagacccttttacccaatcttgcaccactctggttccccagtcttatggttttagatggagtaaaaaggtttatatgtcataaagttcttctgtgtctggttattcgctgcttctggatgccaggatcatggggataaggggaaaacaatgggttctcttatgcgtagagatgcaatcagatggggagaaaaagaaatcttaatctttctgatccatctgacagatattcagtacagccctgaggatgtgggaaataaatctgaagagtttgttggcagttccaaggatttggaatgactaaatcccattcctggtgtctgcacaaagttggctgtgttggaacccagaaagatccatgcaagtgggtcatccctgaaagcattgtgttctgctgtctgctagcggagagaaagacacagaggggaaaattaagtgttttattgttaattattgtacactctgaggtttcaaataccaaatctttaacgagagcggaccacttgatttgagggtgaccatctcagatggggacaactgtacctgatcaggcaaacctgggggaaatttgcctttctgccactcttttgggtgggattttcccttttgaccaccattttctacattctaatcacccattgcagcacttctcccccttttttttgccccatttttctcctgctcagcacttcttaacaatataatataaatcaatatcatatcaatatgattctatgccaatagattaatggggatgaaagacacataaaaacccaagtcctcatttcatctgcttcccatgggatgggtggggaggtggctgtcccctgaggctgtaggatgtggggtcacccttgtctgtgtctcagggacacagcctcagcttggacctgacccctaccacccacagccacggacggaccctctccccagagaaggatgcatgggaaaaaacaaagatgagccccccttcatcagcatcaaaaaatgccaccgtccctccagcgtagtccaagtggacgctgaccctcctgggcacccagcgcagagctaacagggtcaccttgtgggtggtgagtgcccggacctgtcccccccatttctccaccccccaaatcccccctttgggacagaggctgagttgacccttccgagggatggattctcgggccacaccgatggcccagtccccttcatcccccacttccacctcccagcagtgccggccggcagagaagctttggtggcccaaaacaaagggccagtaggcgaatctttcggggttatcaggaaggtcctgttgtccttccccacgtttcacactctttcggtcttcggagaggatgaggtcagggtgagcggtgtcggggtccagggtgatgctggctgtggggtggagaggatgaggagtgtaaggtttgggtcctcggtgctgaggccatgaggatgcggagagcttggatctccagcactaaaggagttggatgtgctctagatggccccacctgagtagggttgtagggtgggaccgtcccttccaacctcagccattctgtggggccatgggttggcatcggaagggtaaaaagtaccaaagaagaaagtaaaaaggtgagaggtggaaacccctctcatgtgcccgtgctatatgacaataaaagtgttttgagcccccagaatgcccagaaataaaggcgtttctgcagaccttctgttccattggtcaaaagaaatggtgaggggaataaaaatggaaggaaggagatctatgggatattacctgcaaagtctgcagtgcttcatctcctagaccaacccggaccagttcagccaaccccatggtttaaaaaacagagctgaaatctgaaggcagggataatgaatgagttcaacccgctcaccatatttgtttatgggaaatggatatttatcaaggcgagggatctgccctggggccatcatcccaaattacagccagactcggcctgcagggtgaagaaaacttgtttggctgccctgatttttgtgtattcctccctcggcatctatttttgtccatttgggtacagcctatgggtccaggcgcgcctccatctaacaggtaatgcggctttaggttctcatgctcagcaaaaggcacttttaggaaaggtgaagctggaggggtgcagagccggagagcagcccgtccttcacccctgagcacttctcaggaattacagcaaaacgtgtaattaagagtggcaaacggggtatcgagtccttcgggtctcaattattttcctgagtgggaataacccgttgctcttccatctctctgcattattctgctgcagaacgagtgatgggctgctggttttcaccaaaataccaccatttcccacccgaaacccttctgagtaccttgaagcctcttcagggtttccttcagagcaccgttcctccatgaggaatggcacagcctctcctccggccctggagaagcgcccgctggcagctggaaggtcacttttccacacctggaggggaaataaatgcattttcaggtggttgtatcacagagcatgccatcacttcaggacagcagaggccagcacacggcggccatccccaaaatacccttcagggctcgcagttcccctggagcagaagagcattcattgatgagctttctcctccatggtcactgcctgatgcaaagctcacagaacagcttttcagagaggccacatacctggtgatggggcttttcacatcctggggacagaagagaggagggggagaggaaactcaggtcagtgcatgacccattttgtctttaaagtatggaaaattgagctgtttgagtgggggtggacctcttgggtcttccaacatgtgcccaattttgactttaagtcatagaaaaagtgaattgtttgactggggatggatctgttgggtctttcaacacatggtccattttgtctttaaatcatagaaataaagaattgtttgaccagagatggacctctggggtcttcctccacgaggaaggtgaaccaactgaggagcatccatgcacggcaatgaatcctgcagatccaccccactgctgctctcccaacccagccgtggatttcccctcttaaaacagaccccatgaggaccttctgcagtaaggtgaaaatactgggaatactgagatgaggataaaacggtggggggaaagaggaggctgcaaacctccatctcctcattgtggtgggggtttcaggctgatggaacggcataaaatgggaggaaaacacccaattaaggcaccatgcaattggtcggggtggggaggacatccctaaaggacttttccccttgaaaaagcttccctggaggaattcactcaccgactgctggctcttctctccctgtgctttcgtatccagcggggaaatctcctccgagtgcttggcggtgcttttctgcctcttctcaatctcatttttcaggtcttccagctgccagagcaagaagggctctgtgttttcctgcctggaatctgagccctccctactggggctcagctttccttctgatgcagaaagtggaaaataaagagcagtgggactggaaataccaggggggactcatgagtggcatcccccactggaggagctcaatggtgagctggaatccttgctaagttttatcgaatgtgggggacaggaggaagaaatcaaactcaaaaagtcatgaacaggtggctgtgaattcggggcagaaagctgagggccctaaaagcacaggaggcaaaaaggatggagagaaacgaccctactgatgacacatcgctgcccagcagctgacacctaccagatcctccaggtttgggcactccagggcgctcttcttcctcggagactttctctctcctcctttggaaacccctgatatccctctgagtttcttccccagtgaacccacagaacctgttgttttcagccctttgatggggttggggttttcccttcctgttccttcccagtctggggtagagctatgggatggctgcgttgagcctgcaggtctgctcctggtggcacccttggcagggcgtgctgggagctctgggtttgtcctttgtctttctcccagttccttgtcccggggagatgctgaacaatgtcactttgcagattttgtcagcttccttttaggatcgagccatcgggagtggggttagggggtgtatatggggaaaccataaggaaatagggaaggagatgcacagccggatccttgtggggatgtggaggagcacaagtgaggatctttgggatttgagtgctctctcagcccagcactaacacagagcactcacagccctggctctgagctctcgaggaaacatttccaaccatttctgccccactgtccttgtgttgagccccatggccaaatacacatgcctagaaaataaagccatgcattacatatgtatttaatttttgcgtggcaaccactgagacccaactggaggagataactgccattcacttgggcaggtttgcaggggtgaactgcacttccagcaaaccctccctgttgggaagagccacagggatggatggcactctgggagctgaagaactggaagcaaactccctgcaaccgctcccctggggcacagagcctttcatcccaaaataaggcgtccatcattgagcaaatgagtcacaccgttgggcaaacgacttgcattgcatcccgaaaagcattaattgcagagcctggaaaactagctgggctggaaacatctgcattgcagatctatggagcagaatagaccctgaacagatccttcacccaaattccccagcaggtgggaccaaatggcagcgatgcgtggggctgaggaaagataccaacacatcaaagagcaatattgaaatttcagctgtaggtttgacctttggaggtggtgaggtggggctttgtcatgggatacccactcatatcgcatctgctattctgagcctgatgtcgcctgctccctcccaccctcttttagttcctcttcttggttctacaatcaccaacctgtgtgtattttggtgctgcctgttcctcttttgggctttctcagaagaaaatgggtttttgagggaatccattcaggtgagtcctcaccccaagcagctcttcttcactttgttggcccaaagctgacccagagccatacacccaaagcaaacccagagccgtacacccataatgaggcaggaagtggagtgtgcagagcacatcttttaattaaaattaactatcagaaacgtaggcagagaccagctccccacaccaggcgttgctatttgcagtgaaaggccgcatacctttgcaggacaccccagatctgccccacgattgatgtcaaatagatgcataaatttccttccaagtcttcagtgctctctggtggtttccccaccctgcagagggaccgccccggggctcccaatggggacagacacagggcagagcagcgggtccccttggcacattgctccaagcaaccacagcacacatcccatcagatgcccctttcataaaggacatctcaaggacagatctttaggggagatctaaacccaacccaatccaaatgggacatcagctgcccactcgtggactgctcctctgaggggggattttgggtgatctcttgcaagcgagcccccagccctatcttgaacaaggggaggaccttctccccattgaacaaagccctggtgtacaccaagatgggggtgtcatcatccgagctgaagaatgccacccgacccccttcgtagtccagggagacccgaatcctcctgggaagtgcattcagacgtaggttggcacggggagacgtgagggagtggtaggcctccagcgcccagacaccctctttggggctgaagctcatgggtcccttcctcttcatcgaagcccgggccacccccagggcccacacccccccctgtcccacctccacctcccagaaatgcctccccgaggtgaagccctggcagcccaacacgcagggctcgaagctgaacctctcggggttctcggggaggtcctgtggcaccagttggccccgggcttgttttcggtcttcagagagatggaggttggggtgagcggtggtggggtccatggtgacgttggctgtgggacatgagggggaatggaggtaggatttaggcttggggggagctggagaggttcctcttccttctgtccttttctctgggtgcttttggacatgggctggtggtggtggtgggttgatggttgggctgggtgatctttggggtcttttccaacctttgtgattctatggggtgtgtggggctccaccagcctcagtgtcccccagtagagatgtaggagaatggggagaggacaaattttagggcagcataatgcgggagggacaaagacatgggaaggggacagcttgacattcacggaggggaaggggaagcacaaacactgttaggttttgccttgaatctgttactggctttgtaggaccaccagcatcaggatgctgtccccattccctcccttccctgtgggactgcgttgttttttcccaagaaaaccactccccaccccacatccaccactgctgacatacctggctcttgcaattgaaacatcaggctgtctgaaaaggagaacaaattcactgcattgggtttatgcttcaggaaaaggggctgggagatggggaagggaaaccatgggggtctgggggcttcgcagtgcaaaagctctgggtttactgcaagagccccacgaccctcccagacctggaggagaccccgaccccattcagtaccttggcacttctgcagcgtcagtctcaccaggacgttcttctgaaggaagtcctccaaccttctttccagagtgggggaaatctctgctggagggctgaacttcatcatctcacagctgcaaagagaggagaagggtggggatggggggactgttgcgttggttggttggctgttcattttattctcaataggagaagctatggggtgaggatatttgcacagggacgaaatccctttcccccctgggatccctctgccttgcagccctcccccagggtgccatccaaaaatcagggtgacaataggaaggagccatgttacctattcaagagcctcctgatgtcctaaaggtgggaggagagaggagagatggatcagaagaggagcaccaagggctgccccttcgtatggcaatgcacagcaaagaccaccctgcccacggtgtgatcccccccagcagcaacacagggagctcccatggggttgagtttgggttctcagggtttgctctgtccccccatttcccaccacccctttgggttctcaccagcaggaatttgctgtcgggctgctggaatttgccctccatctcccagatcagggtgtcaaggtgggacatctcctccatcaccttcgtcaccgcatcctcctgtactttggtgacggctctgtccaggtctgccagctggaccagcaggaagcgctccttctccttcagaaatcgctgcaactgctcgaattcacacactatcctcttcccttccttcttggttttctcctgttgggatgagggagaaagccaatggggtggaatagaggcaggaagaccccccctggggtctcaggatgccgtgttctgggggatatccaaccaaaaccaatggggatgtaacaccaatgccaatgggagcacaacactaatgccaatgggaatttatcaccagtgccaatgggaacgtaacaacagcgccaatgggaacgtaacaccagtgccagtgggaatttatcaccagtgccaatgggaacttaacatcaaaaagccaaagatcatcttgctgggcatttgggagcagcaggaatttttcaggagttttatcccaaaagcaaaaccaaaggagggggtaggagatgagctctgtatgagggatatttacagagtttaggaggatctgctacgttatctctttaacacaggggttcctgcgtaaccccagctgataaacacagccttagcgctttcccagcccagctgcgagccaaaaatgcatgatctgcccccaaaatacaccaaaacaaacaggacagggcggaggggaaggcagacacctcccctgctgcacccaccaaatacaagcccgtccttccaccagtccttctgctttccaggtacttttccctctcctcctttgaagcctggaggcgagcctgaatttcttcctgtgccaaaagaagaaaggcggaaagcctgttttcccacttaaactgcttctgtcagatgggagaggctttgctaaagcctggaatcctctgcaaggtgcagagctgggcagagggaagctctgtgagcacggtgtgctgctctggagctctgtgcaagctgggagtattttgcagagagaaaagaggggagaagggaaggaaaaacacgaacttgctgcaaacgtagagaaaaacgctgcaaaagagcaacaaaaaaatcagcactgacagctgcgcaaggaggtgtggaagggcaagataagcacttggtgagatttccctcataaacaccccaaaacggcggccctggggtgtgtttctgtatttaagagccctcagtggaatggtttttgcagggctgtggtcgaagagcaaagcatcaaaggaaggagagggcagtaatgttgcaaagggctgacggcggtggttgcaaagagggaggatggggggggatgcgccaagcaaagggttgcgtgggttcacccgcagggatgcactgcgcccttggctccgggttttgggaccgtaccttgtactcctgggccgcctggtgggcagggagcacagcgtgggagcggtgcgcctgggacgcgtcgcactgcgcgcagataggctcttggtcctctgtgcagaagagcttcagagcctcgcggtgctgcttgcaccaacccgaggaatgcaaactcagctgccgggcgatgctggcgatatttgccagctctctgctggggcggaaatttttgtgcaacgccgttttcctgcactgcggacaggggaaatttccctccagcccttcccagcagcgggcgatgcactcccggcagaagttgtggccgcaggggatggagacgggatcctggaagtaacccaggcagatggagcaggaggcttcgctctgcaggctgtccaaggggctctgcgtggccatgggcttcctgctgggctccgatccgcagagggaataggggacctttcctccttatctcctcgctgataggagaaatccggccccggaggctgagcctgagccaaacagggctgggagagctcagcccataggggatgctggtgggaatgggggcagctcgcggctccccagcacggagtcaccaaactggggggatctgggggaaattcggaggaaaagtcagattttgtcctctcctcgagcagcaaagagggcaggggaggcgatttttcccttctgtgcgatcactgtaaggaatttccaaagaaaacgcatggaggtctgcttgttgggatggaatatagacgtatattggaataaatacaggaagacgttggaacatgggaaggcactgagatataagcgtgctgtgttggatatgactctgctcgactaaagtgaaggtggttttaatagcactgctcagagccaggcgggttttggtgttgtttggggggaattacgtgggtttggaattgggaaatatgagacggaaaaataagaataatggaagcgcccaacgtggggctcgaacccacgaccctgagattaagagtctcatgctctaccgactgagctagccgggctgatgggcacgcacccttctaagcaatacttcatggtgatcctgcggaggggtgctaataattctacctaattattttgttaattatcccggtaattatgggttctgagcaatcgcgaatccacggggaagagctgcatggggaaaaagcacctatccctacgggaatagccgggaactgcccggcagtgctgcagggcgggggaaagaggggaaaagcaggaaaaaaatgggcaaaatggaacgtttaaaagtggagaaattaacagtgaaaaaaatgcaggaagcgtaaaagtaaaggctgtgtttctgcccggtttcgaaccggggacctttcgcgtgtgaggcgaacgtgataaccactacactacagaaacgcgctgaaggccgcttcgccgcacggagatgtgaaggggcgaatgccggggctcggtgcggagtttgcagataggggccgctccgggccgctcccgcgccggttccggtgagcacagagtgcagcgggtgacaaaatgaagggaaaaatgtaaaactgatgctcccgaatcgaggctcgaaccgccattgtccgactgacagccgcgcgctctaccgactgagctacccggagacagagcagccggaagtcacgcccccgtagagcgcccaccccgttgcctagtgacaggagcgccgcttccggtcaagtgatgagcggagggggcgtggcttgtgtcagataggacggaagttccggtcaggtggtactggaaagggggcgtggcttgcggcaaaggggacggaaagcggaagtgctgccgttggttggcggagttcgcaccatagaagaacgacggcggcggtgggagggcgggaggtagagcggtccccggggagagtgctgaggggagcggcgaggcccgaggagggagcggagcttacggggagtgcggagcctcgaggcgggtcccagcgcttcgctgtggggcaggagaaaggcttcggggcaggaggaagagggcctcggggcctccccatggaggcggtgggcgacgatggggcgtcgtcggggcggctgaacccggtggagacgctgcaggaggaggcgatctgcgccatctgcctggactacttcgtggagccggtgtcgatcggctgcgggcacaacttctgccgggtgtgcatcgcgcagctgtggggtggaggagaggctgaggtggaggagagcggcggggccgcggcgttggaggaggaagaggaagagctggaggaagaggaggaagatgagctgggggaggaagagctggacgtggagcaggaggaggaggaggaggatggaggcggggaggaggaggaggaggacgacatgtggagcgaggaggaagaggatggagagctgtgggaaggtactgggggtcggtttgggcctgccctgttgagtgtctttatggatgagtgagggaattgggtgcaccctcagtcagtttgcagatgatgctaagctgggggggtgtactgatctgcctgagggtaggacggccctacggtggggtctggactgggcccgatgggctgagggcaatggggtggagttcagaaggaccgagtgcctggttctgcactgaggtcacaacaaccccatgcagctctacctggggtagagcggctgaaagctgtgtgagggaaaaggatttgggggtgaatatgagccagcaagaggccaagaaggcccatggcatcctggcttgtatcagaaatagagcagctagtgggagcaggaagtgactgtcactctgtactggcacacctcaatgctgcacccagttctgggtcccctctcactacaagaaagacattgaggcccagtgaggatggtgggggttggactcaatgatccctgaggttttttccaaccttgatgattctgtgattctcagaccccgtggaagaggagctgtgggatggagtggtgcagggagaactctactttggggacgatgattatgatgaggatgtgatggaggaggatgtggaggaagaggaggaggaggaggatgaagcgcagagccctccgccccctgtcctgcctgcccgccctcgccgcctgcagaccttcacctgcccccagtgccgcaaaacctttttccagaggaatttcagacccaacctccagttggcaaacatggtgcagatcatccggcagctccacccgcacccgcagcgcctcgcgccgcccgccggcccctcagcctcagggggtcctggggggaacccagggatcctggtggcaacaggaggtcgggggtgtccgaatctgtgcgagaagcaccaggaacccctgaagctgttctgtgaggtggatgagcaggcgatctgcgtggtgtgcagggagtcacggagccacaagcatcacagtgttgtgcccctggaggaagtcgtgcaggattataaggtggagtttggggaagggtcacggtgggatagtgggtgaggtggggtttggggaagggctgtggtggagaaggcggggtttgagggaagagttatgggagagtggaggcttgaagggaaagtgaggttgggatcaagctaggttcgtcttgctgagctggttgggttggaggcgtgggaggctgggaaaccacacactgcaatgaggaggtggaagggtctgggtacccattttctgcttaaaaacaccttcccagcacagttcctcagagaaagcaaaagggaagtggcgtgaaagttggctctgaggttccgttttcagctctgccaccaaattagggacaaaaagaggcgatgacagaggggattgccccaggcagggtttgctgagttgtgtttccttccctcagtacaaactccagagccatttggagccactgaagaagaagctggacgcggtgctgaagcagaagtcgaatgagcaggagaagatcacagagctgagggtaagagctgaaggtttctgtgcttcatagaatcatacaggagaaccatcagggttggaagagaccacaaagatcatcagttccaaccatcaccgctgctgggagtgtgccttggtggctgagcaaggagagagaagctttgctgctgctctgagctctcacggaggcatcatattccctttcctgcaattattgggctgtgagggcttggaaacggtttcccagttgaattagagcttaatgagagctttgtgtgcctcagtgttgagtgggaattggtggtttgggagctggtattcctcatttgagttgaggatgctctacatctctaaacctgtgcagactttgctcagttctgtctgtggtgcattcaggagatgcgtaagcttatggtgtgtggtgaaactgagagaagcatagcacagcagcccaaaaatgagctgatctctcacctcccccttctgcagcaattcccctaatgcttttcctccctctgcaggaaaagatgaagctggaaatcaaggaatttgagtctgattttgagctgctccaccagttcctcattggggagcacgtgctgctgctgcaccagctggaggagcgctacgagagcctgctggcccggcagagcagcaacatcagccagctggaggagcagagtgcagcccttagccgccttatcacggaggcagaagataagagcaagcaggacgggctacagctgctcaaggtcttcttccatccctttccttgtctttatggcaaagcgatagcacgatggtgggaataatgctccagaaagcttctgtgtcatgagagagtgcctttagttggtgggctgggtgcttctccacccctccttgtggtggtttttgagggaaaatgccggggggggggggggggggggatatgccctgagagatttagggtctgttttggtaaggaaagcctccagcaatgtgtgggctgtgtctttgttctctgtggggaagggaatcatccaggctcagtgctgagttgtggctgataagaggatttattgggagcaacggtgggattggtatcagtcatccctaatcctttccttctctttcccaccttgctgcctccttcccacaggacatcaagggcacttttatcaggtcagtgactttgtttgcatcttttcactttgaataacttttcttttttttaatgtcaaaaaagcatttgagcttttgttttaaatcctgtgtgatgggtacagttggggcctggtaatgcaggggaaagctgtgtcctaacttttgggtgatggaaacttctggctgatggggtgcaaatgggatctggggaacaacttgggaaaagacttgggaacttgggaaacaactctggggccatttgggaaaggggaagggtggggaggagatctcggccctgatttctggaagcgtgggtgtgcccatgcagacctcatgctatagcgaaactcctcactctggagaaacgattctccccatcctgtcagacaaatgggcagcgctgggagttctcagccatgctggacgcacgtggctctaccccagctctgtctgctggctgagggagggtgggggaggctggctgcaccagtgcaaccagtttggccgatccatgcgttgctctggtttttccagagctgcatgcaggccgcctcacttcttttctgctgctgaaattctctgctttcctcctttccccccacccaaaaaagatgtgagaacatcaaattccaggagcccgagatggtgctggtggacgtggggaagaaataccgcaactatttcctgcaggatgtggtgatgagaaagatggagaaagccttcagcaaagttccacagggtgagagagtcctcttccttctacgtgggatggggttccctccacttgggatgggatttctccagctctcttggggttctccttccatctctgtgctcccatggtttgcagcctgatgatcctttaggaaaagcagcatccctctgttctctctgtgcttttcccttttgccttgtcctgggttttcccctattgtagctcctccatagaactggggttgatgtggatctggattcattataaaggagggatgactgcctcaaactcagcatggtgcagatacgcaaccagatgaggatttaggactggggtgcaagggggaaaaaagtgccaggtgaccccctaacgacccccgctctctgcccttccttccagctgacatcacgctggacccggacaccgctcaccctcgcctcagcctctccctggaccgccgcagcgttaagctgggagaacgacgccaggagctccccaacaaccccaaacgcttcgactccgattactgcgtcctgggctcccagggtttcaccacaggccgtcactactgggaggtagaagtcgggggcaagaaaggttgggcggtgggggctgcacgcgagacggctcgacgcaaagaaaaaaccatggggcctcatcaaaaaagggagatctggtgtgttggcaccaatgggaagaagtaccaagcgctgacggccatggagcagatggctttgtcacccagcgagcggccccggcgcttcggtgtctacctggactatgaacggggtcagctttgcttctacaacgctgagagcatgacccacatccacaccttcaacgcttccttccacgagcgcatcttcccctttttccgaatcctggctaagggcactcgtatcaaaatctgcacctgatggccctccagcttctgattttttttttccctttttcccccctgcctcatcctttgggtcccactttgggaccagacgctgcacttgttgtctcgcacctgcttgctcacaaggcctcttccctcctctctcctgtcccagcctctgtccacgtcccaactcttctccggggtcgcgatcccaggctggtttggtttggagaagggatccaatctccttgctggaggttttcccttcagctcttggtgctatgggctcccctctgcctttcccagtcctcgcagcagctttccagtgtgctcttccccgttttgtttaaagcctgtggtcgagctttgcgttgtttgccctctttggatgcagagctcgagctgaggatgctggggtctgtacattgtgacacgagcactgcttgtgccctcttggccattgctttctgaaagtcactcagatgcaccaaggagcctcatttctttttatttttcagttctggggcacaaccctctgcccacctcccacccagccaccatctggacctcaaaccttccacgttctcctattctgccacttgtccaccttccccttttgctcttcttccccctctgggggtctccagctctccctctgccccatcattccctcgccaaccatttcttgtgggcctggcactttatttagggccacgtaggccggggagggtgcaaaaaattgggcaacttccacctctgaggctgctcagagtgcagcatcgcaccaggccgcaccggtgggaagcagccttgtttccccttgcagcttaagagctctctgaggtgggggtatttattttctcttcccttttctcagctgctgttgaatttccagctgaatcctgtcccaccagagagactctgattgcaccctgttgtgttttacttctttttgttggtggattggtattttttttttctgttggcgttacagagctagttcaaaatatttttggctaaaataagaattaaatggagatctagttttttgaaatgtcaagaaataataataataataataaagaataaagaataaagttttaaagctgagcctctcccttattgagagcccccaggggacaggagttgtggtgcaggccccccagtctgctgttaactcctgctggtaagatgtgacttaagccttgcatcgttaatcttaacttaattagcagtaatttggattgggctgcttcccttcagcagcttgtaaagggatagaggctgctgggtgaactgagctctgtgttaccacctctcctgctctccccacatgtttttggtggtggtggttgcttctttttggccacggctctatctccccaggtgtgcactcactgtgggctgctactgctcctgaaagggctcagggagacatttgagtcccttcgtccacacgtgggaggagagcactgatgtccccatccttaaagttgtgggcacagccttggtggcaaatccagaatgggatataatgcagccatgagctcaacagagcgctcttttattgagttttgtgcataaaatctgtgtgttgttaccacatcctcatctggttccaatggtgacttgccacacccggacgaggttatctgtgtagccagcaaacagcgtctggggagagaaatggaggaagtggatcatgaaaagataggaatcagccctcggtgtgaacgtaaaaatctcagaaggcagctcccaaagcggaggtgctggaggaaggtgggagttttaaggctgcaggaggagcagtgaaaagggaaaggagaaggggatatttctacctgcccatctgcagaccacgccagagaggtacactggggaggctcagctttgctgctggtgctgatcacctcctgcttcagctcatccacaatgattttgccttccaggtcctgtgcaggacagaagagagcgtgagggactaaggtcctgcagggagactgctgtagccaaacccaaccattccaactcagaacaggctcagggtgctcagaaacagcctctgggtttccgcacagggatgcagtcagatggcatcgaagtttcatcacagcagagtggtggctgtgccccacaccaccctcccagtccaggggatgacagtgccaccagcatgacccatcccacgtaaccaaaagggctctgcaccaaggcatctgtggggcagggcgaggatttcgaccacaactctgcctccaaacccacaggataagggaagtgattctttaggaggtaaatagggatgtcacatacccagatcttgatgctggggccggtggcagcgcagagccagtagcggttggggctgaagcacagcgcattgatgatgtcccctccatccagcgtgtacaggtgcttgccttcattcaggtcccacagcatggcctggccgtcctggggggcagcaaagaggaatcacagcaaaccatcaaacctgtggctttgttccagttgtccatctaaaaccttccagcttggaaacagcacttgatttgtgactgagatgtgggtgagttgccacaggacagcaagaggcacataactgagctgtgagaacaacagaataagctgcaatttggcctcagctttcccccagggtgtaccttgcctccagaagcacagagggagccatcaggggagacagtcactgtgttcagatatcccgtgtggccgatgtggtttgtcttcagtttgcagttagccaagttccaaacctaaatgagggtaaacgtgacaggctcagaaatatggaggagaaaaaaaacaaccctctcatgatcactgctcaaatattccccagaacgccgcacaaaccccaaaggagctgctcctctcaccttcaccagcttgtcccagccacaggagacaatgatggggttgctgctgttgggggagaagcgcacacaggaaacccactcagagtggctctcgtcctgaggagaggaacagcattgggttgaaagcaatgaaaagcatccccagtccgagctgctgcatcccactgctccctgagcccctcataattgcaggacgtgtcctcagaccccccccagaaagaaaggtcagcaggcactgtgtcacttctaatcattaggacggagctgggagatgtggattacggatcaaaaccaacaaatcaaagagaaatgggggaatacggactcagaaacaagcagaaagaggtttattttcacagtgtggaaactcagatccgttgcctcacctgcaccgtgtatttgcagacacccaaagtgttccagagtttgatggttttgtccctggagcccgaaacgatctggcggttgtcggaggagaaggcgacgctcagcacatccttggtgtggccaacaaagcggcgggtggtggttcctctgcagggacaccaggagggtcgcacgggagggacaaagctcagcaaacccccattaaattaattaaccctcccctaaattgaggagatcgtgctgcagtgcataaattcttaatgaacacaactgatggaagcaggaaggaagctaaaacggagtcatctccacatgggttgaggagtggtggttccttccctccttccgaacaggaacaaaagggtgccaaagcttttgatatagggttggaataatcatgaggagtttaggatataaaactcagcttccgtggacacacagcagcgtaagtgctgaacgcttttggaggattggggtagttctgcttcctgaggagtttcttctcctatagtactcccaaaaatcacagtgcaagaagagccggtgctgctccaacctcaccccaaactctgtaccccaaaatcacaccgaaggaaaagcctgcttgctccagtctgtaccccacagcgatggtgaaggaagaaccaaatccccccctgctgctccacctgcttctctcccatcataattgcaggacgtgtcctcagatcccggaggatcagcagactgtgtcaggtgtaatcactgggagagtgagctgagggaggaaccgctttggtcctccctccaagcatgatttaccacccaacctgagaggaactcacctcattttcacgctgtaccgcacacctctcacccaccccaacacccaaacaaaacacagagcccagctctgccccaaacccccaccccaaagccctttcagtccccaggactcacgtggtgaggtcccacagcctcaaggtgccatcccaggagcccgacagcgcaaactgcccatcggaggagatgaccacatcgctgacaaagtgcgagtggccgcgcagggcgcgctgcgggatcccgtagttggtctcatctcgggtcagcttccacatgatgatggttttgtctgggaagggggaaaggcagcggcctcagctccaacccttctcacattcccgtcctcactgggctttatctccctcatagcaatgggggggttacacagaagcaccgcaccccttcctctcagccccccaaccgcctccctacgtcctcatacacagcagcctccccaccctgcagctctctgtccccgagccctgcaccccattcatcacctcccctcccccatggtcccccccagccccctcctctaccactgacggtctccccttatctcccacagtcccctccataggccccacagttccctgcccccccccaccccacagttccgccccccccgcctcggacgaggcccgaacccctcaggcgcggccctcaccccgcgacgcggagagaatcatgtccgggaactgcggggtggtggcgatctgcgtcacccacccattgtggcccttcagggtaccgcggagggtcatctgctccgtcatggcggcggcggggcggagggatggcggcggattcaataaagggcccggcccggtccggtcctaccgcccgcgatggccgccagcgcggaaagagaaagagggaggtgacttccggcggaagcggaagtagccgctgggttgtacggcaagaggggcaacatggcggcgcgcatagagagcacgctgaatgggggaatgggctttggaggtggggagggaaggttgttctctgccgctgcagggacacgaggtgcgggcagagcaccttctttaacatttgctattatttaacgttttacatttagcatttttattatccctgttgtgccaggacggagaagagcagggtgtgcagcctgtgcttatcacctgcagctgtccctgcaccccacagccaacccaagtttgtgacgcctgagcaggatctgacccaggaaggcaaacagaaggtctgagtcctcctccctttcctttcccatccctcccacgctgcagtttgggggctgtgacccgtccgcgttgctcagtgctcattccgatgagcagtggctgatggtgatgttcacaagtttttggcatccctgtgggttccacccccgttttgtctcaccagcctttttctatccgtccttatcagcagatcatccttgttattagatctgtctttttccagtcacggctttgcattttcaccttggttttaccacctaacatcaagccttttgtccccatctgatgatattcatgcagataaatccgtaaagcagggaagaattaaattctggccccttctacacccatttaggtttagatctttgcagcattcagccaagacgtgcttccagagccaggaataacgtgtcttgatgtgccaacacaccttgaaatccagaaaattgccccaaaataggcatgactcagcaagcaccgtagtgggcatgatttgcttgggtgaccccgtgggtaaggagccatttgttggacaccacgatgtcgtttttcacagccctgtgagcgcagcgtcttaaattgccctccagacattccaaatttggagagctcaaatggcaaaggtgaaatgggcgtcagccctccgggatgaaggaatctctgccggggttttccgttggatcacagcaggaggatttgctttcctaaagcattagagtgacgtggagagcccaaatcggacccagtggccacattctcccaagggaaaacccttcgggtgcccctacggttccttttctagcatgataacaaacttcttttccatccgcccatccccttttgggtttggaggttgacaaatccccactgaaattcctatgttgcacacatgtccttcattctttaagtaggagttagcaaaggttccgcattgacttaattcagagcgagatcaacaattttaggcattctttatgaacttcacattgttttatgctgatcagcagcaaaaaaacatacaggaataggagtgtgtctgtaggagtgctctgcattttcttgctcgtttggctgattaaggaagctgggaggaaatgttgtgaaataatcccaagtgatgagagactgtgggtatgggaggagatgccctctgtcctggtgagcagtagggacagaagacctgagctcatttcatatatctgtatattaaggcaatgctaaccagtgctgtcgtgttatttggggccaggagtggcttctgccccgttggtgcccataaaccagtgctgccccatttgggattggggtctgctcgcagaccacatccaccaaccaacccatggctgatagcagagaggcgaccaggtcaaccctccatatatctctgcagaaacctgttcctgtctatacagggatccccatccctcccccagccctccttccatcctcggcatttgggttggctataattaggctctgggaacgttcccctgctgccagcacagctgtcgtgtctgcaatgatccttccagctctctgcggacacgcaaaccctccagcaatcctaaatacccatttcctgcactcctgggacaaactgggagctgccaaaaatctccagccccccacagacgtgaccatcacagcaccaaggagcagagcaagcgcaacgtgattacggtgcaggtcggggtaagcctttctctttcttcccacagcccaggatttgggggatcctattggctctatgggatctgggagatgcaggagaaatgtgatccctttgctgtagcaaaacaaccttttagagtcctgcacctgaatctggcagtactggaaagcaggagagggattaagagtccttctgcattatcctgctcatagggaaatacagcacagaaatcattggggctgcttcctttgctttcttggcacaaatttaggtcctcattacagcgtttctttgactgagaccccaataggatctacaggggtagaacaaagcagacaaaaagtgattgatgtttcctatgcgatttgttgccttttcccattgagatttctgcttttcctatggggctttttgctttttcacagctttttttattcactgtagtgaatagaaatttttagggcttttaggtcattgatgctgttatgaacacagagatgaactcataacaccttcctggtgtggtttgtctatgggatagaaaggagctcatggtgctgtggacaactaacagaggtgcctgagggctgggccctctttgtgccccttctgggggtcagcaaactccttttattcagatataaatcccctcatccacaatttcaccagtcttcccaatgcagaccccaaaaaacatccccaatgacaaagtccacgaactgagaaaagcagcaaaaagcctcccagccccaaatatttatcccttatcccatttatttctatgggcaaagctattctaggcatcaggaaggtgggagattccaggtcagtttgttcctaattgtgatcttttaatgatgtttctcccatcaggtggacatttggaagtggttctgactgggaagaggacgtgatgatggcatcaggtagagctcagaaagtggtatttatcagcaaagcaattttccaggtctgttttttcccatttttcccatatttttttcttattcagggaagaggaacgcggatcttggtgagtgattttcttccttttaccttcaaaaagtccctttccatgtgtagaaatggatatacgtaccccccactgatacccatttcctttgttctgtccttatatttatacttccccatattttgaacacatgaaaacaaagcccacattaaataaattcataaacagtgcaatttttggactattattttccatagaaaagtattaaatcagtgcagaagtgcctctggaggtgacttctgcagcacccaaagagagaggcgtagggctgagtgctctctctgtctctcttttagaagaatgggatgcaaaaatcagtgagtgcccttttttcctctcccttcacggtgaggtatgggtgtggaggacctgaattaatgtgaattcctctgttttaagggaagctaacagaagattttggtaagtcgcttattttcctcgatctgagtgcatatttctacacctttaccatcagtgatgaccaacgtgtgtatgcatttctctttattccatttagaagagagcgacacagagctcggtgagtgctttggggtcttatcaaggtggaaagatgcccctctgtgcaacagtggggattgggagaagcccttcagctcttccatttatccacatctgatacccagatggagtcaggatgcagaactggaggaggagggccaaagctttgggcattttggggttatttttgttcctcgagagctcccaggattgacccgtgtccatttctgtgttatttccagaggaatgtgacacagaagatggtgagtgtcctccgtgagagggctcagagaaagacttccaccaaatctccctcctttaatgtatattctgatgtatttatttaaggggatctcgcagctgagatcggtaagtcgtgtgtggttatacacccctatttgtgcctcccatcaaacagggctctgtgcagcttgagttggttcccacagggtttgtcccccactcttcacacgaatatgggggtaaaacccaacaaaatggcacagagggattgcagaaagggcgggcgttgggtggcgctgtgttctgatccaagggagggtgaagctcatgagaatggttctttctttctctttttgaagacaatctgactgcagagctcggtgagtgcttccctttcctctctgcttcgtttcactgttgggtttttaggggggaaaaatgcttattccccccataaacacacacatgtaacccaacctgggctggaagaagggtccaaacgttcataactgcagactgcaattatcattcccaattggaaggtgattccatcatgaaccatccacccatcacagtggaattctgacagtgtttctctctgttttccctttcagaggaacgtgataggaaaatcagtaagtgcctttttccttccagaactgatgggaagcgatgggttagggttagggtaagggttagggtaagggttaaggttaggcttggggaaaaataagttaatacatttcattatggcttagaattgaaactaatgttcatctatttctttgttttaaggaaagctcacatcagatcttggtaagggttacttcctttaaactatccttaattctgcaacagtgctgggtatagagtagaaaaatatgcatgtgaaggtgtatgtatgcacatgttaattcattcctatttatgtactcgttagttgctatatatgtattaatttattcacattatatatatatttgtatatatttgcaaatatttgtatgtatgtgtgtatgtgtgaagagattggggtttcctctggttgaagaggggggtgaatgacagcaggtgtccttaataagccttattttcaaaacactaacaaggagaattgggatacacagaaataaagcctaaaaatgggaaaaagaaaagaatgaaatgggtaaaatattgaaaagaacaaaaagtttggagaaaagaaatgacagttttggttgggttggggctgctctgcatttctccgcttattttctccctttgctttcaggtgatgttgacacaaagctcagtgagtggagctgctcttcctgccccacatttaagagtatttttggtatttttaagactgtttaagaatatttggacatttcctgtggaaaatggatttctggtctgtaaaaaaaacctggggcttatttttgaggacggaataaatgtcccaaaaaagggggattttggcatcaattgactgggaggtgaaaaataaaagcagtgatctgagcgtgttggggccaatggatgaacctcaatgatcattgtggtccttttcaatccaggccattctatgattctgtgaaagaaaagaagataattaacatttaattttcttctttctcttctcattccagaggaacgcgacaggaaaatcagtgagtgtcacttttttggggccaaaaccctctgatttggggaagggatccctgatagaagtggttaatcctgttggtttttccctccttgcagccaaactctcagcagaaatacgtaagtccttttcctccccaatctgaactgtttctttgtattcttagacttccttttttttttttttctgttttaattaaaataatgcttttttttggttggttttttttttccctatttgacaggcagactgactgcactgctgggtgagtggtgccattaaatccgtgtgtggttttgggctgaaaacccttaaaaatgggaactctgcacccagacagctcatctctgtgctttgtttcatttgtaatagaataaaaatggggggaaatgggcaaaatgagcattgcagtgagcagagctgctgtcctggggcaagagggcaccgccgtgtaaaaaatacatatatttaaccatttttccttctttttttcccatttaggggaccgtgactcaaagctccgtgagtgccactctcctcctgattaaaatctgagtgaagatgtggattttcctcagtgtgctcctacaatctcactttttcagcacagttttccccaaactttgtgtttctccacccaaccccttacactgatcctaaatgggtgtattgcctgaatcagtggttttcttccctattttttatctatcctgttttattccagtatatgtttttatgacataattttatgacatattttgttctatgatgcccatagaccttattaccattgcctgccctgtgtggatcagaaaatatatttaatataaaacagatatctctactgacagtgatttctgatgcacccatgaaggaaaaggatttaaaataaactttaatttttccctttttaggcaaactgacagcagaactcggtaagccattttcttccccattcccataaaacaaatgaaattatggatggatggatggaaattaaccagttagtagaggtcagctttgctctaggacggtctgaaaagtgaccaaaatctgcttttactcatttttcttcttatttttttgtagcaaagtgcgatgcaacgatcagtaagtgctgctgcatgtgggggtacctccatcttcgggtcattttctgctgtttcagcattgaaaggacatcagaattccttaaatccaacaaaattggggtcactcgaaggaatctttgcagatatgggggaaatcagagccaaattttgagggggggagggaaaatctcaggggtgtttcagaaatccaatgggatctgatggtattttctgctctcaggactgtttacagtggaactcggtgagtccgtttcctttttgttttttttttctaattattatttattagtagtattataaatcaatattactgttgcttatacatattgttgtacattatatacataatacatacattatatacagtatatagtatacagtagtatataatattatgtattatatatatataatgtattataataatgtcatatctaatatatgtctgtattagatataatgcatatatattattgtactacagtcatattataatacatttacttatatctgcctttttccacacgtttcattgacctgattaaaaactaaatcctaaaggcagaagaagatgaaaacccccaaattaacaccaaataattgcagctatagatcatatctatcaaagcaaatttgccttcagtccacatcacgaaattaacaatagaaaggtttaaatttggaacgtacaaacaatgacaaataacccccaatggcttttctcttcttgcaggagagcgtcacaccaaaataggtacgtgaggtgtttgctaccttcgtttggaaggaagaaattgcattaataaaacctctgtccaatatgaagccggggtcaaattactcataaatcaccactgattgtccatgaattaacagggaaaaaaaggctaaacttgaaaataacatttttttcatctctcttttaaggggaactcactgcagaagttggtaagtctctttcccatcagtttaagcaaaaatggttcatcagatatataataatcccttatttctgcttgtttttaggggactacaacaggaaacttcgtaagtgcctttaacttctcccattaagagttaaacctttcaatatttttgatgcttcaatgtgctgaagccaccaaaaatgtgttttaattgtaaaggggctgagcgtcaaacctgaacactgccatgttgggggctgagattcgtgggatttgggttttcagtgtgaaaatgcctctgggtttctgtgcctgagctcagggaaacacgaccagggcttcccagtaggaatgagaccccaaaatatttctacctggggccttttcccattgggaatttattctgtaaatccatatttctccacgtttgagcgtcactcatcaaatgtcacaatcttggcaatgttgagaagatatatagatatctattttaatactgattaatatggaggtgtttgtgttggtcagtgatgtcatcgggaaaagatctgagtcattgaatccccatttcttttctctttattttaagggaaacacgcagcagaacttggtaagggaattccctccctgggtttgttctcttgtttttctcttttggagggggattttttctatgtcttctttctatgtcttctttctatgtcttctttctatgtcttctttctatgtcttctttctatgtcttctttctatgtcttctttctatgtcttctttctatgtcttctttctatgtcttctttctatgtcttctttctatgtcttctttctatgtcttctttctatgtcttctttctatgtcttctttctatgtcttctttctatgtcttctttctatgtcttctttctatgtcttctttctatgtcttctttctatgtcttctttctatgtcttctttctatgtcttctttctttccttctttctttccttctttctttccttctttctttccttggattttgagccaaaaaaatcacctcaaaatgagcctgaatgtttgcactgaggactgagcacagctgggcactaattcatctttatttctctcttatttacagaggaacgcgatctgaaaatcagtaagtgctgccccaaagccatagggctatgctgggcttcatccccacaacatgaattttataaattaaataaataaataaataaataattttatattttatgtatttgatattagcagtatttaaaaaaaagaataaaataactcaagaatcttaggatcaatagtaacacaatgatgcaacgtggatacaaaagcagtaattcctatttctttgggtttttatccttccaggggaacacgaagcagagatacgtgagtgttattttatatactctataatggaaaacttttttctctgtaatataaaaataggctttattatttgaggggttttttggcttaacgcaaatgcgaagtgcttgaaattctacgtatgaaatagaggatttcccatagagaaaaacagcaatttggggctggaataaaagtttcatttccttgctgaaaagtgaatgaaaagggggggaaaagaacataaaaattgagttttttccctcattaatctgtcatgaaatgggttgggttcctgaatggtgatgtcaacacctcgttttgggttcagcccaacataatatgtgtctgtcctttatttctgtatcactggtgttaaagagagctgttttgaactaatatctcttttttaattactttttctttttctttttctttccttttttctcccgtttctctctgttttgctttaagggcgcctcactgagctgctcggtaagtgcatttccttccttgcatctgtcaatccagcaacaaaccaaagcctattttgggggggaaggaggggataaaacacaataatgatgaaatcagtgctttggaaagggtgcaattattatttctcctgcaaatgaatacttccttttcccttttgtttgcagaggaccgcgattccgatgtccgtaagtccttttgtttgtcccggagctgtgaatcctccaatgggaaatgcagaatttcagagtctgccccaaaaatgacctttttgaggctacaagggatgggaaaataaggagaaatgtccttatttattgatctccttgtttatgtgcaaaactgggtgactcttctctgccgaacacgttagaaataagaacacaaaatgggaggaaatggtatttattcatatctgttgtttttctgtttaatttttaggagaacaggacatcctcattagtaagtggcactttggattgataagaaatgcagctcctggggacgtttgggtgctgcgattgctggcactgctggggctttgtgttgtggtggaagtggaattacttcaaaagaagagaagaatggaattatctggagaaaaaggggaataaatggaactgtttgggaaaagaaggaggaatagaatggaaatattggggaaaaaagtgaaatagaatggaattatttcaaaaaaaatggaatgaaatttagggagggggaaggggaagtggaatggaattatttgggggagaaaaaggggaaaattgaatgactggggggggaatggggaaataggatgggagtattttaaaaatacagaattgtgaaggtttcagcccatctcagagagtttggtatcctcgagttccccctttgcaacccattgagcatccttgggatgacaccaaattctgttttctccttttcaagggaaactgtcagaagagctcggtgagttatttccacttcttacatacaaaactgattctggatattctttttgtgtgttttcctgctttgcctctttgtgttttaagaggcaactgcagaaggaatggcacaaagggtgcagaggatctttgggataaataacagggaaaacagggatgggatagcaatgagttggtgcaataatctatggcacaaaaggtgacggcgtgtttcacattttgctttttctcttccttttagaggaattaaggggtcgggaagttggtaagtgagattcctttccctcttctccccaaaaggataaggggtaatttggattctgatctctttttctccctttttgttcctagaggagagtgttctggagaggggtgagtatcattctctttctactgctgcttttgactgaaggaatcccccataagcatgctggtgggatgggaattctacatctgatacacaattattatcatttcttcattttttatacacagaaatagataatttttttcctttcttttctcttttcccccttttttagaggaacatgatgccagaattggtacgtgtccatctccccctgcttttgtggtgtcttcaagaaggccaatggggtcatttgggattgtttgggttgaggattgggttcttgattgaatttgggggaggattcaggtgcccaaacacaacatcaggtcccatctcatgttttcctatgggcttggatccttctgttggatacctaagaatacctgaaatccataatatgccattagaagtaacacatccatcaatgatatatccatagaatacaagagaacggtctacatttacttcagatcccattttcaggttaaccatgaaaaaaatacccaaagactgaatgtcaccattcagggatcccgtgtgtaaaatcatgacttctgctttaattataagaaaaatgaaattcactgtttttattctcttttaagatgaactctcaacagaagttggtgagtatttttctgccctccagcaaaaccaaagcatgcagtttgcagtctgttttggatatatattgtacgtggatatataacctgtatgttataacacctctggtttccttttctccttcttttcctcagaaaaacgagagagaagaattggtgagtatcaaacttccccccagaagtggactttggtgtgttgggaagatccataccaccacgttggtgccaaacttaatggaaatcctttgttttttccttatgttttcagatgaactcactgcagagctcggtaagtcgtgattataactcataacgagttataatgctattgttatatataatatacatattatatattgttgctataattcataatagagcaaacaatcacaaggcacagaaatatgggtttgctttgagagccaaaccttaggaagtgataacacaatgggaagaggacaatgaccatttctgttgttcctcttttcagagcactacaaggcaaaagcaagtgagtgtctccttcctcatcttcagcacgtgagagattttgggggcttttgggacggctatggggatttacacataataaaacagaagatgagaagacagtttgttaacttgaattcaaactggtttgaaattggtgaaattacagtataaataatctccccagtacccaattatacaatgggattaattacagcctgcccaggaaaggagcactgaattttttcctgcgtccatccagcatgaagtccatcagacttaagcttacagcttaaagaatggttcatttttttcatttaaccccctcgtaagttaaaagatggacttcagcatcacagaagtagcccagaaatagtcaaaaaatgggtcatgaatttccagagcacccccccacactttccttggtgaataggaaaacaaatattaaaactaattaattggtttttttttctttttaggaagatgttttgaggaacacagtaagtgcccttttctcccttctttaagcatcacttttcactttaagtctgcatcacagttaataatccatctccttattatgcatttttagggagaggcgaagaaaagttgggtaagtcatttggttaattgggtttctgcttgcagaccccatccaggagctcatgtcctcctcttagtgtctgcactgtagaaatatccaggttagacgtgtaggtaggaaatactggacctgcgtggaggtattgcagaccccatttatgtgtaggggaagcagaacatcaaactattgagccttgagctccacgaagacaagccaccctcttagatttcaagcgaagtcgagctgaatagatttaattctttctttcccatagtaaatgtgactctggacccagagacggcccaccctcgcctcgtcctctccaaggaccagaagagcgtccgatgggaatacagcctgcaggaatcccccgacggccccgagcgcttcgacgccgatccctgcgtgctgggttgtgaaaccttcacctctgggaggcactgctgggtggtggatctcacagaagggcagtactgcgccgttggggtcagcagggagtccctgcccaggaaaggagccgtcagctttaaccctgatgaaggcatctgggctgtgcagcaatgggggttcaagaacagagccctcacctcccctccgaccccactgaaccttccacgggttcccaaaaagatccgcatctctctggactacgaatggggcgaggtggcgttttttgatgtggagaaccaaatgcccatcttcacttttcctctgacctcctttggtggggagcggctccggccgtggttctgggtggagctgggctccctctcactgcccagataaccccggaatccctggaggtgctgtggaggtgccttacagcagctcttccagaccggggtggaaaaactctcaggaaaagcagcattaaaacctcattctccctcttcccagtcaaccattgtcatgcaaaagaaaggaaacccatcctcaatgtcatcagcatcctccgtgtgtcatgtctggtggcccccattgatgtatggggtggctcctgttggtgtctggtgccccctattgacgtatgaggtggcccccattgacgtgaggtggcccccattgacgtgaggtggcccctattgacatatggggtggctcctgttgatgtctggtgccccccattgacatgaggtggtccccatgaccagcccctaccctggatccaatgcctcctgattgcagttccaaactctagggacgttaaacgacccacagagaggatggggtcctctttggtctgatggagagaggttggcaccagggtaagtcgctgcctacatcaccactggtgttttgtctcagcagctggtgtaaatttctgccatctgggctatttctgtagaaagcaaagaagctctgctggtgggcagctcatctcccagtgtgaaaaagcaaaatgcaacgcatgcaccctgctatccatgtggccatccctctccatcagctgttgaaggagaaatctgcactcagaagagattgaattgggctcagatctggcttgggaagatgatgattccaaccagagtccaggagactttggggaatgcatgaatcctataggaaaatggataacccttcatccaagagcaagctggcatgatgctctggggtgaaaacccataatgccacctggttttaaggtttggggtggcttacaatgtgcagctctgcttccggcgaggcactgggagccctaaacccatggagaggtcaaaccagtgctggaggtcattgtgggcccagctgcaatgggaggtaggcaattatggacatcgctgaagccaccccacgctctggggaacttgggttttcacctttcactgcactttaatgggatttctcatcaatgtctgcatgttcttggccacctgtttaaaaatataataataataattaaatcttttgccccactgcgggatgagcagctggtggttcccagctcacaataaaccacacttgagactccctggagaattcgctttctttttgcagctggttccatgtggggctgttcagcccctctgcagctcataggcttttcttcacagcctctgctccacctattgctgaaaagggggaaatttgagatggatcccattttgtgaacatctcccacctgtgggtaatgctcagacctctcagccctgtgggtttaatttctctttctgcagcttaatgggttggggatgttcattactgcaataattagtgatgggataggggaggcaggagaggatccaagcaggggaaaggggaggggaaggacatactgtgtgtgtctgacagccttcagattaggatatagaagctaaaggagcggctcagaataccattcctccctatgccaaagcagaactgagggcaaaaatagtggttatttaaaaatatatatgttttaattgactatcaacagggcgaatggcacaaaggttgcatcacgctgtgtggtgggttttgatgcaactcaaaattgcaactaggagttcctgtgctaagtgctaaggaaaaatgagatgaaaaatgagatgagaagccgcccagctgtttaattaaagcagtttggtgactgtcgtgctatggtgactgaagtaagcaagcactgtgctgcaaatgcccccatctctcctggaagtcgaggatattttcccctgtggccaataatgacagagcattttaagcccaaatctttatccagccaaattccacaatggagacaccgatctgcagggagattccagatagcacattctgtgtttccttaaatagggctgatatttccctctgtcccacatgtgatccatctgagctcacacagcccttaaaaatctgcaaggatcttgttctgctgcctggcggtgagtgtctttttggggctatttggcaccattttggcaacgggagttggcaacgtcaccctgagttcttccagagttctatgaaaagctttaaaatcgggtttttgcactttttccacgatccacctctagggaccaagctggtattgggggggggggggggtgtgctttcaggggggtttgggttctgttttgggggatatttggcatataaaggtggttttcacctttgggatgggccttggaaaggagtgttttgttggctgtcccgttgcctttcttcctacaaagcctttcttcctacagaggctgcaccaaggtctcttttagcagatcacaaagagagggcgcaaagaacgaggttagaattcaagtttttagggttgaaatatgggtaggatgattgagtccttctcctttgtccgtaccaagcctggagataccaatctgagatgtcaaactgcacaatggagccttcagtgggatgaacttcaggccagatgcccaagagaggtgttaagttttggttaaagcccacatcagtagcaatagaaagaaatgggtgattcatcccttccaaaactcaacttttggaagaaattagggaagaaatcccgcttccgtgctgaacccttctgtattttcaccccagggcaaagaaggaggatcgcgagagattggagaccacagcaccatggcctctgctgcttccagagcaaaggaaaagggagaggggggctcccaccaccctatcccagagcatcagatgggcaatggatgcagcagctccgtgggtcgtggaggtggcacgtggcaggagcgaggacggctcggagataccgaggtcatcagccaccgaaaccatctcaggaaagggaatttccacacaaaactccatttggagcacctggcagagaagctgaagcttttggggctggatggagacagaggggagaaggagaaactctgctcgtggcgcaagaggacattcccctccaatggaccacgggatgatggaggtcccactggagcccccataaaggagtcagtgcaggaggatgtggtcagccctgtgttattccctaaagccctgtttaatccttcatgtccatgctgaaaacttcttctctgcgaagtccaacacattgcatctcttcccttctttctcccatcacaatatcctccccaaacccctttttcttcctccaggagcagattcacagcgatctggagaacctcaagaaacaaaaggaggagctcttagaactcaaaaggagtggggagaggcgatgccaagaccttctggtaagaagctgttgccttcaagctggaaaaacagaggtctttttggggtccacgttgttgattttccacaacctacagacacggacggaggctgagaggcagaaaattgtgtcagaattccgtcagctccgccgttttctgaaggagaaggagatggtgctcgtggcacggctgggggagctggacagggctgtgctgaggaggcaggaggaggaggaggccaaggtggagggggacatttctctcctcggcatcctcatctgtgagatggaggagaaactcaagcaacccacacgtggattcctacaggttggattcctacgggttggattcctatgggttggatactccattggaccctctcccttcttgtccaccttctccaaagctgggggagattgaaccattttttcctatctctttcaattccaggatgccagaagcacgctgagcaggtatgtgctccttcaacctcattcaacggggtggaaagggttccccatccccacacccacggattctagcacagaaatgagaaatgcatgtgattgaggcaaggttggaagtcccatgggggtcaaaaagtgcctcagtgtaggaatggcccaagagaaagacctcgtggccattggggcgacccaagggaccgcattctgtacggagcaggttggcatccccaaacctgtgacaaagggacattctggagccaaccacctcaacctccatccccacatcaccagagatccccacactcatcaccaccagtcccagcactgatgagatttgtgtccaggtgggagaggggcagaacacgaaggatgacagagaactttgcagacgtggaacggaggctctgtgtcatctcccagcaacataaaatcctcagggagacgttggggagattccaaggtattgtggaccacagtattcctaccacataggatttgctttgtactgaaggttggggggttttttggtcgtttgaataggagttgtatacactattggaaaacaatttgcattaactcacactatcaatcattcttaggcctaagagcatctgttttttaggaccaaatcccacagatcccacataaaatcctgcacagatatccatgataaacatggtgggaactgaagcaggcagatgtgggacatgacatccaaccttctgttcatccccagatctttttctatctgagctggagaaggaggagggagcatctgtaggagaagagggaaaaggtgagtccttaaagcattttccttttgctccattggtcatttttttagccaaaatactgcgtcagagcatctggaaaatgatggttttgagctcatttctggtttcctaaaggtgatataaagaagctttcctatatttcagcaaaggttttctgagctggaaaatatggagacatcgctgatcccaaagtagatttggggtgctgttccagctttagggtgatgctcacccatttcttctccatccccaacagcgtttgtcaccctggaccccaccactgccactgcagggctcgtcctgtcccgggaccgacgtggggtgagatggatggatatggggcacaacatgtccccttgtccccaacgcttcgatgtctcctgctgtgtgctgggctgtcgaggcttcacctcagggtggcacttttgggatgtggaggtgatgggtggtgccacgtgggcactcggggtggcacgcagctctgtgcccaggaagggttggctcactttccaccccgattatgggatttgggctatgggatgctgtaggaacagcttccgagctttcacatctcccccatcc

SEQ ID NO: 32 - DNA, 356 bp, Gallus sp.
ggggggcgatatgggtggtgggacatgagggggccggggggggtcgggtctcacccgccagcagcagccgcagccccgcagccattgctctccgctgctttcgctttcggctccgctgtggccccaccccctccgtcacttcgtcaatattaattttaaatccctgaaacccattaaaaaaagggtcggagagggaaaactcattcaggaacagtgttggaagaggggacatgggtgggacaacccggctttccccacaggccgacctaaacacagccactgccacccaccccggatccatgggtgacgtaaggatgaggttccagcacatattggacccttctgcgtttgcatgg

SEQ ID NO: 33 - DNA, 37644 bp, Gallus sp.
gtggggggcagcgtccgcgctgacctcgtctcgctgtgtttcagggcgccccgtcgccgccgctccaggtaacgtcccgttcccattcccgttcccgttcccgttcccgttccgcgctgcgcggagcggccccgatcccggcgcggggctcagctctgcccgtctccccgcagggatgctgaattagctgctgccccgccgagccgctgcacccgcaccccccgctctcccggccgtcgcctcggctctccctcgggctgccaccgcgtccgttggagatgtcgccacgatgcacgcttcgtccccatcctaataaacgcgctgactttgaccccgctgttcgctgcccgtgaatcattggggactttccgtcgcgtgggaggaggggagggaagtgaaagcttcgtggagaagtaaacccagcaccctatgggtcccacgggacgtggattggtggggatggggtgggattggactcttggtggtcatttccacccatagggagctcgcggccacccagtggtcctcatatagactccatggtcacaccactgtcaccttttggtcaccccatgatccctgtgttaccctccggggtccctcagtggttaccccacgttcccccagaggctcctcctgtcgccttcatcatctcaccccattgaccacatacccccctccccctatggataacccaaagccatcaccagtggtgttgggatgcaaacacggggccccggacctgtccctacaagcacagggtggtgacacagcccagacagtgatgctgtgtcatttgtcaccaggcagaggacacacagccacagcctggctcaactcgaataatattttctttatttacatgttaaagaatcgaaaggttggaaacatacagtaagatgaaaacacggctctaagggtctaacagtggggcaggagggtgggggggaggaaaaaaaagaaaaaagggaaagaaaaaaccaaaacaagtagaaaaaaaatgatacagtcaacgtaaaaaaggggtggccctccctcccccagtgggaacatgcggcgctgcgtgccggggggttttatgtacaggggccgggcagctccaataaattaaaacctccaaatacaatgaggggggaagggggggtgcagagcccctcgctgggtggttttccttctttaaatgcttttttttttttttgtaattttttttaattttttttttttaatttttcttaaaaaccccaaacctttttctccccccccccctttttttttttttggaaaaaatcccacgagtcaggaggaaaaaaaaaaaaaaagccaaccctaacacaacaaacagtaaaacctgctggggggcaccgccgacccccccttgtccgaccccacagccccacactgccctggggacgctcgggggcctccggtcacaccgggacccccagctgagtccatggggcgtcccctgggctgctggggggctctcggtctgctccatgccggcccggtcctgcagagccgctcgggatgctgccccatgtggtgctgtggggtttaacccgaatccgagtcgctggtgtccgaggacgaggagctggaactggagctgctggagtcggagctggagctggaggcgctgagccgtgaaacagccacctgctgtgctgactcgggcttctcgttggctgcaatgggacaacactgcgctcagcatcaccacagatcacaccccaatcccactccagaccccacactcacccttttttgggggtttcttggctgagttgagctgcccgctgacgtcctgcagccgcttctccagctcccgcttcttctccagcgccagttcttctttcgtcttccccaccggcttcttcatggctgtgaaattcaggttcagccccacaccatcccacctccaccccagggccgccccctgaacgcagccccccactcactctcgctatagggtttgcggggtttcttccgcaggcaggacagcacgtagcgctccagctcacgcagtgtggagggtttgagggtctcgaagtcgatctcgatctcctcggggttggaatcacgcagtgagggctcccgggactggatgatgtgcaccacacggcccagcttctccccgggcagtttgttgatgtccaggctcaactgcctcttctcatcgtacgtcatcggtttgctctcctcttcctcttccgaatcgtagagcgtgggcggaggcggcagcgccgcttttgctgctttctttgagttcctgcaggaagcaaagcaccatcaggaaaatgaacctcaggaatcaccccacagctgaccatcatcccccaaaaaacagcctagactcacttggagctgcccccaccgctccccccgccgccacccttcttggctttgcggagctgtgcctgacgcgcccggctctcttcatctcctcctcgccctttgtgcttctccgatttcttctttttctttttctcccgcttctttttgggtttggaaacggggccctgtgagagggcagccagctgctcgtgcacggcccgcagctgtggggggagacagggggtgaggcgggcatggggagcaggcacaggcagcagcaccggcccagctccggccctcacctgctcctgcagctctgccaggcggttggcacgttcctcttccgagtcagagctctcctcgctgtctgatgagctctcactactgctctcatcctcatcatcatcctcatcatcttcatcttcatcatcctcatcgctggaggattcctcggaggaggatttggagagggctccaagcagtggggcagacactgagggtgggctggcgtcctgcggctcatcaggcatcttggcgtagctgaactcaaagacatcctgagagagaggacacagaggggtaagctgactgggctgggggttacggggctgctgggtgaccccacccacctgcagcttgcgggccatggccaccacatcgtggtcgggagggttgtatttgtagcagttggagaacattaaccggacatcagcggcaaactcctgtgcgtcatggtagtcccggttctccatcttccgctgtgggaagggaaaggcgtgagcagacctcaaagccacccccacaaagcccccatgaggctgtgccaaggcccacggagtccccaagcgaaccttgatggtgctgaggtccatggggtgtttgatgatctcgtggtaatcgtgcagccccagcgccgaggcatcgaccggcttgtagaagggccatgcgtaggcagcgtgcttcttggagagcagctccttcagaatcccattgcagtatttgagctgctccgacaatttgccctttttggaggtctgatgctgctgggaatccggcaagtccttcttggggggtttgatggggcggccgctctcacgccgtgcgggaattttggccgccttggcctccagcagcgtggctgacggggaggattcaccgctggtggctatgatggcggtggtggtaggggtggtggtgtctgctttccgcttcacaccctttttctaccaaaatacagaaaggttgatgaatgggaggcccagcacagcccacagagcctcctcccgtgagcgaagagctcccatctcccaccttggccacgggttgggtgggcgcaggcgcagtcagcacagccggggcagtggagtgcagcgacttgaggagcggagcggagatgacggacgggtggggaatgttgacaatggtggtggcgatgtcggggcttggggtgtacacagcggtgtgggacacagaggagacagctggcacttgctgagccgctgtgagacctgccaggagcgctgcggacaggcagaacccccattagcaccaaggtaccttcagtgctctacctgaaagcgcaacccaaagaaccccaggtacctgctgcccgcgacgctcccttcttgtggctgtttttggccactgggaccacgatctcctgctcttctggtggcatttgggccaccttctgcaggaagatcttctccagggtttgggccatcagcacaatgtcatctgtgggctacagggacaaccgaaacgtcacaggatgcagagatggcatcaaaggcctcaaagcatccatgctgcagtcctcaccttgttatagatatagcagtttgtgaacatggtgttgaagtcctgcatgcactcagctgccccccagtagtagttgttctccaagcgccgtttgatcgtccccatgtccatgggctgcttgatgatcttgtggtaatcctgcataggggatggacagtcagcgccgtgttggtaaccacactgcacccctcccagccccagaagcagtggtttggggtttttaggagctcaacatcccccaaagtatcaggacgttgacacgcacacagatccgctctcgcaccatgcatcaaaagcagggcaaaagggtgcaaagggatggaaaaacacctccgggtctggtccccgcccgagagtgccaccgtgctgctctgtaggggaccttcaggtgctcttgtgggttgcctacgctatagggacagccaaaacactgctgtccacagcatgaggtgcaatgggggccactaatgctaaagtaagagcaaacctatgtggaatttacctctgggctttaaatccttgggcgccacaggtacacagggggctggcgtttaatattggggtcacaagatgtcttctacaaattcatggatgggaatctgcaaaacgcattcagggcacaagagattaggtgaggaaacatccgggttccctctagagcagctgcgtcacctcacccatacccgtgcggtggcactgggaggggacagcagctctgaggacatcaggtcacctactgggggggcttcagagcctgtggagttgggattatgcccctaagagagggcgaggccagcacagcccaggcacctgcagctgcatctctgtggtggagcccatagaggggacaatgctgtccctgtggcactctcaggctggggaccacggctcggggtggccctcagcacccaggggacaagtctggggacacacagccatgctgggggacccacaggaggggacacgttaccggcagacccagcttgacggcgtcgacgggctgacggaaaggccaagcgaactggtgcttccacagggctttcatcaccactttgtgcaggtactgcagctggttggtgacccggccgggctttttggggttcgagacctctggcggaggggggttcgcctggggggtctgtagggccggcaccgaggccatggtggggctctcgaagccctcgtaaagcagcgagggtttgcggatgcgtttgcccggggtcgattccgtcgccaaacccataagcccggcatttccctcccccagaatcctgcaagggagcaaaagacaacatcagcaaggatggggccagcgttccaccaccaaggtgcacaagaacagctcaaaaaaaggcaaaaaaagtaatcaaaaaaaggaaggttggagcaaacaaagagtcagtgcagggggtgacatcagggcccagcagtttcaccacctcggggtacgacagcctgcactacagcatgacaaggcagcacccaaacactgtggccctcagctggatacacaacagtgggctccaaatgtctggggacgggggcagaattatttaagtggggaaaatgaggatttaagcagctgggagaggtgggatgtctgcagcgtgaggagaatttgtcaccgggaaaatacggtgaatgtcgagcactggggctgctttctcaggcagctcccagggtgttccccatcctgccaaggacgtggtgggaatgacaaggaaggaaggtgacagaaggacacagcggccccagtagtggcggtacagggtgggaggacacggtgagacccctcagcatggtgacagtgtccccgaaagcagctcagtcagcagaggtggcagcagggccctaagggcccttgtgatgctgaccccaaggaccaggggtatgaggagtggataaatgggggtggcccagacaggatccatgggaaaacagggctgccaggttccctgtaggatctgtgtccctgcatccctgacagaattcacatggaccacggggctgccgagtcccaacatccctgaaggacccacagaaatgggaagtggataaatgggaacaagcagcagatcaatgggactcagtgaccccaaactcagagctctgtgacagaaaagccccataactctggtggacatccacactgcaccctaatccctgggcaatgaagggatagcagcagggaaccactgtgtccctgtatctctgaccccaaagaatccatggagatggggaatggataaatagggatggctctgtagaatccgggtcccattcccctcaaataatccatgggaatggcactgttggatgcatggccttgagtccctgtccctaaaaatctgtaggaatgactctgtgctatgcacctccccgtgtccctgttaggatccatggggacagcaggctgccaggtcccctgtatgatccacagccctaaaagcagcttggtcaacaaatgggagggaacagcgggtccctaaagagcgccaggtcgccatgtccctgtccccaaaggacccacaggtacaaggaacgaataaacagagacaaggagcactcagtgggatacaactgatgtcaggtgcagagcctttgaacacagaagccccatctccccataggattcaggtccccatgcccctgttggaaccatggggacagggaggctgatggattccctgcaggactgagttcctgtgtccctgaccccgaagaatcgatggggacagagagtagataaacagcgatagccctataagatccaggtccccgcgtccctgtcaggatccgtggggaccgtggggctgccaggtccccacgtccccgtccccaagcaatcgatccacggggatgaggaacacataaacgaggacaaccggcatacaaacgagatccaaccggccccgggtggagcaccgggacgcggcagccccataccgccgtccccgcagcaacgccatccccggttcataactgccaacaccccacagccccccccggcccccattcctgcccctcatcacctacttgctctggggattcacattctgcagcatgccggcggctgcgtgcccggccctggctcccggccttcctcctccacctcctcctcccgccgccgcctcctccgacgtcccccccactttgcccaccgagcagcgcccgttaaggcagcggccctcggccgggcatgaggcggcggctccggccgggccccgccgcgcgccctcacatcagcggagaaaatggcggcggggcctggatggagaggggggaccttcctgctctccgctgcgcacagaacccgccgcgacgccgccgatatagagccgggaaagccggtaggaaccggatagatcctcggaaggacggtgtgaggcggatggaaggcggacagagggcggatggaggcggatggttcagcgggagggctccatcttggctcgtaggccccgaagaggaatcggtgccggcggccgcaggcaggggtcgctacggaggccggggagggtccggtggagccgtccgggagcgcgaaggcgggggctgggcccgcccggtggaggatggaggcggattggggccgccccccagcgcgcgccgagcccgacccgcaccgtcccttcgtccccacgaaatggcgccgctcggcctgcccccggccgcccttatatagacaccacctgggtgctgattggtgggtggacgcgctgacgtcagccacccgcttgcacccgccctgccgctgcctcattggacggcggtgctcaccgcgcagcgctcctcttggccgccccgcacgccactcacccgcgccgctccccccccgcccttccgcccggtactgcgacggtcattggtcggtgctgccattcccggcgcggcgattggccaccacggatcacgtgagggcggcgctgttggctgttgttgtcgcgcgcgaaggtgcagagggagggggagggctaaggcgggcgtcgccattttgtgtggcggcggccaacgggcgcgggcgtcccgggggttcggaccctccgcccaaaggctcctcagggggctcagcagaccccagtcagtccctacggggctcgtgaggctctcccagccgacagcggccatcgggcagcggagccgcggcggtccgcgagccgcgggctgagctgtggtgaggtagtgagctgggtcccggggatcctgaagggtcctgaggtaatcgcggccctcagcgcggtcccggggccttcagtgccaccccacggtggtactggggccctcagaccgtcccctcccccactgccacggcgatccggagggggggggtccgaggccgccccgtgtctattcggaggtgctctgtgctcttcttccccacggcaattctggagggctcacagctactccagagcagccccataaccgtcctgggggcctcactaccaccctacagcaactcagagcctccccccacccccccaaaaaaacaatcctggaatccccaaggccatcccacaccaacgctgaaggtctcaaagccccccctcccccacacacaccggttctagggtcctcagaaccaccccacagcaatcctgggacgttccacagcccctccgtagtaatcttttagtttctcaaggccaacccgtagcacggggggcctccgctgcctcccctcgtggcaatcctggggggctcagtgccacctcacaggaatttcgggtcgctcagaacctctgcaaagcaatcctggggtcctcgaggccaccccacaccgatctcagggtgctcaatgccaccccacagctgcccccggggccgtcacaatcaccccacaccaatcctgagaaactcagtgccaccccacagccaatcccggggtgccccattgcctctctaaagcctccaccccaatccgggggtgttcaatgccaccccacaacccccctcaaagcactcctggataccccacggacaccccaacgccctaaaacaaatcccagggcgctcaatggaacctcccgcgcaacctcgggctgccccacgccccctccaactcaagcacgacccagagaccccccttttctcccaacccctccggccccacacgccaaaggctctcaaagacccccccccagcgatcccggacccgaacagggctttggggtcccccccacggcgctcccggtgccgcccccccccgccccgtgacacagcactttggatccccgcgggccctccccgccgccgcccccgcgcgaacacccaaacatggcgcttttcgccccaaaagcgccgggcacaaagcggcgccgcccattggtcgtctgcccgccgtcctcgcttcccattggccccttcgacggcggaggggcggaaccagatttgatggacagctcatgctcacgtgtcctcccccccccccgattgggtctttttggttaaaaaaataaaataaaatcataaaaaaagggcgaagttgccccatcgtcactcacctgagccgctcccacgcagggccacgacccccaacccgatatcatcctcgcgtcgcccccttaaagccccgtttttggggcaaaaaatcaaaaaaaacatcccagggcagaaaaaggagccacgcgctacgtcagctgcaccgtgattggccacccgccgtcacgtgacggccccgcgccactccgacggccccattcatggagcccgggcggctccgcggggtcctatcgccgctccggagggggtgatggcgcgcatgcgcagtgcagggggcatgtggtggggggagggaggggtggggctgtggggatgcccggtgttgctggggggctgctgtagggttgcatggcattgcgaggatgcagccatgaagattcacggcattgtaagtgtgcatctgtaggggcccctggcattgcaagtgtgcacctatgggagtgcccggcattgcaagggtgcacctctggacgcgtttgtcattgcaaagggtgcagctgtggtggtgaatggcattgcaagggtgtatctatgggagtgtacagcattgcaagggcgcacctatgggtgtgcctggcattgcatgcatgcacgtgtggggatgtatggcactgggggggtgcaccagtgggggtgcttgggattgcaagggtgagcctatagcagtgcctggcattgcagggttgcacgcagggatgcgtacggcattgcaggggtgcagctgccggcattgcagagggccgaacccgcccgtacggttgtgcagcgcttccagctcggagggcgcattgcagtgcggtgcattgcagtgcgaggagccactgctgcagggtgtacagtgcacgccccgaggatgtcccctcggctccaaaccccaaaaccccacgcttattaccccccaaaaacatacttttacacacaagacacatttttaccatcaaacctcacgcttttcccccaaaatccctcacaaaaacaaaatccgcgccgtgatgagacaccccagagatctacggagcctactcgtcccctgcttcattaattagagctgcttattaattgcttgcgggtggctcagcgcctattacagcgtcggggctccccgctagtttcttctatctagtaacaagtgacgcaaggtaactgcggagcgcggccattggttgagccgcacgatcatctcctgtcacagcgctggtgttccccgcagatctgttctgcctagcaaccgatgacgcgtaaagccgcgaggcacggccattggctaaactggttgccggtagcagagggatgggggctgcgagcgggcgcggggcgctcgtggcagcgctgctgggggcggccctggggagcgtcagagccggtaggggacgagggcggggggcggtatgggtgggcacggggtagtgccaggggtgtccaagatgtgtgcatggggagtgcaaggggctgtgcaaggagtgaatgatgcactggggcaaggggtgggcatgcactggggcaagaagttgtgcaaggggtgtttgtgcattgatgcaaggggaggatgagcaggactgtgtttgcatgcatgcagggggttgtgcatggtgtgattagtgcattagtgcagcggggttgagtgtgagatgtggagtgtgtgcagttgtgaaagggttgccatgcacgagctgatgtgcgctcagtgagcgtgcacgcagcctgcagagtgggtacacctgcaacgagcatgcatgcagcagatgtgtccatgcacagtgtgtgtgtgtgcctggtgtgtccccatgtcccacgccctgtctgtgcctgcagagccatcgctgcacactctgtctgaggtgctcttctgccagccggacacgccgtcgctggggctgtcagtggccttcgactcagagcagctcttctcattcgatgtccccaactcgcagtggctgccgcagctccccgatggcccctcgtggcccgcagacatcgagcagccccacgagctgctgcacgacgccgcgctgtgccgtgagctgctcgatttgctcaccagaatcgccaccgggccaaacccaatgcctgaagccaagggtgggtgctgctgtccccgctatgaccccactgatgggtccccagccgtgtgttcccagtgatgctgaccccaatggacatccccagttgatgcatccccattgatgcatcccccacagacatccccattgatgctgtccccattgatgtgttctcagtggatatccccaattgatcctgttcccaatgatgctgtccccaatggacatccccattgatgctgtccttgttgaagttgtctcgattgatgcattcccattgatgtgttcccaatggacatccccagctgatgctgttcccactgatcctgtccccattgatgcatccccaattggtttattccccattgatttattccccatggatgtccccactgatgctatccccagtaatgctgtccccactgatgctgtccccaatgatcctgtccccagtgatgtgtgttcctaatggacatcccaactgatgctatccccaacgatgtgtcctcactgatgtgtccccagtccatgtggttcccagtgatgtgtccccaacaatatgacctcactgatgtctccccagttgatgcaatccccaatgatgcatccccaacaatgcattcccaatgatatttcctcaatatgatgctgtccccaatgatgcattccccattaacgcactcccaccgacgcattcccaccgatgtgtccccactgatgcgtccccactgatgtgtccccactgatgtccccccccacaggcatcccggtggccgacgtcttcctgcagcagcctctgcagctcggctaccccaacactctgatctgtatggtgggcaacatcttccccccagccatcactatcagctggcagcgggatggcatccccgtcaccgatggcgtcacccacctcacctacacccccaccgaggacctgggcttcatgcgcttctcctacctggcggtgacaccgcactctggtgacatctatgcctgcattgtcacccgcgagagggacaacatctctgtggtggcttactggggtgagtggggatgtgggggtcatgctttgtgtccccgcagcggtggctgatgggggcgggtggtgggagcaatgctttgtgtccctgcagtgccacaggaccccatcccttcggacgtgttggccacggcggtgtgcggcgcagtgacggcgctgggcatcctgctggcactgctgggtttggggctgctgctgtccgcccgccggcgcagtatgtggggacaatggagacagcagggacacccgccccgtactcactgatgtcccccataagttgatccctcggtgtgggaacggtgatggtgatgtaattaaagcccttcatttgcagcgcggtgtccttgtttgtccccactccgggaagggtggcaattaatggggttgggcattgtccccatggccccaggtggcaaagtctgatcccattgcaccgcccatggggtgacgtgaggaggaggaggaggaggaggagaaggagtaggaggaggtgaaggtggcacggaggggatgaaggcagcagtgctcctgatggggccaaggggtttcagggtgctgggggcgatggggctggtgctgagctgcgggacagcaggtaggatgtggggaagtgtggggtttttggggtgaagcatgggggttttagggtgcaacatgggctttctggggtgcaacatgtgggtttgggggtgcagcatgggggttttagggatgcagtgtgagtttttagggtgcaaaatggagtttttggggtgcaacgtgggtttgggggtgcagtataagtttttagggtgcaacatggggttttatggtacagcgggggctttggagtgcagcatggggtgctgcatgtatgcatagtgcacaacatggggttcttggtgtgcagtgtgagtttttagggtacagagtgggggatttggggtacagcatggggttttggggtgcagaatgaattattagggtacaacatggggttttagggtgcggcacagggctttggggcacagccccagtgctgtgccctccccatgcccccaacgcaggcgccttcgtggtgcacatggccagctcctgcccactgctggccaatggctccctgggcagcttcgacctcaccatggccttcaacaagaaccctctgctgtgctacgaccccgacgtccaccgcttctacccttgcgattgggggctgctgcacaccgttgccactttgctcgccgccatcctaaatgatgataccacatgggtgcagcgtgcagaggcacgcaggcaggcgtgcactgagctggctgcacagttctggacacacacagcactgcgcaggagtgagcaccgctgcatgcaagtggagcattgcaaacacgggacgttgcatgggggtgttgcatgggggtgttgcaatggggtgatgcacagccggtcattgcatgagacgctgcacggggatgttgcaaagggaactgcatggggacatcgcacagcaggttgaatgggatgttgcatggggactttgcaagggaactttgcacagagcattgcaggggatccacgcaaggaatttgcatagggaatgcacagagatgttgcctgggaatgctgcatggggtcattgcatgaggaactgagagagacattgcacaaggaaatgcaaaggggcatcactaggggacatggcatggggcattctagggagcattgcatggggacattgcaaagggaatgcaaagggacattgcatggggacattgcaaacaaattgagtgggagattgcaccgggatgttgcatggggacattgcatggaatgtcccaccaaccaccctgcagggtgacactgggaccatccccagctctgaccatcccccctttgctgcagcaccaccccaggtccgcatcgtccccatccccatctccaacgaccccgacaccgtccacctcatctgccatgtttggggcttctacccacccgcagtgaccatccagtggctgcacaacggcctcgtggtggcctcaggtgacaccaaactgctgcccaacggggactggacctacaggacacaggtggccctgagggccagcactgcagcagggagcacctacacatgctcagtgtggcactccagcctggagcagccgctgcaggaggactggagtgagtttggggatggggatgtggcacccacaccccacagtcccccacggctcattgtgcccacgctgtccccacaggtcccaatttgtccccggcgatgatggtgaaggtggcagtggcggccatggcgctgacgttggggttggtggcactcagcgccggggttttcagcttctgtcagcggccacggggtgagggatggggatgtggtgctggggacatgtgtgacaccgagggtctggtgtccagtgtggggtgtacctcctcattcatcatcttctgtgtggcagctcctggcgctggtcccagtccccgtcctgatgcgggttctcactccaatcctggtccccaaaatgatcccggtccaagttctggtccccatcccagtcctggtccccattctggtcttggtcctggtcctggttctgctcctggtccctatccctgactctggtcccggtccccatcccgatgccagtcccagtcctggtccccatcctggtcctgctccttggtttggggacctcaatgactggaactcccatgtcccaacatggggacccacagtttggggtgaggggctctcaccccccaataaaaccatctgcagccccaacctcgctccaattcttcgttcccacgttgggtgggtcgggctcccagtgctcccagtgctcccagccgtctatgtcccgtaagcgtcggctccactgcattctgctccggaaacagatgacgctaccacggcgccgcctctgattggctgctccgtgccctctctccgtcccacgtccgtgaagggggggatgtggggtggagggagctgaggggccgcccccttcccccccccgctcccccctccgcgatgttggtgctattggggctgctgctgggagcgcggggggcaggtgggggtttggggttggggtgtttggggggtctctgcctaatgaaactctgggggggggggacgtgggggtctctgctttatgggactgtgtgggcgggcttgaaggggctctgctttacggcgctgggtgtgggttctgggaagactgtgctctatgggatcatggtaggggcttgggggggctctgcttaatggcactgtgtggagggacattgggggtctcagccttataggacgttggggatgatttgtgggggtctcagcctttgcaatattgaggacactctgggtggtggtctgagcccttagggcccccagggagggctttagggtgggctcagccttttgggatactggggtccttttgaggggagggtctcaactttatgggatgttgcaaagagtttggagggggtctcagctctgagggatattggggacaattcgggggatctcagccctttggaactcaatggaggattttggagtgatgctgaggactcagccttttgggttgctgggtatgatttggggatgctcagccttatggaatggtggggacactttgtggggagctcagctctgtgggatattggggccactttgggggagtctcaacctttaggactcccagggagggctttaagaatctcatgggatcccagtgttgagctcagctcgatgttatgtccccatggggacctctgggggctccaaatggggatgaggtcgctgccagcactgccatctcccctctgtccccccaatgcaggtgccttcatggtgcatgtggccaactcctgctcactggcagccaatggctctctgcggggcttcgacctcaccgtggccttcaacaagaaccctctggtgtgctacgaccccgatggccacctcttcaacgcctgcgactgggggctgctgcacggcgtggctggacagattgccattgccctcaacaatgacagcacctgggtgcagcgtgcagaggcacggagacgggcgtgcagcaaactggctgcacagttctgggcacagacggcgctgcgcaggagtgagcattgcaaatggggctgttgcacggggcgttgcgtggggatgatgttgcatggggcattgcatggagatgatgttgcatggggtgttgcatggggacattgcatggggcattgcatgaagatggtgctgcatagggcgtcgcatggggatgttgcatgaagatgtgtagcaatgatgcatggggcattccatggggacgctgcatgagggtgttgtttagcaatgatgcatggggtgctgcatggggatgttgcatggagatattgcatggggcattgcatggggttgatccatgcagcgttacatggggttctcaagcagggggatgttgcatggaaaagttgcatggaaaattgcacagaggtgttgcaaagcatatgcatggggatgttgtatggaggattggacggtggctttgaagaacattctgcatggggcattgcttaagggtcccaagcatggggatgctgcaaggaaatgctgctgcttggtggccttgcagagtgtgttgcatggagtttgcttcaaggagatgttgcatggcatatcatctgcagttttgcagagcacattgcattgcacattgcacactgcacagagcagtgcactgggcatctcccagcgtgtggcacaacgctgttgcaaaggacatcccacgaggtgttgcagcaaacaatgcgcagagcttgcacagaacgtgggatatcccatggggatgtggcacagagcattgcgtggggaatcctacagggaagtgagatggggaagttgcacagagcgttgcaaggggtattgcacagagggaacttgcagagaatggggcaggaaccgtccccatcccctgctgctcaccatccctgtccccactccagctcagccccaggtccgcatcgtccccgcacagacagggaaccccagcgtgcccatccgcctcacctgccacgtgtggggcttctacccccccgaggtgaccatcatctggctgcacaatggggacatcgtgggacctggagaccactcacccatgtttgccatccccaatgggaactggacctaccagacacaggtggccctctcggtggccccagaggtgggggacacctacacgtgctcggtgcagcatgctagcttggaggagcccctcctggaggactggcgtgagttgggatcaagggggtgacacagggacagcggtgtccctgctgtgtcactgctggctgtgtccctgcaggtcctgggctgacgctggaggtgacgctgatggtggctgtggccactgtagtgatggtgttggggctcagcttgctcttcattggtgtctactgctggcgggcccaaccccctgccccaggtgggtgcttgagagggaccctatggggctccatggacctctaaggggtctctgtctggttcctatgggtctctgggttgctgtgaatctttcttttctctgtgggtccgtctggggtatctgttgatccctatgggttgctgtggggcctctgtgggtctctatgggtccttctgttggcctctgtgaggtctctatttgtctctatgcatccctttggatctctatggggtctctgcgggtcattacgtgtctctatgggatgtgaccatttttgacaagaaccccactcacccctcctattcccccaacaggttacgccccgcttcccggtcacaactacccttcaggtaacagtgtccccaaactgtccctgtccccattgccatcaatgagggctgagtgaccccatctctcaccccatgtccctgcaggcagcatctgatggacaccttctgtcaccaactgtccctgcgtgtccccatccctgactctgcgccgtggtgctgacattaaagacactctgcagcctctgttggtgtctctgtgggcttttggggtggggtggtgtcaccggggagaggttgggttggggtcattgcatccatgatggtgatggtgattgacattgtgcacagggagatgtccaggcgcctgtggggtctgtgttttagggccagttctgctcagtgcctccgtaagtgatctggataggtcgtcagtcatcctaattaaggaggggacaacagtgaatggggaggagccgatgactcaggctgggagtggtgatcccagaggtttcctctgctgtcagtgactccgtgctttcgctttcgcttcacaacctgagggagcgcattctgcctggcgcccgatgacgtcacataaacccccgactgccattggcggagaggcgacggaggagccaatgggggcgcggggcggggcggaggagtaggaaaagctgaaggagctgcgctgggtgcggcggacttgagagtgcagcggtgtgaggcgatggggccgtgcggggcgctgggcctggggctgctgctcgccgccgtgtgcggggcggcggccggtgagtgcggccggaccgggacccctccccgcccgtaaccccaccccggggctgtgcccgtgggatcctcagacccccacccgcggctcacggcctcgctgcgctccgcccccgcagagctccattccctgcggtacgtccatacggcgatgacggatcccggccccgggctgccgtggttcgtggacgtggggtacgtggacggggaactcttcgtgcactacaacagcaccgcgcggaggtacgtgccccgcaccgagtggatggcggccaacacggaccagcagtactgggatggacagacgcagatcggacagggcaatgagcggagtgtggaagtgagcttgaacacactgcaggaacgatacaaccagaccggcggtgagcacggccggggccgcggctccgtgggtgtgggatgggctccatggcgcagtgccgcccacaccccccaggcctggccctgcccggcggcaccgtcccggggctgcccgtcacagccccaccgcgctcggggtgccgcgtcccggggggaccccaacccatccccgctgcagtgggagccccggagccggaggggcccctcaccccctgcccggctgtgtttcagggtctcacacggtgcagctgatgtacggctgtgacatcctcgaggatggcaccatccgggggtatcatcagacagcctacgatgggagagacttcattgccttcgacaaaggcacgatgacgttcactgcggcagttccagaggcagttcccaccaagaggaaatgggaggaaggaggtgttgctgagaggtggaagagttacctggaggaaacctgcgtggaggggctgcggagatatgtggaatacgggaaggctgagctgggcaggagaggtgagcggggtcggggtgggggggggggggggcggacgcagtgtggggctggacgtggggcgggggctcatcgtggggagctcagcccggccctcactgccgcccacccacagagcggcctgaggtgcgagtgtgggggaaggaggctgacgggatcctgaccttgtcctgccgcgctcacggcttctacccgcggcccatcgccgtcagctggctgaaggacggcgcggtgcggggccaggacgcccagtcggggggcatcgtgcccaacggcgacggcacctaccacacctgggtcaccatcgatgcgcagccgggggacggggacaagtaccagtgccgcgtggagcacgccagcctgccccagcccggcctctactcgtggggtgagtgaggggatgtggggctggggggctgcgggctgccccttcccctgctgatggccccgctctcccccagagccgccacagcccaacctggtgcccatcgtggcgggggtggccgtcgccattgtggccatcgccatcgtggttggtgttggattcatcatctacagacgccacgcaggtaaaagcagaggggtgcaggcgggcagtgggggctgtagggggatctgggtcccccttgggagcccccaacctggctgtgatgtgaacctgtgatgaagcatctctctgtctgcagggaagaaggggaagggctacaacatcgcgcccggtgagtgatgagggcagcgctgtcccccacctctgcccagtgccagggtggtcctggggtccctgctttctcccaaggtacccattcctggtgcttggggctgctccatgccccatagggagcacagggctggatctcacagctgttcctcccttatagacagggaaggtggatccagcagctcgagcacaggtgcggtgtggggctgtgggttgggaggggtccgtgtgctctctgtggtactgcccagggctgggctatgctggggctctgcggggagacccccggagcagagggttgggatgtgaacctggccccgtgggacatcatcccttctcatccccacagggagcaaccccgccatctgagtgctgtgcttcagcctgcaaggagccaacagtccacaccagcatttggggtcggtgatggacacagccccatcctcctgacctctcacatctcattctgcttcctatgctgactgttatgctttgcctgcactgcttcctgtgaaataaaatgatgggccattctgtgctcagcttgcctgcattctgcactgtgctgtggttggggatggggtgggtgagaggaccgtgtcccagtttggctgctcagggtgcagatgtggccctgtgctgagtacccacagccctccccccctatctgcctgctgctcactcccccttctgtacccccatcccttctcacctctcctctgtgaccccatgctggtggttgcttgctccctgtcctggcagaactctcattttcccaatggcatccctgggtgttgggatgtggtctccttggtcctccccccagcagtcactgcacatatccaccccacttcccccccaggttgttgtcccacagcactcctatttccctctccccccccccccccccccccccgcccatccagctgcctctgcaatcctcacccttgcccacacacaactttgcgcactccacctccctcatcccgcccttcccccagctctcctgtccctgctggcccccctcccccccccccattgtaccctacacccaaataaatatgtttgttctgctgccctccagccgtctcctggtttatttccccccgatttgttgttgttgggggctccgctcttcaccctggggggaaggggctctgggggtccctcattctccctgcacttcttacagcaccgggactcccgcgctgagatcccatcacacccgggtacaaacatgcggctttattcccagttctgtgtcccacccccggccctggtggcactcagtggcaccgcagtccatgcagtggccgttgtgtgtcgtacagcagcggtaccgcagcgcgcccggctcggcatccatgtgcccacggcacagctcttgtggtcccttgtcgctgccccggtgtccccacctccaccctcagtgtccccaacgcgcagtgtccccatcccacatctcaccaccccctgccctccccatccataactcccgctatccctctgtccccctcccccggtgctccctgttgtccccagtcccgcagaaggctgccggggcgcagcacctcgtgggggggtccctcctgccgcacctctcccccctccagcaccaccacccgctctgcccgcgccgccagggctgcccgccccgtcaccatcagcactgcacgccccgaccctttggctgcgaggatctcctgctccacctgtgggaggagaaacggtcagggggatgtcctcagccactgccagggaccgagggacaccaggagtggagataaggggacaccagaacaggggaccatggggacctaggcgtgcaatctgagggaacacagggctcagggggatgtggggacacggggacgtgggggacatcccacctgctgctggctctcagtgtccagggcgctggtgtgctcgtcgagtatgaggatgcgggggtcccgcagcagcgcacgggcaatggccaccgcctgccgctgccccccggagagctgtcctcccaactcgcccacctctgcggggacagcggggtcaggctgggaggggaccatggaggggacccggaacagagaggggacagcttacctgtgtcgtagccttggggcaggcgagtgatgaagtcgtgagcacccacctggcgggcggccgctgtcacctgcgcccggctgcagccccccaacccataggagatgttggcgtggagtgagcgggcaaagagcagcggctcctgggggacgacggccacctgcagtgggggggacagctggggacatgggcacgtggcagtggaggcggtggggatggctggggatgtgtcagggacatctggggacatggtgggatggttggggacacaaggatggttggggacttggccgggacactgcaggggacacagtggtgatatggcagagacatcagggtgtgtggtgatggctggggacccagatatctggtgactcaatgaggatggctgctgatatgcaagaacacagggggacaaccaggagccatggggacatgtggctgctcacctggcggcacaggtaggagtgctggtaggcggggagggggtggccatccagcagcaggcgtccggccgtgggctggtgcaggcgggacacgagggccaccagagtgctcttccctgcgcccgggggtcccagcagtgccagcacctccccggggcgcagctccagtgatacgccctggggacacggatgtcacacccatggtgtccctgtacccacacctccatagtcccacggccttcctgctgtgtccttctgtccctgttcccccccgaccctttgttttgttcccacacctttggtaccacatctacattcccatgccctccccaccatgtctctgtgtcactcatcatgtcccagtgccacaaacccaccatgtgccatgtccccgcgccctcaataccatgatctcatgtccctctccagtgtccccataccaccccctccatgtatctccttgtttcatatcctcacaccctgtccatcacatccatgtccctgaatcaccccactgtgctcccacacctgtcacgctgtcccaccacacccccgtgtcccctggctgtgtctctgtgcccaccttgaggacgggttcctggcgcccagggtaggagaaccagacatcctccagctgaaggtggccctgcaggtcagcgggtgccattgtccctgagggtgcgacctgtggctcccggtccaggaactcaaagatcttctccgaagagcccacagccttcatcagtgtggggaagtagtcgagcaggacctggggacagcgtagtgacgtggccaggagggcaagggcatgtggcatggtgacatggggacgcagaggacacagtgggattggcatagggacaggacggggtaggtatggggacagaggaggtgacacagggatgtggcaggggtcacagagggttagacgtggggacacagggacatggatgggcatggggacacactgcgattggcctggagacacagcagggaggatgtaggaacacagggacactgtgacacactgtgactgtggcagggagacatggggacatgcggatatgggcatcctctcaggctcacctccaggacatcagtgaactgtatctggtagaggaggaaggtgacgaggtccccagtgctgacggtccccgcggccaccagctgccccccatagtagaggatccccatcttcagggccagggctgagaactgaggatgccatgaggtcaccaggggacacctccccctgggactctgagtccccagcaccactctgtcaccttcctgttctgtcaatgtcccccctgtcccagtgtcccacagctgtgctatctctgtgctatgtcccctcatgccatcatgttcccagtgtcctggagcccccatgccgccccattcccacgtcactgcatcctcctgccccagagcccctgaactgttgtgcctgctacatcctgatgtccccatgccatcaaatctatgtcccacagtccccatgccatcatattcccatgacctgccatctccacaccattatggcctccagcccgttggcattctgtccccatctcctgacatctcaattacatcacgtctccacatctcccagccctatcccaccatgtccccatgccccccagtcctatcctatcatgtccccacatcccccagccccatcccatcacgtccccatgctattgagccaccccatcccatacaccactggtccagagagaggcagtgtagagggccacatccttcttctccaggcggtggctctgctgcaggcgctgccggtagtgtgcagccgccccatcctcattggcaaagcttcgcacagtggccatggcctggaaggtctccactgccacctcgctggcccgggcctgcgccttctgcatctgtggtgccagggcctggggacagcagtgtcattgcagggcgggtgggaagggaatgggggctggggaggggacagtgcatgtatggagggaacaaagaacacgtggaggggatggaggggacataaaggggacggtgggcatgtggaggtgacagtgggggcatgcagagaacagaacccatgtggaagggataatggtcacacatagggaataatgagcacatggcacggatggtggacgcatggaaggggcatggggcacgtggagggacagcggtcacacagaggggacaacagcaggaggatggtgggtacatggaggggacagtgggcacatggatagggcaatgagtacatggtggtgacactgggcacatggaggggaccagaggcacatggaggggaccaaaggcacatggaggtgcagacagcagcccatacctgccggaagtgccccacagccctgggcagtgccagcagcagtggcagcgccagcgcggtgagcagcgccatgcgcggggacagccaggccatggtggcgaagaggcagaggccacgtgccagataccacagcaggaggctcagcgcctcacccagcgcctcgcgcacgtcctccgcatcccgcgtcacccgcatggccacatcccctgccgggtgacagcgccgtcagtgccacccctgtcccttatccccgtgtcccctcccagcccggtgcccctcaccggccccgtcggcgcgcagctcggtgatgctctgccgcaggacggcggcgaagacgcggcgctggaggcggctctgcgtgcggctcagtgtccccacgaaggtcacatcacacaccagctcagtgacagcgctgtgacggcagtggggtgtcagggggtcccgcgctgccccctgcccgcaccgcgtcccccctatgccagtacctgctgaggcccagcagcaccatgggcaggatggctgccagctcatcctcgcgggccacccagtcgctggctcgccccatgtagtagggcacggccatctcgcctgtcaccacagcagggtcagggcatgggggacccccccgaggaatggtgccccaggagttctgtgctgcacccccagtttggtgctgcacccccaaagctcagaggtgaacctccgaagctcattgttgccctccagttggctgcagccccccaccccaccgcaaacccatcttcattcccattcaatcaccgcccccaccccaacccgaccccaattctatctccatccccatccctaccccaccccaaccccatttctattcctatcccatttctaccccaccccaatccaaccccagtcccatctccatccacactccatcacattcccattcccaccctgtcttcaatccccattccatctccatcccaaactcagccccagttcccatttctctccccatccccaccccatcctacccagtcccaatcccagttccaaacccacatcattaccattccatcccaaccccattcccagtgcccagcctatacccatccttacccccaccccaatccccatcccattccccatcccattccacggctacttccatccccaatcccattccatccggttcccaatcccatccccatccctacccttatccccagccccaccacaaccccatcctcattcaaatcccaaccccactccgatcccactcccactccccccgccccgtacccagcgccgaagccgccatcagacccatcaccgcggcgcagcgccggcgctccgggctcagcgagaggaggaggcggcgacccgcgcccatcttccccatcgcggccccgatccccctccggcccgatcccaatgcccggcagcggccggagcttctcggaaacgagagcgtctctcattggctgaggcggtgcagcagcgacgctgctcattggtcgagatggtttcgcgtcatcagttgccaggcagatcggaacactgcagtttggagaggggcgggtgattgaaagtgaaagtaacggcggagcgggaaggagatggagagcggcggcggtgaggggctggagggggctggagggggatggactggtaggggctggtggggtctggtggccactggtggggtctggtggttactggtggggactgtcgaggggctggagggatctgatggggactggaggggtttggtgatcgctgttgtgtgctccaggctgggctgtggggagccggactggaagtgggggccgttctaaaagcactgctgtgtgttccaggtgctgaggggagctgaggacctgcaccaggagcaccccggggagcccacctggtccagctgtgcaccagaagctctggggatccccaccccacagccatggcgatgccgccctacattctgcgcctgtcatgcacgctgctcctggccgacctggccctcatgttggccctggcccacttcttcccagcactggcccatttgggctgggtgggctcctggctggaggccgggctgcgcctcctggtgctggggggggccgggcagctgctggcccccaggggaccccgtggggctgcagtgctgctgagcctgggccccgccatcttcctgaccctacggggctatgtaggtctgcctggagctgccccggtgctgctggccatggcaacgccgtcctggctggtgctgacccacgggacagctgtggtggcattgctcacctggagcctcctggtccccactgtggccactggggcaaaggaggcagaggcctgggtgcccctgaggcggctgctggccctcgcctggcccgagtggcccttccttggctgtgccttcctcttcctcgcattggctgcactgggtgagacctcagtgccctactgcaccgggagggctctggatgtcctccgccagggggacggcctcgccgccttcaccgctgctgtcggcctcatgtgcctggcctctgccagcaggtagggaccccacatccctccacaaaaccccatccacctctggtggtcgtctggtgggtttgggggtctctgtccatatctgggggtcatctgatgggttctgggcactccactgaccctttgtgattgtctgaagggttctgggctctccattgacccctgatgggttttggagtcgcccccccaattccttcccagctcgctgtttgccggctgccgcggtggcctcttcaccttcatcaggttccgcttcatcttgcgcacccgcgaccagctcttctccagcctggtgtaccgggacctcgccttcttccagaagaccacagcaggtacagactgggggcacttttgtccctgtccccacaccatacccccagctcaccctactcaactccacagctgagttggcctcccggctgaccaccgatgtgacgctggcaagcaacgtgttggcactcaatatcaacgtcatgctgaggaacctggggcaggtgctggggctctgcgccttcatgctggggctgtccccgcgcctgacaatgctggcactgcttgaagtgccgctcgccgtcaccgcacggaaagtctatgacacccggcaccaggtgatagcagggatgggatggtagggttggggtgacagggatggaggcaatggcaatgggatgggaacagtgggagtggggatagtgaggtggggattgtggggtcagggtggcagggatgagggcagctgcaatgggatgggaacagtgggaatggggagagcaggatggggatcatgggtccaacacagcaaggatgagaggatggagaagagtggagcaggaatggaagtgggatggcgagtacttggccatcccatgggtgctgacacccactgtcccccccagatgctgcagcgggccgtgctggatgcagcagccgacaccggagcggcagtgcaggagtccatctcttccattgagatggtacgggtcttcaatggcgaggaggaggaggagcaccgctacagccaggtgctggacaggaccctacggctgcgggaccagcgggacacagagagggccatttttctcctcatccagcgggtgaggctgacacgaggggacaccctggtgtctgggtgggatcgggacatccccgctgagccccatccccacaggtgctgcagttggccgtgcaggcactggtgctgtactgtgggcaccagcagctccacgaggggaccctcactgccggcggcctcgttgccttcatcctctaccagactaaagctggcagctgcgtgcaggtgaggtcaggcagtgcgtcctctgccaccggatccccatgactgtggccacatccccgtgtccccaccctgggtgctgtgcctgggggtcacatccccatgtccctatcctgggtgctgtgccatgcaggcactggcgtactcctatggtgaccttctgagcaatgcagcggccgcctgcaaggtctttgattacctgaactgggagcgagctgtgggtgctggtggcacctacgtgcccaccagactgcgaggccacgtcaccttccatcgggtgtccttcgcctatcccactcgccctgagcgcctcgtcctgcaagatgtcaccttcgagctgcgccccggtgaggtgacggcgttggcggggctgaatggcagcgggaagagcacctgcgtggcactgctggagagattctatgaacctggggccggggaagtgctgctggacggggtgccgctgcgggactacgagcaccgctacctgcaccgccaggtgagggggtggggggagatgtggctgcactgagcagtgctggggctgagcctctgccctggggcaggtggcactggtggggcaggaacccgtgctcttctctggctccattcgggataacattgcctacgggatggaggactgcgaagaggaggagatcatagcagctgcaagggctgcgggtgctttgggcttcatctctgcactggagcaaggctttggcactggtgagtgctggggagcagggggggacccgggtgtctgaccccactcatccccaccctcatcctgcagacgtaggggagagaggggggcagctgtcagcggggcagaagcagcgcatcgccatcgcccgcgctttggtgcggcatcccaccgtccttatcctcgacgaagccaccagtgctctggatggggacagtgatgcaatggtgagcactgagcagtgggtggggggagggtctggccctgcagtgcatgctgatgggcagctgtgtgtcctacagctacagcagtgggtgaggaacggaggggaccggacggtgctgctcatcacccaccaaccacggatgctggagaaggcagaccgcattgtggtgctggagcatggcacggtggctgagatggggacacccgccgagctgaggacccgcggcggaccctacagccggctgctacagcactgagaaccatggagcagctggagtggcatgcgatgggatatggggagcagtgactgcctctgcttccagctgcaggatgggatgctctgggatttgtgtggaataaagtggagatgctttgtagaggagtgggttgggatgtggggggtgggcagctcatcctcagtgcatgattggttatggaagctgagtgtttgccctcagttgcagcagcactgtaggttatggaggagaggcacagctcagcccgaagtgggacgaagtttccagccatgtctccatatgaaagccatgcagataccaaggagagtgcaagggcaaatgctgggagaagagggagagcagcagtgtgtgatggagtgacaagcaaggagtgtggggtgggcacaggactgcagggggtgggagggacttctgtgttccccaagcatttccctacagcagtcacaccaggtgggctttgagtatctccagaagagacccccacctctggtcagccgttgcagtgttctgtgtgatcaggaatggacaaaatggggctcactgaggtcactgtcagcctcatgtccgggcaagggaaagctggccagaccagaccagtctgtgggagggaaaggaaagaaaaagtggtcttgctagagatgtacctgtggggagaggtgagggggaggaacacggaggctatcccagcacacagggatggcatcacaaaagccagagttcatcctggacagcacttccaggacatgaagctagcaaccatcagcgtgggggcacaggggggaggtgatgtgggatggggggaatggggagggcaaacagcagccggagggaggagggcagggtggtgggcactcagtgcaggtccatgcctgcatccagagaagccgagccaggacacggccccctcacccatcccaacccaaaccacagcacagaacagactgcgggcaggctgagagcagatctgcagggccaccatcagaatggttctgatacacagggcatcagaagcctgtccacaagttggatcctcgtagccagagggtaaggatgtgcagtgctgatggtgacggggcgggcagccctggtacaatgggtagtggtgttggacgggggagaggtgtggcagcagggacccccccacgaggtgctgcgccccggcagccttctgcgggactggggacaacagggagcaccgggggagggggacagagggagagggatagcgggagttatggatggggagggcagggggtggtgggatgtgggatggggacactgcacgttggggacactgagggtggaggtggggacaccggggcagcgacaagggaccacaagagctgtgccgtgggcacatggatgccgagccgggcgcgctgcggtaccgctgctgtacgacacacaacggccactgcatggactgcagtgccactgagtgccaccagggccgggggtgggacacagaactgggaataaagccgcatgtttgtacccgggtgtgttgggatctcagcgcggggagtcccggtgctgtaagaagtgcagggagaatgagggacccccagagccccttccccccagggagaagagcggagcccacaacaacaacaaatcgggggggaaataaaccagaagacggctggagggcagcagaacaaacgtatttatttgggtgtagggtacaatgtggggggagggggcccagcagggacaggagagctgggggaagggcgggatgagggaggtggagtgggcaaggttgtgggtgaggattgcagaggcagctggatgggcggggggggagggcagagaaggaaataggagtgctgtgggacagcaacctgggggggaagtggggtggatatgtgcagtgactgctggggggaggaccaaggagaccacatcccaacaaacagggatgccattgggaaaatgagagttctgccaggacagggagcaagcaaccaccagcatggggtcacagaggagaggtgagaagggatgggggtacacaggggggagtgagcagcaggcagatagaagaggggagggcagtgggtactcagcacagggccacatctgcaccctgagcagccaaactgggacacggtcccctcatccaccccatccccaaccacagcacagtgcagactgcaggcaagctgagcacagaatggcccatcattttatttcacaggaagcagtgcaggcaaagcataacagtcagcataggaagcagaatgagatgtgagaggtcaagaggatggggctgtgcccatcactgaccccaaatgctggtgtggactgttggctccttgcaggctgaagcacagcactcagatggcggggttgctccctgtggggatgagaagggatgatgtcccacggggccaggttcacatcccaaccctctgctccgggggtctccccgcagagccccagcatagcccagccctgggcagtaccacagagagcacacggacccctcccaacccacagccccacaccgcacctgtgctcgagctgctggatccaccttccctgtctataagggaggaacagctgtgagatccagccctgtgctccctatggggcgtggagcagccccaagcaccaggaatgggtaccctgggagaaagtgcagaccccaggaccgccctggcactgggcagaggtgggggacagcgctgccctcatcactcaccgggcgcgatgttgtagcccttccccttcttccctgcagacagagagatgcttcagcacaggttcacatcacagccaggctgagggctcccaagggggacccagatccccccactgcccgcctgcacccctctgcttttacctgcatggcgtctgtagatgatgaatccaacaccaaccatgatggcaatggccacaatggcgacggccacccccgccacgatgggcaccaggttgggctgtggcggctctgggggagagcggggccatcagcaggggaaggggcagcccgcagccccccagccccacatcccctcactcaccccacgagtagaggccgggctggggcaggctggcgtgctccacgcggcactggtacttgtccccgtcccccggctgcgcatcgatggtgacccaggtgtggtaggtgccgtcgccgttgggcacgatgccccccgagtgggcgtcctggccccgcaccgcgccgtccttcagccagctgacaacgatgggccgcgggtagaagccgtgagcgcggcaggacaaggtcaggatcccgtcggcctccttcccccacactcgcacctcgggccgctctgcgggcgggcggcagtgagggccgggctgagctccccacgctgagcccccgccccacgtccagccccacactgcagccgctcccccccccaccccgctcacctctcctgcccagctcagccttcccgtattccacgtatctccgcagccactccacgcaggtttcctccaggtaattcttccacctctcaggttcactctcttcctcccatttcctcttggtgggaactgcctctggaactgccgcagtgaacgtcatcgtgcctttgtcgaaggcagtgaagtctctcccatcgtaggccatctgataatacccccggatggggccgccctcgaggatgtcacagccgtacatccactgcaccgtgtgagaccctgaaacacagccgggcagggggtgaggggcccctccggctccggggctcccactgcagcggggatgggttggggtccccccgggacgcggcaccccgagcgcggtggggctgtgacgggcagccccgggacggtgccgccgggcagggccaggcctggggggtgtgggcggcactgcgccatggagcccatcccacacccacggagccgcggccccggccgtgctcaccgccggtctggttgtagcgccgctgcagtatgcccaggttctcgcggtcaatctgctcattgccctgtccgatctgcgtctgtccatcccagtactgctggtccgccttggccgctatccactcggtgcggggcacgtacctccgcgcggtgctgttgtagtgcacgaagagttccccgtccacgtaccccacagtcacgaaccacggctgcccggggccgggatccgtcatcgccgtttggatgtaccgcagggtatggagctctgcggggacggagcacagcggggccgtgagccgcgggtgtgggtctgaggatcccacggacacagccccggggtggggttacgggcggggaggggtcccggtccggccgcactcaccggccgccgccccgcacacggcggcgagcagcagccccaggcccagcgccccgcacggccccatcgcctcgcaccgctgcactctcaagtccgccgcacccagcgcagctccttcagcttttcctactcctccgccccgccccgcgcccccattggctcctccgtcgcctctccgccaatggtagttggagttttatgtgacgtcatcgggcgccaggcagaatgcgctccctcaggttgtgaagcgaaagcgaaagcgcggagcgggggaggggatgggcgcggtgtgggaacccccggcccttcgagcacgggggggcacccgggctgtgttcgcacggggccgcgtccttaccccgggggagggggccgagggtctctgccgggaggacgggggccgtgagaagaggaggagtcattctccattccagtcaaggaactgtttggggggggggtcacatccatagggttagaggctccgtgtccgggggggagggggtggtgacagtggtgtcccccagggcttcctttgggatcagtgccatttccccacagcgccgccccacaccgcttccccacatccacgtggtccatctgaggtcgatgccctcagggtctgcaggtggaccccaatgtccaccccccaagttaatgattgaccccaaccccgctgtccctgcgccactgctcccatctgccccacactgccggagccatggggcctcactgggccttcagcctcttcctcctcctcttcctcactcccttaatgagggccagctcccaggaccctgagtatggggctatggggtgtttgtggggtagctatgggactatgaatgttctgcagtgcctatgagggactatggggcactggtggggctgggggctgctataggattggggtgtgatggagtctggggggactaagggagatttctgtgtggttgggtggggttatggggccagagctggggggattcctatggacctaaggggtgtctggatgcttatgggatctgggagggcttatggagcagttatggggctggtggctcaagcagtgttccctcaggttggtgctggtggccccccggcgcgtggccttggggacccccatggggctgttgctggcagctgtggggccggtgaccgggacggtgactgcatgggctgagggggaccgtggggctgggccctgcaccctcccagtcccatttgccctcacaccccacaacaacttcaaccagctcctacaaattgaggtatggggacaccgggggatatggggacactgggggatgtcctctggggtgagggggttggggacacccctgtggcacacagggatgtgtgcacccttgggtcccctcctgccatgtcacccatgtcacctcacatctccttccccagagttccccccatgtccccataacccaaacacctcctgctgtgttcccatgtcccttataggtcaccccagtgcaggcagagcgctgtggggcgctgtggggtcgggggttgctcctggaggcccacagctcccatctgcccccccccagtaccaggagtctgagtgtggccctgggggggccgcggggtcacctcattgtgcagacagacaaacctctctacgccccccgacagactggtgagtgctccacgtccaccctaaagccatccctcatctgcccacagttctccccccagtgccccaaatgctccaattcccctaaatcaaccccaaaattctccccaaagcccctccaaatctaccatgaattccccaaatccacccattttcaccctacattcacccatttaccccaaattcacccccagcacaccccaaatacccctggtcacccaaagtcccccaaatccccttcaaattccctaaatccataacccccatctgtccccatgtgtccctttgtccccagtgcgtttccgggtcttctccatggaccccgacctacagccgaaccccgaacctgtcctggtcaccatcacggtatgggccctatagggctggggctgtgggtgaccctgtggggtttgggtgaccctacaaggctgtgtacccccatgtacccccagaacccgttgggtgcacgagtgcgggaggtgcagcgggtgcccctggacacggtgctgagcgaccagctggtgctgcctgacatcgccctgtgagtggggctatagggggctacagagggctgtggggtgcgacagggggctatggggactggggactatggggatttggggctacaggggctgcaggcgggctagagtagtgggggggattatagggttactggggcattacagtggccatagaagctatagagggctgtggagaactataggataccttaggggccataggggtctacaggggttataggtgagcatggggaaacataagggccatagcgactccggagggctgtagcacaccataggggccataagggccctggagggctctagaggaccacagaggtgtatgggaggggctataggggactatagggtatat

SEQ ID NO: 34 - DNA, 716 bp, Gallus sp.
acatgggaacacatgaggacagggagaaactgcagggacacagggacacttgggggatagggggatggtagtgatgcatgggggggggcacatggggatgtgttggggcacactgggatgtgtggggatatggggacacatggggaatatggggatgagtggggacatatggttattatagggatgtatggagacattgggacacatgcagaggaggggacaaatggggacacactgggggacagatagggacatggggacacccagggagggacaccccaagttcccccttaccggcggcagtgatggttccttctgtgcccatccccccctgcagcagcgcagtgacaccgtactgcggggtccccaccgccgccacccaccactgcccccccgcggttggggggctgcgggcgtcggggtgcagagggcggctccatgggtcagagccggtctgggggttcgtggggttcagttcgcagctggggggagtccggggggggaccccgagtggggtcagagtcccccaggggtctgcgagggagagaggagtgagagggatgaaggggtctgagggcatggggttgggaggggtgtggggcgtaatggggtcattttggggttaatggggacactggggacagtttgggagctattggggctaatggggtctctgggggacatggaggggacattggggacatttggggtgtaat

SEQ ID NO: 35 - DNA, 2093 bp, Gallus sp.
tggctgatgggctgtgtcctatgagcgcaaaacaccacaatgggcagaaaaaccttcctccagaggaccaaccccatctctatggcttctttgcacctggccttgcccaaaattgggttatttttgagaaaaaaatgggccatttctctgctggttgtccaagcagcaagagatgctggcatgagtctcaccaagccaagaggtctgtgggaccaagagaactcttttctctcccattaatgatgagtaactccacctttgggcactcttaaggtgaaaatcctcaaaatctgcaattttgaaggcgcagctcccacatttctcatcccctttgttctgtccatggcagtgcaggcattccagccccatccccagccctgtgctcagtgtcccttcgactggattggattcagaggaaaatgctactacttttcagaggatgagagcaattggacgagcagccagaacaactgctctgctcttggtgcttccttggctgtgtttgacagcgctgaggacttggtgagggggacacaaaagagccaccaatgtattttgtccgcttgagggccccttggctgctctttcagtgtttcctttctgattttggggtgaggaggtggataatggttgtcctgagggtaggttgggtctactcctcaaaattcttcaagggatttaagggaaaaaaaagatgttttttctatgaagtaaccacgctggcttagagactgtgagctttggtgatggattgggcagtttcaagcactgagattattggttgaaagggttctgcaggcagtggcatgcaggaaatgtcccagagccccatgatctgttccctctcctcttttccagagcttcacaatgagacacaaaggcagctccccccactgggttggcctctcccgggaaggcaaagagcatccatgggaatgggtgaaccgctctcctttgtctcacctgtgagttcccatccttgtcttggaggctgcagcttctccagccccaaaatgtggatttctggacctcgggagcatttctggaggtggcttatggggtgaggagatgtggggaaggcacttcgcaccgctttgggtcatagaagttcattgagaggcagaagtggcgcaggaaaaagagattcctatttaatcaattattttgtctgtttgatttcaccactgtgatttcctcttccccccccccccaaacctggggtctgcctgtccgtctgtctgtccatccaggttccaggtgcaaggcgatggtctctgtgcatacctgggggatgccgggctcagctcctcccactgcagcacgcggaggaattgggtttgcaccaaacccgcgttgcaaaaaccgaggaagaacttctgcatcagcacctgagcggctcccggacccgaacacgcgatgcaagaggaggaacccaaagcaaaagagctccgctttcagctgtgctcagtagcaacaggagggcggtgcgctcctccagcccaggtccgacagtgccgcctatggggctgcgcggaccgaagcaaatcccaggcggagcttcggctccaaattacatttttttgcaccgtctgactcctaatgaccgctaaaatcccaattttgggggctatccgtgcgctgcttgcaacgaccttcacccctgcgcgatgcagcagcaggtttggggggcggacggtgggaaaatatccatttttcaccggtttttctccaaagggaaatactgggaaagcaatcagcccaaaggaccctgaaatcgatgaataaatcggcaaattatttatgtttcgtgttttcccttctgtgtcactgcagtgcgttctccatgaattcacttttaacggtgttttgtcacaggaaacacttcttcgactctctccaccactcctatatattcaacagaccaattccttctggtgattttatgcaaaaacaaaagagtatatttggttaaagaacccaaaccaccttcttgtactgaagggaatagaagagcacagaccgcccgctcccctcccctgctgccgcacaacagacggtccccgaggatgtgcagacaacgcgacgccgtctga

SEQ ID NO: 36 - DNA, 421 bp, Gallus sp.
  modified_base (1)..(421): “n” represents a, t, c, g, other or unknown
tagnaactagnggatcccccgggctgcagctatgggggagtgggtgcactccttggccatggctttgggtcccgttactctggaggaatttccacagctgccccaggaatcttgtacataaaagtgcacagatcgatcagagatgtcatgttcctgacagaagaaatcctgtctcttctgatgttctctgtgaagagcattgccacgagggagctaccagcagggcaagcagagaaattgaagaaaacgaaagatgggtcgaggtacgggattgggcaggtttcactttctttagcaatgagacgtgtcaagctggcagcttccctgggagcctctctggtgtggatctccggtggccctaaacctggttcaggcactgatcaaggagacattacccgtcttggttcatctcggctcacgg

SEQ ID NO: 37 - DNA, 684 bp, Gallus sp.
cgatggtcctccaatgacctccatggtcatccagtgctcatcccgcggtatggccatggtaaccccatgttcaccctgtggtctcaccccaatgatgccgtggttaccttttcgttaccctattctcatcccatatcccccctttctgtccctctgccccttcatgatcccctcatggttaacagacgtttccctctgcgatcaggtcatgttcagcacaaattcctccagggttccctttatagtgacctcaccattacccaatcatgtccccggtgtccctgaaggggcccagatttcctcagtgggacccagatgtcttcagtggggcgggacctggccattcccaatgtcatccaggtgtccatatggcatgggacacagatgtgcacatgggatgggacccaggtgtccccactgtcatccagatgcctccatgggttgggaaatgaccatcctcgatgtcacccagatgcccacatgtgatgggacgtggccatccttcatggcatcccgatgtccagcttgggatgggatcccaatgtcacccaatgcaatcgcagtgtcacccagatgtccacaagggatggcacccagatgtccccaggtgccactcatctgcctcaccaacccaggacttcctcccactgctcccactgctcccagtttgcccccatttctccc

SEQ ID NO: 38 - DNA, 1920 bp, Gallus sp.
gatcttcagtgattttcagtggtctttggtggtcttcagtgctcttcgttggtctttgacaaagatgcagaggagcaccgctcccagacggaccccccggggaccccatttgtcgccatccccactgggacatgcagccattgaccacagccctccggctgcgaccacccaactgattccttatccaaagtccactctttgcacacttacctccaatttagtgataaggatgtggcgtgggaccgtcccaatggccgcacacaagtccaggtagatgatatgggatgaccatgaagggatcacagagaggaacacggggtgaccacgaggagcaacgaaggaaacgctgagtgaccacgggcagaaaatggtgtgaccattaggggacaacgagagggaacagaagtagtaaggagtgagaatggggtgacaaagaggtgaccatggcataactttgataagaccattgggtgaccgcagggtgatggccataccatggggtgagcactggatgaccatggaggtcattggaggaccatcgggtgggacgagggccgtggggacacccgtggggcggtgggacgggggcagagtgtcagaaggagccccgcggcgcagaactctgcctggagacgggtgacgccgcccggcgccgccgccgctcattggccctccccgcccggccccgggctcgcggctggcgcggggtgccgggtcccccatcgtccggcggcagcagccatggggagcgggcgcgtcccggcggcgggggccgtgctggtggcactgctggcgctgggagcccggccggccgccggcacgcggccctcgggtgagctcggagccgcggcgcggggacggcgctgcgtcccccccggagaaacccccggagcccttctggccgtgcgcagcgctcggggctgcggggggacggagggcgggggggggcggcggagccgtggggggcagcggggccggggagggggcggggggtgtggcggggggcggctgtgtgccctgaccgtgccctctgcccgcagcgttcttcttctgcggtgcgatatccgagtgccactacctgaacggcaccgagcgggtgaggtatctgcaaaggtacatctacaaccggcagcagttcacgcacttcgacagcgacgtggggaaatttgtggccgattcaccgctgggtgagccgcaagctgaatactggaacagcaacgccgagcttctggagaaccgaatgaatgaagtggacaggttctgccggcacaactacgggggtgtggagtccttcacggtgcagaggagcggtgagtgccgcggggcgcagcgcggacggacgggcaggcgccgcgctctggcggtcggtccgcagcgctccccccgtgccccgcagtggagcccaaggtgagggtctcggcgctgcagtcgggctccctgcccgaaaccgaccgtctggcgtgctacgtgacgggcttctacccgccggagatcgaggtgaagtggttcctgaacgggcgggaggagacggagcgcgtggtgtccacggacgtgatgcagaacggggactggacgtaccaggtgctggtggtgctggagaccgtcccgcggcgcggggacagctacgtgtgccgggtggagcacgccagcctgcggcagcccatcagccaggcgtggggtaaggcccccgggccctgccccgccgcggggggagcgggagcgcggcccccggcgctgagccgccgccttcgtccccgcagagccgccggcggacgcgggcaggagcaagctgctgacgggcgtggggggcttcgtgctggggctcgtcttcctggcgctggggctcttcgtgttcctgcgcggtcagaaaggtgagcgctggggaggggggctgcgccggggggggtcgggagcgggggg

SEQ ID NO: 39 - DNA, 628 bp, Gallus sp.
gctctagaactagtggatcccccgggctgcaggattaccagtgtccccaactgtttttgccaatccaagccctgcaaatgtacaaatatattaagtggtttccttagtagacatctttatatctctcaccaatcatttaacgttaaccttactctgctttcttctgtgaacagaaaacaaaatcggaagcctcatatacaggtgttcagaggaaaatagtaagtggtgatgaaacttggagaacttgtgaagtgaaatatgggagctactgcctctggagggaggaaaataaggaaccaatgaaagatgccaaggtgaagcaaatgaaggaccagctgtttgtggctagagcatactatcccagtattgctaaaatgccttctcaaagcaagttgactcgggatatgaaacagaatatccaagagtttgagcgtattcttagtgaaagttctcaagatgctgaccttccaccacagtaagttctctccagtttgggtttaatcatttttgtactgaaagtttagttccttactggaaaagatttttgttggatttctagtcacatgaatctctcctagtttgccttcagtttgccggacatcccgttttctagtggttttacttgctt

SEQ ID NO: 40 - DNA, 372 bp, Gallus sp.
taaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggc

SEQ ID NO: 41 - DNA, 279 bp, Gallus sp.
tccctagtaacggccgccagtgtgctggaattcggcttagcgtggtcgcggccgaggtacataccctgcccgcagtgatgtctccaaggttgatttaagcaaccagctcctccctgccacggctccaggctccacatgcctgggttaaggctgggtttgttttttgagacagtgtcttaactatggagcgctgactgttctggaactcgctctgtagaccagtctggccttgaactcagtgatccccctgactctgtctccagaatgtggattctccca

SEQ ID NO: 42 - DNA, 6706 bp, Gallus sp.
ggattctgacacccctcctcccccacccccaaaggtgttccagcgccgcatggatgggggcaccgacttctggagggggtgggaggagtacgtccatggcttcgggaacgtttctggggagttctggctgggtgaggaccccaaaacttgggaagattgaggtctggggtggggggggggaacacccagggcggagagggctgatggctgcaggacgtggagtgggatccctgacgggggtgtggggtggggggtgtggggcaggggccccaggtgggtgtgtagggtggggatgatgacgatggctgtgggatgtggcgcagggaatgcggcgctgcacacactgacagcttccgggcccacggagctgcgtgtggacctctggacgccgtcagacagcgccttcgcccgctatcgggatttcgccgtcagtggtcctgaggacaatttccgccttcacctcggggcctacagtggcacagctggtgtgtgtggggcagtgggagctcctgggggatattagggttaaccttgacccatgaggggggcttttggggatacccagatcagggggggggggaatcctggggagagtaggggatggtccctttgcccacagtgagggggccttgccttgcagaggtctttaagatcgttgacctgttgggatctcttggggatctccagactgcagggagccccgggggttcttggggggctctgccccacagggtggtctctgtgagggtgtgggggtacctggggggtctgcggctcatccttggggctctgaatgctatgtgggtgtcctggaaggctctctttaggggtccccataaccttgctgtgggtcccacaggggatgcactgtcctaccatgctgggagccccttctccacgcgggaccacgacccccgaggccgccctcggccctgcgccgtcgcctacaccggagcctggtggtaccgcaactgccactacgccaacctcaatgggcgctatggggtgccctacgaccaccaggcatggctatgggggttgtaaaggggtctgtggggattgtaaaggggtctatgggggtataaaatcaacccaatgggacaggagggggtcaccatgaggccatgggggttttgtggggtaaatgtggagggctaccccccccccaaggtccttttagccccatgtctctcctgtatgaatatggagccctacaggagctgtggaagctggaacacaagctggaacagggaggggatactttgggcccccctgtaaggcctatatgtgtctatagggtcactgtaggttgtttaagggcatgaccaagtcccccttcctttctgcagggcatcaactggtacccctggaagggctttgagtactccatccccttcacagagatgaagctgcgaccgcagcgtgactgagagcactagaaaggtcgtgggtcgcagtggagcctttatggggtcaataaagctgcgagtagccagtgctgacccatgtatcccacacactgggctcaggagctatgggggtgggcagggcgtgaggcgcacgcggaacggggcacagcgcagcacggtgccagcagtgacccctaagtggggcagagccccatcagacggtggctccaggcggaatcgctgtaggatgtgccccaaaaacacaaagagctctgcccgagccagcgcctcccccacacacgagcgtgccccacaaccaaagggcagcagcgctcgccatggagcccccggctgcaggaaccgctctgtggggcagaacagagatcagagtgggtgtagggggaggaacccagcctggggttcaaagcccacatctatggggtggacccacacataccgggcaggaactcatcaggacggtcccaaatcttggggtcgtggtgcgcagcaaagaggtttgggataacgatggatcccgcaggcactgggattcccgcaatgctgggaagggacagaatgctgataggatggactgggagagcctacagaggccaagtgggacatactgggacctgctgagctatcctagagcttactgggtgcttgatgagttctactgggaccgacctactggtaggtccatgctggtctgtagtggtccacactattacagactggtctacaatggttcattctagtgcaaaatactgacacgcagtggtgcacggttctgctcgcagacatgtggcccgcactggttggtactgatccccactggtctgtatggccccataccagcccgtactggtgttactggctgtacctggagtggcgccgggcacagtggggcagcgcgaggggcacggggggtcgcaggcggagggtctcggtgacagtggcacggagcagtggcagtcgccccatatcccctggctttggggtccccctggggcccagcacctggcgcagctctgcacgtacctggtcctgcacctgggacaggggacacgtgtcaaagcacgtcaccaagtgccacatcgggtcacttgtggggtggccctcccctgcacggggacacaggcagcagcgtgacacggaagtgacatgagcgtgacattttggcactggccacagtgcaggggacaccaggggcattatgcacacagggttatggacatggatgtgacatgcatatggggaagtgcagtggagctatgggaggggacagccaggacatgggtggggaggcccgaatgggacctggggagaagcaggtgtgggtgtgacacagatgtgatgtggtgtcacctgggggtggtgcagcaggaaggccacagcccatagcagagccactgccgtcgtttcggtgccaccgatgaagagatccacgagggccatgtgcaggcggtccccccccagcggccccatagggacagtggggtccccccccagcagtgctcccagcactgtgtccctggggggagacgcacagccctgtggggacacacgtgttaccccctggggccctgtcccccccctgtacctgtgtccccacgttccccacctggtgccatcggatctgggactccacaaaggcatcgcggcgctccaccaggcgcagcagctcccgcagccctgcgttgggcagcacctgtggggcacagggaccccccccagtgctccacagagcacccctggacccatagggaccccatattccctcccagccccatatataacccccccccagggcgatatagcccatccttagtatagacccctgcagccccatatggacctataccacctcctcttatgactatatcccgcagccccacgccgatcctatatgccctgtagggccctgtagggctcacccttagtgaaggcagcacatccagtgcccgcacactggcccggccccacacctccagcagttccaccacacagcgcgtgaaggagcgcacctccgcctcggggggcatctgtggggcacagggcttggggtcaccccagagagactcctgagtccccccagagactcctgaacccaaagaggtaccgtggtcatttggatccctctagaggtgactgggttcccaaagggacacctcaacacttgtgtccccttcaggggcacctggatatctgggactccaagtggcacctgagcatttgggacccaccctccttggacacctgggtcaccccaaggacacctgggaccccttcaagtggcacgtggacatctgagccccctgtagtggcacttgagtccccctgctcccccaggtgacacccagaccctgcagcccctcgatatccccaccaggtccccgaaggcaaggcggcagatggtgctgcaggtgtggaacgtgaacgcctcaaagaggtccactggggcagccccataagagctcaactcctgtggggtgagaaatggggtcactgagcgggtgcgggtgccccacaaggggggttggggtgagtcaaggggacgggcagcacagccctggggctgatggggtccacctggggttggagggccctgtgttggggtgctcacctggcacagcgcccagccctgcagctccaggaggggctccaggtgcctcacagctcgcgccagtgctccccgcgttgccccccgctgccgtcgccactctggggatgcatcccccagcgccaggtcctgccccccccgcgacaccagggacgctgtggggtgacacccatatcaccctggcacccatgtgacctccgagaacccctcagacagctgtacggatccttggggacacatccagaatcccccaggcacccactgggatcgctccagcacccatggggactgttagagatctcctcccccccaaaaaatacaaccagaccccttcagagatcatggggacccccccagtaccccctccagatacccaacagtgacctatagagacctccctccacccaaaagccatggggacccctcaggccccccccccagacaccaattagtaccccccagaacccttcagaaacctacaaggacccaccagaaccccctcagatacccatagagatctttacagacctcctcctgggaccctccccaggagcacaaatcccaaagaaccccccttgaagattcacagggacccccctctgactcaccccaaaccctcatggggacctcccaacccctacagcccccccatacccaggtaactgtgggggcgtcccacgaagtccccccagcgccgtgccagtgcctcacggatggctgctgcagagctcagcaccaccacctctgggggggtggggggggaggggcaaaaaatgagtgagttggaaggaagggaccccatggggaccccaaaaaccagggagaggggagaggtgaggggtgccagaacggagttgggggggggggggggagggatcccaaattattttttggggggggggagtagaatgagaggacaaatttgaaggggagcagaagggaattgggggacagtatgtgggggttcctccatcctctcaatgggtaattctggggagcctgtgaagttgagggtcctaaagggggaaggctcaaggtcccaaggagggaagggttatggggaaaagggggtaatggtggtcccaaggggtatcagggggatgggggggggggggggtcatgaaggtgccgcccctactcaccacacccccccaagcgcaggcataaggggtccccgtaggtccgggcaaggatgtggaggtgccgtggcccccctgggtgcaggaggtgcagggcccccccccacggtgctcccccccgcctggctgaccccccccagatcagagccaggagaagtagcagcagaagtatcgtcaccgccattgttctgtggggtggggggccccagctctgcccctataacaccttatgaggaggaggtaccccaaaagctccacccccccacatccaaacccctcctaccagaagaggggcattgggttcactcccctaaaattattgtgtgcccacccccctcttcaagtcattataaactttacaggggtgtcctcataaaaatacaggagtgtgtccccccacaaagtgctcccagaaccatcgggtgcccatcccacaagaaaatctctcaaactcccattgtgtgccccccaaccaacaaagattcctcaaactcccccccccgccctctacccatatatcctcccaagcgctccccacccctccgcacaccacctccccaaatcctcccccattaccataatcccccccaccccagcagcagaaccccatcaccgctctgtgcgtctgtgtgtgtgagtaggggacggggtgtttattgaggggaggggggaggggggagagcgctcagaacccctccccctgcagcccccgcaggcgccgtgccagctgcaggtctttggggtacagtgtgacgcggcgcgcatgcagcgagcacaggtaggcgtcctccagcaggtgcaccaggaatgcctccgccgcctgtgggaccccggcgtgggcgtccccacaaagcagggggggagtcaattcccacccccaggccaccccacaaatgccaatcctccaaaataatccctggaacaaccccaaaaaaacccctacccccaacccccctccccaaaaccataacctcaataactccacacctcaaaaacctccaacccctccaaaacaacccccaaccccgaaacacctcacccccaaagaccccttcccaagccccaaagagacccccaggcacaaggggtaccccaaaatccacttcccccttcccccaaaaaagcccttttgggcactagagagctccccagcaccacccaaagggtcccccacggtatggggtaccctaaaacaccccccaaccccaaaccacgggaacttccaaaacaaagctacccccctccccccccccccaaaaaaataaacccatagggccccccacctcctgtagggccaatagggccatagcctgccacctgtagtccacgccccgtgtgaagagcaagcagatctcccgcacctggggggggacaggggggcatggggacactggggggacatgggggggggggggggaggggggggggggggaggggcatgaggacattgaggagagggaacacgagggtggcactgcatcatgggaggtgacgagggggtggggggggctcaaggacatggagggggacactca

SEQ ID NO: 43 - DNA, 254 bp, Gallus sp.
ttgctgcctgcaggtcgatctagtggatccgccgcgacagcgaacaggccagccagctggtgcagtatctttccacttttttccgcaaaaacttaaagcggccttcggagtttgttactctcgccgacgaaattgaacatgtgaatgcttatctgcaaattgaaaaggcgcgcttccagtcgcggttgcaggtcaacattgctattccgcaagaattatcccagcagcaattgcccgcgtttaccctgcaaccc

SEQ ID NO: 44 - DNA, 4846 bp, Gallus sp.
tgctggtggcggggatctgactggaaatggaaacgttctgtggcaaagagtgggaatgtaggaagggggtgggagcatgcagggttggtggagcagggggtagtgatcagtggtgaggatttggtttcttggtctgaaatatggatggaagctttgttgggagagtgaatgacttttcagtgaggacaggtggatgcttgggtgaatgcttggtaagttgttgaacgcctggatagttggatgggtggacatgaactttgtattacagctgcagctccagcacagaaggaaccgccatcccaaccacgcctgggtgagctgacggcctcccacgtcagccccgactccgtccagctggaatggagcgtccccgagggctcctttgactccttcacggtgcagtacaaggatgcacaaggccagccacaggtggtgcccgtggacggtgggttgcgcacagtgaccgtgcccgggctgtcgccgtcccgccgctacaagttcaacctgtatggggtgtgggggcggaagcgtctgggccccatgtccactgatgctgtcacaggtgagcatgctgttgtgctgcatccatgtcttttggctgacggttgtgttggcatatggtaggaacctttcaggcccactcctggttactgtggtcttaatagagagggaagttctttcctgttcttgacgtgggtagcctggagagatgggagtatggaagatgagaggaagaacggaataaggaatgattgataattattgcagaacggatggaagggaggatggatgggcggtgcatgggtacattggtgcttatagcagagctggacggctggttgtacgttggtttggttgttgaagagatgaagagttggatgggcgtgtgctttcactgtgaattcctccccctgtcttgcagctccggcacagaaggaaccaccttcccagccactcttgggtgagctgacagcgtcccacgtcggccccgactccgtccagctggaatggagcgtccccgagggctcctttgactccttcacggtgcagtacaaggatgcacaaggccagccacaggtggtgcccgtggacggtgggttgcgcacagtgaccgtgcccgggctgtcgccgtcccgccgctacaagttcaacctgtatggggtgtgggggcggaagcgtctgggccccatgtccactgatgctgtcacaggtgagcatgctgtgttctgcctccatgttcttttgctttcagtgtagttgtcatgtggcaggaacctttcagggccacttttggttaatgttgccttaatagtcaaggaaacaatttgttcttgttgagtgggaatgcctaacgggatgggagtttggatgatgagaggacaaatcttataagggatgattgataattattgcggaacggatggaaggaaggttggatggatggaatggtgtttggataaatttgtgctcagagcacagctggagtgttggatgaatgttgctttgcttgttgaatagatggatgtttggttgtgtggttgcttccactgagaattcctccctctgtgctgcagcagcagctccagcacaagaggagccaccttccccaccacgtctgggtgagctgacagcgtcccatgtcggccccgactccgtccagctggaatggagcgtccccgagggctcctttgactccttcacggtgcagtacaaggatgcacaaggccagccacaggtggtgcccgtggacggtgggttgcgcacagtgaccgtgcccgggctgtcgccgtcccgccgctacaagttcaacctgtatggggtgtgggggcggaagcgtctgggccccatgtccactgatgctgtcacaggtgagggcaggaattggcacctgttgggctctgggtttgcagcaggtagaaatgtaaacgtggcctgcgctggggatcttgttttcccctggcaatgggaacagctgttgggtgccttttttgggaaggatcccttaatcgcagcatgaagtatgaatggaccaattgggtgtgggtggagtgatggctgttgagatgagttggtggctgcttgagtaattgtctgttggaatggatggacagatatgtgaaggagtgaaaggatggataaagtaatttaggaatcggtggatgaagaatgggtaggtagacccttggtgaagtggtagaatggaaggatttatgaacagatatgagttaattcttgcatcgaagtaggtgtaagtgtctattagcctgttgcactgaacatgcagttgcatagacaaatgagtggggagaagtacggagtaaatccctgcatgaatggtaggacagaaacctgaatgcctggatgctggcagtgtgaagaatggcacttgggatagatggttcgagtatggggtagattaaaagatggatggaaaagaggaacagagagagggtgattggatgaatggatggatggttggatgtgactgattgacaggtaccaagcttttttcctgcactgtgccttctgtgctgcagctgcagaagagacggaggaggaaccaccgtcccagccacgcctaggagagctgacggcatcccatgtcagccccaactccgtccagctggaatggagcatccctgagggctcctttgactccttcacggtgcagtacatagacgtgcaaggccagccgcaggagctgcacttggatagtgggtcgcgcacagtgaccgtgtctggtttgctgccatcccacccctacaagttcaacctttacggggtgtgggggcagacacgtctgggccccatctccactgacaccatcacaggtgagggcccctgcctgctgctgtgctctgggccttgtgcttggcacgtggcaggagctgtgcgatgggctgtgctggtggcggggatctgactggaaatggaaacgttctgtggcaaagagtgggaatgtaggaagggggtgggagcatgcagggttggtggagcagggggtagtgatcagtggtgaggatttggtttcttggtctgaaatatggatggaagctttgttgggagagtgaatgacttttcagtgaggacaggtggatgcttgggtgaatgcttggtaagttgttgaacgcctggatagttggatgggtggacatgaactttgtattacagctgcagctccagcacagaaggaaccgccatcccaaccacgcctgggtgagctgacggcctcccacgtcagccccgactccgtccagctggaatggagcgtccccgagggctcctttgactccttcacggtgcagtacaaggatgcacaaggccagccacaggtggtgcccgtggacggtgggttgcgcacagtgaccgtgcccgggctgtcgccgtcccgccgctacaagttcaacctgtatggggtgtgggggcggaagcgtctgggccccatgtccactgatgctgtcacaggtgagcatgctgttgtgctgcatccatgtcttttggctgacggttgtgttggcatatggtaggaacctttcaggcccactcctggttactgtggtcttaatagagagggaagttctttcctgttcttgacgtgggtagcctggagagatgggagtatggaagatgagaggaagaacggaataaggaatgattgataattattgcagaacggatggaagggaggatggatgggcggtgcatgggtacattggtgcttatagcagagctggacggctggttgtacgttggtttggttgttgaagagatgaagagttggatgggcgtgtgctttcactgtgaattcctccccctgtcttgcagctccggcacagaaggaaccaccttcccagccactcttgggtgagctgacagcgtcccacgtcggccccgactccgtccagctggaatggagcgtccccgagggctcctttgactccttcacggtgcagtacaaggatgcacaaggccagccacaggtggtgcccgtggacggtgggttgcgcacagtgaccgtgcccgggctgtcgccgtcccgccgctacaagttcaacctgtatggggtgtgggggcggaagcgtctgggccccatgtccactgatgctgtcacaggtgagggcaggaattggcacctggtgggctctgggtttgcagcaggtagaaatgtaaacgtggcctgcgctggggatcttgttttcccctggcaatgggaacagctgttgggtgccttttttgggaaggatcccttaatcgcagcatgaagtatgaatggaccaattgggtgtgggtggagtgatggctgttgagatgagttggtggctgcttgagtaattgtctgttggaatggatggacagatatgtgaaggagtgaaaggatggataaagtaatttaggaatcggtggatgaagaatgggtaggtagacccttggtgaagtggtagaatggaaggatttatgaacagatatgagttaattcttgcatcgaagtaggtgtaagtgtctattagcctgttgcactgaacatgcagttgcatagacaaatgagtggggagaagtacggagtaaatccctgcatgaatggtaggacagaaacctgaatgcctggatgctggcagtgtgaagaatggcacttgggatagatggttcgagtatggggtagattaaaagatggatggaaaagaggaacagagagagggtgattggatgaatggatggatggttggatgtgactgattgacaggtaccaagcttttttcctgcactgtgccttctgtgctgcaggactatggtcatagctgtttcctgtgtgaaattgttatccgctcacaattccacacaacatcga

SEQ ID NO: 45 - protein, 546 aa, Gallus sp.
  MOD_RES (530): “Xaa” represents any, other or unknown amino acid
GEMAVPYYMGRASDWVAREDKLAAILPMVLLGLSSAVTELVCDVTFVGTLSRTQSRLQRRVFAAVLRQSITELRADGAGDVAMRVTRDAEDVREALGKALSLLLWYLARGLCLFATMAWLSPRMALLTALALPLLLALPRAVGHFRQALAPQMQKAQARASEVAVETFQAMATVRSFANEDGAAAHYRQRLQQSHRLEKKDVALYTASLWTSGFSALALKMGILYYGGQLVAAGTVSTGDLVTFLLYQIQFTDVLEVLLDYFPTLMKAVGSSEKIFEFLDREPQVSPSGTMAPADLQGHLQLEDVWFSYPGRQETRPQGVSLELRPGEVLALLGPPGAGKSTLVALVSRLHQPTAGRLLLDGHPLPAYQHSYLCRQVAVVPQEPLLFARSLHANISYGLGGCSRAQVTAAARRVGAHDFITRLPQGYDTEVGELGGQLSGGQRQAVAIARALLRDPRILILDEHTSALDNESQQQVEQEILAAKGSGRAVLMVTGRAALAARAQRVVVLEGGEVRQEGPPHEVVRPVSLXAGLGTTREHRGRGTEG

SEQ ID NO: 46 - protein, 600 aa, Gallus sp.
MATPSWLVLTHGTAVVALLTWSLLVPTVATGAKEAKAWVPLRRLLALAWPEWPFLGCAFLFLALAALGETSLPYCTGRAVDVLRQGDGLAAFTAAVGLMCLASASSSLFAGCRGGLRFVLRTRDQLFSSLVYRDLAFFQNTTAAELASRLTTDVTLASNVLALNINVMLRNLGQVLGLCAFMLGLSPRLTMLALLEVPLAVTARKVYDTRHQMLQRAVLDAAADTGAAVQESISSIEMVRVFNGEEEEEHRYSQVLDRTLRLRDQRDTERAIFLLIQRVLQLAVQALVLYCGHQQLREGTLTAGSLVAFILYQTKAGSCVQALAYSYGDLLSNAVAACKVFDYLDWERPVGAGGTYVPTRLRGHITFHRVSFAYPTRPERLVLQDVTFELRPSEVTALAGLNGSGKSTCVALLERFYEPGAGEVLLDGVPLRDYEHRYLHRQVALVGQEPVLFSGSIRDNIAYGMEDCEEEEIIAAARAAGALGFISALEQGFGTDVGERGGQLSAGQKQRIAIARALVRRPTILILDEATSALDGDSDAMLQQWVRNGGDRTVLFITHQPRMLEKADRIVVLEHGTVAEMGTPAELRTRGGPYSRLLQH

SEQ ID NO: 47 - DNA, 21 bp, Artificial Sequence
  Description of Artificial Sequence: Primer
ggccccgggatgccgcggttc

SEQ ID NO: 48 - DNA, 18 bp, Artificial Sequence
  Description of Artificial Sequence: Primer
atccgctcaccgccctgg

SEQ ID NO: 49 - DNA, 20 bp, Artificial Sequence
  Description of Artificial Sequence: Primer
ccagcagtcactgcacatat

SEQ ID NO: 50 - DNA, 19 bp, Artificial Sequence
  Description of Artificial Sequence: Primer
aggtggagtgcgcaaagtt

SEQ ID NO: 51 - DNA, 21 bp, Artificial Sequence
  Description of Artificial Sequence: Primer
agacgcagcagaacttggtaa

SEQ ID NO: 52 - DNA, 18 bp, Artificial Sequence
  Description of Artificial Sequence: Primer
ggaaggaagaccttggaa

SEQ ID NO: 53 - DNA, 21 bp, Artificial Sequence
  Description of Artificial Sequence: Primer
caggatctgcactggccaata

SEQ ID NO: 54 - DNA, 24 bp, Artificial Sequence
  Description of Artificial Sequence: Primer
gaatggcggtgcttccgtgcctgg